(19) United States
(12) Patent Application Publication
(10) Pub. No.: US 2014/0275213 A1
(43) Pub. Date: Sep. 18, 2014

(54) Methods and Compositions for Plant Pest Control

(71) Applicants: Pioneer Hi-Bred International, Inc., Johnston, IA (US); Iowa State University Research Foundation, Inc., Ames, IA (US); The Curators of the University of Missouri, Columbia, MO (US); University of Georgia Research Foundation, Inc., Athens, GA (US); North Carolina State University, Raleigh, NC (US)

(72) Inventors: Jun-zhi Wei, Johnston, IA (US); Gusui Wu, Johnston, IA (US); Carl R. Simmons, Johnston, IA (US); Thomas Baum, Ames, IA (US); Eric Davis, Raleigh, NC (US); Richard Hussey, Athens, GA (US); Melissa G. Mitchum, Columbia, MO (US)

(73) Assignees: Pioneer Hi-Bred International, Inc., Johnston, IA (US); Iowa State University Research Foundation, Inc., Ames, IA (US); The Curators of the University of Missouri, Columbia, MO (US); University of Georgia Research Foundation, Inc., Athens, GA (US); North Carolina State University, Raleigh, NC (US)

(21) Appl. No.: 14/207,026

(22) Filed: Mar. 12, 2014

Related U.S. Application Data
(60) Provisional application No. 61/780,395, filed on Mar. 13, 2013.

(51) Int. Cl. C12N 15/82; C12N 15/113; C07K 14/435

(52) U.S. Cl. CPC C12N 15/8285; C07K 14/4354; C12N 15/113
USPC 514/44 A; 435/320.1; 530/350; 435/418; 435/414; 435/416; 435/415; 435/412; 800/301; 536/24.5; 536/23.5

(57) ABSTRACT

The present invention comprises methods and compositions for controlling nematode parasitism in host plant. The present invention comprises novel polynucleotides and polypeptides encoded by such polynucleotides comprising one or more nucleic acid sequences disclosed herein having a nucleotide sequence comprising any one of SEQ ID NOs: 1-142, a fragment or variant thereof, or a complement thereof, or a polypeptide sequence comprising any one of SEQ ID NOs: 143-159, a fragment or variant thereof.

### CROSS-REFERENCE TO RELATED APPLICATION

This patent application is a nonprovisional patent application claiming priority to and the benefit of the filing date of U.S. Provisional Patent Application Ser. No. 61/780,395, filed Mar. 13, 2013, which is herein incorporated in its entirety.

### REFERENCE TO SEQUENCE LISTING

The Sequence Listing submitted Mar. 12, 2014 as a text file named “36446_{—}0005U3_{—}2014_{—}03_{—}12 Sequences as Filed,” created on Mar. 11, 2014, and having a size of 183,117 bytes is hereby incorporated by reference pursuant to 37 C.F.R. §1.52(e)(5).

### TECHNICAL AREA

The present invention comprises methods and compositions for identifying and isolating genes involved in plant parasitism by nematodes, and use of such identified nucleic acid sequences for inhibiting nematode parasites, particularly in soy bean plants.

### BACKGROUND

Plant-parasitic nematodes (PPNs) are major pathogens that significantly affect the yield and quality of many plant products. It is estimated that annual economic loss due to PPN infection is about $125 billion worldwide. The most devastating nematodes in agriculture are the sedentary endoparasites, which include the genera Heterodera and Globedera (cyst nematodes) and Meloidogyne (root-knot nematodes). Soybean cyst nematode (SCN), Heterodera glycines, is an effective pathogen in soybean plants, and invades the roots of the plants. It is estimated that yearly SCN causes over two billion dollars in soybean losses in the world. Currently, resistance from plant germplasm is the major tool for SCN control, but multiple genes are involved for the resistance and the resistance is race-dependent. With the continuous use of narrow germplasm, a race shift may occur in the nematode population in the field from year to year, with the result that the number of resistant populations of nematodes is growing. Other controls of nematode pests include biocontrol and seed treatment, but these controls are not routinely effective. What is needed are methods and compositions for nematode control that comprise race-independent resistance by the plants.

PPNs enter host plants through the roots and form complex feeding structures inside the roots, such as syncytia, seen in cyst nematodes, and giant cells, seen in root knot nematodes. The formation of the feeding structures is accompanied by significant alterations in local gene expression and cell dedifferentiation in the plant, which converts the feeding structure into the major nutrient source for nematode growth and development. Studies indicate that effector proteins injected from nematodes into the targeted plant cells play important roles in the establishment of feeding structures. What is needed are methods and compositions for identifying major nematode effector peptides and genes, for example, that provide for parasitism activities. What is also needed are methods and compositions for nematode control.

### SUMMARY

The present invention comprises methods and compositions for isolating and identifying nucleic acid sequences of plant-parasitic pests, such as nematodes, and using such sequences to control, for example, by interrupting and/or inhibiting, parasitism by the pest. Methods and compositions of the present invention may be used to control nematode plant-parasitic disease, particularly for example, soybean plant disease due to parasitism by nematodes, for example, Heterodera sp., such as H. glycines, Globedera sp. (cyst nematodes) and Meloidogyne sp. Methods of the present invention comprise using nucleic acid sequences identified from cDNA libraries of nucleic acids extracted from soybean cyst nematode (SCN) esophageal gland cells, such as H. glycines. Such identified nucleic acid sequences may encode SCN effector proteins, other peptides or control elements, and such identified nucleic acid sequences may be used to modulate infection of plants by nematodes. For example, the sequences may be used as a double-stranded RNA (dsRNA) sequence to control nematodes, may be used for RNAi purposes in plant cells, and/or may be used to transform cells, plants and/or seeds. The identified sequences may encode polypeptides to which antibodies may be made. The present invention comprises novel nucleic acid sequences isolated from H. glycines, and compositions comprising novel nucleic acid sequences isolated from H. glycines. Such sequences may encode peptides or proteins, such as effector proteins, proteins involved in parasitism of soybean plants, or other proteins of H. glycines. Nucleic acids of the present invention may include, but are not limited to, DNA, RNA, single-stranded, double-stranded nucleic acids, and/or may comprise natural or synthetic nucleotides.

### DESCRIPTION OF FIGURES

FIG. 1 shows exemplary nucleic acid sequences of the present invention, polypeptide sequences, an indication of the presence of a signal sequence, its homology and subcellular location.

FIG. 2 A-F show transgenic Arabidopsis plants expressing nematode parasitism genes showing morphological irregularities including longer roots (A), large, twisted leaves (B), elongated growth of 1° inflorescence meristem (C), stunted growth (D), smaller rosettes (E), and more rosette leaves (F) than WT.

FIG. 3 A-B are graphs showing treatments of J2 soaked in H2O, small dsRNA plus feeding stimulant (sRNA+Res), small dsRNA without feeding stimulant (sRNA), full-length dsRNA plus feeding stimulant (fRNA+Res), full-length dsRNA without feeding stimulant (fRNA), and feeding stimulant alone (Res).

FIGS. 4 A and B are micrograph (A) and chart (B) where A above demonstrates expression (RT-PCR) of the PDK intron of hairpin RNAi constructs against a nematode GOI in several transgenic plant lines (L6-4, L2-6, and L1-5) only in the presence of reverse-transcriptase (+RT). Panel B shows number of adult female cyst nematodes that developed on the same plant host-derived RNAi lines against a nematode GOI as compared to host-derived RNAi of GFP (a non-nematode gene) as a negative control.

### DETAILED DESCRIPTION

The present invention comprises methods and compositions comprising nucleic acids isolated from nematode esophageal gland cells, particularly H. glycines, for control of parasitic infestations of soybeans by nematodes. The identification of nucleic acid sequences, such as genes, that are involved in the parasitic activities or life stage of a nematode may be used as targets for genetic control of nematode infection to inhibit the transcription, post-transcription steps, translation, expression or utilization of such genes by the nematode or the plant host. For example, dsRNA nucleic acid sequences encoded by nucleic acid sequences of the present invention may be used to inhibit nematode growth and development, pathways, peptides or molecules involved in parasitism, or plant host responses to nematode infection. Methods and compositions of the present inventions comprise plants or cells comprising one or more nucleic acid sequences of the present invention, disclosed herein, comprising a nucleotide sequence comprising any one of SEQ ID NOs: 1-142, a fragment or variant thereof, or a complement thereof the nucleotide sequence comprising any one of SEQ ID NOs: 1-142, a fragment or variant thereof, or a complement thereof;

Methods and compositions of the present invention comprise nucleic acid constructs, comprising DNA, RNA or both, in single or double stranded form, comprising one or more nucleic acid sequences disclosed herein having a nucleotide sequence comprising any one of SEQ ID NOs: 1-142, a fragment or variant thereof, or a complement thereof. The present invention comprises transgenic plants or cells, transgenic plant material, and nucleic acid constructs that modulate, for example, inhibit, the synthesis and activity of proteins, for example, parasitism proteins secreted by cyst nematodes, such as Heterodera glycines (SCN). Modulation of cyst nematode proteins may modulate gene expression of the host plant or host plant cell, modulate formation of a syncytium in the host plant, modulate nematode migration through root tissue of the host plant, modulate cell metabolism of the host plant, modulate signal transduction in the host plant cell, or modulate formation of a nematode feeding tube. For example a nucleic acid of the present invention may be a double or single stranded RNA that modulates, such as inhibits, the synthesis of one or more parasitism gene proteins of a nematode, such as SCN. The present invention comprises methods for transforming a plant cell or plant with one or more nucleic acid sequences of the present invention to result in a transgenic plant or in transgenic plant material that comprises a nucleic acid sequence, such as a dsRNA, that down regulates one or more target cyst nematode parasitism gene transcripts. The present invention comprises transgenic plants that are resistant to disease caused by cyst nematodes, for example SCN.

Target sequences in a nematode, which include nucleic acids or polypeptides found in a nematode plant pest, such as a cyst nematode, for example, H. glycines, and, may include one or more of the proteins encoded by SEQ ID NOs:1-142, one or more of the polypeptides of SEQ ID NOs: 143-159, or one or more of the sequences of SEQ ID NOs:1-142 which may be present in a parasitic nematode. As used herein, a “target sequence” or “target polynucleotide” comprises any sequence in the pest that one desires to reduce the level of expression. In specific embodiments, decreasing the level of the target sequence in the pest controls the pest. For instance, the target sequence can be essential for growth and development. While the target sequence can be expressed in any tissue of the pest, in specific embodiments, the sequences targeted for suppression in the pest are expressed in cells of the gut tissue of the pest, cells in the midgut of the pest, and cells lining the gut lumen or the midgut. Such target sequences can be involved in, for example, gut cell metabolism, growth or differentiation. Non-limiting examples of target sequences of the invention include a polynucleotide set forth in SEQ ID NOs: 1-142, fragments or variants thereof, or complements thereof. As exemplified elsewhere herein, decreasing the level of expression of one or more of these target sequences in a nematode plant pest or a cyst nematode, for example, H. glycines, plant pest controls the pest.

Nucleic acids of the present invention, polypeptides encoded thereby and/or antibodies which bind thereto, may be delivered to a nematode at any stage of the nematode lifecycle, including feeding nucleic acids or polypeptides to one or more nematodes, immersing in or contacting nematodes with nucleic acids, polypeptides or antibodies, or other stages of a nematode life cycle, including entry into a plant or plant cell and/or feeding by a nematode at the plant cell. Nucleic acids of the present invention may be internalized by the cyst nematode where the nucleic acid modulates the transcription, post-transcription, and/or translation of a nematode parasitism gene. Polypeptides of the present invention, including polypeptides encoded by SEQ ID NOs:1-142 and SEQ ID NOs: 143-159, and antibodies to the encoded polypeptides or to nucleic acids having a sequence of SEQ ID NOs:1-142, may be internalized by the cyst nematode to interfere, inhibit or stop plant parasitism by the nematode.

The present invention comprises a plant cell comprising a heterologous nucleic acid comprising one or more nucleic acid sequences disclosed herein having a nucleotide sequence comprising any one of SEQ ID NOs1-142, a fragment or variant thereof, or a complement thereof, wherein the heterologous nucleic acid is expressed in an amount sufficient to modulate, such as reduce or prevent, plant disease caused by plant-parasitic nematodes, such as by SCN. For example, a transgenic plant may express one or more nucleic acids having a nucleotide sequence comprising any one of SEQ ID NOs: 1-142, a fragment or variant thereof, or a complement thereof, and the one or more nucleic acids are delivered to a plant-parasitic nematode when it contacts or feeds on the plant.

The present invention comprises nucleic acid constructs comprising one or more nucleic acid sequences disclosed herein having a nucleotide sequence comprising any one of SEQ ID NOs: 1-142, a fragment or variant thereof, or a complement thereof. For example, a nucleic acid construct may be an expression cassette that encodes a silencing element, for example, one or more dsRNA molecules which may be used to modulate, such as inhibit, suppress or repress, nematode genes that are essential for growth and development of the plant-parasitic nematode, or for parasitic activities.

A nucleic acid construct of the present invention comprises one or more expression cassettes for expression in a plant or organism of interest. It is recognized that multiple silencing elements including multiple identical silencing elements, multiple silencing elements targeting different regions of the target sequence, or multiple silencing elements from different target sequences can be used. In this embodiment, it is recognized that each silencing element can be contained in a single or separate cassette, DNA construct, or vector. As discussed, any means of providing the silencing element is contemplated. A plant or plant cell can be transformed with a single cassette comprising DNA encoding one or more silencing elements or separate cassettes comprising each silencing element can be used to transform a plant or plant cell or host cell. Likewise, a plant transformed with one component can be subsequently transformed with the second component. One or more silencing elements can also be brought together by sexual crossing. That is, a first plant comprising one component is crossed with a second plant comprising the second component. Progeny plants from the cross will comprise both components.

The expression cassette can include 5′ and 3′ regulatory sequences operably linked to the polynucleotide of the invention. “Operably linked” is intended to mean a functional linkage between two or more elements. For example, an operable linkage between a polynucleotide of the invention and a regulatory sequence (i.e., a promoter) is a functional link that allows for expression of the polynucleotide of the invention. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression cassettes can be provided with a plurality of restriction sites and/or recombination sites for insertion of the polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a polynucleotide comprising the silencing element employed in the methods and compositions of the invention, and a transcriptional and translational termination region (i.e., termination region) functional in plants. In another embodiment, the double stranded RNA is expressed from a suppression cassette. Such a cassette can comprise two convergent promoters that drive transcription of an operably linked silencing element. “Convergent promoters” refers to promoters that are oriented on either terminus of the operably linked silencing element such that each promoter drives transcription of the silencing element in opposite directions, yielding two transcripts. In such embodiments, the convergent promoters allow for the transcription of the sense and anti-sense strand and thus allow for the formation of a dsRNA.

By “silencing element” is intended a polynucleotide which when ingested by a pest, or when the pest is exposed to one or more silencing elements, is capable of reducing or eliminating the level or expression of a target polynucleotide or the polypeptide encoded thereby. The silencing element employed can reduce or eliminate the expression level of the target sequence by influencing the level of the target RNA transcript or, alternatively, by influencing translation and thereby affecting the level of the encoded polypeptide. Methods to assay for functional silencing elements that are capable of reducing or eliminating the level of a sequence of interest are disclosed. A single polynucleotide employed in the methods of the invention can comprise one or more silencing elements to the same or different target polynucleotides. The silencing element can be produced in vivo (i.e., in a host cell such as a plant or microorganism) or in vitro.

In specific embodiments, the target sequence is not endogenous to the plant. In other embodiments, while the silencing element controls pests, preferably the silencing element has no effect on the normal plant or plant part.

Silencing elements can include, but are not limited to, a sense suppression element, an antisense suppression element, a double stranded RNA, a siRNA, an amiRNA, a miRNA, or a hairpin suppression element. Non-limiting examples of silencing elements that can be employed to decrease expression of target nematode plant pest sequences or cyst nematode, for example, H. glycines, plant pest sequences comprise fragments and variants of the sense or antisense sequence or consists of the sense or antisense sequence of the sequence set forth in SEQ ID NOs: 1-142, or a variant or fragment thereof. The silencing element can further comprise additional sequences that advantageously effect transcription and/or the stability of a resulting transcript. For example, the silencing elements can comprise at least one thymine residue at the 3′ end. This can aid in stabilization. Thus, the silencing elements can have at least 1, 2, 3, 4, 5, 6, 7, 8, 9, 10 or more thymine residues at the 3′ end.

By “reduces” or “reducing” the expression level of a polynucleotide or a polypeptide encoded thereby is intended to mean, the polynucleotide or polypeptide level of the target sequence is statistically lower than the polynucleotide level or polypeptide level of the same target sequence in an appropriate control pest which is not exposed to (i.e., has not ingested) the silencing element. In particular embodiments of the invention, reducing the polynucleotide level and/or the polypeptide level of the target sequence in a pest according to the invention results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control pest. Methods to assay for the level of the RNA transcript, the level of the encoded polypeptide, or the activity of the polynucleotide or polypeptide are discussed elsewhere herein.

As used herein, a “sense suppression element” comprises a polynucleotide designed to express an RNA molecule corresponding to at least a part of a target messenger RNA in the “sense” orientation. Expression of the RNA molecule comprising the sense suppression element reduces or eliminates the level of the target polynucleotide or the polypeptide encoded thereby. The polynucleotide comprising the sense suppression element may correspond to all or part of the sequence of the target polynucleotide, all or part of the 5′ and/or 3′ untranslated region of the target polynucleotide, all or part of the coding sequence of the target polynucleotide, or all or part of both the coding sequence and the untranslated regions of the target polynucleotide.

Typically, a sense suppression element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. See, U.S. Pat. Nos. 5,283,184 and 5,034,323; herein incorporated by reference. The sense suppression element can be any length so long as it allows for the suppression of the targeted sequence. The sense suppression element can be, for example, 15, 16, 17, 18 19, 20, 22, 25, 30, 50, 100, 150, 200, 250, 300, 350, 400, 450, 500, 600, 700, 900, 1000, 1100, 1200, 1300 nucleotides or longer of the target polynucleotides set forth in any of SEQ ID NOs: 1-142. In other embodiments, the sense suppression element can be, for example, about 15-25, 25-100, 100-150, 150-200, 200-250, 250-300, 300-350, 350-400, 450-500, 500-550, 550-600, 600-650, 650-700, 700-750, 750-800, 800-850, 850-900, 900-950, 950-1000, 1000-1050, 1050-1100, 1100-1200, 1200-1300, 1300-1400, 1400-1500, 1500-1600, 1600-1700, 1700-1800 nucleotides or longer of the target polynucleotides set forth in any of SEQ ID NOs: 1-142.

As used herein, an “antisense suppression element” comprises a polynucleotide which is designed to express an RNA molecule complementary to all or part of a target messenger RNA. Expression of the antisense RNA suppression element reduces or eliminates the level of the target polynucleotide. The polynucleotide for use in antisense suppression may correspond to all or part of the complement of the sequence encoding the target polynucleotide, all or part of the complement of the 5′ and/or 3′ untranslated region of the target polynucleotide, all or part of the complement of the coding sequence of the target polynucleotide, or all or part of the complement of both the coding sequence and the untranslated regions of the target polynucleotide. In addition, the antisense suppression element may be fully complementary (i.e., 100% identical to the complement of the target sequence) or partially complementary (i.e., less than 100% identical to the complement of the target sequence) to the target polynucleotide. In specific embodiments, the antisense suppression element comprises at least 85%, 90%, 91%, 92%, 93%, 94%, 95%, 96%, 97%, 98%, or 99% sequence complementarity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide. Generally, sequences of at least 15, 20, 22, 25, 50, 100, 200, 300, 400, 450 nucleotides or greater of the sequence set forth in any of SEQ ID NO: 1-278 may be used. Methods for using antisense suppression to inhibit the expression of endogenous genes in plants are described, for example, in Liu et at (2002) Plant Physiol. 129:1732-1743 and U.S. Pat. Nos. 5,759,829 and 5,942,657, each of which is herein incorporated by reference.

Methods of the present invention may comprise control of nematode parasitism by sequence-specific inhibition of expression of coding sequences of nematode or host plant genes, for example, by using silencing elements such as RNA molecules, for example, double-stranded RNA (dsRNA) or small interfering RNA (siRNA), or by providing other exogenous nucleic acid constructs to a host plants to modulate, including up-regulating host defense genes, or in other ways to interfere with, suppress, repress or inhibit, nematode infection of a host plant. The present invention comprises methods and compositions for genetic control of parasitic nematodes in host organisms, particularly plant-parasitic nematodes, such as Heterodera sp., soybean cyst nematode (SCN), or H. glycines. A method of the present invention may comprise delivery of a composition comprising polynucleotides to a parasitic nematode. A method of the present invention may comprise delivery of a composition comprising polypeptides to a parasitic nematode. A method of the present invention may comprise delivery of a composition comprising antibodies that bind one or more polypeptides encoded by nucleic acids of the present invention to a parasitic nematode. Compositions described herein may, directly or indirectly, modulate the ability of plant-parasitic nematodes, such as SCN, to feed, grow or otherwise cause disease in a host plant. Methods and compositions of the present invention comprise methods for control of plant disease in a nematode host plant, comprising, in a parasitic nematode or its plant host, modulating the biological activities of genes, peptides, proteins or control elements having a nucleic acid sequence of SEQ ID NOs:1-142, a fragment thereof, a complement of a nucleic acid sequence of SEQ ID NOs:1-142, or a fragment thereof

### Nucleic Acids SEQ ID NOs: 1-142

The present invention comprises compositions comprising novel isolated nucleic acids having a sequence that is identical to at least a portion of one or more native nucleic acid sequences in a plant-parasitic nematode. In an aspect, the nematode is Heterodera sp., such as H. glycines or H. schachtii. Specific examples of nucleic acids of the present invention are SEQ ID NOs:1-142, a fragment thereof, a complement of a nucleic acid sequence of SEQ ID NOs:1-142, or a fragment thereof.

The present invention comprises novel isolated nucleic acids having a nucleotide sequence comprising any one of SEQ ID NOs: 1-142, a fragment or variant thereof, or a complement thereof, which are referred to herein generally as nucleic acids of the present invention. The present invention comprises an isolated polynucleotide, wherein the isolated polynucleotide is (a) a nucleic acid sequence of any of SEQ ID NOs:1-142; (b) a fragment of at least 10, 20, 30, 40, 50, 60, 70, 80 or more contiguous nucleotides of a nucleic acid sequence of any of SEQ ID NOs:1-142; or (c) a complement of the sequence of (a) or (b). A fragment of contiguous nucleotides of a nucleic acid sequence of any of SEQ ID NOs:1-142 may comprise about 10-20 nucleotides, about 15-30 nucleotides, about 20-30 nucleotides, about 20-40 nucleotides of a nucleic acid sequence of any of SEQ ID NOs:1-142, and such a fragment may encode a polynucleotide for RNA silencing. As used herein, fragment refers to contiguous nucleotides.

Nucleic acids of the present invention may be synthesized, either completely or in part, by methods known in the art. Nucleic acids may be synthesized in and by any type of cell, or by mechanical and chemical methods. All or a portion of the nucleic acids of the present invention may be synthesized using codons preferred by a selected host. Species-preferred codons may be determined, for example, from the codons used most frequently in the proteins expressed in a particular host species. Other modifications of the nucleotide sequences may result in mutants having slightly altered activity.

The present invention contemplates fragments and variants of the nucleic acid sequences and/or polypeptide sequences disclosed herein, including an isolated polynucleotide of SEQ ID NOs:1-142, a fragment of an isolated polynucleotide of SEQ ID NOs: 1-142, a complement of an isolated polynucleotide of SEQ ID NOs: 1-142, or a fragment of a complement of an isolated polynucleotide of SEQ ID NOs: 1-142, SEQ ID NOs: 143-159, or fragments thereof. By “fragment” is intended a portion of the polynucleotide or a portion of the amino acid sequence and hence protein encoded thereby. Fragments of a polynucleotide may encode protein fragments that retain the biological activity of the native protein. Alternatively, fragments of a polynucleotide that are useful as a silencing element do not need to encode protein fragments that retain biological activity. Thus, fragments of a nucleotide sequence may range from at least about 10, about 15, about 16, about 17, about 18, about 19, about 20 nucleotides, about 22 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to the full-length polynucleotide employed in the invention. Alternatively, fragments of a nucleotide sequence may range from 1-50, 25-75, 75-125, 50-100, 125-175, 175-225, 100-150, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of any one of SEQ ID NOs: 6, 7, 8, 9, 10, 11, 12, 18, 19 or 20. Methods to assay for the activity of a desired silencing element are described elsewhere herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. A variant of a polynucleotide that is useful as a silencing element will retain the ability to reduce expression of the target polynucleotide and, in some embodiments, thereby control a pest of interest. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the polypeptides employed in the invention. Variant polynucleotides also include synthetically derived polynucleotide, such as those generated, for example, by using site-directed mutagenesis, but continue to retain the desired activity. Generally, variants of a particular polynucleotide of the invention (i.e., a silencing element) will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

A composition of the present invention may comprise a nucleic acid construct comprising a polynucleotide of SEQ ID NOs:1-142, a fragment or variant of an isolated polynucleotide of SEQ ID NOs:1-142, a complement of an isolated polynucleotide of SEQ ID NOs:1-142, or a fragment or variant of a complement of an isolated polynucleotide of SEQ ID NOs:1-142. A nucleic acid construct may comprise a plant transformation vector, comprising one or more nucleic acid sequences, wherein a nucleic acid sequence may be one or more nucleic acid sequences disclosed herein having a nucleotide sequence comprising any one of SEQ ID NOs: 1-142, a fragment or variant thereof, or a complement thereof. A polynucleotide sequence may be operably linked to a promoter, heterologous or homologous, or other control sequences that are functional in a plant cell, or other cell. A promoter may be tissue-specific and, for example, may be specific to a tissue where the plant-parasite nematode interacts with a plant. For example, as nematodes enter soybean plants at the roots, a promoter may provide root-preferred expression. A nucleic acid of the present invention may be placed between two tissue specific promoters, such as two root specific promoters, which are operable in a transgenic plant cell, and may be expressed to produce RNA in the transgenic plant cell that forms dsRNA molecules. Examples of root-specific promoters are known in the art, such as the nematode-induced RB7 promoter, U.S. Pat. No. 5,459,252 and Opperman et al. 1994. A recombinant DNA vector or nucleic acid construct of the present invention may comprise a selectable marker that confers a selectable phenotype on plant cells, which may be used to select plants or plant cells that contain the exogenous nucleic acids encoding nucleic acids, polypeptides or proteins of the present invention. The marker may encode biocide resistance, antibiotic resistance, or herbicide resistance. Such resistance markers are known in the art and may be selected by one skilled in the art. A recombinant vector or construct of the present invention may also include a marker that may be used to monitor expression. Many vectors are available and are known to those skilled in the art. Selection of the appropriate vector is within the skill of those in the art and, for example, may depend mainly on the size of the nucleic acid to be inserted into the vector and the particular host cell to be transformed with the vector. It is contemplated that the appropriate vector will contain components for its adequate functioning in the host cell. The present invention is not limited by the method of transformation of a cell or plants resulting from transformed cells, and any method for introducing nucleic acids into a cell may be used, including, but not limited to, electroporation, introduction of coated particles, gene guns, transformation of protoplasts, by desiccation/inhibition-mediated DNA uptake, microbial-mediated transformation, by agitation with silicon carbide fibers, or by transformation using Agrobacterium. Transformation protocols as well as protocols for introducing polypeptides or polynucleotide sequences into plants are known and may vary depending on the type of plant or plant cell, i.e., monocot or dicot, targeted for transformation.

A number of promoters can be used in the practice of the invention. A nucleic acid construct may comprise at least a nucleic acid sequence of interest and optionally, a promoter such as a promoter known in the art or disclosed herein, including, but not limited to constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

An inducible promoter, for instance, a pathogen-inducible promoter could also be employed. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); system in (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10): 1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2): 343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

In an aspect, the plant-expressed promoter is a vascular-specific promoter such as a phloem-specific promoter. A “vascular-specific” promoter, as used herein, is a promoter which is at least expressed in vascular cells, or a promoter which is preferentially expressed in vascular cells. Expression of a vascular-specific promoter need not be exclusively in vascular cells, expression in other cell types or tissues is possible. A “phloem-specific promoter” as used herein, is a plant-expressible promoter which is at least expressed in phloem cells, or a promoter which is preferentially expressed in phloem cells.

Expression of a phloem-specific promoter need not be exclusively in phloem cells, expression in other cell types or tissues, e.g., xylem tissue, is possible. In one embodiment of this invention, a phloem-specific promoter is a plant-expressible promoter at least expressed in phloem cells, wherein the expression in non-phloem cells is more limited (or absent) compared to the expression in phloem cells. Examples of suitable vascular-specific or phloem-specific promoters in accordance with this invention include but are not limited to the promoters selected from the group consisting of: the SCSV3, SCSV4, SCSV5, and SCSV7 promoters (Schunmann et al. (2003) Plant Functional Biology 30:453-60; the rolC gene promoter of Agrobacterium rhizogenes(Kiyokawa et al. (1994) Plant Physiology 104:801-02; Pandolfini et al. (2003) BioMedCentral (BMC) Biotechnology 3:7, (www.biomedcentral.com/1472-6750/3/7); Graham et al. (1997) Plant Mol. Biol. 33:729-35; Guivarc'h et al. (1996); Almon et al. (1997) Plant Physiol. 115:1599-607; the rolA gene promoter of Agrobacterium rhizogenes (Dehio et al. (1993) Plant Mol. Biol. 23:1199-210); the promoter of the Agrobacterium tumefaciens T-DNA gene 5 (Korber et al. (1991) EMBO J. 10:3983-91); the rice sucrose synthase RSsl gene promoter (Shi et al. (1994) J. Exp. Bot. 45:623-31); the CoYMV or Commelina yellow mottle badnavirus promoter (Medberry et al. (1992) Plant Cell 4:185-92; Zhou et al. (1998) Chin. J. Biotechnol. 14:9-16); the CFDV or coconut foliar decay virus promoter (Rohde et al. (1994) Plant Mol. Biol. 27:623-28; Hehn and Rhode (1998) J. Gen. Prot. 79:1495-99); the RTBV or rice tungro bacilliform virus promoter (Yin and Beachy (1995) Plant J. 7:969-80; Yin et al. (1997) Plant J. 12:1179-80); the pea glutamin synthase GS3A gene (Edwards et al. (1990) Proc. Natl. Acad. Sci. USA 87:3459-63; Brears et al. (1991) Plant J. 1:235-44); the inv CD111 and inv CD141 promoters of the potato invertase genes (Hedley et al. (2000) J. Exp. Botany 51:817-21); the promoter isolated from Arabidopsis shown to have phloem-specific expression in tobacco by Kertbundit et al. (1991) Proc. Natl. Acad. Sci. USA 88:5212-16); the VAHOX1 promoter region (Tornero et al. (1996) Plant J. 9:639-48); the pea cell wall invertase gene promoter (Zhang et al. (1996) Plant Physiol. 112:1111-17); the promoter of the endogenous cotton protein related to chitinase of US published patent application 20030106097, an acid invertase gene promoter from carrot (Ramloch-Lorenz et al. (1993) The Plant J. 4:545-54); the promoter of the sulfate transporter geneSultrl; 3 (Yoshimoto et al. (2003) Plant Physiol. 131:1511-17); a promoter of a sucrose synthase gene (Nolte and Koch (1993) Plant Physiol. 101:899-905); and the promoter of a tobacco sucrose transporter gene (Kuhn et al. (1997) Science 275-1298-1300).

Possible promoters also include the Black Cherry promoter for Prunasin Hydrolase (PH DL1.4 PRO) (U.S. Pat. No. 6,797,859), Thioredoxin H promoter from cucumber and rice (Fukuda A et al. (2005). Plant Cell Physiol. 46(11):1779-86), Rice (RSsl) (Shi, T. Wang et al. (1994). J. Exp. Bot. 45(274): 623-631) and maize sucrose synthese −1 promoters (Yang., N-S. et al. (1990) PNAS 87:4144-4148), PP2 promoter from pumpkin Guo, H. et al. (2004) Transgenic Research 13:559-566), At SUC2 promoter (Truernit, E. et al. (1995) Planta 196(3):564-70., At SAM-1 (S-adenosylmethionine synthetase) (Mijnsbrugge K V. et al. (1996) Planr. Cell. Physiol. 37(8): 1108-1115), and the Rice tungro bacilliform virus (RTBV) promoter (Bhattacharyya-Pakrasi et al. (1993) Plant J. 4(1):71-79).

The polynucleotide encoding the silencing element or in specific embodiments employed in the methods and compositions of the invention can be provided in expression cassettes for expression in a plant or organism of interest. It is recognized that multiple silencing elements including multiple identical silencing elements, multiple silencing elements targeting different regions of the target sequence, or multiple silencing elements from different target sequences can be used. In this embodiment, it is recognized that each silencing element can be contained in a single or separate cassette, DNA construct, or vector. As discussed, any means of providing the silencing element is contemplated. A plant or plant cell can be transformed with a single cassette comprising DNA encoding one or more silencing elements or separate cassettes comprising each silencing element can be used to transform a plant or plant cell or host cell. Likewise, a plant transformed with one component can be subsequently transformed with the second component. One or more silencing elements can also be brought together by sexual crossing. That is, a first plant comprising one component is crossed with a second plant comprising the second component. Progeny plants from the cross will comprise both components.

The expression cassette can include 5′ and 3′ regulatory sequences operably linked to the polynucleotide of the invention. “Operably linked” is intended to mean a functional linkage between two or more elements. For example, an operable linkage between a polynucleotide of the invention and a regulatory sequence (i.e., a promoter) is a functional link that allows for expression of the polynucleotide of the invention. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression cassettes can be provided with a plurality of restriction sites and/or recombination sites for insertion of the polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a polynucleotide comprising the silencing element employed in the methods and compositions of the invention, and a transcriptional and translational termination region (i.e., termination region) functional in plants. In other embodiment, the double stranded RNA is expressed from a suppression cassette. Such a cassette can comprise two convergent promoters that drive transcription of an operably linked silencing element. “Convergent promoters” refers to promoters that are oriented on either terminus of the operably linked silencing element such that each promoter drives transcription of the silencing element in opposite directions, yielding two transcripts. In such embodiments, the convergent promoters allow for the transcription of the sense and anti-sense strand and thus allow for the formation of a dsRNA. The present invention comprises cells transformed with a nucleic acid construct such as a nucleic acid construct comprising a nucleotide sequence of one or more of SEQ ID NOs:1-142, a fragment or variant of one or more of SEQ ID NOs:1-142, a complement of one or more of SEQ ID NOs:1-142, and/or a fragment or variant of a complement of one or more of SEQ ID NOs:1-142. The cells may be prokaryotic or eukaryotic cells. The cells may be plant cells. The present invention comprises plants and seeds derived from plant cells transformed by a nucleic acid construct of the present invention. The present invention comprises a product produced from a transformed plant, wherein a product comprises a detectable amount of a polynucleotide having a sequence or a fragment or variant of a SEQ ID NOs:1-142, or a complement thereof, wherein the polynucleotide may be DNA or RNA. A product may be transformed plants, roots, cells, seeds, food, feed, oil, meal, protein, starch, flour or silage.

The present invention comprises recombinant nucleic acid constructs for use in achieving stable or transient transformation of particular host organisms such as plants. “Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant. Transformed hosts may express effective levels of proteins, peptides, nucleic acids, dsRNA or ssRNA molecules from the recombinant nucleic acid constructs. The isolated and purified nucleotide sequences may be provided from cDNA libraries disclosed herein and/or genomic library information, and may include polynucleotides having a nucleotide sequence comprising any one of SEQ ID NOs: 1-142, a fragment or variant thereof, or a complement thereof. In an aspect, a recombinant nucleic acid construct may comprise sequences encoding a binding region of an antibody, an antibody fragment or a binding peptide that binds to a polypeptide encoded by one or more of SEQ ID NOs:1-142, a fragment or variant thereof, or a complement thereof or a polypeptide of SEQ ID NOs: 143-159, a fragment or variant thereof.

A transformed cell may comprise a nucleic acid sequence of the present invention in its genome or genetic material of an organelle, so that the nucleic acid sequence of the present invention is found in daughter cells, progeny, plants or seeds derived from plants of the transformed cells. A nucleic acid molecule comprising a nucleic acid sequence of the present invention may be found in the transgenic plant cell, not incorporated into the genome or genetic material of an organelle, for example, it may be found in the cytoplasm or in an apoplastic space. A plant transformed by the nucleic acids of the present invention may be more resistant to or tolerant of nematode infection than non-transformed plants.

The present invention comprises nucleic acid sequences capable of being expressed as RNA in a cell or microorganism to inhibit gene expression in a cell, tissue or organ of a plant-parasitic nematode. A dsDNA molecule may be placed so that it operates under the control of a promoter sequence which functions in the cell, tissue or organ of the host expressing the dsDNA to produce dsRNA molecules. In an aspect, the DNA sequence may be one or more nucleic acid sequences disclosed herein having a nucleotide sequence comprising any one of SEQ ID NOs: 1-142, a fragment or variant thereof, or a complement thereof.

The present invention comprises a nucleic acid sequence that is expressed in a plant cell as RNA wherein the RNA suppresses or represses a target gene in a plant-parasitic nematode. Methods to express a gene suppression molecule in plants are known to those skilled in the art and such methods may be used to express a nucleotide sequence of the present invention. Nucleic acids comprising one or more nucleic acid sequences disclosed herein having a nucleotide sequence comprising any one of SEQ ID NOs: 1-142, a fragment or variant thereof, or a complement thereof, are capable of specifically hybridizing to other nucleic acid molecules under certain circumstances. As used herein, a target gene may be a gene that performs at least one function in a nematode and includes, but is not limited to, DNA replication, cell cycle control, transcription, RNA processing, translation, ribosome function, tRNA synthesis, tRNA function, protein trafficking, secretion, protein modification, protein stability, protein degradation, energy production, mitochondrial function, intermediary metabolism, cell structure, signal transduction, endocytosis, ion regulation and transport.

The present invention comprises a nucleic acid sequence that is expressed in a plant cell as a polypeptide wherein the polypeptide modulates, such as by interfering, blocking, suppressing or repressing cellular, tissue or whole body activities associated with parasitism by a plant-parasitic nematode. Methods to express a polypeptide molecule in plants are known to those skilled in the art and such methods may be used to express a nucleotide sequence encoding a polypeptide sequence of the present invention or an antibody binding sequence. Polypeptides encoded by one or more nucleic acid sequences disclosed herein having a nucleotide sequence comprising any one of SEQ ID NOs: 1-142, a fragment or variant thereof, or a complement thereof, are capable of specifically binding to polypeptide or polynucleotide molecules under certain circumstances.

The present invention contemplates that one or more nucleic acid constructs comprising one or more nucleic acid sequences disclosed herein having a sequence of the present invention comprising a nucleotide sequence comprising any one of SEQ ID NOs: 1-142, a fragment or variant thereof, or a complement thereof, may be present in a cell, plant, a transformed cell or a transformed plant. One or more target genes to which the sequences of the present invention hybridize may be modulated by the presence of the nucleic acid constructs in a cell or plant. There may be present in a cell or plant one or more nucleic acid constructs, each having a nucleic acid sequence of the present invention, or there may be present one nucleic acid construct having more than one sequence of the present invention, or there may be present in a cell or plant, one or more nucleic acid constructs each having more than one nucleic acid sequence of the present invention. The nucleic acid sequences in the nucleic acid constructs may be under the control of one or multiple promoters.

The present invention comprises a ribonucleic acid expressed from a nucleic acid of the present invention which may comprise one or more nucleic acid sequences disclosed herein having a nucleotide sequence comprising any one of SEQ ID NOs: 1-142, a fragment or variant thereof, or a complement thereof. For example, a ribonucleic acid may be a dsRNA. For example, a ribonucleic acid may be a ssRNA. Isolated and substantially purified nucleic acid molecules including, but not limited to, non-naturally occurring nucleotide sequences, recombinant DNA constructs for transcribing dsRNA and ssRNA molecules, and nucleic acid constructs of the present invention, may be used in methods for modulating, such as suppressing or inhibiting, the expression of an endogenous coding sequence or a target coding sequence in a plant-parasitic nematode. Compositions comprising nucleic acid constructs comprising one or more nucleic acid sequences disclosed herein having a nucleotide sequence comprising any one of SEQ ID NOs: 1-142, a fragment or variant thereof, or a complement thereof, may be provided topically to host plants or to nematodes, or may be provided to the environment, such as the soil, where planting may occur or where nematodes are present. Nucleic acid molecules, such as dsRNA or ssRNA, partially or entirely encoded by a nucleotide sequence comprising any one of SEQ ID NOs: 1-142, a fragment or variant thereof, or a complement thereof, may be provided topically to host plants or to nematodes, or may be provided to the environment, such as the soil, where planting may occur or where nematodes are present. Such nucleic acid compositions may be provided in delivery vehicles that are appropriate for protecting and transferring nucleic acids to organisms.

Methods and compositions of the present invention comprise a fragment of a nucleic acid sequence of one or more nucleic acid sequences disclosed herein having a nucleic acid sequence of SEQ ID NOs:1-142, or a complement of a nucleic acid sequence of SEQ ID NOs:1-142. A fragment may be capable of modulating the cellular activities of a plant-parasitic nematode, such as when the fragment is expressed in a plant cell as dsRNA or ssRNA which when contacted by or is ingested by the nematode may provide for modulation of the nematode. For example, a fragment may comprise at least about 10, 12, 15, 17, 19, 21, 23, 25, 40, 60, 80, 100, 125, 200, 300 or more contiguous nucleotides of any of one or more nucleic acid sequences disclosed herein having a nucleic acid sequence of SEQ ID NOs:1-142, or a complement of a nucleic acid sequence of SEQ ID NOs:1-142. One fragment may be at least from about 12-20 nucleotides, from about 15 to about 23, or about 23 to about 100 nucleotides, but less than about 3000 nucleotides, in length. dsRNA and/or ssRNA sequences from a fragment of about 10 to about 400 nucleotides that are homologous to a plant-parasitic nematode target sequence are contemplated by the present invention.

Methods and compositions of the present invention comprise use of nucleic acids of the present invention in assays for detecting or determining parasitism by nematodes. The presence of nematode specific polynucleotides may be determined by hybridizing one or more nucleic acid sequences disclosed herein having a nucleotide sequence comprising any one of SEQ ID NOs: 1-142, a fragment or variant thereof, or a complement thereof, to a sample comprising nucleic acids. Such a sample may be taken from a nematode or plant. Such nucleic acid assays are known in the art.

### Polypeptides of the Present Invention

Polypeptides of the present invention comprise polypeptides that may be encoded by any one of a nucleotide sequence comprising any one of SEQ ID NOs: 1-142, a fragment or variant thereof, or a complement thereof. Polypeptides of the present invention comprise polypeptides having a sequence of any one of SEQ ID NOs: 143-159, or variants or fragments thereof. Such a polypeptide may comprise a leader sequence for secretion, terminal sequences, signal sequences, or control element sequences. Polypeptides may comprise non-active forms, which may be cleaved to provide a biologically active form. Isolated polypeptides of the present invention may be homologous to proteins found in cyst nematodes or other nematodes.

In an aspect, polypeptides of the present invention comprise antibodies or antibody fragments that were produced in response to polypeptides encoded by a nucleic acid of the present invention, or polypeptides having a sequence of any one of SEQ ID NOs: 143-159, or antigen binding sites of antibodies that were produced in response to polypeptides encoded by one or more nucleic acid sequences disclosed herein having a nucleotide sequence comprising any one of SEQ ID NOs: 1-142, a fragment or variant thereof, or a complement thereof.

Methods of using polypeptides of the present invention comprise providing nucleic acid constructs comprising a nucleotide sequence comprising any one of SEQ ID NOs: 1-142, a fragment or variant thereof, or a complement thereof, encoding polypeptides or fragments so that the polypeptide or fragment is expressed within a host cell, and optionally, modulating plant parasitism by a nematode, such as SCN. Methods of the present invention comprise providing compositions comprising polypeptides or fragments of such polypeptides encoded by a nucleotide sequence comprising any one of SEQ ID NOs: 1-142, a fragment or variant thereof, or a complement thereof; or any one of SEQ ID NOs: 143-159, or a fragment thereof, to nematodes or plants to modulate plant parasitism by a nematode, such as SCN. For example, such polypeptides or fragments may provide a blocking function by binding in a site where a native protein binds and/or interferes with activities by the native protein in the nematode. A polypeptide of the present invention or a fragment thereof may be mutated in such a manner so that its activity or function is modulated from that of a native protein. Methods for mutating polypeptides are known in the art and may be selected by a skilled artisan.

Methods and compositions of the present invention comprise disclosed polypeptides of the present invention and antibodies to polypeptides of the present invention for use in diagnosing or detecting nematode presence, infection or parasitism. Such polypeptides and antibodies may be used in assays, including immunoassays, for detecting polypeptides in a sample taken from nematodes or plants. Such assays are known in the art.

### Modulating Expression of a Target Gene in a Nematode Cell

The present invention comprises methods for modulating the expression of a target gene in a nematode cell. In an aspect, a method may comprise (a) transforming a plant cell with a nucleic acid construct comprising one or more nucleic acid sequences encoding a gene, the complementary sequences of a gene, a protein, a control sequence such as an enhancer or promotor, dsRNA, or ssRNA, having a sequence selected from the group consisting of one or more nucleic acid sequences disclosed herein having a nucleotide sequence comprising any one of SEQ ID NOs: 1-142, a fragment or variant thereof, or a complement thereof, optionally, the sequence or sequences may be operatively linked to a promoter and a transcription termination sequence; (b) culturing the transformed plant cell under conditions sufficient to allow for development of a plant cell culture comprising a plurality of transformed plant cells; (c) selecting for transformed plant cells that have integrated the nucleic acid sequence into their genomes or wherein the nucleic acid is expressed or is present. Plants may also be regenerated from such plant cells. A method for modulating target gene expression may result in the cessation of growth, development, reproduction, feeding, and/or death of a plant-parasitic nematode, including but not limited to, SCN. The method may limit or eliminate nematode parasitism of plants or host tissues, or may limit or eliminate nematode survival in an environment.

The present invention comprises transformation of a plant with a nucleotide sequence of the present invention comprising one or more nucleic acid sequences disclosed herein having a nucleotide sequence comprising any one of SEQ ID NOs: 1-142, a fragment or variant thereof, or a complement thereof, to provide nematode inhibitory levels of expression of one or more dsRNAs. Methods for transformation of a plant cell and its resulting plants are known to those skilled in the art, such as by using a transformation vector or nucleic acid construct described herein. Transformation may occur by site-specific or non-specific integration of the exogenous nucleic acid sequences. A nucleic acid construct may comprise one or more nucleotide sequences of the present invention, and optionally control elements such as enhancers or promoters, expression sequences and other known sequences for entry of the vector or construct into a cell and utilization of the sequences, such as transcription and expression. The sequences of the nucleic acid construct may be used for the down-regulation of expression of at least one nucleotide sequences of a nematode. A nucleic acid construct may provide one or more sequences that are expressed in a host cell as RNA which may assemble to form ssRNA or dsRNA that will function to inhibit the functioning of RNA in a nematode, to reduce or inhibit expression of proteins or nucleotides in a nematode. The inhibition may be sequence specific inhibition or may be generally inhibitory to the nematode cells. A nucleotide sequence of the nematode to which a ssRNA or dsRNA is inhibitory may have 80%, 85%, 90%, 95%, 96%, 97%, 98%, 99% 99.9% or 100% sequence identity to one or more nucleic acid sequences disclosed herein having a nucleotide sequence comprising any one of SEQ ID NOs: 1-142, a fragment or variant thereof, or a complement thereof. In an aspect, a method of transforming a cell with nematode inhibitory levels of one or more dsRNA molecules may be used to target one nematode gene or more than one nematode genes, or both. In an aspect, a method of transforming a cell with nematode inhibitory levels of one or more dsRNA molecules may be used to target one plant gene or more than one plant genes, or both nematode and plant genes. In specific embodiments, the silencing element sequences of the invention can be provided to a plant using a variety of transient transformation methods. Such transient transformation methods include, but are not limited to, the introduction of the protein or variants and fragments thereof directly into the plant or the introduction of the transcript into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, polynucleotides can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylimine (PEI; Sigma #P3143).

In other embodiments, the polynucleotide of the invention may be introduced into plants by contacting plants with a virus or viral nucleic acids. Generally, such methods involve incorporating a nucleotide construct of the invention within a viral DNA or RNA molecule. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

As used herein, the term plant also includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants and sugarcane plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The present invention comprises a transformed host plant of a plant-parasite nematode, and includes transformed plant cells and transformed plants and their progeny, such as by methods described herein. The transformed plant cells and transformed plants may express one or more nucleic acid sequences disclosed herein having a nucleotide sequence comprising any one of SEQ ID NOs: 1-142, a fragment or variant thereof, or a complement thereof, under the control of a heterologous promoter, described herein to provide a protection to the plant cells or plant from the infection of nematodes, particularly cyst nematodes, such as Heterodera sp., such as SCN. These sequences may be used for gene suppression in a nematode, which reduces the level or incidence of disease caused by the nematode in a host plant. Gene suppression may include modulation, such as reduction, of replication, transcription, post-transcription processing, or translation of gene products of the nematode. Gene suppression may also be effective for host genes.

Gene suppression or gene expression inhibition may be in all cells of a nematode or in one or more subsets of cells of a nematode. Similarly, gene suppression or expression inhibition may occur in all cells of a plant or one or more subsets of cells of a host plant. Gene suppression may be quantified by measuring amounts of target RNA or protein gene product in cells without a gene suppressing sequence of the present invention with cells comprising a gene suppressing sequence of the present invention, or by phenotypical changes in transformed cells or plants. Methods for quantifying nucleic acids and proteins are well known to one of ordinary skill in the art, as measurements of phenotypical changes. In an aspect, gene suppression or inhibition may be 10%, 20%, 30%, 40%, 50%, 60%, 70%, 80%, 90% or 100% of normal gene levels or activity.

A transformed plant cells and transformed plants of the present invention may express one or more polypeptides encoded by nucleic acid sequences disclosed herein having a nucleotide sequence comprising any one of SEQ ID NOs: 1-142, a fragment or variant thereof, or a complement thereof, or a mutant thereof, described herein to provide a protection to the plant cells or plant from the parasitism by nematodes, particularly cyst nematodes, such as Heterodera sp., such as SCN. These peptides may be used to reduce the level or incidence of disease caused by the nematode in a host plant. In an aspect, an expressed polypeptide may be an antigen-binding region of an antibody, an antibody fragment or binding peptides made in response to a polypeptide encoded by one or more nucleic acid sequences disclosed herein having a nucleic acid sequence of SEQ ID NOs:1-142, a fragment thereof, a complement of the nucleic acid sequence of SEQ ID NOs:1-142, or a complement of a fragment thereof

### RNA Interference

The present invention comprises methods and compositions involving RNA interference (RNAi) in host plant cells, which comprises cellular pathways where a sequence specific double stranded RNA (dsRNA) results in the degradation of a mRNA of interest. RNAi is effective in gene knockdown in a number of species including nematodes. Though not wishing to be bound by any particular theory, it is currently believed that RNAi works through a cellular pathway comprising RNAse III enzyme or the Dicer protein complex that generates about 21-nucleotide small interfering RNAs (siRNAs) from the original dsRNA and the RNA-induced silencing complex (RISC) that uses siRNA guides to recognize and degrade the corresponding mRNAs. Only transcripts complementary to the siRNA are cleaved and degraded, and the knockdown of mRNA expression is usually sequence specific. The gene silencing effect of RNAi may last for days and may lead to a large decline in amount of the targeted transcript, with the coincident decline in levels of the corresponding protein. In a method of the present invention, a polynucleotide having a nucleotide sequence of the present invention present in a host plant cell may encode a polynucleotide capable of functioning as a dsRNA or siRNA to knockdown nematode-specific genes or mRNAs. The nematode-specific gene or mRNAs may be one or more nucleic acid sequences disclosed herein having a nucleotide sequence comprising any one of SEQ ID NOs: 1-142, a fragment or variant thereof, or a complement thereof. The polynucleotide having a nucleotide sequence of the present invention that is present in a host plant cell that encodes the polynucleotide capable of functioning as a dsRNA or siRNA to knockdown nematode-specific sequences may have a sequence such that the encoded polynucleotide hybridizes to one or more nucleic acid sequences disclosed herein having a nucleotide sequence comprising any one of SEQ ID NOs: 1-142, a fragment or variant thereof, or a complement thereof. RNAi methods are known in the art, for example, see WO 00/44895; WO 01/36646; WO 99/32619; WO 00/01846; Mello and US20040098761.

A method of the present invention may use a recombinant DICER or RNAse III introduced into the cells of a nematode or a host plant through recombinant DNA techniques that are readily known to those skilled in the art. Both the DICER enzyme and RNAse III, which may be naturally found in a nematode or may be present due to recombinant DNA techniques, cleave larger dsRNA strands into smaller oligonucleotides. The DICER enzymes specifically cut the dsRNA molecules into siRNA fragments of about 19-25 nucleotides in length while the RNAse III enzymes normally cleave the dsRNA molecules into 12-15 base-pair siRNA.

dsRNA molecules having a sequence of one or more nucleic acid sequences disclosed herein having a nucleotide sequence comprising any one of SEQ ID NOs: 1-142, a fragment or variant thereof, or a complement thereof, may be synthesized either in vivo or in vitro. The dsRNA may be formed by a single self-complementary RNA strand which may be formed by a sequence of the present invention in nucleic acid construct in the forward direction (5′ to 3′) followed by its complementary sequence (5′ to 3′) so that an RNA transcript would form a hairpin structure, or from two complementary RNA strands. Optionally, a linking sequence may be found between the first sequence and the sequence encoding the complement to the first sequence. Endogenous RNA polymerases of the cell may mediate transcription in vivo, or a cloned RNA polymerase, provided for example by a vector, can be used for transcription in vivo or in vitro. The RNA molecules synthesized may or may not be polyadenylated, and the RNA strands may or may not be capable of being translated into a polypeptide.

The sequence of at least one strand of the dsRNA contains a region complementary to at least a part of a target gene mRNA, such as a nematode parasitism gene, sufficient for the dsRNA to specifically hybridize to the target mRNA. A target gene, such as a nematode parasitism gene or mRNA, may have a nucleotide sequence of any one of SEQ ID NOs: 1-142, a fragment or variant thereof, or a complement thereof. In an aspect, the siRNA is substantially identical to at least a portion of the target mRNA. In an aspect, a nucleic acid having one or more nucleic acid sequences disclosed herein having a nucleotide sequence comprising any one of SEQ ID NOs: 1-142, a fragment or variant thereof, or a complement thereof, has 100% sequence identity with at least a part of the target mRNA. A nucleic acid of the present invention may have 70%, 80% or greater than 90% or 95% sequence identity and may be used in methods disclosed herein. Sequence variations that might be expected due to genetic mutation, strain polymorphism, or evolutionary divergence can be tolerated. The duplex region of a dsRNA may have a nucleotide sequence that is capable of hybridizing with a portion of the target gene transcript. While the optimum length of the dsRNA may vary according to the target gene and experimental conditions, the duplex region of the RNA may be at least 10, 12, 13, 15, 19, 20, 21-23, 25, 50, 100, 200, 300, 400, 500 or more bases long.

As used herein, a target gene may be a cyst nematode gene encoding a protein, such as a protein that modulates gene expression of the host plant or host cell, formation of a syncytium, nematode migration through root tissue of the plant, cell metabolism of the plant, a protein that elicits signal transduction in the plant cell, or forms a feeding tube that enables the nematode to feed from syncytia formed in the plant. dsRNA or a nucleic acid of the present invention may be substantially identical to the entire target gene, such as the coding portion of the gene, or may be substantially identical to a part of a target gene. Those skilled in the art can select adequately sized sequences and sequences having adequate sequence homology and/or complementarity to provide nucleic acid of the present invention that can modulate gene expression of a host cell or of a nematode. A nucleic acid of the present invention may be an antisense nucleic acid specific for mRNA encoding a protein encoded by one or more nucleic acid sequences disclosed herein having a nucleotide sequence comprising any one of SEQ ID NOs: 1-142, a fragment or variant thereof, or a complement thereof. The present invention comprises a dsRNA molecule that is a silencing element. A “double stranded RNA silencing element” or “dsRNA” comprises at least one transcript that is capable of forming a dsRNA either before or after ingestion by a pest. Thus, a “dsRNA silencing element” includes a dsRNA, a transcript or polyribonucleotide capable of forming a dsRNA or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of at least two distinct RNA strands. The dsRNA molecule(s) employed in the methods and compositions of the invention mediate the reduction of expression of a target sequence, for example, by mediating RNA interference “RNAi” or gene silencing in a sequence-specific manner. In the context of the present invention, the dsRNA is capable of reducing or eliminating the level or expression of a target polynucleotide or the polypeptide encoded thereby in a pest. The dsRNA can reduce or eliminate the expression level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, for example, Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional dsRNA that are capable of reducing or eliminating the level of a sequence of interest are disclosed elsewhere herein. Accordingly, as used herein, the term “dsRNA” is meant to encompass other terms used to describe nucleic acid molecules that are capable of mediating RNA interference or gene silencing, including, for example, short-interfering RNA (siRNA), double-stranded RNA (dsRNA), micro-RNA (miRNA), hairpin RNA, short hairpin RNA (shRNA), post-transcriptional gene silencing RNA (ptgsRNA), and others.

In specific embodiments, at least one strand of the duplex or double-stranded region of the dsRNA shares sufficient sequence identity or sequence complementarity to a target polynucleotide to allow for the dsRNA to reduce the level of expression of the target sequence. As used herein, the strand that is complementary to the target polynucleotide is the “antisense strand” and the strand homologous to the target polynucleotide is the “sense strand.”

In another embodiment, the dsRNA comprises a hairpin RNA. A hairpin RNA comprises an RNA molecule that is capable of folding back onto itself to form a double stranded structure. Multiple structures can be employed as hairpin elements. In specific embodiments, the dsRNA suppression element comprises a hairpin element which comprises in the following order, a first segment, a second segment, and a third segment, where the first and the third segment share sufficient complementarity to allow the transcribed RNA to form a double-stranded stem-loop structure.

The “second segment” of the hairpin comprises a “loop” or a “loop region.” These terms are used synonymously herein and are to be construed broadly to comprise any nucleotide sequence that confers enough flexibility to allow self-pairing to occur between complementary regions of a polynucleotide (i.e., segments 1 and 3 which form the stem of the hairpin). For example, in some embodiments, the loop region may be substantially single stranded and act as a spacer between the self-complementary regions of the hairpin stem-loop. In some embodiments, the loop region can comprise a random or nonsense nucleotide sequence and thus not share sequence identity to a target polynucleotide. In other embodiments, the loop region comprises a sense or an antisense RNA sequence or fragment thereof that shares identity to a target polynucleotide. See, for example, International Patent Publication No. WO 02/00904, herein incorporated by reference. In specific embodiments, the loop region can be optimized to be as short as possible while still providing enough intramolecular flexibility to allow the formation of the base-paired stem region. Accordingly, the loop sequence is generally less than 1000, 900, 800, 700, 600, 500, 400, 300, 200, 100, 50, 25, 20, 15, 10 nucleotides or less.

The “first” and the “third” segment of the hairpin RNA molecule comprise the base-paired stem of the hairpin structure. The first and the third segments are inverted repeats of one another and share sufficient complementarity to allow the formation of the base-paired stem region. In specific embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

The first and the third segment are at least about 1000, 500, 400, 300, 200, 100, 50, 40, 30, 25, 22, 20, 19, 18, 17, 16, 15 or 10 nucleotides in length. In specific embodiments, the length of the first and/or the third segment is about 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 19 nucleotides, about 50 nucleotides to about 100 nucleotides, about 100 nucleotides to about 150 nucleotides, about 150 nucleotides to about 200 nucleotides, about 200 nucleotides to about 250 nucleotides, about 250 nucleotides to about 300 nucleotides, about 300 nucleotides to about 350 nucleotides, about 350 nucleotides to about 400 nucleotides, about 400 nucleotide to about 500 nucleotides, about 600 nt, about 700 nt, about 800 nt, about 900 nt, about 1000 nt, about 1100 nt, about 1200 nt, 1300 nt, 1400 nt, 1500 nt, 1600 nt, 1700 nt, 1800 nt, 1900 nt, 2000 nt or longer. In other embodiments, the length of the first and/or the third segment comprises at least 10-19 nucleotides; 19-35 nucleotides; 30-45 nucleotides; 40-50 nucleotides; 50-100 nucleotides; 100-300 nucleotides; about 500-700 nucleotides; about 700-900 nucleotides; about 900-1100 nucleotides; about 1300-1500 nucleotides; about 1500-1700 nucleotides; about 1700-1900 nucleotides; about 1900-2100 nucleotides; about 2100-2300 nucleotides; or about 2300-2500 nucleotides. See, for example, International Publication No. WO 0200904. In specific embodiments, the first and the third segment comprise at least 19 nucleotides having at least 85% complementary to the first segment. In still other embodiments, the first and the third segments which form the stem-loop structure of the hairpin comprises 3′ or 5′ overhang regions having unpaired nucleotide residues.

In specific embodiments, the sequences used in the first, the second, and/or the third segments comprise domains that are designed to have sufficient sequence identity to a target polynucleotide of interest and thereby have the ability to decrease the level of expression of the target polynucleotide. The specificity of the inhibitory RNA transcripts is therefore generally conferred by these domains of the silencing element. Thus, in some embodiments of the invention, the first, second and/or third segment of the silencing element comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000, or more than 1000 nucleotides that share sufficient sequence identity to the target polynucleotide to allow for a decrease in expression levels of the target polynucleotide when expressed in an appropriate cell. In other embodiments, the domain is between about 15 to 50 nucleotides, about 19-35 nucleotides, about 25-50 nucleotides, about 19 to 75 nucleotides, about 40-90 nucleotides about 15-100 nucleotides 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 19 nucleotides, about 50 nucleotides to about 100 nucleotides, about 100 nucleotides to about 150 nucleotides, about 150 nucleotides to about 200 nucleotides, about 200 nucleotides to about 250 nucleotides, about 250 nucleotides to about 300 nucleotides, about 300 nucleotides to about 350 nucleotides, about 350 nucleotides to about 400 nucleotides, about 400 nucleotide to about 500 nucleotides or longer. In other embodiments, the length of the first and/or the third segment comprises at least 10-19 nucleotides, 19-35 nucleotides, 30-45 nucleotides, 40-50 nucleotides, 50-100 nucleotides, or about 100-300 nucleotides.

In specific embodiments, the domain of the first, the second, and/or the third segment has 100% sequence identity to the target polynucleotide. In other embodiments, the domain of the first, the second and/or the third segment having homology to the target polypeptide have at least 50%, 60%, 70%, 80%, 85%, 90%, 91%, 92%, 93%, 94%, 95%, 96%, 97%, 98%, 99%, or greater sequence identity to a region of the target polynucleotide. The sequence identity of the domains of the first, the second and/or the third segments to the target polynucleotide need only be sufficient to decrease expression of the target polynucleotide of interest. See, for example, Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

The amount of complementarity shared between the first, second, and/or third segment and the target polynucleotide or the amount of complementarity shared between the first segment and the third segment (i.e., the stem of the hairpin structure) may vary depending on the organism in which gene expression is to be controlled. Some organisms or cell types may require exact pairing or 100% identity, while other organisms or cell types may tolerate some mismatching. In some cells, for example, a single nucleotide mismatch in the targeting sequence abrogates the ability to suppress gene expression. In these cells, the suppression cassettes of the invention can be used to target the suppression of mutant genes, for example, oncogenes whose transcripts comprise point mutations and therefore they can be specifically targeted using the methods and compositions of the invention without altering the expression of the remaining wild-type allele.

Any region of the target polynucleotide can be used to design the domain of the silencing element that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In specific embodiments, a domain of the silencing element shares sufficient homology to at least about 15, 16, 17, 18, 19, 20, 22, 25 or 30 consecutive nucleotides from about nucleotides 1-50, 25-75, 75-125, 50-100, 125-175, 175-225, 100-150, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of the target sequence. In some instances to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers et al. (2003) J. Biol. Chem 278:7108-7118 and Yang et al. (2002) Proc. Natl. Acad. Sci. USA 99:9442-9447, herein incorporated by reference. These studies indicate that there is a significant correlation between the RNase-H-sensitive sites and sites that promote efficient siRNA-directed mRNA degradation.

The hairpin silencing element may also be designed such that the sense sequence or the antisense sequence do not correspond to a target polynucleotide. In this embodiment, the sense and antisense sequence flank a loop sequence that comprises a nucleotide sequence corresponding to all or part of the target polynucleotide. Thus, it is the loop region that determines the specificity of the RNA interference. See, for example, WO 02/00904, herein incorporated by reference.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz et al. (2002) PNAS 99 (Suppl. 4):16499-16506 and Mette et al. (2000) EMBO J 19(19):5194-5201.

In other embodiments, the dsRNA can comprise a small RNA (sRNA). sRNAs can comprise both micro RNA (miRNA) and short-interfering RNA (siRNA) (Meister and Tuschl (2004) Nature 431:343-349 and Bonetta et al. (2004) Nature Methods 1:79-86). miRNAs are regulatory agents comprising about 19 ribonucleotides which are highly efficient at inhibiting the expression of target polynucleotides. See, for example Javier et al. (2003) Nature 425: 257-263, herein incorporated by reference. For miRNA interference, the silencing element can be designed to express a dsRNA molecule that forms a hairpin structure containing a 19-nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence.

The present invention comprises introducing heterologous genes, such as one or more nucleic acid sequences disclosed herein having a nucleotide sequence comprising any one of SEQ ID NOs: 1-142, a fragment or variant thereof, or a complement thereof, into a cellular host. Expression of the heterologous sequences results, directly or indirectly, in the intracellular production of the silencing element. These compositions may then be formulated in accordance with conventional techniques for application to the environment hosting a target pest, e.g., soil, water, and foliage of plants. See, for example, EPA 0192319, and the references cited therein.

In the present invention, a transformed microorganism can be formulated with an acceptable carrier into separate or combined compositions that are, for example, a suspension, a solution, an emulsion, a dusting powder, a dispersible granule, a wettable powder, and an emulsifiable concentrate, an aerosol, an impregnated granule, an adjuvant, a coatable paste, and also encapsulations in, for example, polymer substances.

Suitable surface-active agents include, but are not limited to, anionic compounds such as a carboxylate of, for example, a metal; carboxylate of a long chain fatty acid; an N-acylsarcosinate; mono- or di-esters of phosphoric acid with fatty alcohol ethoxylates or salts of such esters; fatty alcohol sulfates such as sodium dodecyl sulfate, sodium octadecyl sulfate, or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitan fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The compositions comprising the silencing element can be in a suitable form for direct application or as a concentrate of primary composition that requires dilution with a suitable quantity of water or other dilutent before application.

The compositions (including the transformed microorganisms) can be applied to the environment of an insect pest (such as a nematode plant pest or a cyst nematode, for example, H. glycines plant pest) by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure. For example, the composition(s) and/or transformed microorganism(s) may be mixed with grain to protect the grain during storage. It is generally important to obtain good control of pests in the early stages of plant growth, as this is the time when the plant can be most severely damaged. The compositions can conveniently contain another insecticide if this is thought necessary. In an embodiment of the invention, the composition(s) is applied directly to the soil, at a time of planting, in granular form of a composition of a carrier and dead cells of a Bacillus strain or transformed microorganism of the invention. Another embodiment is a granular form of a composition comprising an agrochemical such as, for example, a herbicide, an insecticide, a fertilizer, in an inert carrier, and dead cells of a Bacillus strain or transformed microorganism of the invention.

In an aspect, a method of the present invention comprises a transgenic plant or transgenic cell expressing a nucleic acid having one or more nucleic acid sequences disclosed herein having a nucleotide sequence comprising any one of SEQ ID NOs: 1-142, a fragment or variant thereof, or a complement thereof in an amount effective to modulate the expression of a nematode polypeptide or protein in a nematode or a plant when the nucleic acid is delivered to the nematode or the plant. Expression levels can be decreased by about 10, 20, 30, 40, 50, 60, 70, 80, or 90% compared to a control. Levels of expression of the nucleic acid used for inhibiting nematode protein expression in a transgenic plant or cell can be modulated using methods known in the art, for example using vectors with strong promoters or constitutively active promoters, high copy number vectors, or other methods known in the art. The plant or cell can be stably or transiently transformed with a nucleic acid of the present invention. In an aspect, the transformed cell may be a transgenic seed comprising or capable of expressing a nucleic acid having a sequence specific for a nematode polypeptide.

A method of the present invention comprises a method for reducing the number of nematode feeding sites established in the root tissue of a host plant, comprising providing in the host plant of a Heterodera sp. a transformed plant cell expressing a polynucleotide sequence of, or a polypeptide encoded by, any of one or more nucleic acid sequences disclosed herein having a nucleotide sequence comprising any one of SEQ ID NOs: 1-142, a fragment or variant thereof, or a complement thereof, wherein the polynucleotide is expressed to produce a double stranded ribonucleic acid that functions upon being taken up by the Heterodera sp. to inhibit the expression of a target sequence within said nematode, wherein a polynucleotide is expressed as a polypeptide, and wherein expression results in a decrease in the number of feeding sites established, relative to growth on a host lacking the transformed plant cell.

A method of the present invention comprises a method for improving the yield of a crop produced from a crop plant subjected to plant-parasitic nematode infection, which comprises a) introducing a polynucleotide selected from one or more nucleic acid sequences disclosed herein having a nucleotide sequence comprising any one of SEQ ID NOs: 1-142, a fragment or variant thereof, or a complement thereof, into a crop plant or into a cell to make a transformed cell which is grown to provide a crop plant; and b) cultivating the crop plant to allow the expression of the polynucleotide, or expression of a polypeptide encoded by the polynucleotide, wherein expression of the polynucleotide or polypeptide inhibits plant-parasitic nematode infection or growth and loss of yield due to plant-parasitic nematode infection. For example, the crop plant may be soybean (Glycine max), and the plant-parastic nematode is a Tylenchid nematode such as H. glycines.

### Controlling a Nematode Population

A method of the present invention comprises methods for controlling a population of a plant-parasitic nematode, such as H. glycines, comprising providing a composition comprising a double stranded ribonucleotide sequence that when taken up by a nematode functions to inhibit a biological function of the nematode. A composition comprises one or more nucleic acid sequences disclosed herein having a nucleotide sequence comprising any one of SEQ ID NOs: 1-142, a fragment or variant thereof, or a complement thereof. The polynucleotide sequence may exhibit from about 95 to about 100% nucleotide sequence identity along at least from about 12 to about 25 contiguous nucleotides to a target gene coding sequence derived from a nematode.

A method of the present invention comprises methods for controlling a population of a plant-parasitic nematode, such as H. glycines, comprising providing a composition comprising a polypeptide encoded by a nucleic acid of the present invention that when taken up by a nematode functions to inhibit a biological function of the nematode. A composition comprises a polypeptide, or a mutant thereof, encoded by one or more nucleic acid sequences disclosed herein having a nucleotide sequence comprising any one of SEQ ID NOs: 1-142, a fragment or variant thereof, or a complement thereof.

A nucleic acid or polypeptide of the present invention may be topically administered to one or more nematodes, or may be placed in the environment where nematodes are present so that a nucleic acid or polypeptide of the present invention may be ingested by a nematode. A plant-parasitic nematode may ingest of one or more polynucleotides or polypeptides, for example, by feeding. A plant-parasitic nematode may be contacted with a composition comprising one or more nucleic acids or polypeptides of the present invention, such as by soaking plant-parasitic nematodes with a solution comprising the nucleic acids and/or polypeptides. The uptake of a polynucleotide or polypeptide of the present invention by a plant-parasitic nematode inhibits the growth, feeding, or development of the nematode, for example by inhibiting expression of a nucleotide sequence in the plant-parasitic nematode that is substantially complementary to the sequence of the first polynucleotide, or by interfering with a biological activity of the nematode.

### Antibodies, Antibody Fragments and Binding Peptides

The present invention comprises methods and compositions comprising antibodies, antibody fragments, and binding peptides to polypeptides encoded by one or more nucleic acid sequences disclosed herein having a nucleotide sequence comprising any one of SEQ ID NOs: 1-142, a fragment or variant thereof, or a complement thereof. Such antibodies may be used in methods for inhibiting biological activity of a nematode parasitism gene product. An antibody or fragment thereof may be encoded by a vector present in a transformed cell and expressed therein and specifically bind to a target gene polypeptide of a nematode to inhibit the biological activity or expression of the nematode parasitism gene product. An antibody or antibody fragment specifically binds to a parasitic nematode gene product. The generation of antibodies is known in the art. Based on the nucleic acid sequences provided herein, one of skill in the art could readily produce antibodies to the polypeptides encoded by one or more nucleic acid sequences disclosed herein having a nucleotide sequence comprising any one of SEQ ID NOs: 1-142, a fragment or variant thereof, or a complement thereof. The antibody sequence could be cloned and one or more of the antibodies or antigen binding antibody fragments can be expressed in a plant or plant cell so that the antibody binds the polypeptide encoded by one or more of one or more nucleic acid sequences disclosed herein having a nucleic acid sequence of SEQ ID NOs:1-142. Binding of the nematode protein or nucleic acid by the antibody or antibody fragment may inhibit the activity of the parasitic nematode gene product and thereby provide the plant expressing the antibody or antigen binding antibody fragment with resistance to the parasitic nematode. The present invention also contemplates antibodies to functional mutants of the polypeptides of the present invention.

Methods of the invention comprise methods for controlling a pest, i.e., a nematode plant pest, such as, a cyst nematode, for example, H. glycines, plant pest. A method comprises feeding to a pest a composition comprising a nucleic acid construct, such as a silencing element of the invention, or a polypeptide of the present invention, wherein the nucleic acid or polypeptide, when ingested by a pest (i.e., a nematode plant pest, such as, a cyst nematode, for example, H. glycines), control the pest, for example by reducing the level of a target polynucleotide of the pest. The pest can be fed a nucleic acid or polypeptide of the present invention in a variety of ways. For example, in one embodiment, a polynucleotide comprising a silencing element is introduced into a plant. As the nematode plant pest, such as, a cyst nematode, for example, H. glycines, plant pest feeds on the plant or part thereof expressing these sequences, the silencing element is delivered to the pest. When the silencing element is delivered to the plant in this manner, it is recognized that the silencing element can be expressed constitutively or alternatively, it may be produced in a stage-specific manner by employing the various inducible or tissue-preferred or developmentally regulated promoters that are discussed elsewhere herein. In specific embodiments, the silencing element expressed in the roots, stalk or stem, leaf including pedicel, xylem and phloem, fruit or reproductive tissue, silk, flowers and all parts therein or any combination thereof.

In another method, a composition comprising at least one silencing element of the invention is applied to a plant. In such embodiments, the silencing element can be formulated in an agronomically suitable and/or environmentally acceptable carrier, which is preferably, suitable for dispersal in fields. In addition, the carrier can also include compounds that increase the half-life of the composition. In specific embodiments, the composition comprising at least one silencing element is formulated in such a manner such that it persists in the environment for a length of time sufficient to allow it to be delivered to a pest. In such embodiments, the composition can be applied to an area inhabited by a pest. In one embodiment, the composition is applied externally to a plant (i.e., by spraying a field) to protect the plant from pests.

In certain embodiments, the nucleic acid constructs of the present invention can be stacked with any combination of polynucleotide sequences of interest in order to create plants with a desired trait. A trait, as used herein, refers to the phenotype derived from a particular sequence or groups of sequences. For example, the polynucleotides of the present invention may be stacked with any other polynucleotides encoding polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48:109), lectins (Van Damme et al. (1994) Plant Mol. Biol. 24:825, pentin (described in U.S. Pat. No. 5,981,722), and the like. The combinations generated can also include multiple copies of any one of the polynucleotides of interest. The polynucleotides of the present invention can also be stacked with any other gene or combination of genes to produce plants with a variety of desired trait combinations including, but not limited to, traits desirable for animal feed such as high oil genes (e.g., U.S. Pat. No. 6,232,529); balanced amino acids (e.g., hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122) and high methionine proteins (Pedersen et al. (1986) J. Biol. Chem. 261:6279; Kirihara et al. (1988) Gene 71:359; and Musumura et al. (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)); the disclosures of which are herein incorporated by reference.

The polynucleotides of the present invention can also be stacked with traits desirable for disease or herbicide resistance (e.g., fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)); the disclosures of which are herein incorporated by reference. One could also combine the polynucleotides of the present invention with polynucleotides providing agronomic traits such as male sterility (e.g., see U.S. Pat. No. 5,583,210), stalk strength, flowering time, or transformation technology traits such as cell cycle regulation or gene targeting (e.g., WO 99/61619, WO 00/17364, and WO 99/25821); the disclosures of which are herein incorporated by reference.

These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

The present invention comprises methods and compositions that may be used with any monocot and/or dicot plant, depending on the nematode control desired. The present invention comprises control of plant disease in soybean plants by modulating the activity of a parasitic nematode SCN or Heterodera sp., or H. glycines. Host plants of parasitic nematodes include, but are not limited to, monocots, dicots, alfalfa, artichoke, asparagus, banana, barley, beans, beet, broccoli, cabbage, canola, carrot, cassava, cauliflower, cereals, corn, cotton, cucumber, grape, oat, onion, pea, peanut, potato, rice, rye, sorghum, soybean, spinach, squash, sugarbeet, sugarcane, sunflower, tobacco, tomato, turfgrass, and wheat plants, and members of the phylogenic family Leguminosae, Chenopodiaceae, Cruciferae, and Solanaceae.

### DEFINITIONS

Throughout this disclosure, various publications, patents and published patent specifications are referenced. Each of these is hereby incorporated by reference in its entirety into the present disclosure to more fully describe the state of the art. Unless otherwise indicated, the disclosure encompasses conventional techniques of plant breeding, immunology, molecular biology, microbiology, cell biology and recombinant DNA, which are within the skill of the art.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience, 1999; and Robert A. Meyers (ed.), Molecular Biology and Biotechnology, a Comprehensive Desk Reference, published by VCH Publishers, Inc., 1995; Ausubel et al. (1987) Current Protocols in Molecular Biology, Green Publishing; Sambrook and Russell. (2001) Molecular Cloning: A Laboratory Manual 3rd. edition.

As used herein, to “modulate” the expression of a target gene in a plant or nematode cell means that the level of expression of the target gene in the cell after applying a method of the present invention is different from its expression in the cell before applying the method. To modulate gene expression may mean that the expression of the target gene in the plant or nematode is reduced, preferably strongly reduced, or that the expression of the gene is not detectable. The modulation of the expression of an essential gene may result in a knockout mutant phenotype in host plant or nematode cells or plants or nematodes derived therefrom. Modulated expression can include up-regulating or down-regulating the expression of plant or nematode genes.

As used herein, “antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA to lead to the production of siRNA. Antisense and sense RNAs may be synthesized with an RNA synthesizer. Antisense and sense RNAs may be expressed intracellularly from DNAs coding for antisense and sense RNAs (antisense and sense DNAs) which provide for intracellular accumulation of dsRNA and siRNA.

As used herein, “control sequences” means DNA sequences necessary for the expression of an operably linked coding sequence in a particular host organism. Control sequences that are suitable for prokaryotes, for example, include a promoter, optionally an operator sequence, a ribosome binding site, and the like. Eukaryotic cells are known to use promoters, polyadenylation signals, and enhancers.

As used herein, the term “cell” refers to a membrane-bound biological unit capable of replication or division.

As used herein, the term “nucleic acid construct” refers to a recombinant genetic molecule comprising one or more polynucleotide sequences, and may comprise a polynucleotide of the present invention. For example, genetic constructs used for transgene expression in a host organism may comprise in the 5′-3′ direction, a promoter sequence; a sequence encoding a nucleic acid disclosed herein, and a termination sequence. If present, the open reading frame of a nucleic acid of the present invention may be orientated in either a sense or anti-sense direction. A construct may also comprise selectable marker(s) and other regulatory elements for expression.

As used herein, two nucleic acid molecules are said to be capable of specifically hybridizing to one another if the two molecules are capable of forming an anti-parallel, double-stranded nucleic acid structure. A nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional low-stringency conditions. Similarly, the molecules are said to be complementary if they can hybridize to one another with sufficient stability to permit them to remain annealed to one another under conventional high-stringency conditions. Conventional stringency conditions are described by Sambrook, et al. (1989), and by Haymes et al. (1985). Departures from complete complementarity are permissible, as long as such departures do not completely preclude the capacity of the molecules to form a double-stranded structure. For a nucleic acid molecule or a fragment of the nucleic acid molecule to serve as a primer or probe it needs only be sufficiently complementary in sequence to be able to form a stable double-stranded structure under the particular solvent and salt concentrations employed.

As used herein, the term “control element” or “regulatory element” are used interchangably herein to mean sequences positioned within or adjacent to a promoter sequence so as to influence promoter activity. Control elements may be positive or negative control elements. Positive control elements require binding of a regulatory element for initiation of transcription. Many such positive and negative control elements are known.

The term “cyst nematode” refers to a member of Heterodera or Globodera spp. and includes, but is not limited to Heterodera glycines and Heterodera schachtii. Additional Heterodera species include but are not limited to H. avenae, H. bifenestra, H. cajani. H. carotae, H. ciceri, H. cruciferae, H. cynodontis, H. cyperi, H. davert, H. elachista, H. fii, H. galeopsidis, H. goettingiana, H. graminis, H. hordecalis, H. humuli, H. iri, H. latipons, H. lespedeza, H. leucilyma, H. Iongicaudata, H. mani, H. maydis, H. medicaginis, H. oryzae, H. oryzicola, H. sacchari, H. salixophila, H. sorghii, H. trifoii, H. urticae, H. vigna, H. zeae. Representative Globodera species include but are not limited to G. achilleae, G. artemisiae, G. hypolysi, G. leptonepia, G. mali, G. pallida, G. rostochiensis, G. tabacum, and G. zelandica.

The term “heterologous” refers to elements occurring where they are not normally found. For example, a promoter may be linked to a heterologous nucleic acid sequence, e.g., a sequence that is not normally found operably linked to the promoter.

The term “host plant” refers to a plant that is susceptible to nematode infection.

As used herein, “identity”, as known in the art, is the relationship between two or more polynucleotide or polypeptide sequences, as determined by comparing the nucleic acid or amino acid sequences, respectively. In the art, identity also means the degree of sequence relatedness between polynucleotide sequences, as determined by the match between strings of such sequences. Identity can be readily calculated (Sequence Analysis in Molecular Biology, von Heinje, G., Academic Press, 1987; and Sequence Analysis Primer, Gribskov, M. and Devereux, J., eds., M Stockton Press, New York, 1991). While there exist methods to measure identity between two polynucleotide sequences, the term is well known to those skilled in the art. Methods commonly employed to determine identity between sequences include, but are not limited to those disclosed in Carillo, H., and Lipman, D., SIAM J. Applied Math., 48:1073 (1988). Preferred methods to determine identity are designed to give the largest match between the sequences tested. Methods to determine identity are codified in computer programs. Computer program methods to determine identity between two sequences include, but are not limited to, GCG program package, BLASTP, BLASTN, FASTA, and CLUSTAL program. It compares the sequences of two polynucleotides and finds the optimal alignment by inserting spaces in either sequence as appropriate. The identity for an optimal alignment can also be calculated using a software package such as BLASTx. This program aligns the largest stretch of similar sequence and assigns a value to the fit.

As used herein, the phrase “induce expression” means to increase the amount or rate of transcription and/or translation from specific genes by exposure of the cells containing such genes to an effector or inducer reagent or condition.

As used herein, the term “isolated,” when used to describe the nucleic acid molecules or polypeptides disclosed herein, means a substance that has been identified and separated and/or recovered from a component of its natural environment. For example an isolated polypeptide or polynucleotide is free of association with at least one component with which it is naturally associated. An isolated substance includes the substance in situ within recombinant cells. Ordinarily, however, an isolated substance will be prepared by at least one purification step. An isolated nucleic acid molecule is other than in the form or setting in which it is found in nature.

As used herein, the term “nematode esophageal glands” or “nematode esophageal gland cell” refers to three large, transcriptionally active gland cells, one dorsal and two subventral, located in the esophagus of a nematode and that are the principal sources of secretions (parasitism proteins) involved in infection and parasitism of plants by plant-parasitic nematodes in the orders Tylenchida and Aphelenchida.

As used herein, a nucleic acid sequence or polynucleotide is “operably linked” when it is placed into a functional relationship with another nucleic acid sequence. Generally, “operably linked” means that the DNA sequences being linked are contiguous and may be contiguous and in reading frame. Linking can be accomplished by ligation at convenient restriction sites. If such sites do not exist, synthetic oligonucleotide adaptors or linkers are used in accordance with conventional practice.

As used herein, the terms “parasitism proteins, parasitism polypeptides” refers to molecules involved in nematode parasitism of plants. Products of parasitism genes are present in plant-parasitic nematode esophageal gland cells, where they may be expressed or may control aspects of cellular activities, and are involved in mediating parasitism of plants.

As used herein, the term “percent (%) nucleic acid sequence identity” is defined as the percentage of nucleotides in a candidate sequence that are identical with the nucleotides in a reference nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

As used herein, the term “sequence identity”, “sequence similarity” or “homology” is used to describe sequence relationships between two or more nucleotide sequences. The percentage of “sequence identity” between two sequences is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As used herein, the term “substantially homologous” or “substantial homology”, with reference to a nucleic acid sequence, includes a nucleotide sequence that hybridizes under stringent conditions to the coding sequence of any of SEQ ID NOs:1-142 as set forth in the sequence listing, or the complements thereof. Sequences that hybridize under stringent conditions to any of SEQ ID NOs:1-142 or the complements thereof, are those that allow an antiparallel alignment to take place between the two sequences, and the two sequences are then able, under stringent conditions, to form hydrogen bonds with corresponding bases on the opposite strand to form a duplex molecule that is sufficiently stable under the stringent conditions to be detectable using methods well known in the art. Substantially homologous sequences have preferably from about 70% to about 80% sequence identity, or from about 80% to about 85% sequence identity, or from about 90% to about 95% sequence identity, to about 99% sequence identity, to the referent nucleotide sequences as set forth in any of SEQ ID NOs:1-142, in the sequence listing, or the sequences complementary to SEQ ID NOs:1-142.

As used herein, the term “plant” is used in it broadest sense. It includes, but is not limited to, any species of woody, ornamental or decorative, crop or cereal, fruit or vegetable plant, and photosynthetic green algae (e.g., Chlamydomonas reinhardtii). It also refers to a plurality of plant cells that are largely differentiated into a structure that is present at any stage of a plant's development. Such structures include, but are not limited to, a fruit, shoot, stem, leaf, flower petal, etc. The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

As used herein, the term non-naturally occurring plant refers to a plant that does not occur in nature without human intervention. Non-naturally occurring plants include transgenic plants and plants produced by non-transgenic means such as plant breeding.

As used herein, the term “plant cell” refers to a structural and physiological unit of a plant, comprising a protoplast and a cell wall. The plant cell may be in the form of an isolated single cell or a cultured cell, or as a part of higher organized unit such as, for example, a plant tissue, a plant organ, or a whole plant.

As used herein, the term “plant cell culture” refers to cultures of plant units such as, for example, protoplasts, cell culture cells, cells in plant tissues, pollen, pollen tubes, ovules, embryo sacs, zygotes and embryos at various stages of development.

As used herein, the term “plant material” refers to leaves, stems, roots, flowers or flower parts, fruits, pollen, egg cells, zygotes, seeds, cuttings, cell or tissue cultures, or any other part or product of a plant.

As used herein, the term “plant organ” refers to a distinct and visibly structured and differentiated part of a plant such as a root, stem, leaf, flower bud, or embryo.

As used herein, the term “plant tissue” refers to a group of plant cells organized into a structural and functional unit. Any tissue of a plant whether in a plant or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

As used herein, the term “promoter” refers to a regulatory nucleic acid sequence, typically located upstream (5′) of a gene or protein coding sequence that, in conjunction with various elements, is responsible for regulating the expression of the gene or protein coding sequence. The promoters suitable for use in the constructs of this disclosure are functional in plants and in host organisms used for expressing the inventive polynucleotides. Many plant promoters are publicly known. These include constitutive promoters, inducible promoters, tissue- and cell-specific promoters and developmentally-regulated promoters.

As used herein, the term “purifying” means increasing the degree of purity of a substance in a composition by removing (completely or partially) at least one contaminant from the composition. A “purification step” may be part of an overall purification process resulting in an “essentially pure” composition. An essentially pure composition contains at least about 90% by weight of the substance of interest, based on total weight of the composition, and can contain at least about 95% by weight.

As used herein, the term “small RNA molecules” refer to single stranded or double stranded RNA molecules generally less than 200 nucleotides in length. Such molecules are generally less than 100 nucleotides and usually vary from 10 to 100 nucleotides in length. In an aspect, small RNA molecules have 11, 12, 13, 14, 15, 16, 17, 18, 19, 20, 21, 22, 23, 24, 25, 26, 27, 28, 29 or 30 nucleotides. Small RNAs include microRNAs (miRNA) and small interfering RNAs (siRNAs). MiRNAs may be produced by the cleavage of short stem-loop precursors by Dicer-like enzymes; whereas, siRNAs may be produced by the cleavage of long double-stranded RNA molecules. MiRNAs are single-stranded, whereas siRNAs are double-stranded. The term “siRNA” means a small interfering RNA that is a short-length double-stranded RNA that is not toxic. Generally, there is no particular limitation in the length of siRNA as long as it does not show toxicity. “siRNAs” can be, for example, 15 to 49 bp, 15 to 35 bp, or 21 to 30 bp long. Alternatively, the double-stranded RNA portion of a final transcription product of siRNA to be expressed can be, for example, 15 to 49 bp, 15 to 35 bp, or 21 to 30 bp long. The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. Nonpairing portions can be contained to the extent that they do not interfere with siRNA formation. The “bulge” used herein preferably comprises 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number. The structures of siRNAs are known to those skilled in the art. As long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

As used herein, the term “signal peptide” refers to a short (15-60 amino acids long) amino terminal peptide chain that directs the post translational transport of a protein; usually directs the peptide to the secretory pathway of the cell.

As used herein, the term “genome” as it applies to cells of a plant-parasitic nematode or a host encompasses not only chromosomal DNA found within the nucleus, but organelle DNA found within subcellular components of the cell. The nucleic acids of the present invention when introduced into plant cells may be either chromosomally integrated or organelle-localized. The term “genome” as it applies to bacteria encompasses both the chromosome and plasmids within a bacterial host cell. The nucleic acids of the present invention when introduced into bacterial host cells can therefore be either chromosomally integrated or plasmid-localized.

As used herein, the term “plant-parasitic nematode” refers to those nematodes that may infect, colonize, parasitize, or cause disease on host plant material. As used herein, a “nematode resistance” trait is a characteristic of a transgenic plant, transgenic animal, or other transgenic host that causes the host to be resistant to attack from a nematode that typically is capable of inflicting damage or loss to the host. Such resistance can arise from a natural mutation or more typically from incorporation of recombinant DNA that confers plant-parasitic nematode resistance. A method of conferring nematode resistance to a transgenic plant comprises a recombinant DNA entering a plant cell and being transcribed into a RNA molecule that forms a dsRNA molecule within the tissues or fluids of the recombinant plant. The dsRNA molecule is comprised in part of a segment of RNA that is identical to a corresponding RNA segment encoded from a DNA sequence within a plant-parasitic nematode that may cause disease on the host plant. Expression of the gene within the target plant-parasitic nematode is suppressed by the dsRNA, and the suppression of expression of the gene in the target plant-parasitic nematode results in the plant being resistant to the nematode. Fire et al. (U.S. Pat. No. 6,506,599) generically describes inhibition of pest infestation, providing specifics only about several nucleotide sequences that were effective for inhibition of gene function in the nematode species Caenorhabditis elegans. US 2003/0061626 describes the use of dsRNA for inhibiting gene function in a variety of nematode pests. US 2003/0150017 describes using dsDNA sequences to transform host cells to express corresponding dsRNA sequences that are substantially identical to target sequences in specific pests, and particularly describe constructing recombinant plants expressing such dsRNA sequences for ingestion by various plant-parasitic nematode, facilitating down-regulation of a gene in the genome of the target organism and improving the resistance of the plant to the plant-parasitic nematode.

As used herein, the term “soybean cyst nematode” or “SCN” refers to a nematode belonging to Heterodera glycines.

As used herein, the term “transformed,” “transgenic,” “transfected” and “recombinant” refer to a host organism such as a prokaryotic or eukaryotic cell, for example a bacterium or a plant cell, into which a heterologous nucleic acid molecule has been introduced, for example by molecular biology techniques known to those skilled in the art for introducing nucleic acids into a cell, plant, bacterium or animal cell, including transfection with viral vectors, transformation by Agrobacterium, with plasmid vectors, and introduction of naked DNA by electroporation, lipofection, and particle gun acceleration, and includes transient as well as stable transformants. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule. A “transformed cell” refers to a cell into which has been introduced a nucleic acid molecule, for example by molecular biology techniques. The term “transgenic plant” refers to a plant or tree that contains recombinant genetic material not normally found in plants or trees of its type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems and any other parts of the plant, its products and offspring.

As used herein, the term “translation initiation enhancer sequence”, as used herein, refers to a nucleic acid sequence that can determine a site and efficiency of initiation of translation of a gene (See, for example, McCarthy et al., 1990, Trends in Genetics, 6: 78-85). A translation initiation enhancer sequence can extend to include sequences 5′ and 3′ to the ribosome binding site. The ribosome binding site is defined to include, minimally, the Shine-Dalgarno region and the start codon, in addition to any bases in between. In addition, the translation initiation enhancer sequence can include an untranslated leader or the end of an upstream cistron, and thus a translational stop codon. See, for example, U.S. Pat. No. 5,840,523.

As used herein, the term “vector” refers to a nucleic acid molecule which is used to introduce a polynucleotide sequence into a host cell, thereby producing a transformed host cell. A “vector” may comprise genetic material in addition to the above-described genetic construct, e.g., one or more nucleic acid sequences that permit it to replicate in one or more host cells, such as origin(s) of replication, selectable marker genes and other genetic elements known in the art (e.g., sequences for integrating the genetic material into the genome of the host cell, and so on).

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight; temperature is in degrees centigrade; and pressure is at or near atmospheric.

In general, in the following claims, the terms used should not be construed to limit the invention to the specific embodiments disclosed in the specification and the claims.

The entire disclosure of each document cited (including patents, patent applications, journal articles, abstracts, manuals, books, or other disclosures) in the Background of the Invention, Detailed Description, and Examples is herein incorporated by reference in their entireties.

### REFERENCES

Chen P Y, Wang C K, Soong S C, To K Y. 2003. Complete sequence of the binary vector pBI121 and its application in cloning T-DNA insertion from transgenic plants. Molecular Breeding 11, 287-293.
Clough S J, Bent A F. 1998. Floral dip: a simplified method for Agrobacterium-mediated transformation of Arabidopsis thaliana. Plant Journal. 16, 735-743.
deBoer, J. M., Y. Yan, J. Bakker, E. L. Davis, and T. J. Baum. 1998. In situ hybridization to messenger RNA of Heterodera glycines. Journal of Nematology: 30:309-312.
De Boer J M, Yan Y T, Wang X H, Smant G, Hussey R S, Davis E L, Baum T J. 1999. Developmental expression of secretory beta-1,4-endoglucanases in the subventral esophageal glands of Heterodera glycines. Molecular Plant-Microbe Interactions 12, 663-669.
Gleave A P. 1992. A versatile binary vector system with a T-DNA organizational-structure conducive to efficient integration of cloned DNA into the plant genome. Plant Molecular Biology 20, 1203-1207.
Livak K J, Schmittgen T D. 2001. Analysis of relative gene expression data using real-time quantitative PCR and the 2(T)(-Delta Delta C) method. Methods 25, 402-408.
Maeda, I., Kohara, Y. Yamamoto, M. & Sugimoto, A. Large-scale analysis of gene function in Caenorhabditis elegans by high-throughput RNAi. Curr. Biol. 11, 171-176 (2001).
Murashige, T, Skoog F. 1962. A revised medium for rapid growth and bioassays with tobacco tissue cultures. Physiologia Plantarum 15, 473-497.
Sijmons P C, Grundler F M W, von Mende N, Burrows P R, Wyss U. 1991. Arabidopsis thaliana as a new model for plant-parasitic nematodes. Plant Journal 1, 245-254.
Wesley S V, Helliwell C A, Smith N A, Wang M B, Rouse D T, Liu Q, Gooding P S, Singh S P, Abbott D, Stoutjesdijk, P A, Robinson S P, Gleave A P, Green A G, Waterhouse P M. 2001. Construct design for efficient, effective and high-throughput gene silencing in plants. Plant Journal 27, 581-590.

### EXAMPLES

### Example 1

Nucleic acid sequences of the present invention comprise the following nucleic acid sequences. These sequences are exemplary cyst nematode genes derived from esophageal glands. Such sequences or their complements may be the targets for binding with inhibitory nucleic acid sequences having the same or a complementary sequence. Methods for making inhibitory sequences are known in the art. DNA constructs, vectors, transgenic cells, plants, seeds or products described herein may comprise one or more of the following nucleic acid or amino acid sequences, or a portion of one or more of the disclosed sequences. These nucleic acids may encode polypeptides which may be involved in parasitism activities of a nematode, or may be involved in the infection cycle of a nematode in a host plant. Other than a parasitism function, a polypeptide encoded by a nucleic acid sequence of the present invention may have other functions in a plant or nematode. The present invention comprises SEQ ID. No. 1-142 nucleic acid sequences and SEQ ID NO. 143-159 amino acid sequences, provided below. The official copy of the sequence listing is submitted with the specification as a text file via EFS-Web, in compliance with the American Standard Code for Information Interchange (ASCII). The sequence listing filed via EFS-Web is part of the specification and is hereby incorporated in its entirety by reference herein, and includes comprises SEQ ID. No. 1-142 nucleic acid sequences and SEQ ID NO. 143-159 amino acid sequences,

1. hgg1c.pk001.e18
caaaagctcggcactccgacgtggacgagatcatacatttgcgcaaccggcttacatgcgtgacccgttgcgtgccgaccttctcgcgggc
tccaaactgaaggaggtgaagaagacggactacaaccagtgcaagtccatgctgctcgacctgttcgacggcacgcgcgtgattttggtgg
gcgaaacgcgggaccgaagcggacgcaagcggttgatctcctgctttcaactgtaccgacaaagcagagccgcggcaaatttcggcatgt
tcgctgtccatccctttttccaagcgtccggacttggcaagcgattgttgactgttgctgaacgctatgcccgtattgtgtggggcagtgacga
gatgcatttggatgttggcgggagtttggccgaattaaagttgggcatgggacgactgcagagatactacaagcggcgcgggttcctatcaa
ccggcattcttcgccccttcaatggggctgtggcgcgcttcatcacggtagaccgaaacgatctgtggattgagctgatggtcaaggacata
cgtggagcattggatgacatcggcggagatccagagaaacggatgaaaagagtgaacagtcgggggagattggccagagaagcagac
aaagacgacggcggcagagatccacaaaaaaggatggagagagtgcgaagctttgggagattaaccatagaagcagacagggacgac
atcggcagagacgcgcaaaaaaggatggagagagtgcgcagtttagggagattggcaagagaagcagacaaatcggatgagagtaaa
ggcaaagatggggaggaaaagaaaaagacaacacaggcagagggggaagagagtaaaggcaaagatggagaggaaaagaaaaag
acaacacaggcagagggggaagagagaattaagcctttggctgattgaagaagcattcaaacagttgtgtctcctcgaaaaatacagactct
gaagcttcaatacagtaaatacagtatgcttgtcccggaataatttaatgaatgtcatcgttttttttattaaaaatttttcaaatcgttgccagttggc
gtttcgtcgtagttatactgtagaaagattggcaaaaataaatgtttctggcttaa
2. hgg1c.pk002.a5
catccattgatttagcccctattattggatttatcccgcttttccttctttcgctcctccccttctgaactcttatttatacctctttttgcccccatataatt
attctgccaattttccattggcatggctctctctgcccttctcctcctccttcctctgcttctcaatgtgcaaaatatcccagatgagtccgttcaatc
ggatgtgaaagccgttgattcggccatttcgtcgctggaacaatggaaggacccgcgcaattcgttggcatcactcgactcacagctgacag
agccccaacgagcactggccaaaatgttttgggaattggagaccatcgaaaaggaaaagccgaaggcaccgccacaattcgacttggga
cttttcttggaagctttggaagcgatggtcgaaatgaacgaagaagcaaaggaagtgaagctgagaaaggacaaactgaccgaatgggca
ggcggagagaaagcaaacgaagggaaagaagggaagacgaaggaggaggagacagtgccggaagtgagagttaatgagaatgtaaa
ggtggaagtgacgaacggcgccggaggggacggaaagatggaagtcaagcgaggaaaggacgagaacggaaacgagcaggtggtg
gtcacctttgtgaagagggacggaacggagggaaagacggaggaggaacagaagaaagaggagaaggacaacctacggaagggac
gggaggaggtcaagatggagcaggacaacgtagaaggggcaccgaaaacggactcggccaacagtgccaagtcacccattccaatgc
ccaccattttgtcctccccggccgcaccggcagaggaggaggaaaaggcgaacgatgcgttcacagaagcaaatgtgaggaaaaaggt
gaaaaaggacgaagaaatgttcataattatgactgatgacaacggaaggacgggaaatgcgaatgaaagacaaatggaatttgtcagaatg
ccaaaaaaagttgggagagacttcggcagcgaattgttcggtttgccacaaccttcgaacggcggacaaagcccaatggaaatgtttttcaa
tttgtttggacgaaaaaaaagggaaacggtgcaggaaggaagaaagaaacggagcatcgaaaatttagccaatttggggaagccgggct
cagagtttgtg
3. hgg1c.pk003.d19
acgcgggaaagggaaaaatgccgctaagaaagacaaaacaaagaacaaaaaggcaccagcagcagccaagccaaaagctgagcctg
ttgagactgaagagccatccagtgctcaagttgtagctgaacaggacggaagcgatgagtcagctaacaaccaagaaatggatgccggcg
aagagattgcagaggaggagcagactgatttggcacaggatgaacagcttgaagacgatgccacggacggtgaagaaggaaatggtatg
gctgaggaagaacagccggagatcaactaataaactatttttagaaaaatatttaggaaaataattttctatgggtgaaatgtagctgtagttttc
cactgatgtgtaaatgtatattttac
4. hgg1c.pk003.g23
aattccatcaaatctgccaaagatccttcaaaaatgtcttctccttcttcgtccgtctctctactcgccatcgtcacaattttctgtttgctgtgcaaa
tgttgcgtttcggcaccgcatccgtgctgtcccggcagtcaaaaagtggtttcgctgatggccaattacgttggcactttcgcccattccttttca
aaggcatcgctttgttcggatgcccaaagtgttgcgggcgcattgaaaggccaactgatcggctgctcgaagggcggcgacgcaactctttt
ggccgacatcgaagcatctcttgccactcattctgctgatgagtgtgcccacagcctcggcttcgtccgtgccatgttcgccattgccgcctcc
gcttcttcccatgccagcaacaacaacgaatggcaggcattgagtgcccagtttggtcagcaaatcagtgaaattgactcgaaatgtgccga
gtttggcattggcattgccaaagtgccatatgacggccccaagggtgatcactcccaacgaaatgtgcatggcacggacagtgtaattgcca
tgcctggattggccggctcacacaaacaatgaatagaatcaatgggtcactgaatggaacgaaatgattgtggagttcgtttttgatattgtcct
tcttttagttgatgaatagtaaaaataaatttaag
5. hgg1c.pk004.a14
gacatcattaatatattttattcattattaaataaaaaatctttttgccatgttttctctgatgctctccatcttcccaattgtctttttggtctgttgcaag
gcaatgccaaatttcccgtgctgcccgggaagtcagcaagtggttgctgtgatgtccaattacattggcactttcactagtgaggacaaatcta
cagtatgctcaaccgcaaaaaatactgtggaaggaataaaaagtgaactttcatctcgcgtgggatgcccaagcggaggagaagcacaaa
ttgtgaacgaaatcgaccgacagctgactaacattgcgaaaatggaaatcaattatgaggacgagtgcccgtacaatttgggctttgcccgtg
ccatgttcgacttggccgctgctgctggccatgcgggcaacgacacagaatggcaaaacatgaaaagcaaatttgtacaggaaagccaag
caatcaaagcaattggccaagaaatgaacattgaagttacggatgtgcacattggacacccaagcaaagggatttccgcgcaccaaaatgt
gccaagtccaagccatgtgattgccaaccctggccaacacagttcggttggccatggaaaggaagacacaccgttgtcatcggatttcgatt
tttgagggcatagaaa
6. hgg1c.pk004.a16
tatatatttattaattctctttaaatctttaaaatgaaaataatttctattctcatcaattttattctggctatctatgaagcaaaaggtggaggaattgttt
ctttactatcaagaagacaagcaccaaagcgtcatttagctagttcactgcgtcaacaacgcaccgaggacaatcacatttcaattaatggac
aaaattacgcggttgacggacctaatgttaatgttggtgttgaagggcatgatttgagtgtgaatgggagagtttatcaaaacagggccacag
agcagtatctggaaattatacaagacaaaaacataagaaatgtaattgtcagtgtgccattatcgttattttctcgcgaaaacataatcgatggg
caaataaacgctaaatgcaatggaaatttatacatcgatcaatcgtcagatggatgttctcgcataatatgcgtcgacgataaaaagaatggcg
ttgaaaataactttggacagacacgtgatattttcctgaccggtgatgtcaatatttttgagtctgcaaatggaattatctacaactctatgatggg
aggaactttacatatccataattcgtcacttgagtgtgctaacattgaatgtgatgcatctttaaatgtaactcactcaccaatagaacgtaatgcg
caaatgaaatgtggtgggagtttaagtattgatgagtcaccaatgggaaatattcggcttaactgtgatggatctttgcggatcgaaaaatcga
aaatggaaagcagtcagattgatgttggtggaagcattgggattgttgagtcaccaatgggaagtattgggattgactgtggtggatctttacg
gatcgaaaagtcgaaaatggaaattggcaacctagactgtggaggaagtttaaccattgtagaatcgacagcgcaaagtctaaagttaaact
gtggaggaagtttaaatatgaaggagtcgccaatgaaaaatgttggcattaattgtgatggaagtgcaaccattaagaagtcgaaaatggaa
agtggtcgcattaattgtggtggcaatttttctattgatagttcgccaacgggaagtgttcgaattgattacggtggaagaagaattaatttatga
ggtcaaacgaatgatcttgttcggaac
7. hgg1c.pk004.a22
ttttacaaaaaaaagaatattttttaataaaaaccattaagcaactaacataatggccattcttctgaagtttgttctgttcatctcaataatggcaatt
ttctgcgattgtatggaccccggcaaaaatgggaaaaacgaaaaaaaagacgttgtaaaacaaaaagtggacgaaacgaaagttgagcgc
gccagtgaaatgaacaaaggcaaaagcatcgttatggctgactccaaaaaggaaggcacaacgacagtgaaaattccgcaccgttatgga
gcagtgtcggggatgagtggccaaaatgccagtccagaagcctctcaaattggcagtccaaaaaacagtccaaagggcactcaaattggc
agtccaagatccattagcagtcctaaatcaacacaaattggaagcccaaaaggcattcaaattggcagtccacgaaaagaaaagaccaaat
tatcttcagctgttggctcttctgatttcaatgttatcgacgaatcaaaagaagcgaaaaaaaccaagccaattcaaaccgagtccgtccagaa
gccaaaataaacgcgaacagcagcgactcaatgttactattggagaagcgggaagagttcaatcatcaaaaagagtgtcgagcaaagaca
cctttagtccgtcaaggaaaaaaaaaaaaaaaaaaaaaaaaa
8. hgg1c.pk004.h22
acgcgggggacagattgctgactatgcaggaaattcatctgagagtgccgaaggacgtttactccgaataccacaaattggtgaaggaccc
cgaagacagcaaaaaaaataatcccatccaaaacgattccgggaagagttgtcgaaatccaacggaccagcgacaatttgtacagagcgtt
ggcttatgcactgacgggcaccgaaatgcttcacaaggcgactcggatggttgtgctcgaatactttgagagtttcttcggacaatgggacaa
aaagcaggcgcagccgtggatggacgaatacgaagtgcgaagtgtgcgcagacaggcggagaaaataaaggcgggcaaagccgggg
gcacggtcgagctgatagcggcggcgaaaaagttcaacatgaacgtgctggtctacaagacggacaaggacatgtggctgtgcatgtcgc
caaagacggcgcacaaatgggacttggacaagaactgccaaagcaaggatgcgatgaccattgcgttggaattgtacgacaacgaaaatt
acgacgtgattatggacgtgcaacaaaagaagtgaacggagaggcggacggacggtcaactcaaaaagaagaatgaaatgagaaaatg
agtgaagattttgttcgtagtgattaggggcttaatgatcgtcggatgatacaaatcactttataagcaaatgtaaagtaatcatcgaaaatcatt
cggcagccgtattcccaccaaataaatgagcattcgctg
9. hgg1c.pk004.l14
caagtttgagttcgttccttttccatgcgtttttcttcattttcctccccttttctccccctctttttcctttctttgccaattgcgtttgttttgtccggccga
actttgccgttcaccggttcgcaattggccaatgaagtggccagggcattttttaattccgtcaacacttgggacatgtcaattttcggagccgg
gactaagcagggcgaggaccgttacaagatcagcttggacggcctggacagaatgaagaacagattcagagtgccgttgccggcgggg
caggggttggaaaagctgctcagatcgtacagagtggagcctctcagagaggattaccttggggtgaacaaagccagagaaagagtgttg
gcaccgagtaaactgatggaactgatggaaaagctgggcaatgtgctggttacggacccaaaaatgcgccaaaagatcgacaaatacgac
aaaaaaagagcggatgaggcggcgcgaagggcggcgatgatgccaccaaggcaagacccacaagcgattgcaaaacgcaggacgtg
gccgaaggaggacggattggcattagaaaggggccatttgcctcaaggcaacaaccagagtccgacgcgactccagtcgacgcccagg
atttggattcaagaagatgaccggtggcgccaaccgatgactttctcccgaaaagacgtgcgggaaagaagttggctcgagtcggacacc
gactcggacttggacagcccaacttcggtgttgcgctcgcggcgaaggagtcgagtgaacattttggacgacgaccaaccgacaagaag
aacggcctggggaaggtcgccgacgccatcgccaaatggacgtgctgttgtacaacgaacaacgaccacaacgacgacgacaactgag
gaggaggaaggggggcgaagaacggtcagatttggcgaagtggtggtcgttgagccggaagagagaacagtgaacagacggacgga
agtacggacacaacagcgggagaccgaagtggagaggacgtcggaatataccctaattctgcgaattgatttcatcgatgcctccgttttttt
ggacaaatcgttggcttactttggaagtctgaacactgccaggaaagacgaaaggagtgtgcagcgattgtgctacgtactgaaggcatttg
acccgaggcacgaaagactgaattcggtgctcgccactccgtcggtggccaatgctttcgtcgaatacaaaaaggcactgaacgacgtgg
gactgaactcacagcccgaactgcgacttgttgaaaaaagcaacgcctgtgccttcgacttggctttgatttacgaattggcccaattcaccaa
agatttgctgttgaagcttaaggccgagcgaatggtggcggcggaggagttggaggacgtcaaagaagaagtgatcggacgactgctga
agcttttgcccaaagttttggaaggactgaaggcaaagcctgccgaactatcgacggaagtcgaccgacgcattcaggcacttgacgtagt
ggaagagcaactgaatgtggtcaaaagagctcgagcgaccgacgaaatggtgacgggggcaatggccaaagtgatggcacagctgag
aaatgcgtcacgaggaatgggaacaatggacatgagcacactgagttctcttcaatcgaattgggacaatctgatgagaaaggacacccatt
ggcaaattcggaaggcaattaacagcctggggggatgcccgaaagacccgcagggcaacacgctaatgaagcaatgcatggaggaag
cgatcaccaaagtggaccgatacattgacgacgtgaacgactggttcaaatcccagcgaccaatcgacatggacgactggaagtggctgg
ctgctgagattcaaatgataattcgttggaagagcccttga
10. hgg1c.pk006.c4
caaaggaatcaacaccagacatggccattctgctgaagtgtgtgctgctcctctcaatcatggcgattttctgcgactgtatggaccccggca
aaaaaggaaagagcaaagatccgatcccaatcccgaaacaggaaggctcagatccgatcccaatcccgaaacaggaaggctcagatcc
gatcccaatcccgaaacaggaaggaaagccgagcagcagtgcagcgaatagcccgacagtaacaaaaggcactccgaaacgtggcga
acttgatacccccgaattttacaaaacgagcccaaagaacaaaattaatagcccgagaaagcccaacaacggctctccgagaaaggataa
aaaagctctacaaaaggaacgtcaagaagaaagaaagcaaaaagaaagagaaagagaaaaccgtttcctgcgaacgaaatcaacagca
ggtaatacgactgacgcgactgacgtggaaaccgaaagcgaagtgattccgacatttgttgccgaactcgaagattctacggtggaatatcc
aacagacattgaatgatcatgttgcaacaaaaactgaccttggacggaaatgatcagcagaaagcactgcaagaatgaggaaaaaagagg
cacggaaagaatgatttgtgatagattctttcttctgtgcattttttctgttgcgtaaatgttgagagc
11. hgg1c.pk006.e12
gattcaactttaatttgactgtgcttccgaattgtcaaaatcattaataatttatcgcgcaaataatggccaacaaatttttaattgctgcttttattttg
acaattgccatttttgtcaatgggcaaagtgaggcgccgaacaattcgtcggaaatggcatcggaggagagcaattcggaagagtcgagca
gtgaggagcagcagttcaacccattcaaatttcggccattttttggtccctcgtcgtccaacagttcggcaccgccgccctttgcctttttgccct
tttttggacgaatgccgtcgctatttaaccgcccctccaacaagagcgtcgtctgacaattgatcactttttgagtgatttgtgggcgtcgagca
gtgtgaaatgaaaccgatgatgagcaaatgaattacattccatttatcgttcatttttgacttttaaaagaaagaatacttgcataaatttattcagg
cg
12. hgg1c.pk007.j13
gactcccaaataaaataaaattaattaaaataaatacaataatccacataaaataaaacaatgaacaaatttgtgggcatatttgtcgctgttttgc
tccaatttgtttcgccattttcggcattttcccgcgtgccaacgacgaccaccgaacgaccgataatttatgacccaaaagaaatggtggaaat
ccaagtgaatttggtgaacaacaccaacaacaactgcacaaatgatgttcttcgaaaataccgtgtggagatcactaattatgtgttctttttggt
gtgcgatttgaaaattcgagtccaattgccggaaggggcaactttggagaatgtcgtcaacctgaaaccgttcaatggcaccaccgatcaatt
catttttcccgattccttgcgctacctttacgtttccaaaacgctcgaagccgaactgagcgtcaaaggcggcgagggggaaccgaaaatca
ctgttttggatgcaaaggccgctttttcgccgaagaaatgccgaatttcgaaattttaatggcaatttaaagaaaggacgaaaatgaaaggag
aaataggatagaaaacgtaataatttctaaagggatttgtatcaataaatatggaataaatgttgatgaaccagaaaaaaaaaaaaaaaaaaa
aaaaaaaaa
13. hgg1c.pk008.i22
aagggaggcgaccgtgctgaaacacgtgggtaaccagaccaacgcggccggcatcgacgcggaatttgctgtgaacttcctcctggcac
agatggaggccaacaaaatgattcagcgaggatatatcgaccggtggaattcggatcactctttcgagtcaaaatatgtgccggattttgaga
aagaaattcaacctaaattttatacgcaacgaatgcattgattttggcactgattccattggtcgatgcgggccaccaaatgcacaacgacca
aaactgtgttgagcatgtggaagacgtgttggaatcgatggagcatttgcgagccagcgaattggagccgaacggaaaggaagccatgga
aaaagcggtcaaagcaatttgtgaaaaaatatcgacacatgagggacaaagcaacgcagaagatcaatcaaaatcgaaaaaacggaaac
attctgacaatcacaaaatggaagagggaaagcatggggaagaaaaagaaattcgacccacaaaaagaacacggaaagcgaacacaga
tgaaagcaaaacaccagcagcaggggaaaataggagaaatcatcgcagagaaaactatgtggatagtg
14. hgg1c.pk013.j16
aatgggtactgtcatatgtgtcggacaaaggctcataccctgtacttggcaaggacgcggagggaagggaacgaatgaatgctctgattgtt
ggacattttgatggccatacgtttgagaagttgtttgaacagcaaatggactttgttggcggctcatttgatatcagggcttccatgaccaaca
gtcgggcagatcatttaccatcggatggatctgcgacattggctggatcggcgacaacactggtgacgcgaactttgatggccgaggtggc
gtcacgtcgatgactttgcctaaggaatttgttctgaaggacgaccatttgattgtcagaccgttgcccgagttggcccaactccgtcagagca
aacaaccgcaccaaataagaaagggtgaaaaatacagtttggaaaaagggcatgccgaacttttgttccaattcaaatggtccaataatgat
gatggttcagcagaggagaaattcgtgttggacttgacccgaacacggttaaaagatggcaaattggagttcacaattgacagcaaaggcat
tgagctgaagaggacttgggtaaaacccaacaaacgtctggtggtgtacaatgttaagccgggtcaaatccatgtgttcatcgacttggaca
ctgtggaatattttgcggataatggccgatggtcgggcgccgttcgggtgccaaatgcaagccaagaaaatcgaatcggaacagttgaact
gaaaagtactccgctggtgcttgagcagtccagcttatggtatctgaaatacggatcacacaaatccgcgcggcttcaaccaaacggcattc
catttgcaatgaacgctggaacgtcgtcattcaaacaggatgaagcctaaagaagacataaattgtgcctcataatctttgattatccaagata
gaaattgatagattaatgggagatcagtagtacttttaattggatatatattaatttcctcacaatttaatggctttgtaaaatttgattgttccaa
15. hgg1c.pk014.i6
gattatccagagatgaataataattttttattgttgctcatcacctttacattcatagttggtgcacgtgctttttggatccaattgccaggcacctttt
ggggatatggtgatgcacgccagcaacagcaccgggggtggcttaatggatggcacagttggcacaaccaaaaacataatggtgccaat
accggtggttattggcccatttatggccacgggcatggacattttggtaatggaaatgcattgccagcagatgatagatcttccaacgaagaa
gacgacaacgaaacatcggaggaacagcagctaacaacagatgatccgccagagaatgcttcatctgacataatggagccgaatgatgg
gattactgatcagccaactgatcaagatgggagtgatacagaagcaaccgattcgacgacagttggatcggatccaggaccaaatgacaat
gatcagaatgccactgggccaactgatgaagatgaaacaggaacggaagcaaccgattcgacgacaacaacaactgaatcaaatgcaat
aggtgaagaaggtactgatcaggatgctacaaactcatctgatcagggagaaagtgatgcagaagcagaagcaaccgattcgacaacaaa
tggatcggatctggaaccaaatgatcaggatgaaaatggtgcggatgctgattcgacgacaacaaacggaatttgatcaaaatttactgaaa
ccaaactggcaatgatcagttgaattttttgatttgcagcgtggttgatcaattaatgacgaatgtcaatatcattttgatattgcaattaaaaccga
tgtagttcatttgcgatacaattttttttcatgtgtacaacgaaa
16. hgg1c.pk015.h1
ggagaaaaagcaaaatgtgttcgacgatttcattgcggctgccgagtatctcatcaacaaacagtacaccaacagctcgaagctggctatttt
cggcgcctccaacgggggtttgttgaccgccgtctgcagtcagcagcgacctgatctcttcggagctgtgatcacccaacttggattgttgg
atatgctgcgcttcaacaaattaggcattggctcagattgggtgtcggagtacggcgacccggacaatgccacagacttttcgtacatttaca
agtattcgccgcttcagcagctcagcgtcactcccgggaagcagtggccggcgactcttttgctctcggctgaccatgacgatcttgttgatg
tgtctcacacactcaaatatacggcacaactgtatcatttgttgcgcaccaatgctgagagttggcagcgcaaccccgtggtggcaaagatttt
ggtggaccaagggcacgcgttcaccggcacaccgaccgagaaaaaaatcaaagagaaggttgacatttacactttcatcgcgcgagcgct
tgggctgaaatggaccgaatgattaagaacaaatccatctgtgtgatcactgatcagatcattatcgatgcaatatttttaggattttcttttcataa
atttgcattccataaaattttgggacaactg
17. hgg1c.pk048.e18
cggggggaacaccggctgtacntgcatatgtttatgatcgaaaaggaacacattatgaaaagaaaatacgcgttgacgattgggacaatcat
tacattgtggatttggccactaatgatgtacaagatgtgttaaaacaaaatttggacttggaatttctaaagctaagagacagtgttgccagtgg
agaaacgaaagaattgacattctatggccgagtttggcccgaaggcaagtacaaacttttttgggacgtaaaaggctttgaaatggatgaag
cgcaaagattgatcaaatcggaattaaatgtgccacacgattgcttcaccgatgagaatggaaaattcaaattggaatatgaaattgagaataa
gagcagagaagtggcacgatggcgtctcccgcctgtgcatttgtacatttttggggcaagcgtttggacaaaagaatatgtgcatgtgacag
attggcatcatgtgcatatctttgatttgaaaaatgggaaaaaacatgcacttccggcggataaagtcgctgaaaaattatacgaattaagtaaa
agggaccaaatgaatgaacgaacaaagttggcagaaacaaatgaaaaaaacgaaaatgagatcacgttcacgcgttcgttttgcccattca
gacagtgactattagaaatttcgatgtcaccgaagtttttcgctggatgtgttggaacgggaattggagaatggctgatgaatttttggaatttat
aatcataaacaatttgttagattagagttca
18. hgg1c.pk052.h11
atggcccctctcttccatcgcttctcatctctctttgtctttctgatgccgttcctttccgttgtgcttctcccgtcaactgtttgtaccggctctgaca
gtgccgccgcgccgttcgaccgaaagaattatccgaaaatcgatttgcgactgttcgagtggcccattgcttcacattcgggctcgtccgctg
aggtctcttttatcgccgtcgactgctacacccaattggaccgttctttcatctcgaccgatgccgtgctccgtctcaacaattcgttagcacttc
ggcaccgcgcctgtctcttgcgcattccgacggggacgcggctgacagtgaccgaaatgcaaacgaccaacagaaaggtaaataagaca
aaaccaaaacttcggcccatggcacgtgccgtgccaacaggcgtatgtgctgttcaactcgcgcgggcgcaaaatggaatgggtcgaattt
cgtctggacgacgaaacggaggcggacaaagagatggcgagcgcggacgaatgtttggcggacgaagaggaggacgaagaggaag
aggagaagggataccgcaaaaagcgagctcattgagccgctgggcagcagactcttttggctttgatgagcattga
19. hgg1c.pk001.a19
caaaggaaagaggaaggtgaggaagaagatgaagaagaggagggggaagaagaagagggagaggggcaacggaaacgccaaag
aggcaacgaaagcacattggaacttctgcgctgtcaggacaaaaacggcaattttctgcccattgcgacagtttgccagaacagagagacg
aaagatttctgcgagagagtgttcccctcgcgcgacacaaattcgcacgggcggccgcgcaattgcgacttgcccgggttgaaggaagcg
gtttacgggtgtgcacatcactgcaaagtgtgctgcgagttgaaggagcacggctgtggcgacgattcgggttatcagatcaactgtgctgc
gcaaagacatttatgtaaaaatatgacggcaatgatgtctacgacttgtgcgtccacgtgcggtctgtgcgcgacgggcgcgtgcgcggac
actcaggacggatgcatcggactaaggcacatgtgcgaccagaaggagttcgaggaggacatgcaaaagtgcgcacgcacttgcaaatt
ctgcacaccaaaatgtgctgatctgaccaacgattgtcagatcgccgatgaaagttcgtgcgaaccgccaccgcccgatcacttggaagtg
aatccctattacgaggaaatggccaaagtgtgccgcaaacggtgccatttatgtgac
20. hgg1c.pk001.c9
ttgctccgccggccgccgccgcttcatttcttcggccattagtgaaaatcatcagcaaccgcttggctcatttgtgacatcatttggaaggcgg
cggagtgtgcgcgtggccaagaaaatgaataaacgaattttgaggaaaatttgggtttgtaacgatgtttggctgcacattttgccctttttgga
ccatgcacaactcggtctcaaaatggcattgctttcgccccgtttcaatgcgttggtggacaaacatttcgacagcaaaagcgaattgacaatt
tggagacgtttcaaaattcacaacaaggacaatggaacaacaccaaaactttctgtgcgtatggaaaacaaaagttttgtggattttccgctgc
cggagcgtccgttgcccagcaaaatccgatttgaataccttcagattgattacatcgaccacagtgtcgtcgcatttctccgttccaataagca
agcttttgaccgaggcaccaactttgatttgtcaataatacattccatcgacgaaactgctaaacacaagcagatttgggatgttatggctcaac
aaatttggcccatttttgcgccaaacattcgccatttggaattttccaaaatcgaatatcgggacaatttgcttcgcctcatttcatcaacaattcag
tccaatcccaatctgagttcaatttatgccggtggtcagttctccgacatgtttgctgatgatggtgggacagatggaaaaattggcaaagcgtt
gtccaaatggttgcacattccgtccaccgatggtcgccctaaacgattgacatgcggaatgagttgttatagcaaaggaccaccaccaaactt
cgaatggatcaacaaattgaaaaaggcatttctccgtgccacctcttctgccaattacattattacaattcaacttcgcgcattggcaccaattgt
gccgtttgaagtggtgaatgaaagaacccaagaaaagctggcagtgaaaaaagaacgcgaatttggctgtgtgaatgattgggtgttgaag
cgaagcccaattggggagacggatcagcataaagatgaggaagattta
21. hgg1c.pk001.f5
ggtcaccaaaaggcgctgtctcagcaaaagaaccagcaaaaacaacagcagcagaagaagggtcagggcaacgatcagagagcggct
gccgccaaagcactgacattcaaatgctccgtttgcatgtcattgatgcccgacccgaagacgtacaagcagcactttgagtcaaaacatnn
caagaacgaactaccgcctgaattggtcggtgttgaggcatgacaattgtggaattttgtggactacgatgttttgggggaccattggaaatca
tcgaatgtatttgtttggcgtacggattgttttcattgcattttctttattttttcaaacaattttattttctggtgatggtgtatttttgaatttccaaaagtt
22. hgg1c.pk001.h1
ttcatgaaagatggacgaaacatggaaaaaatgctaaaatattgtgttcaagtttcgaaaaattataaacattacatttttgagaataaaagcga
aaaacagattaattcacgaaaaacaaaaatatttcttgaaaattttgggtttcaaggagtttttttcgattttcttttggaaataatgccagaaaatg
gatgaaaatggaattgttttcaaaattcattattcaaatttggcattcgccttctctgtccgtcatacagttgtagcatccgtccggacaattctttgc
gtatttcttcaagtcattctgtcaactattgaactgaataagaaaatcagtttttcaaattacgtgaaatttattttcataaaaacataagctcttaaaa
aaacaacaatgttgtttttgagatttattgagttgaagagttgcgatcccaaaatttaaaatcctcatttgagcactaaaaatatttcttt
23. hgg1c.pk001.i14
gggaagggacgaaatggacaagggaagggacgagtcgcagcagagggaattggcggaggaggcgaaggccgacaaacgacggaa
gagtttttccatcgcccgtccgagtcggcacgacgactgcacttggtttggccattccatcgccgcttcacttcgtcaaatgcccattcatacaa
aggaattggccaaaactcgcattcaacaggtcatttatgagtgcacttcgccaatcatccaaaatgacaaagacaaagaagaagcacaacg
aaatgggaccattaaatgtgatgggacggacaatggcaaagggcgcacttcgatcatttaaaacggaatgccattctttcgcttctcatattgg
cagagattatttttgttat
24. hgg1c.pk001.k2
gaagcagtgcaacaataatgctaacaacggaagcaacggctccaccattgcaaacagcaacgtcttttgcgatgatgacgacgacgacaa
tggtgcagctgctgatgatcatcgacaaggacaaagccaagtggagctgccaccgaaatggaaatgggcaccaccagaagaggaggcg
gaggaggagggaaagcagcatgaccaaggaggaggagggcaagaagcggcagcacatcgatgtcaagcggggcccggtggaaaag
aagggcagacgcggtgcggttccgtgccggagtgtcggaaaggatggcaccaccaaagggtcgaaatatttcataccgaaggatgtttgg
cgtgactatttgggcactgaatgggtggacatggacagcctcgaattggaggaggtggacgagccgcaatatgagccgatgatgccactc
aacccggacaagtcgggcgaggtcgactgttgggtcaaggaactgcaggacgttgagggcaacgggctcgccaggggatgggaggtg
gagagtgtcattggggtcagtgcaaaggctgcggatgggacgcgtcagtgttttgtcaaatttgtcggcttcaaattgccacagcaaattccg
ctggctgttgtccaggaaatggcacccgaggccttcatccagtggtgcacttggcanaacgacatggacaatttggacaaatgtggcgcct
attgggaggaacagttgcggcagccgccctcctggatgtgccgacggtcgttggacgcctttgctgcatggaaggcgtccaagttgaagc
agtgcaacaataatgctaacaacggaagcaacggctccaccattgcaaacagcaacgtcttttgcgatgatgacgacgacgacaatggtgc
agctgctgatgatcatcgacaaggacaaagccaagtggagctgccaccgaaatggaaatgggcaccaccagaagaggaggcggagga
ggaggaagagcaggaagatgacattgaggag
25. hgg1c.pk001.p14
tcgcagcatggagcgcagtctgtcccttgcgctgcccatccacaaagtcgtcggtttgggcgcccgactgttcggttttgctcccgacacatt
aacaggggtcgaacttcgacgagcggaccccgcgtatccgtccgaattgctttgtcgcaccagggacaatttgttgcgacaattcgacatcg
acgacggggacgtactcgcctttgtttagtggttcattacgagtgacagttctcggcaaaaaacaatcccaaaatgtgattcactttaaaattgtt
ttctcatcccttttgtttctttccgatcccttcattttttaaatggataaaatattttaaatg
26. hgg1c.pk001.p16
cggatgaaaaaagcggaagaacgattgaagggacgaaaaatggaggaggagaacgacagagaacagcggggaagggccaaaagac
tggtggaaaagttggccaaactgctgacagcgggggatttgccctttctgaccgccagcagaaagacaatgcccaaagccaaaaagcag
aacaacacgaagaagttgcagctgcatcaacagcagcagcagcggtcacgcaattcgtcccagtcgaatctcttcgaaccgatgccgaca
attagggaggagacggacaccgaactaatgggggaggacgcgcagaacggagaagagacggtgcagccacggaaaaacgacacgg
aaacgtggggagaatggaggacggagggagatgccaaaaagtgccacggtgacaaatattgcacaaaggcacagcaatttggcaccac
ccagccaatgctgcagacagccacctgattattgttgttttgtcgaagcaaatgcccaatacaattcttaattgcttcaattagtaaatactcggc
gattttctttcatatcatttcaaatatttattctatttttactgtaaatacaaatgaaattgt
27. hgg1c.pk001.p7
gagccgactttttgtcaccaacaaaacaactcgataattcaattggattcgaggaagaagcatttggtggctcaaagaaccacgacacaattg
gcattgttttggtcgattctgagggaaatgttgcggccggcacttcttccaatggcgcaaagaacaaaatagcgggtcgtgtgggggacgcg
cccattgttggtgccggggcttttgtggacaacgaagtcggcggagcagtggccacgggggacggcgatgtgatgatgcgatttgtgcca
agttttttggcagttgaacaaatgcgttatggaaagtcaccttcgcaggcaacgcgcgaagccattgaaagaattaaacgaaattacccaaat
tttatgggggcggtggtggcggctaacgtcggaggcaaattcggagcggcatgctcaggaataaaaggaggctttgggtattcggtggtca
attcaaaccatgaaaaagtgtgggtggagagagtgaattgcgaatgaaaagaaattaatcgttttgttaggctctcaactaatttattttcgttttt
atttaaaaagagaaatacctgcg
28. hgg1c.pk002.d17
gatggcccttcggatgacttgcattttccgttccattttgcaatttgttttcaaaacgacaaatgccccggggggcaatgcgtcgccgctttgtcc
tcagatgggtgccttagcggagcaatgcatccgcgccatcggccacttcgccgttggggacattcaaaatcagcttttctgtgtgttcggatg
gcgtcgttcccttctctcaatgctttgcacgtctctcccgctgaacttcgtccactccgaaccccaaaagcactttctcctccccactctgatcgc
cgtgctgcgcaattcgccgatcggagtgaaccaaatccgaacggagttttgtcttcaatatttggtcggatatttgaaggcagcaatttaggcg
aaaacctccgaaagaaagtccgattcttccaaattcgactttctttctcttcttgagccatccgtcggaaaatggaattcagcaaaggaatttttc
gaatccatcagcaaaacaaatgattttttgctttgaaatgtgttacccttttttactaaaaaaaattgctcaaaaaataattgtataattactatgttaa
aataatttcaataaaaatatagc
29. hgg1c.pk002.e14
taagcagtggtatcaacgcagagtacgcggggagcgtctacatgggagcctcgcccgcgtacgagccaccagcgcaggagaagtcccc
ggatcagagcgcctacatgtgagaagatgcaacaacgaccggcggatggatggacgaaacctgaagagcgagcgacctgtcagaagat
gcaaagataaagaagatgtctcataatcgtgatctgtatttattgatgtattgtacatttgtatgcatatatcatttgctgtgtattatcactttatttcc
atctgtgttccgaaataaattgaattgatggc
30. hgg1c.pk002.h6
cgagccgcccgtccgttgcatccgtcccactcgcttgacgcgtcgttcgactccgatggactcgcgcttagacagccaattgagtggaggc
ctcaaacactcgccaattgaccaccgatacaggtccgttaagaattacgaccttgccactgcactaaaagagcgacacaatcggagtggtg
gcattggcattgaacatcgctattacgccgaccattcgtccgacttcctcgcgcattcgtcgtcgctcagtcttcgttttctgctgaatggcctcg
cacgcagtttcactggatgtctggccgaccctgacgaggaaatgaacacgcagcagggggaaagtgacgcctcccaggaaaatactggt
gagaaaaaagctggtgcggacttcaaaacctcggcggaatttctgaccgatgcttcggaaaaccgtcgcagaaatgaaatggtcgtggagt
ctgttctggagaacgatgccgtacagaaactgaatgccaattcgtccattgagaaagtgccgttaccgatgccgattttcgacgacgccgcc
actgccttttaccacgcgtagagtgacactgaccatgccattgacacttttcaattgaccataattactaactgaacctttcatgtgccctctgaa
attagtgaattataaagtaaaatatttc
31. hgg1c.pk002.j21
caaagcgcatgaaactcgaggaagagccgcagcaaacgagccgaactctgcgggggatgggccatggactcagtaacaaatgtttgcg
atttggatgtttggatgtaaagctctcttaaataattttcattcgcatttgtatgtgtgcttcggtggctcagtcggtagagcgtcagtctcataatct
gaaggtcgagagttcgaccctctcccggagcaaaattttttgattatattttttatgctgttatatttcgaatttttttctaagtacactaattgcgctg
atttgatcattgtaaacgaataaatgattcctggct
32. hgg1c.pk002.k14
ttttgaggaggcgctttctctcacccattccctcgttttggtgcacatttcgcctgagatgtggacggtttttgaccatatttacaaggcttttctgg
aggaaggcacttcatttttctcagattgtgcaccagtgctccacgcttttttgaccaatgacactgacaattttctgtctgtttttgaccgagtgcaa
cattttctggcgatgtgtgaaaaaacattgaacgatgagggtgaggacggctgtgatgagagcacaaaggcacatgcggcaaaaatgctg
gaggtttttgtgctccaatgtcaaggacgtgcaagtcatttcatcccggacatattgcgtttggttttcaatcaattgcagaaagagtcggccgat
ttaaaattgggccaactgaagccacaactattaattattttgatcgctgctttgtattccgattttcaattatgctccaatttgtttggtcagctgcaat
tcaaaacggagattggcactttcgaatggcttattcatgagctctattcaaatcggaaggactttgagggtgtgcacgaccgcaaaatgctcat
ttggttgctctgtcgcattttggctgatggaaatttgcccgctttgttcattaatcagcctgaaaagtttatggagtggcttctgactctttttgagga
actccaacggtgcatcaaagaaatagccgaacggagggaggacgactcggactcggaggacgaggagtccagcgaggaggacgacg
atcggatgaacggagagttgaaagactcagacgacgatgtggacgaagagaactcgcaatatctgatggcattggagcacgaacgaaat
gagcgaaaagaacggaggacgcgaaggaagtcgagcaccaacaaaagcatggacgatcagacagagggagcaccgggcgacatcct
ttcccttgcatcggaaaccaccgactcggaagagcaccaccattttgaggaagagactgaccttgaggcattttctacaccattggacgacc
aaggcgacaataagccatgtctgaatgtgtttgttttgttcaaacacacattggaagaaatgaatacccgcaattcgcctcttttggtcagcattt
ctgatcagcaacgaattggcgaggcacgagttgcaaagcttaaccatttgatggaaatttgcacgagagaggaaaatttggagaggtcaaa
gcgcttggcgcaggccggcggctattcttttgacgccaatgcgccggtgccgacaacattcagcttcagctgatcgaagagagagaaaga
attctaatccattcattcgtttgtctttgatcactttgggtgtaaaataa
33. hgg1c.pk002.k5
gggacggagtctccctctgtctcccggtctggagtgcagtggtgtgatctcagctcactgcaacctctgcctcccgggttcaagctatgctcc
agcctcagcctccagagtagctgggattacagtgtgcgccactgcgtttggctaatttttgtatttttagtagagacagggtttcaccatattggc
caggctgatctcaaactcctgacctcaggtgatccgcccatcttggcctcccaaagtgctgggattacaggcatgagccagtgcaccgggc
ctttccaaacaaatttttaaaaatcttttgtaccttatgtttttttcaacttcataaaagttttaaatttatagaaaaattgtggaaatagtagagctccc
atattctccatgtccagtttcccctattaacatattagtatggtacatttgttataattaacaagccaatattgatatattaggtttctttagtttttgccta
atgtcctttttctgatctaggatcccatccaggatacctcattacatttagttgttatgtctccttaagctcatcttgattatgac
34. hgg1c.pk002.m5
caagacgaaaaggaccaacaagtgccataaatgaatgtaattcaaagtaaaactgtaattaagaagaaatcccaatggaaaccgctggaga
taggaagtaagtctgagagattaaggaacaaagtccggaactttcagtccaaaacgagattttttgttcagcaacgaaaattccggacccaga
agaattgcctttcggaattgtacagactgcattccgagcttcagatcggatgcgcacgacctcagaagattcggccgagtcttttgacgcctat
ggaccggagtgggaagggaagggacggggaattgggaacagaagggaattcagttcgccaaatatgaccagttcggggagacgaatg
agcatcacagaacgcttatttggacgtccagtgccccaagaacgaagaaactcattgggagaggaacaaatggggcaggaaaagccgaa
aagcatcgcggagaacaaagacttcaaagaattaatgaagcgtcagcgaaaaattttgggcgatgatgagtggcaataaagaaaggcaaa
agaaaagaagtcattagaggaaaacaaagtcggaatggatcaaagggtagaaaagggaatgacaatttatttatttgtttattttatttaacactt
cttctgatttttcaataatgaaataaagacaaacccactt
35. hgg1c.pk003.b22
aggagcgcgtggaagtttggtttaaaaaccgacgcgccaaacaaagaaaaaaaaacgcgggagattcaaaacaaccagcaacagctca
acaaaagtcatagcatgtgctctccgaggccgtcatctgatggaactccaaaaaatggacattctgaagaggaagacgaatcaggggatga
ttcgttggacacatcgccaatgttgaatgtgccaacaaagcgattcaaggtgtcagcagagtgccgtgagcagccaatcgagcatgacaaa
atgccacacttaaaacaactacaacaacagcagcagcagcagcagcagcagaaacatgttcccgttgcacatccccaaaaaattgtgccg
atgccaccgcatcctcaacaaatgtccacaatgacaccgcagcaataccaccagcaacaacaacagtttttttgattttgccaaatgtttcggc
acctttggcacggcgcctggcctagccgtcacaaccgacccaatgttaatgcatcagcagcatttggcacttgcgcattcgttgggtgtggc
cgctgccgcgggtggtgccggcggcgctttgatgcagcaaattccggcggcaataatggcggaacagctgctagcgttccatcatccatg
atcagccgacaataaaaattccattgaaaaatgggtcaaaaatcggctggccctgctggtgggcacttgtgagcttgtgaccgatctcgaatt
gatttttataattgtttttggtatatctgtttcgggtgtccaat
36. hgg1c.pk003.d14
ggcgaaggcgaaaattgggaggaagcgaagaaagcgaatggacgggagcatttggacggtggaatcagtgctggacagagaagaaga
atgagcggagggatggcagacaatgggaggaaatgatggaaatcaaacacggcacaaccattcgaaaacccaaataagaaagggccttt
tgccattgtccgccgtttcccaattattcccaaatgcttttccccctctccctccattgcttaaacctctctct
37. hgg1c.pk003.e13
cctattgattaattaacagtacttgcattaagaacaaatcattaagaagatagaagctgagtaaaatgagaaatattcatgacaaggagataaat
tggtaaaatgagaaatgatcgatcagtgaccagtgaaacacacccgacaataattctaaatattagaatgggtgggtattattcattcattccca
aggaatgcttgaaaacatttctaatcctttaagttgtcgggtttcttgttcattcccgtcaataatttcgcaatttgccaatatcccacagttcgacc
gtttccgccgaattcccttctgtattccagccgagcgaaaagtccgaattttccacggtgttgtttcaaataggacttcaattcatctgtcggaatc
aattttttgatgaacgggatatggttgttcttcggcttgaaacggcacgtccaaatttcattgtgcacttctgaatccgggtcgtagtc
38. hgg1c.pk003.f12
ttttgaagtcagatccggatcggaccatgttggagaagaaggcagtcccggatcagttgatcatcattttgaagaagtcaatgccgaatcgga
tgaacgtgaagaaaaagccagtgcccgatcagttgatcatcattttgaagtcagatccggatcggaccatgttggagaagaagacagtccc
ggatcagttgatcatcattttgaagaagtcaaatccggatcggatgaacgtgaagaaaaagccagtgcccgatcagttgatcatcattttgaa
gtcagatccggatcggaccatgttggagaagaagccagtcccggatcagttgatcatcattttgaagaagtcaatgccgaatcggatgaacg
tgaagaaaaagccagtgcccgatcagttgatcatcattttgaagtcagatccggatcggaccatgttggagaagaagacagtcccggatca
attgatcatcattttgaagaagtcaaatccggatcggaccatgttggagaagaaggcagtcccggatcaattgatcatcattttgaagaagtca
atgccgaatcggatgaacgtgaagaaaaagccagtgcccgatcagttgatcattttgaagaagtcaatgccggatcggatgaacgtggaga
agaaatcggcgccggatctgttgatcatctttttggaacggctttntcagttgatcatcagcactttgaagatcccgattccggatcacaaaaac
ttgaccaatcttgggaacacaaatcatttgaagaggacaacgatgaagagcctaaaaaattgacaattccggatgaatatgaccagagcgat
tttttaatagaaaacaaaagtgttggagaacaagaaaaggaaattattcgagaagaaatcggatttaatggccaagcagagaacggcgaaa
agccatcatttgaggaggaaaagtgtcccccggagggatgccgactttaccgagatgatttggtagagagcgaagaggttttgagaaatga
gcatgatt
39. hgg1c.pk003.j11
agcgaagaaacaatgccaattcactactcattgtgaacccattggaacaacagcagaaacgaattgacaacgaggacgaaatgggaggg
cagaacgaaagagtggcgagggtccgaggggcaaagggaaaacagacgacggaggagtggaggaaagtgccaattgctgtgccaca
gcaaaggtttggcaacgcttccacaacttcgagccaaatgaggttggacactttgcaaggcgagcagagtcccaccaacagttactcgctc
gacatcggttcgattgaacatttacggacagaattggattcggcccactccaaccttttccaattacacgaacgttttgaaaatctgttggagat
gtatggcggttgcctggaaaccatcgaggaagtgaagtacgacaacgaggatttgcggaagctgtgcaaggagcaggctctcaaattggc
cgagtttcaatccgttggtcccccgtcctaacgaagaaaatcgccaaaaagagaggaagcgaatgatgacagaagaaggaacgtattgtgt
gaagacacaaaaaaacatgcaatatttatttcaaagcatttatattggttgtgatatttttggaactcataattctaaaatacagcagaaaatgg
40. hgg1c.pk003.13
tgcagaggccccgcgtgactatcggcgtcgacggctccgtgttccgcttccatccaaccttcaaattcaacctcgaccagaagatcaaggc
gctgttggccgtcaaatgcgaattcttcatggtgctcagcgaggacggaagtggacgaggcgcagcagtcgcagcaacagtcgcattgcg
gatgaatcgccttgtgggagcgtgaacagcctgtgacgatgccgtccgatgtcagatgtgtgaatcttaggccccataatgtcatatgtattgt
aatgttaggcattttgtcccatgtctgtctgtatataaggttgaattcctaagcacaatgatgttccattattcacaatttgtatcaattgttcatttgta
ttgtaggtgtgataaatgagaaaacattt
41. hgg1c.pk003.m24
aggctcgttgggacttgcctgagggtgaggagttgctgataattgacaagtcgaatgttggcggtggtgccgtggccacgtccccaaatgc
cgaattgatgggcatagagcgccaagtgcgccgggcggagttcaaacggcacgtttcggcacttgtggccaaatgcatcgacccttaccg
aaggcgcttcttccacgccaacggggaatacgccaactttttgcgaaagataacgcacaaagtgttggacaatcagccaaagtcgggcaat
gtcgagctgctgttcaacgagcaggtgcagaagaacacgcaaagactggtcgacgaatacatccgacacttcaaaaaccgcgaatcgcat
cagttgctgcagcaccggacagattctcagggactttccccaaaatgatcattctttcaatattccatttaaactgagtgctgattttatcaaatta
aataatacattttctgtattgcgtataaaatcgcgttaac
42. hgg1c.pk003.m8
gcgtccgtcgactgatccgctggcggtgctcgacggtggcggactgttgccattgggcggagtgtcggaggaggacggctcacacaaag
gcaccggaattgcgatgatgggcgaacttttttgcggtcttttgggaggcgcaagttttggcaaaaacgtgcgatcgtggcgagaagtgcaa
aaggcagccaacctgggccaatgcttcgtggccattgaccccgaatgctttgctccaacatttgtggacaatttgcagttgttcctggaccaaa
cgcgtgggcttaagccgcgcgacccctccaaatcggtgttagtgcccggtgaccccgaaagaatgaacagcgaacggagcgcaaaggc
tggcggagttatttactcagaaggacaaattcgggatttggagaaattggcaaaaaggcaaaacgttggcatgttcccttacaaggcaaattt
gtagcagaacaaaaaaactgttttctttttgttccaaagcgatgactttcaattgaattgcattcatttccattattgaacaattaattcgttcccattt
gctgctgctgataaag
43. hgg1c.pk003.o13
gggactgctgcaaacgttcaagctgccgacaggcgccccatttgtccgatctgcttgaagaagatgaccggagtccgtcaggtgcgcacc
atctgtgaccacgtgttccactacgtctgcttccaccgttggctcaaatatcgcctgttttgtcccgtctgtgagcgcaactttcgcacggaattg
tatcatgctggaaacgccgtggttgagggagcgtacgccgacggacacgttgtgctccgaactgatggtgaacagagcaacagtggctaa
ttgatcattgatcggcactcacctccaattgtgatcggacaaaaatgatattaattgtatatgtacatatatatcaacactcggacaataaagtata
atgtgcg
44. hgg1c.pk003.p3
taagcagtggtatcaacgcagagtgatttcttttttaactttaaaatttttttatttcccgacaaaaaacttcaaataaaatggttttatttgaaattcta
aaatatttgaatgtatttggctggtcctttttcatttacaccttaaaccccgcctcattcgttgagttgcttcgatgaatcctaacgaaaatctcaatg
aatttaatggattttattatgaacttatcaaacaagttttgagcaatgtcaaagatgcatttatggacgatggcgcggacagcgaggcgctgagt
cagcttaaattgaggtgggagcataaactcaaaagttctgaaatgattggacgtcagcgtattattacatacaaaaaactccaaacgaaagg
45. hgg1c.pk004.a13
aacggagggcagacgcagagacgaaactgcagcagctgacagtccttgcacaacaatggcaaatcgaagctgatcgatacaagggatg
ggctttgcaatggcagtcctaccaaatatcgcagttgcctaacccaactgatacggtaatccaacaattggagcagcaaaaaacagagcttg
aactacaaatccaatatggatggcaggcctttgaagcgcaaagtgctcaattaggcgaattagtacgaatttcggaagcaaatgcgaacaaa
ctgaaccaggtggagcgtgaattgtccgaagttagcagtgagcgagaaactttgcggcagcaattagagagccagcaaaatgtcccgcag
ggatcagccgttgccacacacagcgaggagttgacactgctgaagcgtgaacacgaggacttgttgctactgttggcagagcaggacagg
aaaatacatgactaccgtcggcggttggcctcccatggagaagcattaagtgacgcggacgaagagccatgaacctgcccagaagaaga
agaacacgagctcttcccagtcttaatgaggcctacaaaatttgatgctgacaaagaaattctttggttccttttcctgttgtgaatcttgattcgttt
tttttcttttaaatcgactaacaaaaagctggactgtttacaatttattgtttccccttgttgcgaattgccttgagtttggttgtgttattacggtttcaa
ctgaataagagacaactttgtataggcgaatcatgtctgtgattgtttatttaattttgataaagcaaatatgtgcaaaa
46. hgg1c.pk004.a5
gcgaaattgtggaggaaggggcggaggatgccaatgagtgcaacggtaaaatcgcaaaacgccgagtggatgaagagcacgatgacg
aagaaattgatggcgggagtgacgaacaggaagaggatgaaatggaagacaatgttgatgaaaaggaggagggagaagagagcggtt
acgaggaagacattgaggacccaaaagtggtgcagcaaaaacgcgggaaattgccaaagtcagccgtggacgacaaattcttcaatttgg
ctgaaatgaatgcatttttggactccgaagacaaaaaagaggaggataaaatgcgacggcgaagtgtcagaaatttgggacaaatcgaaaa
cgctgaagagttggagcaactaatcagtgcacatgagcagtcaaacttgcagccgcgtgagtgggcgctgtcaggtgaagcaaaggcgg
aagaacggccgaaggacgcgctgttggagcagtatgtggacgcggactaccgaatggccgcaccaccgacaattgacgcagaaaagat
ggcacagcttgagggaatcatcaccaaacggatcaaagacgggttgtttgacgacgtcgttcgcaaagtgcgcgtcaacgaatcgcttcag
cccgcagcgccctatcgaaacgctactgntaatggcacaacggagcaaaaagtgcgcaagtcattggcggaggtgtacggcgacaaatt
atctgatgggctaaacgacgaacacgaacttggaggagaggggaaaaaggaagaggaacagtccaaattggacccggcggttgaagag
atcaaaagcgacttggacactctttttctgaagttggacgcgctcagtcattttcaatttcgaccccagccaatccaagaggaagtgaaaattgt
caataacatgccgagcttgcacttggaggaagttggaccccaagcagcggttggaccagaggtgaatttgttggcaccggaggaagtgaa
gcgacgcgtgaaaagtgcgccgaaagggacagacgaacgaacggagacggaccgaaagcggcagagacggcagaagaagaagaa
gcaacgcattttggcctccatcggcgca
47. hgg1c.pk004.e11
gactgaagagaaaaaagaggaaaagaaagaggaggggaagactgaaaacaaaaaagaggagggaaaggaagagaaaaaagagga
aaagaaagaagagggaaagcaggaagagaaaaaagaggagggaaaggaagagaaaaaagaggaaaagaaagaagagggaaaga
ctgaagagaaaaaagaggagggaaaggaagagaaaaaggaggaaaagaaagaagagggaaagactgaagagaaaaaagaggaaa
agaaagaggaggggaagact
48. hgg1c.pk004.11
aataatgtttgtccatgttgaagcaataaaattctcatgaaatttgttgtgtctaaacgtcatctccatcatcgatgtcctccgcctcctcaatgtcat
cttcctgctcttcctcctccgcctcctcctcctcctcttctggtggtgcccatttccatttcggtggcagctccacttggctttgtccttgtcgatgat
catcagcagctgcaccattgtcgtcgtcgtcatcatcgcaaaagacgttgctgtttgcaatggtggagccgttacttccgttgttagcattattgt
tgcactgcttcaacttggacgccttccatgcagcaaaggcgtccaacgaccgacggcacatccaggagggcggctgccgcaactgttcct
cccaataggcgccacatttgtccaaattgtccatgccgttctgcca
49. hgg1c.pk004.l12
aattgtttgttgcttgtgtgtacgtttggtgatggacaaaaataataagacaaaatgtgttgtgtgccgtcatcaccatcatttgtaaacaccgcca
cccaaattgttcgttttgtccgccgagaggaccagtccgccattgaagtcgaacgccggaatttgaacgttgaacacaaaagcgccagtgtt
cgggtcctgtttgcacgcgtcgtacaccgcctgcatgtccgccgcggtggtaaagtcaataaagccgaacgcagtgcgatactcacgacg
caaagaattcggtttgatgcccacaaactccacaccgtcggtaatggcttgaatttcgcgcatcagctcctgctggccc
50. hgg1c.pk004.n20
atgttcctgacgatcctaagtcttcacctactgacccactttcaccatacgacccgtcccatccgcgtcctacacaaccggactatcccgacat
gcgtgattatgatccgcgttcatttaaacctcctgagccggacgatgacccgcttcgactgcatccgatactgcccgctgcgccgtatgcacc
accggcacggcctcgaccttcacagcctaatgcgcccactcgaccgccacctacttaccccgacattggcagaccgtattttgatcctcttcc
gcaccagccgacgaacccgtacagcgactggccctatggacctgcagcaccgactggatccggcggatatatgggcggttatgatggtg
gagtgtatgaacctcgtcctgaccagcctgggaactcgaattatgaccatatggaagaggaggatcggacgacggcaaccacggacacg
atggtccaggatcttctggtggcggtggcggcggattttttggtccttatctttaagaagttcggatgtaagtctatttgcttgttgatatgcaattg
tttccattgtataatatgtaatgtggttaacgggtattcattcatttaacacatacattggcatatgtcaaccatactatttgtttcaataaaatatatca
c
51. hgg1c.pk005.a3
ggtgactgattattttgatcagttgtctgatcggcttccgactgattctgatcactacttttctctgcgtcttcctcttccgctgccgtttgaccattttt
atgctcgttttgcccatcttcttccgccttctgctgctgttgttcttgttcttcattctgatcactatgctcttcctgtgctgtttgactctgatcagtttgt
tgtccttggttttcttccttgtccttttctaatggttcttgttcttcattctgatcactatgctcttcctgtgctgttggactctgatcagtttgttgtccttg
attttcttccttatcattttgactctcctcctcctctccatgttgtccttgttgatcagcatcaggtgatgtttctgttggtcgttcttccgccggttgatc
accagacttttcttcttccgctgttggctcttcatttggcttttcctccttcttctcctccccctcttcctgctgctggtgttgatcacggtctcttggtg
cttgatcaggctctttccgcgtgttctgatcatcctcctccatcccatgctcttgatcatgctcttcctcatgcgtcttctggtcatcttcctccgtctg
atgctatcatttccttctgccgtttcgccattctgttgctgatcatcattgtcgtccgtgttatgctgatccggattttgttcttcgccgggtggatttt
gctctgctgatttggctgcttctcctcccccattgagttgcatcatcttctcctccttattcctttcctgctgcaggtgttgatcacggtctcttg
52. hgg1c.pk005.d17
acagaggcaacgaaaagcaacaagaagaggaggaggagcaggaagaagcgagacaattacagcaaatgatggctcttctgtagtccta
aaggtcaaatatttaaatttaattaaaaaagatatggcactctctctattccttctgttggtcggaacaatcattgctaattgcaatggtgacccaa
agatgaaatctgttgaagagaaaagtgtgccgcctgccgccttttggccttacattttgcatccaaaaacacctcggcataaatcagaagaga
gggatgattactacgatgccgtacgagcagaagaggaggaggcggagaaggcaacattgacaagcagtacagcagcaaacagaggca
acgaaaagcaacaagaagaggaggaggagcaggaagaagcgagacaattacagcaaatgatggcacttctgttggccaacattgaccc
ggtgccaatggttaccgccaacagcgaaaagccaaaaacgatagcacaaacgatggcaccgacaaaggcagcaaccgcgttgacaatg
tctaaagtggacggggaaacgtacgacgaacgtacagaagcgggcaaagacgacgaagagacagacgatgatgatgacgaagagcat
gaaacccgcaaaatggttgacacggaattgaagaagcacaaattggttgtgctgccgaacggatcggactctgacgatgtccgagaagcg
gatgcagaggcagacggagtcgaacaaatgccttcaaaagggacggtggacggacaaacgcactttttgg
53. hgg1c.pk005.d22
acgaaacaccgcnggcggcatgcggcacgatcgcgatgtgtccgcgtcgtttgatgatgacgcgaaatacttgtacatcttggacaccgaa
ggaatggacccaaaaacaatttntgaacagaccatcaaagcgctgcatgccaatgtgatgtcgggggagaaggaatcgatgccggggga
atacagagtggacgaagtgactgtgggcggacagaaggtggaagcaactgctgcggaagtgcctgagaaaatgacgcaatttgtggaat
ggctcaatgccgaagacgcccaaacaaatgacgttgccactttcgccgcaactgctcactataaaatgaggattctggccctgcaccatacg
ataatggtcggggaaaaggaccgtgctgccgcagcaggcgtttatcgaatgacggatgtgtttgttggtgaagacccgattggcgtgccag
tatgggaaatcccgggcgccatgacggaattttgtcagtggctgaaggaggaagaggaaaagctgcatgaaggagaaggagaactggc
gagatttgctgctatggctcatctccgtctgaa
54. hgg1c.pk005.e16
ggagataaaacagcttatctcatcggtgttcacaatttgtcaatttgcatttggcctttaattcacacgtgtctctccctagatggagcgtgggcc
caatcgattgtttgacccggttctgcgacacaacccaatggcttattggaccccacgtcgtgttcgagctctcgaatatgtcatgcgagcgtac
acacgtccgcgttatcggaccgtggcaacccagaccgagcctatgaacgtctggccaatcttctcgacaacctctccgcgatatatccgtcc
tcctccacaataagccaattacaccgcccgtttcccatgacacttcatcgtcccgagtacacaacccaactgtgcataattggttcagtctattct
caattcccctttcccgtgaccatactcaacatcaagtcataagtcttgttatcttgtagtccatcatcaccctatactcaactctataaaccaactga
tgcattcgacaaagaaaccaatagtcaaacgttagtagaacatcagtcacaaaattatgagacccgctaatgtttatgcgtcatcatctc
55. hgg1c.pk005.l21
gagagataaaagaggagagagaaatagatatacccaaaagaaaatccaaatctctaatcagttggtcaaagtgtttccattttccgatatggtc
gctgtgacgctcggcacatttttacaaggcagcattggcactgcggtggtcattgagcttaaggacgaaactgcgctcgaagggtcagtgg
acagtgttgacccgaagtcgctgaacacgcagctgagcaacgttgtgttgtacagacgacggcagaaagggctaaaacccgcgcatttgc
ccagttttttttgtaagggcaaacacattcgcttcgtgcattttgagaattacgcttgtgcgctgcatttgttgaaaaagtcgttgcgcaaattgtaa
aagccatgcccaaaagaagcaacaacaaacacccgtaaagctcatctccgtgtcttctgtctaattggaaatattccatagcttttgatttttcta
atttattgtctttgtgcctgagttatcatttaatcatttctttatcaaatttctctacaattcaaagacaaaatttccat
56. hgg1c.pk005.m5
acaacaacaacagcagcaatcgcatgaccaaggagcaggaggagggcacaagaagatgaagctggacggcggtgatgaccacgagg
gattgccgtcttcggcaacgacgacgatggctgaacaacaaagacagcagcaacaacagcaagaacagtcgcatttgatggacgaagaa
atgatggtgatggacgagcatagccttggcggcgtggatgctcatggcgatgtggaggcggaagaagtgttgcaccatccggacgtgccg
aacccgccgatgacgccgcctgtgccggaacgaatgtcgccctcggacagctatgggctgaagtttgacagcgatgtgcaggacattgtt
ggtggtgacgatgatgacggagtggaggaggtggaggacggtgctgacgaagtgttgtacgctcatgaagaagttgaaggaggcgagg
aggagggcgtggatgaatatgatgaagatgaagaggaggaagaagttgaggatgaagcgggtgaag
57. hgg1c.pk005.n1
acggactcgagattcgtgtgctaagtctgcagaaaacagttggacattcctctactgatccattgtcacaacaaccggggccgagcgttggat
ttggcgggaatcttccgtttggaatgccggccgcgaaccctaatttggccaccgcgttttcgatgtatgggtcgaaggcaactacgatgcag
gggacacaggcggacccgggggttccgactgagtcccaacaggaaattcttgaccgcttgactaaaatggggctttgaaatataaagcgat
tgttatattttctcctttccctgttctcgcctgttgaccccatgcttcgtccagtctcgacaccaatagcgagtcatcctcgctcttaag
58. hgg1c.pk005.o16
aagtgagaataaataaataaatatttcgcaaattcggacccatcacttttattttgttcggatccaattgtgaaggtgttctccaatctgataacggt
cctcctcaacaattgccctttccacccattcggaagtgacgaaagtgccgagccaccccttctgttcttcattccacacgaacagtgcctcggc
atggtcggggtccattgaactgatcacaacatgggtaactgatccgtccagcacttcgctgattttgccgtttcgtgcctcgattttgtcgttcaa
ttggacgacgttctgttgcttctccaccgccttcaccgacccatggacaaagaacacaaagcccgagaaaaggttttccggttcgactggtg
cagaatcgaagtcgtggatcatctcctcaatttggtcacacatccgattctcctcgtccaacgctctgtttgcaacttcaattgctccagtctcttc
ttcttcgctgccactcctctctctcttccgtctctctttcgctcccctctttctcttccgtgtctctttccctctcctcctctcccgcttccctctcgcctt
cttccgtctctcgctcttctcctctgtccattcttccctcgccctcgtcgtccgattcttcctcatcattttgttgatccgttgttggaagagaaaaca
aatcgaacggtgcgcgtgatgaaatatgtaccatgtccgaccgttcccatggtattagccttccatgttctttgcatttgcgcagccaattgccat
gtacgatgtggtaattgtccgcttttattgccgccacacagctcaccggtctgttgtccattgccacgaggaaatcggcagttttaccaggattt
gaaattggcgtggcacccaacgaaatgacaattttctgcaaatcttgcgctgtcacaccgggaccaccgttcaaaacgcacactttgcgtcc
cctcaacgcatcactgagtgtcccctccaaagtgccattgtcg
59. hgg1c.pk006.f5
tgactgtcacttttcggctgtccctcgcctctcctccggccgttgtccctccccccgccgtctctccccctctgttcgtcattccgtccattctcca
cgtcttcgtcccggctccctccgtttgctgctccattcctttttcttccttttccaaagtgccatgtttcttgtcgccctcttccaactccttccgcaac
attcgtcagcctctgtcgaccatttcgagcagtatatgcccaccaaatgtgaagcatgtcaactgtttgctcgggagttggaaagcaatgccc
gccgattgtcttcaaaaatgccccgagatgaagcagaagcttggcttgtcgacgaattggaacaactttgccctcggatgctcgactatcgct
tacacaaagaccgcaagggattggcacgttttgcgaaggagcgaaccggcacggcaaatgccattaaacggctgaaggaacgcggagt
gcaggtaaaactggatgttgacgatgcgctgctcgaccgtccgtccgtcgagtcggccaaactgaaggagcactgtgagtggatggtcga
agagttcgagcaggacattgaccgatggttcatcaacctcagacataggaaaactttagaagaattcctttgttcggggcgactcgccgacg
aatttgacggaacangcgcagaaagcgatagacgagaagaattgaaataagactatttccctcaacatttttataatttattttttgtaatttcgcg
c
60. hgg1c.pk006.g7
gaaaacgaacaatgctatggaggcatcacacctccaattttcgcgtggtctagtccatcacccatctttgtcagactttctagcagctattctgg
atgatgttgacaagcaggtggacatcgcaagatctgcccgagtgttcccgcacaaacgccgcatcaaatacattttgaaggagcaattgatc
ggagatgcattggacgaggcggagtacaacaccgacgaagacgtcatgaacatcctttcactgctgagtctgcagatgcaaggatatgtgg
gtggcctgcgtgcacgaggggctcaacacgaacatgaggaccgtgaatgattgatcgctttataccattggcaaaaaccatgtcttattccg
cacaagtgattggatttttaaacctcaatttccgtgattttcaacattttcatttgattcgaactattatttttgatgtttattataataaattttcgatttcc
61. hgg1c.pk006.i10
attgcactaatttttgctaagctcacgccactctccgctcctccagcagtcgttccctcgacgcggagctgttcatcatcaaacacttgctgata
ctgcgcgaacaaatcagtcccttccgacagcacaacaaacagcagaatcgatcagtctctacagcgccattctcaagacaatcgtcgctttat
gatgtgcaaattaacccgcagtacgactactccttggacctgagcaagtacacccagtcgatgtttcagctgctgaacgccgagaacagag
ctcgttggttcgagttcagctccaacaatgcgtttctctccctcctccttttgtcgcccgtccacgtcagcgaactccaaacggactcacgacg
gatcatcgaagcacacctgagacgatggtgccatagcatgatcggacacgtctccgcaattctgttgggaccgttggccaaatttcagtcga
acattgagcaattgcaggcggagcaagaacagcgggcccaggggcagaaaagtccgttggatgtgaccaccagcgaacgcttcagccc
caaggcattgcacgaatgttgcgcggacgcattcaaacggctgaaacagcactggccagaagttcgcgctgccttcaccctttacattgga
gtccgcgaaactgaggaaatccttctccagccaatacgaaaggcggtggccaacgcattcggcgcattgaatgcatttgctgaacgacatta
tgacacagagc
62. hgg1c.pk006.o15
gttcgccccgacggactacggccgactgatcgaatgcacgacgccattcagtgcccaaggggacaaccaactgagtttggcgatcgggg
agagagtgttgctggtgaagagcggaacgaggggatgggtgttgggacggagcacggacggagtgagaagtggttggttcccggcgaa
gttcgtgaagttggtctgacgaagagcggactgtgaagcatctgacctttcccaatacattcgaattgtttttcccattccattggtattttcttcac
acaatggcaaatgttgtgcttttggcacactaattaacgttttccccgaagcaggtgatccccgcaagaacattcagttcccttcccttctctccc
cctccttaattattaatgtctttgcttatgccattaataaaaaagtccttccgt
63. hgg1c.pk006.p21
ggaggaggtggcactgtcccaggagatgccatctgcacggacaaaggcaccggatgtgagccaggcttttgtagcagcacagactttgct
cgggcgcactgtgccggcacatgcaaacacgttttgcaagagtgcagtcatttggcttcggtgcccgacccagccaaatcatgcaccgaaa
cggccgagaactgtggcactataccggacatttgcaccgatgacactttggccgtttgtggttgtgctcacacgtgcaatcgttgccatcacc
aggcttcatatatggcacaaggaaggtgcaagaatgtgcagtaatgggatcaattagcacacagattacagtaatgatgtaaaagcattcga
ctctaacgttccctatcgtatatttctaccgtacatacaacaaaaagcgcttttgtagttttatggcatacagtaacccattatgctattcatgctttg
attcatttaaactttgaactattttcgataaacaatttaaaccatataaattaattatgt
64. hgg1c.pk007.a21
ataccgcgaggaggctcggatttacactcaattggaattggacaaacttcgccagcacattcagtctcgacaagtgtgtgacacgctgagac
tcatttatcaacttcacactccgaatagaacatcaagctttatcgcgggtaatgctgaacatatttcgccggaggaaagaaggagtcactgcg
aattgttcggcttttccgaagcccaacgaaacggcgacgacgaaacggacgaaataacggatgaatacataaacgaatacgaaaatgatg
aatacataacggatgacgacgaatgacggagaagggacacttaacacacttttgttatccgattaatataatatttatgttttttcactttacaaca
aaagttgccattaattccaaaataaacacttc
65. hgg1c.pk007.b23
gaatgcgacatgttggagctgtacacaaaggcccaagcgcatcaggcgaaccaaggtcctttgtccaaaatccccaacatggagccttcgc
gggtccgcgcatcgttcattcgctttgagaagttcctcgactgccccgagagttacaactgtcctcagatgataaaaatcacggctgcaagaa
tccgcgagtccgtccaaagacgcacgtttgaacacatcgtcggcgcttatcgcactatttgggagaaggtgacgacgccagagaatgagta
ccaacaaatggagcagatgagaagcgttgaagaggtggaaaagacgctatgaagaagtgatttttaatatgaacactcccgtttaactgtga
tgtttttaaatggtcgctataataaattatttctccgcc
66. hgg1c.pk007.b6
acaatgaccaaaacgaaggtcagggagacggagcaattgctgncggaggtgttaacctcgacgatattgacgtggatttaattgacggag
aaattgattaccaagccacttgggggcataacccttttgagcatggaggcggtaatttgttgcagaacctgcaagagcaaaacattgacgag
caagaggaggagaaagatccgtgttgtcccggcagtcaaaaaatggtttcgctgatggccaattacgttgacactttcgctcattccttttcca
agtcatcgctttttgatcgaatgtttccccaatctctttctctctccgtcctttggcttttggcactgtccaatttcgctaccgcttcgggtgccgttca
acactacgatggtttcaaattgcttcgtgtcatcccacaaacattggaacagctcgccgcccttcgcaacttcagcgaatatgtcggccttcag
cccaattcgggtgccgaagtttggaactttcgcccattcgttggccaaccgtccgaattttttgccgcgcctgacaatgccaaaagagtcacc
gatttcatcaaattcgactccatcggcaaaacctccgagggccgtgaaattcccttcctgacgctcggctacccctcgaaaacctccaaaaa
gcccgctctgttcctcgatgctggcatccacgcccgcgaatggattgcgcccgcgattgcccttcactttatcaacgcgctgatcaatgagcc
caaattccattctctgctctccgacatcgatgtgcacgtccttccgtcgcttaacccggacggatacactcacagtgcgaattcacagacccaa
gccggcgttaacaaatgcccgtgcagtttcgtcaatttgctggtcgaccgttccgtcaatttcgacgccgacgcgatgcagctcaaatacgcg
ctcatttgtcgcttggttgaggccgcgccgtccgcgctgaacgacggacagatggaaatgctgcgcgactattgcgcgaaagggcctttttg
gggggcgccggtggtggaagtcgcaaaggaagaggcggcataaatgaaaaggaacgaatggatggacaaagaggcggagatgttcac
aaataaaat
67. hgg1c.pk007.d10
ttggacaatggcagtcgcataccattgcccgacgttaaaccgggatatatccgcgcgctgatcccagacgaagcgccaaaaacagccgaa
gaatgggaaaggattttcgcggacattgaaccgattgtgttgcgagggaacacccattggcatcatcccaatttcttcgcttattactcaaccg
cgtgcagttacgccgccattattggcgacattctaagcggcggaatctcatcgcttggctttacctggaattcgagccctgcaattacagaatt
ggagcagaaaatgttggattggctggccaaggcaatcggattgcccaaggccttttggaattcggaccctgggcccggcatcggaatgatc
caatgtaccgcaagcgacgcaactttagtcgctttgctcaacgccagggcccgagccgtggagaaaatgaaacgcaatggcagcggcac
attgttggcatcgatgggtgccaacagcagtgttttgatcccgaatttgctgagagatccgatcgcaaaggcaatgaatcgattgaatggaat
gagcgagacgcttcggaacagaataaaaacgaatggaaatgtattgacacgaatgtttggagttgaaatgaaaggggaagaaagttacgc
ggcaacaaacggacaactgacaaccttcgaggctcacgacccgaagtatttcagccgattggtcgcttactgttccgatcagtcccattcatc
cgttgacaaaggaataatgttaagcggcgtcaaaatgcgaaaattgccaacaaaccgagaaaagggcggaaatttcgtgctgagcgcaga
agtgttggaggcggcgataaaagaggacaaagccagcggactgacccctttcgttttggtggtcagcgtcggcacgacaaacacttgcgc
ggtggaatcgtgccgcgagttggggccaatttgcaacagagagggcatttggctgcacgtcgacgccgcttatgcaggcagttttttgatttg
cgatgaattccgccatttgtcggacggtgttgaat
68. hgg1c.pk007.f21
tgcagaggcaggggagcagaaagaaggaggagaaaagaaaggagaagaaaagcccaagggaaagaaggagaagcgcgctgcaga
gaaagaggaaaagaagacggaaaacaaagaagcagagaaaaaagagaatgaggagcaaaagcctgctggcaagaaggagaagcgc
gccgcagagaaggaggaaaagaagtcagaaagcaaggaagcagagaaaaaggagaatgaggagcaaaagcctgctggtaagaagg
agaagcgcgccgcagagaaagaggaaaagaagtcagaaagcaatgaagcag
69. hgg1c.pk007.h12
ccgcccatcattccatcatcttgtggggaagaaggcaacagtggtaagtgttccggaatgtcgcgcatcggactgccactgccacctccgg
cggtgcagcaataatggtgatgatgatggtggtgatgattatttgttgtggtggaagagtgatgtggtgatgatgaggatgaacaacaacggt
acggtgaggagggggcgtacggtagcggagcagtaccagcagcagtaccaccaccaccgccactgtgctgtggcgagttgccgttgctt
cggccagaaccactggccaagtttaacgccaaacttctgcttgatctgttcaacctcagatggctcttctgtagtcctaaagccgccaataaatt
tgacgcgcttatcgctcttcaaaattttggactgggcaaaatcgatttcgggcagcgaattgatgaagaaaaagtggctcttggcgaacagtg
cgtcccaacccgggaaattcaaatggaatttttcctccgccattttttgctttttggcactttttacccgtcggcattccttccgactgtttcattttgc
tcaaatggcccatcaaatgcagctgaaacggcgacatcggctggctttgggtggcgattgtgaccgcaatttgcagaaagtggaaaagtgc
caacgaatactccgtcaaacagaatgcctctgccattcccacgtcaaacttttcggcacgtaatccgtccatcagttcaaagtcctcggcaattt
ctganttgattcaaaaattgcattaaacaattttc
70. hgg1c.pk007.h5
gggggggtacgaaccccgcgtacctcccccagggaccatccctgggtgaaagtgcatccaaagatttttttgtgccaacccattcagatgtt
cctttctgtccgagccggacatgcccaccaggagtttttctttgtcgtgtgatcatcaccctacggcttcgtccgcaaaaccaatgagattagc
gcatgagaaagaacaatttgcttcctctgcgaattcgttgtgtgcttccccattgccaaagcaatcggccgattcggccagtgcctttttgcgga
aaccaaaacaattggcggattcgcagccgaatcagacacatgcccgaaacacagttgggatccc
71. hgg1c.pk007.j24
aggaaatcggccgaagacgatgacgacgatcttccagaggaaaatgctgtcaatttggtcgttttagatgaagtgactgcggcagctggag
gaaaagcattctgcaaaggagttttggcagggcacagtcccacttcaacttcaatggaccaccctttgcgaaagcgacacgcgactttcgag
agggattcgctgaaaatggaggtgaaaagtcgcgaaagcgggccggccaacgcggaggaaaagggcaaaaatgaatttgaggaggct
gagggaaagttggaggacgacggagggaggggcggagagataaacggaagcgacactttggctgacaaaaaagatcgatcgcagaa
ccgcgagcaatgtcaaaagtcaattgtgaagtcaatgagcgatttgtttggaaatcttcaaaaattggaaactgttgcctttccgattgacaatta
cacggatgggcgcagtgacgggaattttttagaggatatgacgcaacgcgtaaatgaacttaaactagaggaaggacaagcaacggttgg
gcatggaagaggcgaatgggcaaagcaattggtggaggagaggaagacaaaagcggaacaaalgcaacaacggaatgagtacggaa
acagcgaaggtaglgggctcaattgcacatcggcgaatgccatgcgaattcccttgtcatcggttttcgagggtatttcaacggaaggtcaaa
aaattgacaacgaagaaaaggaacgaagaaatgaagaggaa
72. hgg1c.pk007.k17
atttccaagcaaatcaactccaaattctgaacactcgcggcgaattaacgcacgccgtcccctttgaaaaggccaagcaaatttcggctattgt
ttacggcactcaattcgtcgccattggcaattcccacggtgtcatttcgttgctcacttcgcccgccctccaatccctttacagcatcgaagccc
attcgatgaaagtgcgctgcttaacttttctcactgaccattgcaaattgctgagcggttccgacgacaaaaccatcaaactctttgcgttgggc
gaaacgcgtgcacagcttttgcgcattttctgtggccacaaaggcattgtcacggggttggccgtctgcgaagcatccgaaagcgaacggtt
tgcgagttgcgggacggacaattgcgcaattgtatgggacacggagagcggagagcaaagacatgtgttttccgaatgcacgggaatggc
caacgacgtgccccgttgtgtcgcatttactcccaacggtcgatttttggttgccggttccgaggaggcgagcattttggcctttcgcgtcccg
caacccaaaaattatgtggaacaattgccattgtggacagaggaacaacagagggaatcggttggagaggcaaacggcgaaggaatggc
cgacgaatgggcggaagagagaatgtcgccatttgccgaattcgactcgccgcatgcaaattcgaagcaaaaccgacaaaaaagggaga
atgggcgccacttcttccggtggagagacgccgaatgatgcggcggaatttggggatgacaatggaggagacgacaatcggcaaacgg
aagaggcggcggcgatggacgtggaagagatggaaagacgcgagttggaaatgcaattgggcatc
73. hgg1c.pk007.l12
taagcagtggtatcaacgcagagtacgcggggcggcgggtgacgacgtggtgatggtgacggccgttgagggggaggacgcaaacgg
agagaaagtggttgttgaaaagttggagacgcgggaggaaatgacggggagcagtgacagtcagccgaagctgacggtggagatgcgc
aaggaaagcactgacaacgaatcgctcacggccgcctgcacatccgctgttgcaatgatgctgaacatcaaggaaaaccatccttcgatgt
cgactgtgacgccgggcgctaccatcagtccggtgatcggtggctttggtcggcgtcgtaaataatttgttggtgtcgtcgacagaaaatcg
ggcgtaatctttgatcatcaattgttgattaictttattcaataaatacctatatttaatgcccaaaagagagataaaagccat
74. hgg1c.pk007.n20
tttatttggccttttgattcttttttattgtggatgatcgaatgttgaacgcttttgctgaccatttgtttgaaactagttcct
75. hgg1c.pk007.o8
aacgaatagatattattgtcctgtgtcactgtgcggaaaccgttttccgaaacggttcgaatgaaattccattaggaaaaaagcggagagaca
atgggatcgaggggactgagcgaaattctgatgctgatcgacgattatgccgaaacacttccattgcacgtcgaaccttacaactataaaaa
ggcagaactggcgcaaaaacgtccgatttcggctttgtgcacgccgctggtcggctccattcctctcccggacacggaggaagttccaattg
gcagtttagtggcggtgtggaagaaggaggaggaccagcgggaaicgaaatggattttggccgaagtcattgaccaaagcgcgggagtg
cgcggacgaggccgttacacactgttggaccatgtcgcggaatacgaatattaccgcaactatttcatactcagtcggacgcctcccgtcac
gccaaatgtcaaatattcgctagtgcgccaaaagctcccctttctgctgaagaaagttccccgccaagacattatcccattgccccgttttcgtg
ccgatcctcggcacaatgccagcgcattatttggtcccggttcactcgtgatggcacgcttcccaaaaacgtcggtgttctatcgcgcttgtgt
gatcgcgccacctgagcgtttacgtgacgggtattgtgttacatttgacatgaaatctgaattcaattgtcaagggaatggaagtaaaaacgaa
actgtgcaaagttacgtcattccccagctgtatgttgttcagaatccgccggggaagcgccactcgcgaatgccgcacgagaggcaaactg
atgaggaataaggatttgtgttgtgttttcttaatggttacactgtgctttccggatcaccaattttgtacttcctga
76. hgg1c.pk007.p17
gagggaaaggaaggcgttgtcaatttggtcatttccttttcgcctgtcccacagcaacaccaacaacaaacggaacaggtgcccgcgcctc
cgcagcaaagcgacggacaacaaacggcggctgccgcgcagactcaagttgctttctcggagaaggatttggacgaaatgcaggaaatg
tttccgaccattgaccgagaagtgatccgatcagttttggaggccaatcgaggggccaaagactcgacggtgaacgcactgatcgaaatgg
ccaattgaatggacagaaaaagagacgaacggaggaaggggggggggacttgtgagaaattgaattgtgattggaccaatgctttttaaaa
g
77. hgg1c.pk007.p4
acaaggaacgaccattgaggagctgttcggcgatggcatttattgggcgggctgtgccatcgttcgtctgctgggccaacatcggcgctttg
aagtgctcgacttctcctaccatttgttgcgcgtgaatcgggcggttgggtcggcgcctaatcagcagcagcagcaacaacacggcacaac
ggcagggacaaaggaagggggaagcggcaacaaagcgcagcaacagcagcagcaacaacagcgaaatattgtgcggctcatcgacc
gaatccgtcgagttcaggcgcaacacaaccaggtgttcgccctgctcggcaatttctgcgttcacttggaggaacaggagcagaaaattcg
gcattttgcaccgcccgtctatcagccgctgcaaaatccgtacgcaaatggccacgaaggcattgcgttgtgacaaatgggcggcgctgtg
aatgaacacggcaaaaaagaagcgacagcaataaaataataaataataatgcacatacgtaaacataattaattacacactgcctaattaatta
ctaattaattaacattattcccgattaactgttcactttttaatttattattttgtaattatttttaacacatgaaaattaaatgccatacaaaaacct
78. hgg1c.pk007.p9
taagcagtggtatcaacgcagagtacgggggacaaccccgtcagcaacagaaccgcatgccgggcatgggcggaggtcatcagcagc
aaggaatgcgctaccagggacagccgaaaggaatgcagcaacaacaccaccagcaggctcagcaaccacaaattgcctattcgtcgtat
ccgcagcagcagagccgtggcatggcaccgcaaatgggtggcggaggcggtggaggagtcaaagctggccatgcgatcactgcaaca
caccaggaaccgttgaacacacagatacttaccgaggctgacatgaccggacaaaagcaaatgcttggtgagcgtctgtacgcgatggttg
cgcgttgcttccgggacggtgatgtcgagaaagttggcaagatcacgggaatgcttctcgagatggagaatgccgagattttgctgttgcttg
gagacgaggaaatgttgcgtttgcgcgtggacgaagcagcaacggtgctttaccaggctacggggcagaaggaagcgcaataggatga
atgaaggaaagaatggatgaataaattgtgagttaaaaaaagaaaattcataaaaatcgatatgctatttggtttctttgtctgaagtaaatgttttt
ctgt
79. hgg1c.pk008.d11
ctggcacaagtagaagaaaagggttttattcttgtccgttgtccaattgcgcttgctgaggaaaaatgtgggaatgagatggctgaatcgctta
atggacaaaagaacaaaaaaattggggttgctgtcagcaaggacaaagtcatcattggttattacgaccctaattccactatggtgattcacca
gttggagcacgagatgcagtgtttgaagcacgaggtgcagaagtgttatattggttgatgcgtttatgcgatctaaatgttattctctgcaattta
cgtgcaattgtatttgatttttgcataagaacattattggtttctcccaaattttaaaagtactttgtcactagtaaatcgag
80. hgg1c.pk008.e15
cgaagaaagccaaaaaggccgtcccgaagaagtcgccggctgcgaagaaggcgaagcccaccgctgctgcaaaacccgaagttcctct
gcccgtttcgccggcagtgacaaagtctaagaccgcgaaggcactgaagaaagacgtcccgaaaaagtctaagatggccaagagatctc
ctaagatcgctaagaagtcgaagactccgaaaaaggcgacggggggtgcgaaaacttcgcggaaggtcaagaaggtggtggcgtcaaa
atctgccaagaaggatgtcggcgttgatggtgcttcgtgaatatttcttttcctcccttccatcgatctccaaaatgtaaaattcgtttatgtatctct
caattaccttgcattttccactc
81. hgg1c.pk008.h22
ggaacagtgcagcgtcgatcaggcaattgtcgagtttcagcgatgccgtaacgacaacaaattgacacgtttgctcaaccagctcaaaggg
atgatggaatgccaaatccgtgctgtgcaacaagcagaggaatcaatgcgagtgaccaacagcaaattggtggacgaaatcaacgagttg
gagttcagcaaagagcagcttctgtcaaagcacaagcttgaactggaacgggccaagcgcaagtttggggacaatcagcgttcattggtcg
acgcgaagcacagtttggatgtgatcaggcagaaacaccaggccaccgtcgacgaactgggcaaaattgacctgagattgggcaaactc
catgacgaattggccgagaagcacaaaaattatttggactacaaaaatcgtttggacgcacaatacaacgaattgttgacggcggtgctgga
gaaagtcacgaaactttgcgaccactttcagcagatcgaggaccagaagaaacgcttcgcagaattggccaatgaaatgctcacgaagaa
caaggaggatttgaagcaattggagacgaagaaaaaggccgaaacggaagattaattggccgtcatggctggaattatataatgtctgactt
tattacctattttgtatcgtgattgtagaaccatatttatgtgtcctgactttttttttgctgtgtataaaaatgaagcatccaaa
82. hgg1c.pk008.n8
gaccaaaaaaaaaaaaattttgtttatcgatggcagaaattttcttaccatttttttagcgaaaaatgaagtccggtggtgccattaggcaaagcc
aggaaattcttcaaaatttatggatttgtatatatttttaccaaaaattatgttttttacaaaaactatcacccaataataggcaaaaattttattctgac
tttttgaggtattctcaatccatcggagccaatttctgtgagtgctcagccaaaaccaacgaaggggtcagcgaactgttctcgaagttggcaa
ttgaaatgttaaataaatcttccgaagagacggaagacaccgatggaatcggcacgactcctttccaacggcattacgggtcgaggcggag
ccttagaattgcggacgaagatgaaacacatgcggaacggaaacgacgcggaaaatgttgccgatgatgcataaaaggaaataagatag
acgaacattccaactaattgtattatacttatggacaaagttctataa
83. hgg1c.pk009.a7
ccgtacgtgacatcaccgaccttatggtcaaacagctcgagtccaaggaccgtcaaatcgtcgacaaagactttgaactggcacaaaaaga
tgtgctgctggaagaaaaagaccggctgttacgcgagaaggacgagatgatcgcacgtcttcagggttatatcaacggacttggtgttccat
tgcccgcgccggcagaacagcagcagcagggcggcggcggccaatgagagagagaggcctctgacagagtccgactgaccggaag
aaaaaaattcgcggacttttctttgatgtggaatgtttttgttttggttttttgatgctttgcgcct
84. hgg1c.pk009.b23
taagcagtggtatcaacgcagagtacgcgggccgcgggtggacacgctgaaaaagacggccaaaccattattggacatagtagggctca
aatcggtcgcactgtccgtacacaacagagaaattcccttccgacccgtgccgataggggcggaagcatttcgggaaatattcggcgacgc
ggggggagccatggcggagagaaatgaatcgaacgaaactgaggaggaggaggaattgatggagttgggggaagaagagcaaatcat
ctgctgacgaatggaggaacaacgaaaaatgattgggccaaaacaaatggcagagggaacgatttgggccgaaagtgaccacgagtcg
aggctcctcacctttttattttccaaccggctgtttgtactgttttgaaccattccgtcgataaatttctctgtgtac
85. hgg1c.pk009.e14
taagcagtggtatcaacgcagagtacgcgggggagccaatcggacgacgacgaggaggacggccgattggcacgacgacaaaagac
cgaaccgcaattcgtcatttcgcttggattttctccgcctcatcagcacaattccgaaagtgtcatcaatttttgtgagagtgacgaaagcggcg
acgatgacaaatttggacaaccgcagcagaggatcgtttgcattttgccctctccgtctccaacagcgacaattgtcgtcggaattccctctgc
tgatgagagtcagccaggcgacgaggaggaggacaaccgcagcaaactgagaactcctcctccgactgatcattacgaatttgaagagc
aatcagtgaaagttcaaataacagtacctgacgatggggaggaggacgaaaatgaattggacgaggaagagagagggaggaggcagc
ggaaggggagtgccgaaccgacagacggagcggaagaagctcccgccgcaagggaaaatgttaaaggtgacaaaacgccaacagag
gaacaaattgaagatgatgatgatgacttgctgatcgaactggttgatgaatgagtcctcaattcattttgtccaaattacattttgcatattaattg
tacccttttaattacgaaatatttgttgaaacgt
86. hgg1c.pk009.g14
catggacgggaacagcacaatacacaggacagacattcgagcatgttacaacgtctctacacttatttctacacgcccatctttcctgtgtcgc
gggatgagcagcaaaggaatcagttggacttgccttcttgggtaaattcgccgcgaatgggaggagaagccgaggcgaatgggccaagc
gtacaaagtgccagcaatgccgcgcaactccgacagtcttcttcgtctcatgtgggcaccttcgcccaccaaccagggccatcatcaaatgc
acaagcgacttcttcctcaatggtcactttggaggacgactccgacgacgatgaggcgggggacgcggtggaatttttgcaagagataaaa
cgatccaaaagcttgcacaatctcgaggaggaagagtcggaaggcgacgaagaaatggacgtagaaaatggtcatgggaatgacagtga
tgacgaagatgatgataataatgacgaaattgtcgacacggtctctgctagggatgtatgaatttaatgcaaataaatcgcttgagattg
87. hgg1c.pk009.j14
aagtggacattgtgtcaaaaacggagtttggcacaatgccaaagggtgccagtgaagaaccatcctctggttcaacatcagaagccgacca
ggaaatgacaattacgccaggggaaaacacgaaatgggcattgtgccaaaaagggaattggacacaatgccaaaagatgccagtgaaga
agcttcatcgggctcaacatcagaagccgattccattctcgaaactgagtttgatttcgttttgcccgctgaagagcatccgatgcaagaagag
attgtaaatgctgaccatcatcatttggacaatgaaaccaaaaacgacggcattttgtcaaaagagaagtgggaaatggacaaaatgccaaa
aaaaagagattggcattgtgccaaaaaaggaaattattttgattgttgtgccgccaaaggaccaacccgtgaatggagaaagagatgcagc
agcaatgactttctcctcctccatttcaacatcgagtgccgattcagtcgaaaacgacgacgaatcagaagccgatttctgtgatgaatttgattt
taaaaaaagtgccaaaagaagctgaagaagatggggaaaaagccgatcagaaagcattagcactaaatgatggccaagaaaaaaaaga
agccgagaaaaatgagggaaaaagcgcgacggattttgattggatcaaagaaattgtggaagcaaccctaaacgaagaggaaagcaaaa
aaa
88. hgg1c.pk009.j18
catgatgcacaagaaggacgatttcatctattttgaggacgatcagcttcaactgctcggcatgccttaccagggcgaaaatgtgttcatgttc
gtgatgctgcccaaggaacgcttcgggttggccaaactgttggccgaattggacggcaaaaagttgctggaactgaccaaaaagcgggga
aaacgcgaagtgcaggtggtgttgcccaagttcaagttggaatccacgcaccaattgaacaaaccgttggccaacatgggcatggccacc
gctttctccgacagtgccaattttgagggcattgccaatgggccgttgaaaatcagcgaagtggtgcagaaggcgttcattgaggttaacga
gcagggcactgaggccgcggctgccacaattgtccatgttatggcacttagcttaatgatagagccaccgcctccccaatttgtggccgacc
gcccattcgtcgcttttctcgtcaatcacagccaaactgtgcttttcaactccattttctttggctgaacgaagagaacaaaaagagctttttgttg
gatctgtcctccaattttcgaaaacttcgttgctattttatttttggatataattttccttattttgtaatgtattaattggcttttccataataaattggcttt
gtaaaa
89. hgg1c.pk009.j9
tggcgcacaacgcgtgaacatttctgcgctggaccttttgagttgcgagccgcacagtttcggatgccgtggcgggtgggaggacaaagc
gttcgaacattacgtgaagcggggcctttgcacgggctccgacttcggggccaaccgcggctgcaagccgtacccattcgcaccggtgcc
gcatccgagcaacgtgccattgcacaaaacgccaaaatgtacacaccgttgcccaaatggcgagtacaattcgacctatgccaaggacaa
attctacggccaaaacatgggagtgcttgacgacggcaatgtcgaggcaatccaagcggaaataatgcgggcgggccccgtcaccgccg
ccttccgcgtctacgaggactttggccactacgcaagtggcgtttatcagcacgtggcgggcaaatatcgcgggggccacgcggtgcgag
tcatcggctggggatacgacacggacagcaaattgccatattggctggtggccaattcgtggaataccgcgtggggcgatggcggcttctt
caaaattcggatgggctccgacgagtgcggcttcgaaacttcgggcatttgctttgcggacccggaccaatccaactgaaatcctcctccaa
ataaacgctttaattatggaggaaattaagattaattgttataatttaatggatggaataaataatccacataattagttaaagtcaaataaaaaga
gacga
90. hgg1c.pk009.k16
agaattttttggacaaaaaagtgttgaaaatgttgaaggactttgagactgaacagcatgccaaaaaggaaaagcgtaaagaaatcagccgt
aaaagcattgaatatactcgaagcaaacaggaagaagacgatcaggaggatgcaacagacgaaaagaacgaaagttggtttggcattaag
ccggatgaaggcacgctcgaacagaggcgactttttgtacctgaccgacgtctctccaagacagaaaagttattgaccgaaattggttcatc
accaggagtgcgaatattgcgaagacatttgaaagaacattctgttgaacgtttaatttcacctaaaattgaggaatcgtctccgcaaaaatgg
gaaattcataaaccaagaaagagaagacgtaacgatgagatttttacatctgagtcgtcagcacaagaagtcttcgaaacaaatgaaaaggg
tgcagttgctatcccggaaaagcccaaaggaatatcccaaaaaatgccaaatgacaagaagcggattaaagaagaagtgggaactcatga
gatgaaaatggtatttcacaaaaggccaaggcttggcgaggaggaaactcacaattccgaattgggcatcagtaaaagtctgaagccgaag
aaattagccaaactcggtcaaaaattgaagttaaaagccgcttcaaaaagagaagacattattcacatgaagaaga
91. hgg1c.pk009.l16
agcgtcacctccaccatcgtccaatccaacgtcacttcccccacgccgtccagcacggcccaacaggaccttcacgcactcgccctccatg
gcactttgccctccccaacggcactcccaccgacgaacggaggcacgagcagtacaaaagcagaagaaaggcggcaacagcagacga
actccccgtgggtgttggtcgcgcccactccgctccacccagccaattctctgctgttcactacgagtgccaatgctatccatcacaaaattgg
caaatgaatgtggcaaacgatgggcgaaccaacggccaataacgtcaaaggaaataaggatttttggaagatctttagctggctattaaact
cagcaaaaatccgtacttttagtttttaaaggggctcagtatgggataaatct
92. hgg1c.pk010.e7
atgaccgtcgttctttgccaacctatttaagttctcctccggcgcgttcttcggcctttttcccctccggtagtgttcatatgcatttcagcgcggg
aaaggcggaagatgcgcgattgctttacacaattggccgtcccgaggctgaatggagttttggcgcgggcgaatcgattcagattagtgac
agcgaaacgaaaaaacgcgaggcattccccgcgacaatgggcagcacaataatagcaaaatcgccgcaagaaacaacgacgacgaaa
cagccgcgaggaatgggacaaatggaacacacagcccggaggggagggagagcacaaaaacagacaacgcagcagcagcccacga
tgagaagggggagaggagaaagtgagcagaggggacccccctcgtcttcgatgtttggcgggccgtcttcgccacaatcgcagcctttcg
gcgcttcttctcctcaatttcggcctggtcccatcctcagccaccatcagacacgttcctatgaaagcaatgaagaatcgaaaatgtcggaag
agaaggctggaagaagagcaaatgaacagatttatagaggaagagcagataaggaaagaagaggaggaatataaaatcagaatgtggg
aagagaaaaggaaggtggaagatgagaggcggagacggatagagaaagagaagcaagaagaagaagggagaaggatggaagact
93. hgg1c.pk011.j16
aagtacggaaacatgggctgcaacggaggcatcatggacaacgccttccaatacattaaggacaacaaaggcatcgacaaagagacggc
ctacccctacaaggccaagaccggcaaaaagtgtttgttcaagcacaacgaggcgtacaaagagggcaaagtgtccttccgagtgggag
agactcatattgccgacctgcccttttccgaataccaaaagctgaacggattccgtcgtttgatgggcgacagtttgcgccgcaatgcatcca
cttttctggcgccaatgaatgtgggcgatttgccggaatcggtggactggcgggacaaaggatgggtgaccgaagtgaaaaaccagggaa
tgtgcggctcgtgctgggcattcagtgccaccggcgcattggagggacaacacgtgcgcgacaagggacatcttgtttcactgtcggaaca
aaatctgatcgactgctcgaagaagtacggaaacatgggctgcaacggaggcatcatggacaacgccttccaatacattaaggacaacaa
aggcatcgacaaagagacggcctacccctacaaggccaagaccggcaaaaagtgtttgttcaagcgcaacgacgtgggggcaaccgac
tcgggttataacgacatagccgaaggggacgaggaggacctgaagatggctgttgcaacgcaagggcccgtctcagttgccattgatgct
ggtcaccgttcctttcaattgtacaccaacggcgtttactttgagaaggaatgcgacccggaaaatttggaccatggtgtgctcgtaaaatttg
gaccatggtgtgctcgtggtgggctacggcaccgacccaacccaaggcgactattggattgtgaagaacagctggggcacccgctgggg
cgagcagggatacattcgcatggcacgcaatcgcaacaacaattgcggcatcgcttcccacgcctctttcccattggtctgatcggagtgaa
tttgttgcccttgcgctgattcagagacatttcatttgattaatcgtgcaaaa
94. hgg1c.pk011.j6
gatcattgtttttttccactataaagtataaattttaattattctaaagtgatcactgatcagttcattttctgcctattagaggccatatccgtcccatat
ttcctccaattgttgcatcatttggtagtatttttcgctatcttttatccttattgtatttctggtgggatattggatatattccgaaaagtccgtccagcc
aatggcaccttccaaaagcatcctgatgaatacgatcagtaccggcgcctcgcttccgaaagcgatgaatgccctgtcgatgatgccgtaaa
aagggatgtttcggcgtctccttttccccttttgccagtcccaatacattctgagcagttcgaacttctcgtgcagttcgttgtacttctccaactcg
acgcctttaaatgcggtgtattgaatgttttgtcctccaatcgttcaaacagttcgaattccttctcaattttctcttgcattcccgcgtactctgcgt
tgataccactgctta
95. hgg1c.pk011.n19
aacacaccaaaaaaaagaaaaggaagtcgctgctttcttcggccggtcgtattcaaatcggtcttaaatacgacggggaccgtttcaaactg
atcgtttcagtgattgcggcaaaagatttgagtccaatagaaaaagagggccacgcggacccatacgtaacacttcggctgatgccttccgc
aaatggccacccggccacgacaaaagtgcaaaagggcagaaagcacacggaaatggtgcccaattcgttggacccgcaatttaaccaaa
acttcgagtttgacattcactgctccgacttccccaatttcaagctgcacttggctgtgaaagacggcataaattacggtcttttgcacagtacg
cccactttcggtgttgctgaagtgccgttgcataacttcgacccgttgaaacccatcgtcagccaatggttggatttgtcgtcgcctcagagat
gaac
96. hgg1c.pk011.o2
atcaaatcggtgaggggaaggggtgcaaagtgatcagacgatcgacgacactccaaaagcgggacagcaaaacgcatgagaacggatt
tgcacgggtggcgccacaatttgtgttccctttgctccatttctacaatttcgatagtttttgcgatgtcgcgcagtccaacgtctttttggggaaat
tggcgctgacccttggcgagttggttggatgtgcggcccattcgccctccattctctccattttgagctcctccttcgcttggctgtccgtcctcc
gtcgtttcggtgcccttcgccaccctttcgtccgccactgttccatctgcgcttatttggccatttgtgatacattttggccatcgtcggaagtgct
gcgcgaacatttcactgacgaactaagagattgcagagaatggttgggagagatggcgacggaatttgtacttggcgaaatggacgaaac
aattcgacaatccatcggaattttgtccgttaaaattgacaacattctcagactgatttaacttttgtttaatccaattaataaatggaaatc
97. hgg1c.pk012.c8
tcaacgcagagtgcgcggggagcccggacaacccggagagaagggagagcacggacactgcgaccattgcccaccgccacggactg
cgcctggctattgagcattgggaagccaattttggagatgacaattgggaagaggagaagaaaagtgggagaaaaa
98. hgg1c.pk012.e11
gaagtgattggcaagctgacacaacaattccagcagttggacagtgccgaggagcgccgggcagtgcgggacaaatttgtttctcgttttgc
aaaggagagcaggcaatgtttggcggcgcaaagcgggcacaaagagacggcggcgccggcactgaaacgatcgaaccgccgcgtgg
agacgatcaaaccggcaaacaaacggcttggtgactgcccgaacattttatggggagagccgatcccagacgatgtcataactgcttttttg
gctgactgcaatcaaaggcgcaacgaggaatcttgggttccgggcctggaggagcgcctgccaaacaactccctcatttggttgagtaaac
gggggcagtggctcgacgcgccgatggtcgattattatctcgacctgatctgcaagcactcttcgacgaagcgcgccgtccacattccggt
cgtggacattttgtgttttcggcaaaagagggccgtaaaaacaccatggtattgggacttgagcaatgttgagctcatcttcgccccgggcca
ccacggcaaccattggattatggttgtgtgtgacatggcgaatcgaacattgacgcttttcgactcattgagcaacaacgacggcggcggca
caaccgaaaatcgtgcgttcgcagaggacgtaatgtgcattttgcgcacaatttcgctcaagcaacgaacccaaattgtacgcgaacagtgg
agggtgatcctggatcggaaggcgcccagacaggccaactcgaccgactgtgccgtatttgctctcctctacgcgcaatactcactgacgg
gcgcaagaatggactttgggcagcagcacattcgtgagatgaggcgacaaatgtgtatgaatgtgatctcttcaattgttgttagtgatcagt
99. hgg1c.pk012.k10
tttcgccgccgcgcgttctttcgccctctccgccgcccctaaaaaagtcgaagaaagagaagcgaaagaaggagaagcggcagcgtcgc
agcaagcacatgtcgccttcgttggccgtccaccgccagccccactcgggctcggacatggaaatgggcggggaggaggaggacgga
ctgtccaacggcggagggcgcaaaatgctcaaaatggagtcatcgtcgcccgtcaacaacagcaagatgattttcattggtccggtcaagc
ccattgtccaacagtcgccgactgcaccacaaaagctgcccaaggtggagctgcgcaaccagtccatcatgttggacctttccgatccaaa
gactgtttcgccaccgaaattcaaaccgattggcgaactgtcggactatcagcagcaaatcaacggatcaaaaggcatgcaagaactttcca
acattcgattggccactcctccgccacaaccgccgccggtgcttcgcaatgttccgttgcctcccgtgacgtcgaataacaacgaaatgatg
gtggaccgcaattacattgcaaagggagcctcgccgccgaagttcaaaccgattggcgaactgtcggattaccagcaacaaatcaacgga
tcacaagaagtttcaaacattcgattcgccactcctccgccacaaccgccgccggtgcttcgcaatgttccgttgcctcccgtgacgtcnaat
aacaacgaaatgatggtggaccgcacttgtcgaaacaacacagcaaagccgtccgcttcggattttgggctcgctgcttcatcatcatcgac
atcgacaggagaggaggatgatgcgcaactgttcggacggcgagtggtcaaatttctgcgcagtttaaacaacggacgccgtaggcgcc
gcgcatgcattggcattgagcaagtgatgatcgaatttgaaacggaggaggaagaggagcaaaagcgatnatcacctaattatttgttttgat
cggaatcacctctgctggatatttttg
100. hgg1c.pk012.l10
agaagattcagctaagctcagtgaaatcagcgacaacatcgagcaggaggattacgcgggacgtccaaaaggcaaaggactttcgggat
ggatccaaatcatcaaaccactggtacagggccgagtcatgttgacattgacggttgtcaatttcattttgttcattttgacgctcattctgctcat
atacctcctcgttttcgcggcgctcatcagcacgtcgagtgagaagagaaaggagcttcagggcaagatcaatacggtggattactgttcgg
tgagttggcaccctctgtcggcatgttctgcaaagtgtaaaggagtcggcgatcgggtggaagactatcctacgcgctcttcttacatcgacc
atgtgactggccaatgtcctgaatatttcaacaaagcgcctgaagaccttgagcagattaagtacagtgttccgtgcaatgtttgggcgtgtga
atgaacgacgaatgcttatattatacaatatttatcttaatgtttgtgttttctgtgttgctgtctaaatatctgtgttcgatatttacattgatagtaaat
gttctgttgttaataaattctatatttgataaaacatattatcc
101. hgg1c.pk012.m22
gagaagcagcaggaagaaaaggaaacacaagtggaggagaagaaaatgcagttggatgaggaggaaaagcagcgggaagaggaag
agaagaagcaggaagagaaggaaacgcaacagaaagaggaagagaagaagcagaacgaggaagagaaaatgcatgaagagaagg
aggttgaagaaataattatgttggacagcaacgacgatgaagccatggaaaaggatggggaggagaaggaaaagcaattggaagataag
gaaaagccggaggaagcgcaaggggagcaggaggaagagaaggagaagcagttgggagagaaggaaatgcaaattgatgaattaatt
gtgttagacagcgatgatgaagagaaggaaaagcaaggggaagagaaggaggagacgcaaaggaaaaagttggaagagaaggaaaa
gcaattggaagagagggagatgccggaggaagagagcgcaatgcaagggaaggtgaaggaaaaacaagcggaagagaaggagaca
caaggggaaaaggagaagcagcaggaagaaaaggaaacgcaagtggaggagaagaaaatgcagttggatgaggaggaaaagcagc
aggaagagaaggaaaagaagcagacagagaaggatgttgaagaaataattatgttggacagcaacgatgatgaagaaatagaaaagga
aggggaagacaaggaaaggcaagaggaagagaaggagacgcaaggagaggagaaggaaaagcgagaggaagagaaggagaagc
aatcggaagaggaggaaaaaacggagaaaatggaaaagcagcaggaagagaaggaaatgcaagttgaagaagtaat
102. hgg1c.pk012.o24
aagggaccgactgttgctgatcaggaagaagagcaacagcaacggctaactgatcagccgaggagttctgatggcggacacggtgctatt
tgccgacatgacagccaaacaatggcaatgcgtgcgacagatttgcgggtcgaatgtgcgaatacccgtggtcgaagccgtgaagcgaa
ggacggacaaaagtggcaaaagacgggggaaacagcgggggcagagaagaagaagcacagcaaaacgaatggcg
103. hgg1c.pk013.e8
taagcagtggtatcaacgcagagtccgtgaaaccattgaaaaacaaaagaaaattaccagtgaaattgaccgattttacaaagaggtcgaag
aattggaggttcagcgagaagatgaacacgaggaattgaatgaacagtcggcactccgttcgggcatcgaaatgattgacgaacaaatcg
aacggtggaaaatggtcaacgaactcaagaagaaaaaggaaaacattgtcgagtcggtggcaaccaaatttgagcaaaagccgaactttg
accctgtggaaatgtctgatgacgatgattccgatattgttgattttgataggataagttggagaacaaaagctttttaaaggataaatttttct
104. hgg1c.pk014.c5
gcagggcacgtccactgagaaacggatggaaagggacgcagaagcaatgcgcctaaaacagcagaaagctgcagcgaaaaaagccg
aagaggaaaaggcaaatgcacaagcgccgaaggtggtgaaagtggaccctttgaagggactgtgacaagcgaaaagaaccactcggct
atgggatgcacaaactgacgcttttccttgctttatttagtcaatttttcgaattcttttcagcaaaaaccataattaacaaaacttctgcccataaca
aaagcatcgcatttaagctatgtagttgaacgccttcatattctttcaaatgcttcatgtttttatatgtgtgttacgcttcaataaaggctatccgttt
105. hgg1c.pk014.m2
ctgtttgtatttcttccttctccatttgctgtctttctccttgcagttgttctccttgctgttcgttttgctctttacgttgttcaacatgcccttcttcttct
tgctgtacgttttgttcttcttggcgatccccttgttgttctacttcttgttcggtttcttcttgccaatcccttcttggatttcatctacttggtgttcaact
tgtctcggttgttcttgctgctgcccttcttgctgttctaattgttggtgctcatctgcttgttgttcaacttgacgatgtccgtcttgctcttcttgttgttg
cgcaattggctttgctcttctgagaattgaaattttaaatgtctgtgtgcgacaaattggacaattattgtgttgtttaacccatttatcgatacagtc
ggtgtgaaatttgtgctgacatggcggaatcggccgaactttctgatccttttcaaagggattcaaacagattgcacattcttcttcgccgt
106. hgg1c.pk014.m9
gctgtccaaatggttgcacacgccgagcaaagatggtcaacagcagaagcgattgttttgcgttggttttgacgaagaaggaaacttcaact
gggtcaacagtttcaaggagacatttctgcgtgccaccacttctgtcagttacaaaattgaatttaaagcgcgggcaacatcaattgagcctttt
gaatcggtgaatgaacgaaccaaagaaaagctgacactggacaaaatgccaagttgtgatatttactggctgttgaagcgatgcccaattag
tgagaaggcgacggcgttcccatgggacgacgacgaaaattgggatgtcacattgaacagtgtccaatttgatttgcggggtggcaaaagt
tcgactggccattgcagccaccagcggacgaaaaaagaagaagcaggtcaaagcatcgaaacgtcgtatgactgtgcggaagcgaatta
attaattaaatttgagaaatgctcgacgatctcaacagttggaatttgaatttatgctctgtcatttttttctaagaaatgctttgttgattctttttgttc
gaatatatgtttatttatg
107. hgg1c.pk015.e9
agtttgaagtagctttatcatttaaatttaaagcacaatgcatttcgttaaagtttgctcatcttttctcaatcagagaagtcaggtcatttcttctcact
ctcctctctttctctccccttctctcatctctcttcttatcctctcctctcacttcctcactctcctctctttctctcatcttctctcactctcctctccccttc
tctcatctctcttcttatcctctcctccacccatttttctcattctttcacttcctcactctcctctcttatcttctctcatctctcttcttatcctctcctcca
108. hgg1c.pk015.16
gtgtggacgcctaacgtcggatgcacgctcttaaaggaggtgatccagccgcaccttccgatacggctaccttgttacgacttcaccccagt
catgaaccctaccgtggtaatcgccctccttgcggttaggctaactacttctggtaaagcccactcccatggtgtgacgggcggtgtgtacaa
gacccgggaacgtattcaccgcggcatgctgatccgcgattactagcgattccagcttcacgtagtcgagttgcagactacgatccggacta
cgatgcattttctgggattagctccacctcgcggcttggcaaccctctgtatgcaccattgtatgacgtgtgaagccctacccataagggccat
gaggacttgacgtcatccccaccttcccccggtttgtcaccggcagtctctctagagtgccctttcgtagcaactagagacaagggttgcgct
cgttgcgggacttaacccaacatctcacgacacgagctgacgacagccatgcagcacctgtgtccactttctctttcgagcacctaatgcatc
tctgcttcgttagtggcatgtcaagggtaggtaaggtttttcgcgttgcatcgaattaatccacatcatccaccgcttgtgcgggtccccgccaa
ttcctttgagttttaatcttgcgaccgtactccccaggcggtcaacttcacgcgttagctacgttactaaggaaatgaatccccaacaactagttg
acatcgtttagggcgtggactaccagggtatctaatcctgtttgctcccca
109. hgg1c.pk048.a12
gccgtaacgggcaaatgccaattcaaaaatgagaccgtgggcggcactgtcgttagcttcaaagacttgaagaaaggcgacgaagagca
gctgaagattgccgtcgccacaattgggcccatttccgttgcgctcgatgccagcaatttgtccttccaattttacaaagccggcgtttattacg
agcggtggtgcagcaaccgataacggcacaacatggcaactctggcgggaaacagcagtactttgccggaaaagttggactggcgcga
gaaaggggcggtgaccgaggtcaaagatcagggggactgcggctcgtgttgggcattcagtgccaccggtgccattgagggagcattgg
cacagaaaaaagcgtcgaaaattatttcattgtccgaacaaaacctggtcgactgttcgtccaagtacggtaacgagggctgtgacggtgga
ctgatggacagcgcatttgaatatgtgcgagacaacaacgggttggacacggaggagtcgtacccgtacgaggccgtaacgggcaaatg
ccaattcaaaaatgagaccgtgggcggcactgtcgttagatcaaagacttgaagaaaggcgacgaagagcagctgaagattgccgtcgc
cacaattgggcccatttccgttgcgctcgatgccagcaatttgtccttccaattttacaaaaccggcgtttattacgagcggtggtgcagcaac
cgatacttggaccacggcgttctcctcgtcggctacggtaccgacgaaacgcacggtgactattggctggtgaagaacagttggggcccg
cattggggagagaacggttacattcgaattgcgcgcaacaaacaaaaccattgtggcattgcgacgatggcatcgtaccccgtggtctgag
aaagcgtgggaatgaatgggacgagaagggatcagaagaagaagcaggcagaccaaatagaagcaattcacaatcattatcatt
110.hgg1c.pk048.a17
gcgaatttttgtacaacagcagcagcagcaacagatggtgcctacattgccaccccaaagtgcgcatgacccgtccctgcacccgccccct
cttccgcacccgcacctttacatcggatcgcaacggtttaccgctgcgataatggccgaaatggaagcgcaaccgaacgtttccccgaagc
agaaatatcgggacttgaagaagaagttcaaataccttgtttatgagaatgaatattaccaagaagagctaaggaacctgcagcggaaattg
cttaaactgtcgcgtgacaaaaacttcctcctcgaccgtcttggccaatatgaacagctcagcgagtccagcgacgattcggacgcgtcgac
gaaaacactcgaagaacgcggagtcacaaaacagaaaaggaaaccaaagccttccaacaaccgaaaaagggcagccccaaatccgag
cggagggcccacaggacaaccgaagcgaatcggcaacaaaacgacgccagcaaaatgcaaagtttctggagacgcattcaaagaaatg
atgcaaatgcatcagccaattcattcgcaagtgaaggaggaaatggaccaattcggaagtgagcccccggcaaaacgccgtgccgacgat
tcgttggcatcgccaccgacgacgacgacccaaaggcaaagcgatggtcacganggttcgctggaaagtggggacaaaacgaacgaa
gttgcgaattgttcgtcggtgatcagtgcgatttctgtggaatgatttgaattttggcacttccattttaaagtt
111. hgg1c.pk048.b4
aaaatttcgaattttttttttcgctaattgtcaacaacaaacaggtggcaaagtgtcgtcgtccaatttccatgaaattgtaataaaggggaacaa
aacaaaaagaaaaaaaatgaaattggtaaagttgatgatcattggtgtttggttggtattatttgttcaattttcggcgcgctgaattttcgattcac
ttatcgcagcagccttatcttcccacccgtggccttcttcttcggcgatttcttggcagcctt
112. hgg1c.pk048.c21
ttcccaacaataaatttgtttgatcgtttttcccagtgatcaagtgatccatcgatgtttcacagtaaaaatgagccaagccgcgcattcattcggt
ccaactccctcaccaattcctcaattgtaccgcgcaacggtgcacgcgtggaacacgcggtgcaccgtccgttgtgcaacatttgggtcagc
accagtcggcactgggcattcacccgtctctgttcctgcatttcccgtcgttcccacgtactctgcgttgataccactgctta
113. hgg1c.pk048.c6
tgaacgacgtgctgttgaccaactccaacgccacctcctcctccacggccgccaccgtacggttcaacaaacagcgcgaggcgctggcac
tggacggatgccatgccaaactgttgtacgacgcgttgtgccaactgttacggagtgacctgaaccggcacttaaccaccaacgaggtggt
gcgcgaactgttcgacttgggccccgtgctgaatgaggaggaacaggcacaaaagatgtccaaggcacagaagttggagcggcgaacc
caattgggtgagcagcaaaagcagcggaacatcagccgatgcaagggccgcaacaagaaaatgggtggcaaacacgactttgaggacg
actgactgatcaatctgatcggaccggaccatttgattgattgatcacttttactgatcctatacaaaaattatatattattttcacccaatttttcccg
ccttaattttgggcactttccccccatcacatattaactactattatctgtctcttctctgttctgtgctttctctgtagtaaataggtattg
114. hgg1c.pk048.c7
aaggctaatggggcgccgagtgacccggtgcagccgaggaaggcggacaagttcagaaaggaggtgttagtgccaaagaaatgcatcg
tgcacgtaatcggaaatggtggtgagaacatccgccatttgcaggagaaattcggggtcaaaatgcactttttgggcaacaattatttggagt
acccaaacggacgcactttggccataattggggacacggaggagaaggtggaaagtgcgcgggaccacgtggacagggaattcattttg
aagagatgggaggcatggaacacgggccaacacgacaatgttgaggaagaagcgacgacctacgaagaaatttaccaattgtcaccgaa
attcgcactgcgcgaggacctcgaattggtgatgaagcaaattaaggaccaatccggcatcgtctcctattgctaccgccaattcagcacgg
gccatcgtccgattattctcagagggactgaacaggcagtggcggaagc
115. hgg1c.pk048.d5
ggaagagagcggaaatgccgaaatggtggacatttttcgtgcaaccgcgacggacattgggcggcacgcggcggagggcaccgatgg
acagcaaaacgatcaacagcagatgtgacagcacagagagagtgaatcaatggccaaaagcggcggatggatttcttcggaagacattaa
ttgatcactaattgtattgtatttgattatgctcatcattcccatttgatccgatttgtctgtaatatgttccaaatatctctgattgtacagtgagtcggt
gtataaatgtcggatgaatttgg
116. hgg1c.pk048.e15
cggcatatcctcaacatgcctccttgcatctctacctaattgggcaatgcgcttttcatgagcctgcaaccacatcctctgtcaatagctgttgcc
ggacaaggctacaatcatgactgtgcctgcgattacaggcgcctgtatcaatactcgactgaagctttggtcgtgtgccctttttctacttcaatc
gtgctctggcgtcttttgattcactttctctgttgcatcactatgcaaaactgtctactgctatagtagataccacccggtgatggccatcgaaata
atcctcttcaatccggtagataagaaaggctaaccactatcttagcggccangagcaatctatcgccagatcccgcaacccattcatagaaa
ccgctctgacatatgcattaacatatatccacgc
117. hgg1c.pk048.e22
cggtttaatttttatctatgcaaatattatgaattaaatcgcatctttgctcttttattctccgtaaattgtcatttttccatttttttcggccattaattttcg
aattcgacttcgctgtagacaaattgttataatgattgaagtcaccgtaacggtggctccgaaacttacagcgacaacggcgagcatttcgaa
gctgccgatgcgttgagagtccaagtgggcattgagcggactgtactcggcccgtttgatggacaacacgcgaacaaaagggtctgttgtc
cctcctcttccgtgctccatccttgccgtgctttctccgtttccccgactttcgcccatttgcggcccttccgcgtcttcaccttcccttggctttgg
cacaaccgtcaaatccgcgctcaaatccgtctccattcggcccatcactgctccaaatgcgtcgtcttcttcgatttgcgcgtcccttccggcg
cgacttcgatttgtctcagaaagcacttcgaagagcagcaactgatcggcgattcgctcgtcactcgcccccccgcgtactctgcgttgatac
cactgctta
118. hgg1c.pk048.g19
ggacttcgaatttccgttcgccttgggtccgtccgctgtggacaaagacatttccaatgtgttggcacctccgcccattttcaccgcctcaatta
gttacgatgggatgagcgactcgtggggagggcgcagttattcagacgaaggcacaacaaactccacttcgtacactgagccctccgcgg
atgaggtggaagttggcttcacgttggtccaacagtgtgcgatgcgcgggtcggacgaggagttcaccagcagcagcagcacttcgtccg
gttcctacacttcgggcacttatacttcatcctcgtcaatcgacgaggacgaagaagaggaggaggaagatgaggaggtggaggaggaa
gaagtctcaggcgatgaacacggcagcagaccctcttctcgcgcagtttcgccctctcatcgtagtcggtccgtttcatcgacttcttcgtccg
gagaaagtgccgaaagtgtgtgtagcgaagaagagcagcagaagccggcggaagagacggagctgaaagccatcgtggaggatgag
gaaaagcccgttgcgacggaagagacttctcccattgcaaagaaaagtccttctccaatgtttgtccaaatggcggaagagtcagaacaaat
gttggaaggacacgcgacggtcgaagaattggacggagaagagatgaacatggaagaaatggaacagatcgaagaggagcaaagcatt
gatgggagcaaagagatttgccgagaggagacttatgttcgggtgcaagaactcagggatggccgaacggaagaagcgacacgtccgct
gacaggacagagcaaaccgcgcacagattttgctaagaaagcggtggtacaaccgatgcgccaagagaagctaagcgtgacagaacag
aagaccc
119. hgg1c.pk048.g22
aatgccgccggcattttgtctttgctctcctccgcttccttctcttctccttcatccatctgctgctgtttttggttttcctcggaaaatttgaacagatt
gccactgtcctccgatgtcgccaaatttcgtccgtcttcctcctgttcttcctccttcaactgtcgatggtcgtcaacggattttgccagtgccggt
gcgtcgatgccggcattgtccgttgccgatggcaatttctgtttgtccgatttttcgtcgccattcttttgtcgcccattttccttttctttgacgtcttc
ggacgcaaccaaagcggcattgtccagcagatcagtgtccattggctgctgcttctggcccatctcttccagtttttccgattcttgtgccaatg
aaagttcttgttccaatgtgaactgttcattttttgctccgattggtgccaattcgtccagcgccttttcattttgttcttcttcttcttccggtgatgaaa
tggtcctggtggaagtcgtgaacgaaaagtggccaattgccgaatgagaacgttgctaatgaatggcgctggtgacatgctgaaccaattct
aggtcgctttccaaacggcgaatttcagccgagttttgcgcatcattctcggctttctgtcgtcggactttgtcgagatcatctttcagttccttgtt
gtttttatttgctttctcaagagctgcttccactttgccaagtttgtcagcgtagtcacacttttgttggcattcctcggcgactgcctgtctgagcg
ctgccttcgcgacttcgtctttgtgtgccttctgttgcaattcttccaattttttgctctgctcct
120. hgg1c.pk048.h1
gggaccggaatatcgtagcaaagtgtttgctatgatcccacagctgaaatacttggacggatttgacataaacgatgtcgaggcagaaatttc
ggatgaggaagaggaggagggagctgaggacgcgctcgaagatgaagacgactcagaggaagaggaggagggagtggacacggac
gacgaggcggcgcttgcctatttgaactcatcgaaagctctcaatgatgaggacgaatcagaggactatgtggaacaacggaagaaacca
aatgacactgtgaaagaggcaacgaacggggaacagaaagccaatgccaacaaaaattctggcgataacagaaagcgtaaactcagcg
acaatggcgaggcggccgatggtgagccgggaacgaagcaggcgcagtgagcgggggaaaagaaagtgctacggattggtcttgcgc
tatcattttgttgtggccgttgtgaggctgcattttattcgaattgtttttgttttggagcactttcttccccaccgtaatttatttgtctcttctctgaacc
gtcgtccaaccgattatgttctgaattgtcagatgaataataaaatgtttccg
121. hgg1c.pk048.h23
gcattggccaaatggctgttcactccacttcaaaacaatgtgccaaagatgctcaactgctcgttgaatacggatgatggaattttgtcgtcga
atattgaaccgttcaaagcggcttttgcctcggcttcttcccccgtcaatttcatcatttacatttcgtttgcgtcgtcttttgctgcttccgttgtgcc
atttgatctgaccaacgaaatgactcgggaacaattggcattgaaaaggactaacaataaccgccgttttctgttggtccgttgtccaattgcg
cgagacgaaagtaaatggacaaaatgggaaaaggaagcgattgcctggcgaatttatgatcaatggaacaaaattgagattcaaatttatga
tgagggcgaaatcggagatgggcttctcgacgcaacttccggcccaagtgatcagcagaagtgaatgaattgttgggaagtgatcgatcaa
tttgaaattgcgaagttggagaatgtgaatttgatgtttgtaaaatggacggattatatatgtaaataaattgttttaaatgg
122. hgg1c.pk048.h5
gcgctttcgaaattgaagttcagatcggtgagatgtcgactggttccatcagttcccatgggacaaattcgtttcccattttgatttgctgattttg
agtgtcgcactcattttgttgtttggagaatgaagtgccagacaaatcattggcctcctgtttcaatggtaaatccgcggcctttttttgtcgcattt
ggatcgtccgcagttgagttgattgtttggttgagccgcgacgtggacgcatctctcttttttgccgaatcagcaacaggacgtgaagagaga
ttcgcattggcaaagagctcaacacgatcattcgctgtatttggagagtccgcgaaggccttgtcacggtattcagcaatcttggccattttgtt
cgcatctttggccaattcgggtgaaatgaaacagcccaaggccttttccccaatgcatttcataatggcacccaacgcacccgcgtactctgc
gttgataccactgctta
123. hgg1c.pk048.i10
ggacagcaatagcgaggagcgcactctgttcaactacgagttgtctgtcatgttgaacagtgcgcacaagttggagctggaggcgctctgt
gccatttcggccaattatttgagcaccgtttatctggacaacaaattaatgccgctgaatgtcgccgtcgcttacccacacaactgtcaattcaa
caacaacggtgatcaacaacagcaacagaaccatcaaaaagacttctcagaggacagcgattggagtgataataacggtgatcaacaaca
gcaacagaaccatcaaaaagacttctcagaagacagcgattggagtgataataacgacgacgaagacgatgattttggaagtgattggtcg
tgattgtctattttcttttattattcctgtgattttttaattggtaaatttatataaattatgctttctttac
124. hgg1c.pk048.i20
gccgacggaaggagtcacagggacggaagaagagggcaaaaacggaagggaagaggagcaactggcgagagcaacggaatagga
agagagcgcgataatggctgagagtatggaagaaatggacagatcgacagatgttgaccgagaaattgacaagggcgagtttcgccaag
cgcaggtcaattaacctcatattaaaagtgtgtggaaagccaatcatttgacgagtcggggtatgccaagttggaacatttgacgctggataat
aacctgaagaaagaggtggtcaacatggcgagacgcttgcagaaggctcgcatttccttgaattctttgccggacactgaggccattgcgcc
ggtaattgcgaaaattgatgaaacgttcggccagctgatcgcactttccaaggagtccagcgaattttccgctcgaaacatcaaattaccgga
gtacagcagggcggacgtggaagcactgctcggcaaaatgggacctgag
125. hgg1c.pk048.j10
caagcaatcccttatttgttaccaaaaccatggcttgtaaacaaaatacatatcattgagcattcattcgggtttttgtgacatcaaagaaatgaa
acaataaataggtgacacaatttcaacataatgataaggcaatgggtcaccaaaaaggcgaaagtcgtggaacaaaaatcggtgacgaact
gccaaaagacatcaaaaaatcagcgcacccaaacggacactgcgttcattgaggccacagacgagcaaaaaaatttgtcatcagcgaatc
ctgggcattgagtgtgacgccatttgaaaaacggaagcgcaggaaaaattggcaagcgatgaacacggggagaaaagtcaatggaacg
gatcggaccaagtcaaagtggacgagcaaacgcaacaaaacgccaaacgaagcggcggcggacaaacggtggcagtcgaaacgctc
gcggcaattgaccaggcggcactgcacgtcaccgtcatccatcactgacgacccctcgttgctggtcagttccaccggtgcagtttcgccac
cactgtccgccctttcttccccgtacattccatcaatcgttggcgcctgtcttctttcgccaccatgcgctgtcaaaattttcgagattcgatcctc
cgcattctgaa
126. hgg1c.pk048.j14
acattggcaacgcatttatacgcagaattttgacagtgccgaagtgaaatccgacttgttgtcgtttatccaccaattcgttgtcacattgtccgt
ggacccatcggccgaccaaaattctgcgtctgaccaattggcaataatcgcaagtgcaaatgaattgcttgacataatagagcaaatgttgga
ggaggaggaatctgaacaatgcctacagaaaggagctattctgtgtactgaattgtccaaatgcattccttcgacggacgaaggtcaaagag
tgcgtggagaacagcgcaaaatggcatttcaatgcagacaagaagaattggcacggcaaaaacagtggcgacggaaggaacaacagag
ggaggatttgagtgcaatggtggaaacacttataccaattgtgaataacagctgcacagtgagcatttcaagcggagaagaggcaatgagc
aaagattgctattaatatcgttttgcaataaataattttatgtttgattcaataaaaggttcacataa
127. hgg1c.pk048.j21
ggggctggcatttgccaaagtgtctgcgaaagttcacggccaacatttgaatgaatgaaaagccattgcatgccaaccgaaaatgccatgg
catctataccaactgctgtccctacgaatttcatttattttaataattttagtaccaattccaaacccccataaaaaacaggtcttaaaaaggagcg
agaaacaacaaaaacccttttcataattgtaataaaaaagagagcattttgtgccatttttgttactacactcatcacactgatcacttaattgggt
gcggatttatatttangaaaaataatttttaaaaataattaaaatggttgaaaatttgccggaaatgccttagaattaatgccattatcatcataaaa
tcc
128. hgg1c.pk048.l15
ggagggacaattctttggagagtcaaaatggcacgaaacggcagagaaagtgagggaacagatcagccaggcatgtgaagaaggcgaa
gagagtgccgccgttgaaggcggagaaagtgaggggacgacgaagaagacaagcgaagacattcagtcggaagtcacaaattacatgg
aaactgcccgtctcgagttgggccaaccgtccaccagcggcaactgcgtcgacccgtcctctccaccactcgtcacacattttaactcaatg
gccgaactgctcttttggaggcggattaatgccgaacgcttcccacgccttgtgcagctcgcccgtcagttctgcgcagttccaatggccaac
agcagtgaccagaggaaggcactaagcaacgacaatgcggaggaagagaaagtgacgcggagatacgcagccgaacagatggcaga
agatggcgacatggcacaattggagagcggacggacagaacagttacagctgctcacacagctgatgaccgtgagaatggcactcagag
agggggcgacagaaacgaacgaaaatggcaaaaaacgaaacgaatgcatggcaccaaatgaagaaattacaactatgg
129. hgg1c.pk048.13
cggtgcttatttgtggggacacgcattctgacttaccgatggtggaatttggcaagtcgaaaaactcgacgggtgtgatggcgctgtttgtcac
ttgcgacaaagggttgcaggaaagcgtgagggaaattgtggaggacagtgaccgctgctgtttcgtttcgactcccgacgttattcatgctgc
gatgatgactgttcttttgaaggcgaagaaaatggcagaagttgacagcgaattgggagggaaaaattgaaggagacgaagagaatccga
tggaatggaagtgaagggaatctgtgtggtctcataaagtcgtagaaatccgaattgacttctaaacataatgctatatttttgtttgtttaattggt
ttaagattcttcgttcgtttgttcttttcttttgacgattggttgttatgacatttctgttcgggaacaatttcatgattacggtttaatcgagttattcttgt
cttttcatgtttttttctaattgaaattaaaaagt
130. hgg1c.pk048.18
aagcaacgcgtcgacgacaagtggcggaagctgtgccggtctttacaacggtgtacgaaagtcggcagcggctgttagttcggacgagg
cggggaactgtgtgtcggaggacgatgacgtcgacagccatgaagatgagaacgaatgtgttgtaaatggcagcgacgacggaggcag
gcgaataatcacgtcgaagatggtcagcagcagcaacaacatgaggaagatgacggagaggcgccgcagaggcaaattcaatcgaag
ggtccgtcatccgattacctgcccttctcttccgttcttcatcactgacaggaccgttcagcggtgtgcagcacctgtctataagaaaaacaac
agcaaaacatagcatcatcaacgctccaatgtcgaagagtctcaccattttcaattgttttgttttcctttgttgtgcccttttgtgctccactttaaat
ttaaattattatataaatttttgttttacg
131. hgg1c.pk048.m13
gtccgcattttctcttttagcaaacgtccaaattcgtttagcatttttttatggaaactctgctggacaaaaccattttttcggtcatcgacccgccca
atttgcgcattaatttgccgcgatggtttacgttcccctcgcccatgcaaacctttttcttcattcttctcacttatttcctcgtctccggtggcattgtt
tacgatgtgatcaacgaacctccgtccatcggttccacggtggatgaacgcggaaacagtcggccagtggccataatgccctaccgcgtca
atggccaatacattatggagggcctcgtcgcttcgttgatgttttgtctcggaggccttggcattatcattttggacaagtgcacccatccgttga
ctgccaaaaacaaccgaatgatgcttttcggactcggcttctccttactgtgcatcggcttcttcaccacgcgaatgtttatgaagatgaaattg
cccgattaccttcagtcctaatttgaaatctgtaataaaaactgttaaatttgatttttgtaattttttatttaatataataaattgttcattttt
132. hgg1c.pk048.n11
gaaattgtaagaggaacagaaagtggaatataaagtggaagagaaagtggaagaggaacagaaagaggaagaggagcagaaagtgga
agagaaagtggaagagaaagtggaagagaaagtggaagaggaacagaaagaggaacagaaagtggaagaggaacagaaagtggaa
gaggaacagaaagcggaagagaaagtggaagataaagtggaagaggaacagaaagtggaagataaagtggaagaggaacagaaagt
ggaagataaagtggaagaggaacagaaagtggaagagaaagtggaagaggaacagaaagaggaacagaaagtggaagagaaattgt
aagaggaacagaaagtgg
133. hgg1c.pk048.n22
tacgcggggcacaccaaaagtttttggacgagaaaaaaatttgccctccgactgacgagcaaatgttgcattcggattttcaaaaaacaatga
acaaaattgtgcccgaaatttccggcactctcccgcaaaagcctgtcgaaactagctttcgcgacactgacattagaagctttttgcagagtgt
aaagccaaataaaaacgcaaaacgagaaaagaccccggaaaaagggactttttccatttcgaagtcggaaccgcagacgccggtcaaaa
caaatttgggtaacgtaaaggcggagccacaaactccaaagacaccgatggaacaacgggtgatgccaaaaaaaggaaatccgaaataa
aaatgacccgttcagttgagggccaaatttgccgcgaaagccgcctttcaaaatgtcgatgaagatggaattcgaaagcgacgggcgatgc
tttaccgattgaaggaagacattttggaagtgattagagcgcatttgaatttgaacaaagcttcaacggttgcgcttggtaatcgcgaattgttg
cagaaattgggacgtgaagtcaatcctggaatccttttggaacatttgcaacttctttgtgaaattgtgcctaaaaatgtttgtcgaattgagtcaa
ctaattcttcggctgaccaacatcacttcaaactccacagcgacgtgggccctgattggctacaaaaagtgctaaacccgataaaggaagaa
attgacagtttggatctaaaattgggcccgccaacaattccgaaatcgccttccagtcttttctgaatatcgatacccaaaaattaacattttgaat
tttgtttgatttt
134. hgg1c.pk048.n3
gacggggttcggaggcacggcacactttgggtgttgacgtgagttggctgaagcgtttggtgaccagcaaacatgagcaaaaagagcaac
aacaacaacaaatgggcataaatgaagtcaatgggagcggatgtggagaagttctgttgaatgggcagccccaagcgaattgcaacggg
aagtgtccaaaggggtggccatcggcaaatggtggtcttatgaaaaatggcgataactacagcatgaatttctcattacgaaagttgcgattgt
ttggacgacctcagcaacagcctattgcatcggttgacaatgagttgcaacatcaaagacatacacatgaaaaggaggagacagatcaaga
acagttggacgaccaaatcactgcttgtacggaccagcagaattgacaacatttggcatcaaatggcgccgtgaaaacagcaacacatcca
ccatctgtgccgcggatgggattggggcaactgataacgatggaagacgaacgccaccagcaggactattgtgaaagcgaaatgatgac
acaacaaatggcagcgaatggcgaagacgaaccgttgcgggaaagagaggaaaatggagggagagaaaatccatttgacaagaaataa
tgccgaacattctctgtattagtcaaacccacaatactttcatttaaactttaaatcacctctctgataatctcaaccatttcatctttcaacaaaaag
ttttgtataaagtataatagcgtgtggata
135. hgg1c.pk048.n6
tggaggaggaagagcaacaaaaagaagtggaggaaagggagcgaaaacaaccgccgacggagggacgacaacggcggagcacac
tttcccatcgaattttcaccctttgcgattcggaagcaacgctaatatgtgctaaacaagcgcaaaatgagaagcaaatcccggaaaaaaaga
aaagtgggccaaaaaggaacgttttcatcgactctgaacaattcaattcaatttttgtcttctgaactgcgaagccaaaaccgttcaatcgtcgg
aagtgaacaaagaatgcaaattgtggcatatttgggcagcgatcacatctacgacccgtcggaggagtatttgctgtggaatttgcgagtgg
cgaacggtcgccgattggtcaccgattgggccatggacagaacacgcggcgtacaatctgccaaaacagggaaagtgttcagggcacgg
ctgacagtcaaagagccgacactttcggacgaaattggggagaaaggacagcaaaaaggggccgaagaagcgg
136. hgg1c.pk048.o4
gatggcaccgttttccgagtcagtagtgacagaagagatcgttgaggtggattgatgctgttaagttttacggatgaatatgaccctatgtgtta
ctctatttccctcatcaattcatttgatgtatctgtaaagtattttgtagtccgatacacgttcttttaaattaaataaacaaaatgtcagc
137. hgg1c.pk048.o9
caggatcataaaatgattatacacgcggcttatggaagcagtaaagacatatctgtttatagtgctttggatttaaaacccaaacagatttttatta
taggcaaagtcggtcgtaaacatcacagtatggccactgtgttggccgatggttatgctgcacatttgtctgctctacagtgtcatggaggatct
agaccagctcaggggaatgcccgaatacttttgacctcgcgtggaagatttggacacaatgcttctatgaggcgtagaaggtatgtattttatg
taattcattatcaataatacattcatggatgatttaagataagtatttttcttttctttttgaaaacaaagttttgaattagtcaagaaattagaaatgtg
gtatttatgggaaaaaccatatatagactataataatgcatttcagtattaatattcatcaaatatattgttaacaacttgaattatacaagttaaatca
agtgttaaatcaaaatatttcttagggcgttcaaaagataggttcattttttttccttttttcaagaataaaccaatttaatctgagtaaaaaaattaat
aattaaaggcttctcttaaaattatcgttacttaaacttgtcttaatcaggtgtccagagaagagatctggcgatacacatggttccaataattgat
ccatacttgaccccgcgtactctgcgttgataccactgctta
138. hgg1c.pk050.d1
gcggtgggaccagcgctggagccggcggaggagtgatgacgggtggtcaggacgcagcgctcgttgcagtgagcgcccaggacagat
tggcaatcacccggatcgcttcaatgggatttccagaagcgttggtggttgaagcttatttcgcctgcgacaaaaacgaggatttggctgtca
attacatcttggcgaggatggacgagtctcagaatggacgtgcgggtgccgggcagcagggcggacgataagaagtgcaacagagatg
ccgcagtgatcgcaaattcctcatgtcgtttccctaaattatgatcattgtttgcccctaaagtgcatgttctgttctcgccctttggctatttgttgtg
tttgattatgaccatattaaattgtttatg
139. hgg1c.pk051.h11
taattagggggtgacaaattatcaaaataataattaaacaaaaaacccaaaaacggaggtctaaacaaatttagaaggagcccgtgtgcgat
gcgcacgaccaaatccgcccatgtcatcattgtcggcatcaccaccatccggcaccacttcatcttctggcacagcagcgcctttttcttccac
cagacggcccattcgttggtcccccctcatcggctgattgtccctcgtcatcggctgattgtccctcgtcatcggctggttgtccctcgtcatcg
gctggttatgagtggcatttcgtcctccgccaaagccccgctgctgtccaatccgtcggcctctgtcccctctctgtccctccgcctcttctccg
ttcgggcgactgtcttggccataacgcatataatttcccctggacgacttcggtccttgttctcgctgctctctgtacatgtcgtgctggttccaac
cgcctttttcttccatcagacggcccattcgttggtcccccctcatcggctgattgtccctcgtcatcggctggttatgagtggcatttcgtcctcc
gccaaagccccgctgctgtccaatccgtcggcctctgtcccctctctgtccctccgcctcttctccgttcgggcgactgtcttggccataacgc
atataatttcccctggacgacttcngtccttgttctcgctgctctctgtacatgtcgtgctggttccaaccgcctttttcttccaccagacggccca
ttcgttggtcccccctcatcggctgattgtccctcgtcatcggctgattgtccctcgtcatcggctggttatgagtgacatttcgtcctccgccaa
agccccgctgctgtccaatccgtcggcctctgtcccattctgtccctccgcctcttctccgttcgggggactgtcttggccataacgcatataa
tttcccctggacgacttcggtccttgttct
140. hgg1c.pk051.i9
agaccactgtcacttctctgctcaacaacaaccaaaatgacatctcaattctgaagagcttgcaattagaacaagaggcgaatgccggattac
tggtccaaaaagttgacggacttctggctggaaatgcagcggatataactgccatggttttgtcgaatggcttcgaagcgaagactcatcaaa
atttattgaaacaacttcgtgacgcaactgactctgccaatgatgaggctgatcgtttggaaaacgaatacttcgcattacaggaacatatttct
gcaatgaagcagcgtctgatggaaaagaaacgtcgtcagctcgagcagaaacaaaagatggaggaggaagagcgaaagatgagggag
gaggaagagcggaagaagtgggaggaggaagagcggaagaagagggaggaggaagagcggaagaagtgggaggaggaagagc
gtaaaaagagtgaggaggaagagcggaagaagtgggaggaggaagagcggaagaagtgggaaag
141. hgg1c.pk051.j12
tggcgacgccgctcacaataagcgaagtttgtgcgtgtacattctccggttgaacaacgcactgacaaaccaccgggtcagatgggaacag
ttcgatgttgaggaggaagcgccggacgacaaattgattattccttcgtggcctgctgcggcgaaattgttttatttggcggaatgcaaagcg
atgggagcggaatggaaagtctgaatgttggactggaaaggcgcgcaatgagctccgacacttacattttgcgaccgcgttacaacgaaat
gttctgctgatcgggatgattttgaaaaagggaattagatactacctgtagtaactaaatggaataaaactttcgtattctaataattgtaatttttg
ataaattctttttattac
142. hgg1c.pk052.e20
cacaacggctggcaatcatccacagcagcaaatgctaggctgcgccggacagccacaggacccgaaggcgcgcaagttgatccaacaa
cagttggtgctgctgctgcacgcacacaagtgtcagcagatcgagcggtctgaaccgctacaaaaccgtgcgccctgcacattgccctact
gctcggtgatgaagggcgttttggaccatatggtcgactgttcggccggccggcagtgtcagtacgcgcactgcgcctcctcccggcaaat
cattgcgcattggaagaactgtaacaaggacgactgtccggtgtgcaacgttcacatcaacgagacaatggtggtcgacccgcgacaagct
ggcattatgctgagtgctgtcggttttccctctgtaactttggctcaaggcgcgattggccaacagcagcagtcgatgaacaatgcaaacagt
ggaggaccaccgcaaatgcgcgggggtggcataacgcagcaacaacaaacggctggcaatcatccacagcaaatgctctgcgcgggc
agcggcggtggacagccgcaggaaacggtgaagcgcaagctgatccagcaacagttggtgctgctgctgcacgcacacaagtgtcagc
agatcgaacggtctgaactgcgacaaaaccgtgcgccctgcacattgccctactgctcggtgatgaaggccgttttggagcatatggtcgg
ctgttcggccgggcggcagtgtcagtacgcgcactgcgcctcctcccgggaaatcattgcgcattggaaggactgtcacaaggacgactg
tccggtgtgcaacatggtcaaacggtacaccaacggaacagcggctgaccggcgacaagctgacattatgctgggtgctatcggttttccct
ctgtgactttgcctcaagacgcggttgggcaacagcaaccctcaagttcggcaagtgtttgtagtggaccgttctctgtcggaagcactcctat
tttattgaagaatttacatttatagaatttcacttttgtatttggagaaagtgatcggc
143. hgg1c.pk001.e18 (Amino Acid)
KSSALRRGRDHTFAQPAYMRDPLRADLLAGSKLKEVKKTDYNQCKSMLLDLFDGTRVI
LVGETRDRSGRKRLISCFQLYRQSRAAAYFGMFAVHPFFQASGLGKRLLTVAERYARIV
WGSDEMHLDVGGSLAELKLGMGRLQRYYKRRGFLSTGILRPFNGAVARFITVDRNDL
WIELMVKDIRGALDDIGGDPEKRMKRVNSRGRLAREADKDDGGRDPQKRMERVRSFG
RLTIEADRDDIGRDAQKRMERVRSLGRLAREADKSDESKGKDGEEKKKTTQAEGEESK
GKDGEEKKKTTQAEGEERIKPLAD
144. hgg1c.pk013.j16 (Amino Acid)
WVLSYVSDKGSYPVLGKDAEGRERMNALIVGHFDGHTFEKLFEQQMDFVGGSFAYQG
FHDQQSGRSFTIGWICDIGWIGDNTGDANFDGRGGVTSMTLPKEFVLKDDHLIVRPLPEL
AQLRQSKQPHQIRKGEKYSLEKGHAELLFQFKWSNNDDGSAEEKFVLDLTRTRLKDGK
LEFTIDSKGIELKRTWVKPNKRLVVYNVKPGQIHVFIDLDTVEYFADNGRWSGAVRVPN
ASQENRIGTVELKSTPLVLEQSSLWYLKYGSHKSARLQPNGIPFAMNAGTSSFKQDEA
145. hgg1c.pk01416 (Amino Acid)
MNNNFLLLLITFTFIVGARAFWIQLPGTFWGYGDARQQQHRGWLNGWHSWHNQKHNG
ANTGGYWPIYGHGHGHFGNGNALPADDRSSNEEDDNETSEEQQLTTDDPPENASSDIM
EPNDGITDQPTDQDGSDTEATDSTTVGSDPGPNDNDQNATGPTDEDETGTEATDSTTTT
TESNAIGEEGTDQDATNSSDQGESDAEAEATDSTTNGSDLEPNDQDENGADADSTTTNG
I
146. hgg1c.pk015.h1 (Amino Acid)
EKKQNVFDDFIAAAEYLINKQYTNSSKLAIFGASNGGLLTAVCSQQRPDLFGAVITQLGL
LDMLRFNKLGIGSDWVSEYGDPDNATDFSYIYKYSPLQQLSVTPGKQWPATLLLSADH
DDLVDVSHTLKYTAQLYHLLRTNAESWQRNPVVAKILVDQGHAFTGTPTEKKIKEKVD
IYTFIARALGLKWTE
147. hgg1c.pk048.e18 (Amino Acid)
GGTPAVXAYVYDRKGTHYEKKIRVDDWDNHYIVDLATNDVQDVLKQNLDLEFLKLRD
SVASGETKELTFYGRVWPEGKYKLFWDVKGFEMDEAQRLIKSELNVPHDCFTDENGKF
KLEYEIENKSREVARWRLPPVHLYIFGASVWTKEYVHVTDWHHVHIFDLKNGKKHALP
ADKVAEKLYELSKRDQMNERTKLAETNEKNENEITFTRSFCPFRQ
148. hgg1c.pk002.a5
MALSALLLLLPLLLNVQNIPDESVQSDVKAVDSAISSLEQWKDPRNSLASLDSQLTEPQR
ALAKMFWELETIEKEKPKAPPQFDLGLFLEALEAMVEMNEEAKEVKLRKDKLTEWAG
GEKANEGKEGKTKEEETVPEVRVNENVKVEVTNGAGGDGKMEVKRGKDENGNEQVV
VTFVKRDGTEGKTEEEQKKEEKDNLRKGREEVKMEQDNVEGAPKTDSANSAKSPIPMP
TILSSPAAPAEEEEKANDAFTEANVRKKVKKDEEMFIIMTDDNGRTGNANERQMEFVR
MPKKVGRDFGSELFGLPQPSNGGQSPMEMFFNLFGRKKRETVQEGRKKRSIENLANLG
KPGSEFVTKMAEQAKNDDKQDEKAEIKQYLEKGVATAEGNKKAEKLAYVWYSELLY
WTNKWIEVDTPAEPQKFSTFLRH
149. hgg1c.pk003.d19
RGKGKNAAKKDKTKNKKAPAAAKPKAEPVETEEPSSAQVVAEQDGSDESANNQEMDA
GEEIAEEEQTDLAQDEQLEDDATDGEEGNGMAEEEQPEIN
150. hgg1c.pk003.g23
MSSPSSSVSLLAIVTIFCLLCKCCVSAPHPCCPGSQKVVSLMANYVGTFAHSFSKASLCS
DAQSVAGALKGQLIGCSKGGDATLLADIEASLATHSADECAHSLGFVRAMFAIAASASS
HASNNNEWQALSAQFGQQISEIDSKCAEFGIGIAKVPYDGPKGDHSQRNVHGTDSVIAM
PGLAGSHKQ
151. hgg1c.pk004.a14
MFSLMLSIFPIVFLVCCKAMPNFPCCPGSQQVVAVMSNYIGTFTSEDKSTVCSTAKNTVE
GIKSELSSRVGCPSGGEAQIVNEIDRQLTNIAKMEINYEDECPYNLGFARAMFDLAAAAG
HAGNDTEWQNMKSKFVQESQAIKAIGQEMNIEVTDVHIGHPSKGISAHQNVPSPSHVIA
NPGQHSSVGHGKEDTPLSSDFDF
152. hgg1c.pk004.a16
MKIISILINFILAIYEAKGGGIVSLLSRRQAPKRHLASSLRQQRTEDNHISINGQNYAVDGP
NVNVGVEGHDLSVNGRVYQNRATEQYLEIIQDKNIRNVIVSVPLSLFSRENIIDGQINAK
CNGNLYIDQSSDGCSRIICVDDKKNGVENNFGQTRDIFLTGDVNIFESANGIIYNSMMGG
TLHIHNSSLECANIECDASLNVTHSPIERNAQMKCGGSLSIDESPMGNIRLNCDGSLRIEK
SKMESSQIDVGGSIGIVESPMGSIGIDCGGSLRIEKSKMEIGNLDCGGSLTIVESTAQSLKL
NCGGSLNMKESPMKNVGINCDGSATIKKSKMESGRINCGGNFSIDSSPTGSVRIDYGGRR
INL
153. hgg1c.pk006.e12
MANKFLIAAFILTIAIFVNGQSEAPNNSSEMASEESNSEESSSEEQQFNPFKFRPFFGPSSS
NSSAPPPFAFLPFFGRMPSLFNRPSNKSVV
154. hgg1c.pk004.l14
MRFSSFSSPFLPLFFLSLPIAFVLSGRTLPFTGSQLANEVARAFFNSVNTWDMSIFGAGTK
QGEDRYKISLDGLDRMKNRFRVPLPAGQGLEKLLRSYRVEPLREDYLGVNKARERVLA
PSKLMELMEKLGNVLVTDPKMRQKIDKYDKKRADEAARRAAMMPPRQDPQAIAKRRT
WPKEDGLALERGHLPQGNNQSPTRLQSTPRIWIQEDDRWRQPMTFSRKDVRERSWLES
DTDSDLDSPTSVLRSRRRSRVNILDDDQPTRRTAWGRSPTPSPNGRAVVQRTTTTTTTTT
EEEEGGRRTVRFGEVVVVEPEERTVNRRTEVRTQQRETEVERTSEYTLILRIDFIDASVFL
DKSLAYFGSLNTARKDERSVQRLCYVLKAFDPRHERLNSVLATPSVANAFVEYKKALN
DVGLNSQPELRLVEKSNACAFDLALIYELAQFTKDLLLKLKAERMVAAEELEDVKEEVI
GRLLKLLPKVLEGLKAKPAELSTEVDRRIQALDVVEEQLNVVKRARATDEMVTGAMA
KVMAQLRNASRGMGTMDMSTLSSLQSNWDNLMRKDTHWQIRKAINSLGGCPKDPQG
NTLMKQCMEEAITKVDRYIDDVNDWFKSQRPIDMDDWKWLAAEIQMIIRWKSP
155. hgg1c.pk006.c4
MAILLKCVLLLSIMAIFCDCMDPGKKGKSKDPIPIPKQEGSDPIPIPKQEGSDPIPIPKQEGK
PSSSAANSPTVTKGTPKRGELDTPEFYKTSPKNKINSPRKPNNGSPRKDKKALQKERQEE
RKQKERERENRFLRTKSTAGNTTDATDVETESEVIPTFVAELEDSTVEYPTDIE
156. hgg1c.pk052.h11
MAPLFHRFSSLFVFLMPFLSVVLLPSTVCTGSDSAAAPFDRKNYPKIDLRLFEWPIASHSG
SSAEVSFIAVDCYTQLDRSFISTDAVLRLNNSLALRHRACLLRIPTGTRLTVTEMQTTNR
KVNKTKPKLRPMARAVPTGVCAVQLARAQNGMGRISSGRRNGGGQRDGERGRMFGG
RRGGRRGRGEGIPQKASSLSRWAADSFGFDEH
157. hgg1c.pk004.a22
MAILLKFVLFISIMAIFCDCMDPGKNGKNEKKDVVKQKVDETKVERASEMNKGKSIVM
ADSKKEGTTTVKIPHRYGAVSGMSGQNASPEASQIGSPKNSPKGTQIGSPRSISSPKSTQI
GSPKGIQIGSPRKEKTKLSSAVGSSDFNVIDESKEAKKTKPIQTESVQKPK
158. hgg1c.pk008.i22
REATVLKHVGNQTNAAGIDAEFAVNFLLAQMEANKMIQRGYIDRWNSDHSFESKYVPD
FEKEIQPKFSYATNALILALIPLVDAGHQMHNDQNCVEHVEDVLESMEHLRASELEPNG
KEAMEKAVKAICEKISTHEGQSNAEDQSKSKKRKHSDNHKMEEGKHGEEKEIRPTKRT
RKANTDESKTPAAGENRRNHRRENYVDS
159. hgg1c.pk007.j13
MNKFVGIFVAVLLQFVSPFSAFSRVPTTTTERPIIYDPKEMVEIQVNLVNNTNNNCTNDV
LRKYRVEITNYVFFLVCDLKIRVQLPEGATLENVVNLKPFNGTTDQFIFPDSLRYLYVSK
TLEAELSVKGGEGEPKITVLDAKAAFSPKKCRISKF

### Example 2

### Construction of cDNA Libraries from SCN Esophageal Gland mRNA

In general, two cDNA libraries were constructed by the methods described in Methods Mol Biol. 2011; 712:89-107, Hussey R S, Huang G, Allen R., which is incorporated herein in its entirety.

Two SCN gland-cell cDNA libraries were constructed by microaspirating contents of SCN secretory gland cells from 100 nematodes to provide mRNA for first-strand cDNA synthesis. Two experiments were conducted: Each experiment used the SCN gland contents from the equivalent of 50 nematodes, which were subsequently divided into two tubes of 25 nematode equivalents each. First-strand cDNA synthesis from isolated nematode gland cell mRNA and subsequent LD-PCR was performed using the Clontech Super SMART kit to generate 2 full-length cDNA pools. The LD-PCR products synthesized from the first cDNA pool showed a standard normal distribution, ranging from 0.4-3.5 kb in size. 27 amplification cycles was optimized for large-scale LD-PCR amplification of the first cDNA pool for library construction. The large-scale LD-PCR reactions for each cDNA pool were performed, purified and then cloned into the Promega pGEM-T Easy vector. EcoRI digestion of a random sampling of gland-cell cDNA library clones showed that the insert sizes ranged from 0.4-2.4 kb. Colonies were then picked and re-arrayed into 96 well plates for cDNA sequencing [Example 3] and glycerol stocks of each cDNA clone were generated.

### Example 3

### Sequencing of the 2 New Libraries

Culture clones in 96-well plates and re-array clone s into 384-well plates. For sequencing, cDNA clones first were recovered from archived glycerol cultures grown/frozen in 384-well freezing media plates, and replicated with a sterile 384 pin replicator (Genetix) in 384-well microtiter plates containing LB+100 μg/ml Ampicillin (replicated plates). Plasmids then were isolated, using the Templiphi DNA sequencing template amplification kit method (GE Healthcare). Briefly, the Templiphi method uses bacteriophage (p29 DNA polymerase to amplify circular single-stranded or double-stranded DNA by isothermal rolling circle amplification (M. J. Reagin, T. L. Giesler, A. L. Merla, J. M. Resetar-Gerke, K. M. Kapolka, J. A. Mamone. Templiphi: a sequencing template preparation procedure that eliminates overnight cultures and DNA purification. J. Biomol. Techniques 14 (2003) 143-148). Cells were added to 5 μl of dilution buffer and partially lysed at 95° C. for 3 min to release the denatured template. 5 μl of Templiphi premix then were added to each sample and the resulting reaction mixture was incubated at 30° C. for 16 hours, then at 65° C. for 10 min to inactivate the φ29 DNA polymerase activity. DNA quantification with the PicoGreen® dsDNA Quantitation Reagent (Molecular Probes) was performed after diluting the amplified samples 1:3 in distilled water. The amplified products then were denatured at 95° C. for 10 min and end-sequenced in 384-well plates, using vector-primed oligonucleotides and the ABI BigDye version 3.1 Prism sequencing kit. After ethanol-based cleanup, cycle sequencing reaction products were resolved and detected on Perkin-Elmer ABI 3730xl automated sequencers. Over 7000 clones were sequenced, ultimately resulting in a total of 11,814 sequences.

### Example 4

### Bionformatics to Identify Genes, Proteins, Structures, Peptides and Functionality of Proteins Identified

The sequences determined in Example 3 were examined with known computer programs and by trained scientists' observation of particular sequences to determine functional protein domains and structural proteins of nematodes.

Sequence Cleanup and Assembling.

These sequences were quality trimmed to PHRED scores of at least 20, and further trimmed using the ‘seqclean’, a vector-linker cleanup script, to remove non-subject sequences. The resulting set of 11,814 sequences averaged 509 nts. These sequences were assembled into contigs using the CAP3 program, resulting 3392 multi-sequence contigs with ranging from 100-1825 nts, and 728 nts on average. Manual inspection of sequences for low-quality or low-complexity sequences was done to remove additional sequences.

Tissue Enrichment Filters.

The assembled contigs were then annotated and analyzed in multiple ways aimed at enabling filtering and gene selection. The publicly obtained set of 73K EST sequences from SCN whole body and diverse tissue cDNA libraries (not gland cell specific), were assembled and used to cross-BLASTed to the 3392 contigs to determine matches of high identity, and hence essentially same gene matches. Considering the hgg1c contigs and singletons that matched the 73K whole body ESTs, an index ratio of gland EST count to whole body EST count was developed and used to filter for gland expression preferred transcripts. Generally, all contigs with less than 1.5 fold enrichment were immediately set aside, whereas those generally with 3.0 fold ratios or higher were kept, with those in between filtered against the factors below. In addition, the hgg1c contigs were BLASTed against proprietary ESTS derived from parasitic stage SCN (stages J2, J3 and J4), and parsed at 98% id_{—}100nts) to identify which of the contigs overlapped genes expressed at these stages. An hgg1c contig match to transcripts of these J2-J4 stages was selected for, as these represent pathogenic stages where in genes of interest were sought.

ORF Predictions and Curations.

Six-frame translations were done on the transcripts. Transcript ORF completeness was analyses several ways. First, a proprietary pipeline analysis was carried out to determine how many of the assembled contigs likely have full-length ORFs. This method relies upon the best reference protein hit among C. elegans (hit must be at 1e-10 or more significant) or broader reference proteins such as NR top BLAST hits about 1e-10, and infers likely start and stop locations if present on the transcript. Novel ORFs would not be assayed this way. Nonetheless it is a measure of ORF completeness. Otherwise the longest methionine to stop ORF was used. Manual curations on the transcripts were done with improvements made where possible. This included computational ‘walking’ through the available SCN transcript and public genomic and EST sequences to try to extend transcripts in order to make the ORFs complete if they were not already in the hgg1c assembly. Open reading frames were manually curated and extended, and corrections made against other genomic or transcript sequences if possible. These ORF corrections were used to improve the predicted protein identification, the subcellular localization prediction, whether secreted (signal peptide bearing) or transmembrane localized, and top BLAST hits and functional roles.

Subcellular Localizations Filters.

Signal peptide predictions (using the SignalP program) were made upon the longest predicted ORF in the hgglc contig, determined following 6-frame translation, or upon the best top BLAST hit for the gene from the NR BLAST analysis, or the top gene BLAST hit against C. elegans genome genes. Since many of the contigs may be partials, that is not encoding a complete protein sequence, and not containing the N-terminus needed to ascertain signal peptides presence, the best matches from public NR and/or C. elegans BLAST hits provided surrogate insights into whether the protein is likely secreted or not. Generally, those with positive signal peptide scores were kept, unless otherwise already removed by other filters. Singletons (one sequence contigs) with no annotation or signal peptide predicted were set aside. In addition, the HMTMM program was used to predict whether the protein likely has a transmembrane domain or not. Those having transmembrane predictions were set aside.

Annotations and Novelty Filters.

Functional analyses were done by analysis of the description of top BLAST hits against the NR database, and by analysis of top BLAST hits against the KOGs (Eukaryotic Clusters of Orthologous Groups) databases, in order to infer likely functions for the hgg1c contigs. Manuals inspections of predicted functions were done, and conserved well-known functions were de-emphasized, and novel ORFS or annotations indicating hits from pathogenic nematodes were favored. The gland hgg1c contigs were BLASTed against the public NR database and the soybean genome Glymal transcripts or gene predictions. Those contigs that had strong BLAST hits to NR or to soybean (less than 1e-30 score, or 97% id, respectively) were generally set aside, unless in some cases where they had strong EST enrichment for gland cells and had gland-cell protein top hit annotations. Further those contigs that had top BLAST hits that were well known protein functions that are not of interest, and/or clearly intracellular locations, were set aside. Transcripts with strong matches to genes from plants and C. elegans were selected against or de-emphasized in the prioritization. A further analysis was done on the NR top hits looking at the species source of the top BLAST hit, with positive filtering for those with hits to nematodes, in particular pathogenic (host-colonizing) nematodes, whether they were plant or animal infecting. Generally, selection was against contigs hitting nematodes that are non-pathogenic such as C. elegans. Novel ORFs, with no good BLAST hits other than from pathogenic nematodes, and that that looked like good ORFs (i.e., long ORFs with credible methionine start), were considered good candidates to keep. Further annotations were done to match the gene sequences to those candidates from the Gao et al paper (Gao, Allen, Maier, Davis, Baum, and Hussey. (2003). The Parasitome of the Phytonematode Heterodera glycines. MPMI 16: 720-726). Sequences directly matching these sequences from Gao et al were set aside, and those that did not, were novel, were retained. Through these various analyses and prioritizations, as set of 142 novel sequences were identified as candidate SCN parasitism genes.

### Example 5

### In Situ Hybridzation of Selected Genes (18)

A subset of genes identified in Example 4, numbering 18 genes and shown in FIG. 1 were subjected to in situ hybridization. Photographs of the in situ hybridization and localizations of the sequences in the nematode H. glycines are shown in FIG. 1.

For in situ hybridizations, DIG-labeled sense and antisense cDNA probes were synthesized by asymmetric PCR amplification. The asymmetric PCR labeling was performed in a 20-μl reaction mixture (20 mM Tris-HCl, pH 8.4; 50 mM KCl; 1.5 mM MgCl2; 75 μM of dATP, dGTP, and dCTP; 26.25 μM of DIG-11-dUTP; 48.75 μM of dTTP; 2 mM of gene-specific forward or reverse primer; and 150 ng of cDNA template). The PCR cycling profiles were 94° C. for 2 min, followed by 35 cycles of 94° C. for 30 s, 57° C. or 61° C. for 30 s, 72° C. for 90 s, and a final step of 72° C. for 10 min. The DIG-labeled probe was purified through a PCR purification column (Qiagen) to remove any unincorporated DIG. Mixed parasitic stages of H. glycines were collected at 11 to 15 days after inoculation of soybean roots with hatched juveniles by a root blending and sieving method (De Boer et al. 1999). Parasitic nematodes were fixed in 2% paraformaldehyde in M9 buffer (42.3 mM Na2HPO4; 22 mM KH2PO4, 85.6 mM NaCl, and 1 mM MgSO4) at 4° C. for 18 hours, followed by fixation in 2% paraformaldehyde in M9 buffer at room temperature for 24 h. The fixed parasitic nematodes were cut into sections in 0.2% paraformaldehyde buffer, with progress observed under a dissecting microscope. Nematode sections were then permeabilized in 0.5 mg/ml proteinase K in M9 buffer at room temperature for 30 minutes, as previously described (De Boer et al. 1998). The nematode sections were hybridized separately with DIG labeled sense and antisense cDNA probes at 50° C. overnight. After stringent washes (De Boer et al. 1998), cDNA probes that had hybridized within nematode specimens were detected by alkaline phosphatase-conjugated anti-DIG antibody, BCIP-NBT substrate staining, and compound light microscope observation. Positive clones of nematode parasitism genes display observable (dark stained) hybridization to transcripts expressed exclusively within the esophageal gland secretory cells of nematodes as shown in FIG. 1.

### Example 6

### Expression of Genes in Soybean Plants

Soybean plant cells were transformed with sequences of the present invention using techniques known to those skilled in the art.

### Example 7

### Soybean Embryo Transformation

Culture Conditions

Soybean embryogenic suspension cultures (cv. Jack) are maintained in 35 ml liquid medium SB196 (see recipes below) on rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 ml of fresh liquid SB196 (the preferred subculture interval is every 7 days).

Soybean embryogenic suspension cultures are transformed with the plasmids and DNA fragments described in the examples above by the method of particle gun bombardment (Klein et al. (1987) Nature, 327:70).

### Soybean Embryogenic Suspension Culture Initiation

Soybean cultures are initiated twice each month with 5-7 days between each initiation.

Pods with immature seeds from available soybean plants 45-55 days after planting are picked, removed from their shells and placed into a sterilized magenta box. The soybean seeds are sterilized by shaking them for 15 minutes in a 5% Clorox solution with 1 drop of ivory soap (95 ml of autoclaved distilled water plus 5 ml Clorox and 1 drop of soap). Mix well. Seeds are rinsed using 2 1-liter bottles of sterile distilled water and those less than 4 mm are placed on individual microscope slides. The small end of the seed are cut and the cotyledons pressed out of the seed coat. Cotyledons are transferred to plates containing SB1 medium (25-30 cotyledons per plate). Plates are wrapped with fiber tape and stored for 8 weeks. After this time secondary embryos are cut and placed into SB196 liquid media for 7 days.

### Preparation of DNA for Bombardment

Either an intact plasmid or a DNA plasmid fragment containing the genes of interest and the selectable marker gene are used for bombardment. Plasmid DNA for bombardment are routinely prepared and purified using the method described in the Promega™ Protocols and Applications Guide, Second Edition (page 106). Fragments of the plasmids carrying the silencing element of interest are obtained by gel isolation of double digested plasmids. In each case, 100 ug of plasmid DNA is digested in 0.5 ml of the specific enzyme mix that is appropriate for the plasmid of interest. The resulting DNA fragments are separated by gel electrophoresis on 1% SeaPlaque GTG agarose (BioWhitaker Molecular Applications) and the DNA fragments containing silencing element of interest are cut from the agarose gel. DNA is purified from the agarose using the GELase digesting enzyme following the manufacturer's protocol.

A 50 μl aliquot of sterile distilled water containing 3 mg of gold particles (3 mg gold) is added to 5 μl of a 1 μg/μl DNA solution (either intact plasmid or DNA fragment prepared as described above), 50 μl 2.5M CaCl₂ and 20 μl of 0.1 M spermidine. The mixture is shaken 3 min on level 3 of a vortex shaker and spun for 10 sec in a bench microfuge. After a wash with 400 μl 100% ethanol the pellet is suspended by sonication in 40 μl of 100% ethanol. Five μl of DNA suspension is dispensed to each flying disk of the Biolistic PDS1000/HE instrument disk. Each 5 μl aliquot contains approximately 0.375 mg gold per bombardment (i.e. per disk).

Tissue Preparation and Bombardment with DNA

Approximately 150-200 mg of 7 day old embryonic suspension cultures are placed in an empty, sterile 60×15 mm petri dish and the dish covered with plastic mesh. Tissue is bombarded 1 or 2 shots per plate with membrane rupture pressure set at 1100 PSI and the chamber evacuated to a vacuum of 27-28 inches of mercury. Tissue is placed approximately 3.5 inches from the retaining/stopping screen.

### Selection of Transformed Embryos

Transformed embryos are selected either using hygromycin (when the hygromycin phosphotransferase, HPT, gene was used as the selectable marker) or chlorsulfuron (when the acetolactate synthase, ALS, gene was used as the selectable marker).

### Hygromycin (HPT) Selection

Following bombardment, the tissue is placed into fresh SB196 media and cultured as described above. Six days post-bombardment, the SB196 is exchanged with fresh SB196 containing a selection agent of 30 mg/L hygromycin. The selection media is refreshed weekly. Four to six weeks post selection, green, transformed tissue may be observed growing from untransformed, necrotic embryogenic clusters. Isolated, green tissue is removed and inoculated into multiwell plates to generate new, clonally propagated, transformed embryogenic suspension cultures.

### Chlorsulfuron (ALS) Selection

Following bombardment, the tissue is divided between 2 flasks with fresh SB196 media and cultured as described above. Six to seven days post-bombardment, the SB196 is exchanged with fresh SB196 containing selection agent of 100 ng/ml Chlorsulfuron. The selection media is refreshed weekly. Four to six weeks post selection, green, transformed tissue may be observed growing from untransformed, necrotic embryogenic clusters. Isolated, green tissue is removed and inoculated into multiwell plates containing SB196 to generate new, clonally propagated, transformed embryogenic suspension cultures.

Regeneration of Soybean Somatic Embryos into Plants

In order to obtain whole plants from embryogenic suspension cultures, the tissue must be regenerated.

### Embryo Maturation

Embryos are cultured for 4-6 weeks at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 uE/m2s. After this time embryo clusters are removed to a solid agar media, SB166, for 1-2 weeks. Clusters are then subcultured to medium SB103 for 3 weeks. During this period, individual embryos can be removed from the clusters and screened for the appropriate marker or the ability of the plant, when injected with the silencing elements, to control the Coleopteran plant pest or the Diabrotica plant pest.

### Embryo Desiccation and Germination

Matured individual embryos are desiccated by placing them into an empty, small petri dish (35×10 mm) for approximately 4-7 days. The plates are sealed with fiber tape (creating a small humidity chamber). Desiccated embryos are planted into SB71-4 medium where they were left to germinate under the same culture conditions described above. Germinated plantlets are removed from germination medium and rinsed thoroughly with water and then planted in Redi-Earth in 24-cell pack tray, covered with clear plastic dome. After 2 weeks the dome is removed and plants hardened off for a further week. If plantlets looked hardy they are transplanted to 10″ pot of Redi-Earth with up to 3 plantlets per pot.

### Media Recipes

0.1 SB 196—FN Lite liquid proliferation medium (per liter)—

| | | |
|---|---|---|
| MS FeEDTA-100× Stock 1 | 10 | ml |
| MS Sulfate-100× Stock 2 | 10 | ml |
| FN Lite Halides-100× Stock 3 | 10 | ml |
| FN Lite P, B, Mo-100× Stock 4 | 10 | ml |
| B5 vitamins (1 ml/L) | 1.0 | ml |
| 2,4-D (10 mg/L final concentration) | 1.0 | ml |
| KNO3 | 2.83 | gm |
| (NH4 )2 SO 4 | 0.463 | gm |
| Asparagine | 1.0 | gm |
| Sucrose (1%) | 10 | gm |
| pH 5.8 | | |

### FN Lite Stock Solutions

| Stock # | | | 1000 ml | | 500 ml | |
|---|---|---|---|---|---|---|
| .1.1 | 1 | MS Fe EDTA 100× Stock | | | | |
| | | Na₂ EDTA* | 3.724 | g | 1.862 | g |
| | | FeSO₄—7H₂O | 2.784 | g | 1.392 | g |
| | 2 | MS Sulfate 100× stock | | | | |
| | | MgSO₄—7H₂O | 37.0 | g | 18.5 | g |
| | | MnSO₄—H₂O | 1.69 | g | 0.845 | g |
| | | ZnSO₄—7H₂O | 0.86 | g | 0.43 | g |
| | | CuSO₄ —5H₂O | 0.0025 | g | 0.00125 | g |
| .1.2 | 3 | FN Lite Halides 100× Stock | | | | |
| | | CaCl₂—2H₂O | 30.0 | g | 15.0 | g |
| | | KI | 0.083 | g | 0.0715 | g |
| | | CoCl₂—6H₂O | 0.0025 | g | 0.00125 | g |
| | 4 | FN Lite P, B, Mo 100× Stock | | | | |
| | | KH₂PO₄ | 18.5 | g | 9.25 | g |
| | | H₃BO₃ | 0.62 | g | 0.31 | g |
| | | Na₂MoO₄—2H₂O | 0.025 | g | 0.0125 | g |

*Add first, dissolve in dark bottle while stirring

SB1 solid medium (per liter) comprises: 1 pkg. MS salts (Gibco/BRL—Cat#11117-066); 1 ml B5 vitamins 1000× stock; 31.5 g sucrose; 2 ml 2,4-D (20 mg/L final concentration); pH 5.7; and, 8 g TC agar.

SB 166 solid medium (per liter) comprises: 1 pkg. MS salts (Gibco/BRL—Cat#11117-066); 1 ml B5 vitamins 1000× stock; 60 g maltose; 750 mg MgCl2 hexahydrate; 5 g activated charcoal; pH 5.7; and, 2 g gelrite.

SB 103 solid medium (per liter) comprises: 1 pkg. MS salts (Gibco/BRL—Cat#11117-066); 1 ml B5 vitamins 1000× stock; 60 g maltose; 750 mg MgCl2 hexahydrate; pH 5.7; and, 2 g gelrite.

SB 71-4 solid medium (per liter) comprises: 1 bottle Gamborg's B5 salts w/sucrose (Gibco/BRL—Cat#21153-036); pH 5.7; and, 5 g TC agar.

2,4-D stock is obtained premade from Phytotech cat# D 295—concentration is 1 mg/ml.

B5 Vitamins Stock (per 100 ml) which is stored in aliquots at −20 C comprises: 10 g myo-inositol; 100 mg nicotinic acid; 100 mg pyridoxine HCl; and, 1 g thiamine. If the solution does not dissolve quickly enough, apply a low level of heat via the hot stir plate. Chlorsulfuron Stock comprises 1 mg/ml in 0.01 N Ammonium Hydroxide

### Example 8

### Expression of Genes in Arabidopsis

Constitutive expression of single nematode genes in transgenic Arabidopsis thaliana plants can provide an observable phenotype and information as to the potential function of the nematode gene product within host plants. The cDNA of the nematode gene of interest (GOI) can be excised from pGEM-T Easy vector by digestion with SacII and SacI, and sub-cloned into pBC plasmid digested with SacII and SacI. The CaMV 35S promoter could be excised from pBI121 using HindIII and BamHI, and then sub-cloned into pBC plasmid up-stream of the nematode GOI coding sequence. The identity, orientation, and junctions of the resulting construct would be confirmed by PCR and sequencing. The 35S: GUS gene of pBI121 plasmid (Chen et al., 2003) could be excised with HindIII and SacI, and replaced with the 35S::Nematode GOI construct resulting in the pBI-GOI vector. pBI-GOI would be introduced into Agrobacterium tumefaciens strain GV3101 via electroporation and verified by PCR. Arabidopsis thaliana plants (ecotype Columbia) would be transformed with A. tumefaciens-containing the GOI construct using the floral dipping method (Clough and Bent, 1998) and seeds would be selected on MS media (Murashige and Skoog, 1962), supplemented with 50 mg/L kanamycin. Segregation analyses would identify homozygous Arabidopsis lines of the GOI, PCR analysis used to confirm the presence of the gene constructs in the genome of the transformed plants, and expression of the GOI confirmed by RT-PCR. Positive homozygous GOI Arabidopsis lines would be grown in soil media in small pots under controlled growth chamber conditions to assess potential observable effects of expressed nematode genes on Arabidopsis shoot phenotype. To assess potential observable effects of expressed nematode genes on Arabidopsis root phenotype, seeds of the same GOI lines would be grown on slanted plates of MS media (minus antibiotics) to observe root growth under controlled conditions. Examples of potential observable phenotypes of Arabidopsis plants that constitutively express nematode GOI are presented in FIG. 2 A-F.

### Example 9

### Preparation of Antibodies to Peptides of the Identified Sequences

Polypeptides expressed from genes identified in Example 4 are injected in a mammal, such as a rabbit, to raise antibodies to the polypeptides. Such techniques are known to those of skill in the art. Monoclonal and polyclonal antibodies are contemplated by the present invention and both techniques are well known in the art.

### Example 10

### Expression in Transformed Plants

Plants are grown from transformed cells comprising one or more nucleic acid sequences disclosed herein having a nucleic acid sequence of SEQ ID NOs:1-142, a fragment thereof, a complement of the nucleic acid sequence of SEQ ID NOs:1-142, or a complement of a fragment thereof, particular a plant comprising one of the eighteen sequences identified in Example 4. Expression of a polynucleotide of the present invention may be detected by known methods, such as by in situ hybridization (Northern blot) and RT-PCR. Expression of a polypeptide may be detected by known methods, such as by in situ binding of antibodies specific for a polypeptide of the present invention and mass spectrometry.

### Example 11

### Inhibition of Nematode Infestation by Sequences

Post-transcriptional silencing of each targeted nematode genes of interest (GOI) using double-stranded RNA (dsRNA) complementary to specific target nematode gene sequences can result in RNA interference (RNAi) of the nematode gene and potential adverse effects on nematode infestation of host plant roots. Potential RNAi of nematode genes requires that the nematodes ingest the complementary dsRNA and can be achieved by two primary methods: 1) RNAi-soaking of hatched nematode second-stage juveniles (J2) in a feeding solution containing the target dsRNA and subsequent infection assays of treated J2 in host plant roots to measure potential effects on infestation, or; 2) Expression of host-derived dsRNA complementary to the target nematode gene in transgenic plant tissues for ingestion by wild-type nematodes during the infection process of plant roots and potential subsequent RNAi effects on nematode infestation of host roots.

For RNAi-soaking, the cDNA clone of the nematode GOI can be amplified with gene-specific primers that incorporate the RNA primer site T7. The gel-purified PCR products are used as templates for synthesis of sense and antisense GOI RNAs in a single reaction in vitro using a MEGAscript RNAi kit (Ambion) according to manufacturer's instructions. Alternatively, dsRNA complementary to the GOI sequence can be synthesized by automation using a custom service (Ambion) and diluted to appropriate concentration. The soaking protocol involves dissolution of RNAs in soaking buffer as previously described (Maeda et al., 2001). Ten microliter aliquots of the nematode suspension containing 1,000 J2 are mixed with 5-10 μl of dsRNA solution (final concentration 5 mg/ml), 50 mM final concentration of the feeding stimulant octopamine (Q-0250, Sigma), 0.05% gelatin, 1 mM Spermidine (S-2626, Sigma) and sufficient soaking buffer to make a 30 μl total volume reaction. The mixture is incubated in a mixture chamber for 24 hrs at 28° C. to allow for turnover of the target protein following transcript silencing. Control treatments can include dsRNA complementary to green fluorescent protein (GFP) or other non-nematode gene as a negative control, and soaking solution with dsRNA or octopamine. After treatment, one sub-sample of nematodes are prepared for quantitative RT-PCR (qRT-PCR) analyses by thoroughly washing J2 five times with nuclease free water by centrifugation using standard procedures. Total RNA from 1000 pre-parasitic J2 can be isolated using the RNeasy mini Kit from Qiagen (Valencia, Calif., USA) according to the manufacture's instructions. Trace amounts of genomic DNA are removed using the RNase-Free DNase set from Qiagen (Valencia, Calif., USA) and the Turbo DNA free kit (Ambion, Tex., USA). First-strand cDNA was synthesized from 2-3 μg of total RNA using SuperScript-II RT (Invitrogen, Carlbard, Calif.) and oligo-dT₁₈ primers following the manufacturer's instructions. qRT-PCR analyses can performed in a DNA Engine Mx3000P (Agilent Technologies, Santa Clara, Calif.). A single 20 μl PCR reaction would include 1× Brilliant II SYBR Green qPCR Master Mix (Agilent Technologies, Santa Clara, Calif.), 2 μl cDNA template and 5 μM each forward and reverse primers designed from the nematode GOI sequence. The qRT-PCR reactions are performed in triplicate and the negative controls included water and mRNA extracted from the nematodes to check for DNA contamination in the analyzed samples. Nematode qRT-PCR samples are normalized against a nematode actin gene (ie. AY443352) that serves as a stable baseline expression level. The fold-change relative to control treatments is calculated according to the 2^{-ΔΔCT} method (Livak and Schmittgen, 2001) to assess the potential effects of RNAi-soaking on target nematode GOI transcript levels. A second subset of dsRNA-soaked nematode J2 is prepared for plant root infection assays by being suspended in 0.001% chlorhexidine diacetate for 30 min and then sterilized with 0.01% HgCl₂ for 7 min followed by three 2-min washes with sterile H₂O. Twelve A. thaliana wild-type Col-0 plants in each of the three repeats are in vitro cultured MS medium and inoculated with 50 surface-sterilized J2 on each plant at the root tips. The numbers of adult females that develop on roots and number of eggs produced by reproductive females are counted as a measure of nematode infestation of plant hosts following J2 RNAi-soaking. The data on relative expression of target GOI after dsRNA treatment can be related to nematode infestation levels similar to data shown in FIG. 3.

For plant host-derived RNAi assays, the nematode GOI cDNA can be isolated from the pGEM-T easy vector by EcoRI restriction digestion and subcloned as full-length or truncated into the antisense orientation in the pHANNIBAL vector (Wesley et al., 2001) previously digested with EcoRI enzyme. The sense strand of the GOI is amplified using appropriate gene-specific primers that introduced HindIII and XbaI restriction sites and cloned into pHANNIBAL vector separated by an Arabidopsis PDK gene intron. Both sense and antisense strands of the nematode GOI would be expressed constitutively under the control of a single CaMV35S promoter to form a hairpin dsRNA. A RNAi vector containing the sense and antisense strands of the green fluorescent protein (GFP) can be used as a negative control similar to soaking experiments. The nematode GOI-RNAi and GFP-RNAi constructs made in pHANNIBAL are isolated by restriction digestion with NotI enzyme and cloned into the pART27 binary vector (Gleave, 1992) and introduced into Agrobacterium tumefaciens strain GV3101 via electroporation and verified by PCR. Arabidopsis thaliana plants (ecotype Columbia-O) are transformed with A. tumefaciens-containing the gene construct using the floral dipping method (Clough and Bent, 1998) and seeds are selected on MS media (Murashige and Skoog, 1962), supplemented with 50 mg/L kanamycin. Segregation analyses identify homozygous transgenic plant lines and PCR analysis confirm the presence of the gene constructs in the genome of the transformed plants. RT-PCR (PDK intron transcripts) can be used to assess RNAi construct expression in transgenic plants as well as target GOI transcript expression in infective nematodes that are dissected from roots of transgenic RNAi plants. Seeds of test plants are surface-sterilized and transferred (one seed per well) in six-well culture plates (Falcon, Lincoln Park, N.J.) containing 6 mls of sterile modified Knops medium (Sijmons, et al., 1991) solidified with 0.8% Daishin agar (Brunschwig Chemie BV, Amsterdam, Netherlands). Plates are placed in a 24° C. growth chamber under 16 hour light/8 hour dark cycle for 2 weeks. After nematode surface-sterilization, J2 nematodes are suspended in 1.5% low melting point agarose to allow even distribution and to facilitate their movement into the solid Knops medium. Twelve plants per treatment are inoculated with approximately 50 J2 per plant and placed back in the growth chamber. The numbers of adult females that develop on roots and/or number of eggs produced by reproductive females are counted at 3-4 weeks post-inoculation as a measure of nematode infestation of host-derived RNAi plant lines such as is shown in FIG. 4.

What is claimed is:

1. A nucleic acid construct, comprising, a nucleotide sequence selected from the group consisting of:
(a) a nucleotide sequence comprising any one of SEQ ID NOs: 1-142, a fragment or variant thereof, or a complement thereof;
(b) a nucleotide sequence comprising at least 90% sequence identity to any one of SEQ ID NOs: 1-142, a fragment or variant thereof, or a complement thereof, wherein said nucleotide sequence encodes a silencing element having nematocidal activity against a nematode plant pest;
(c) a nucleotide sequence comprising at least 19 consecutive nucleotides of any one of SEQ ID NOs: 1-142, a fragment or variant thereof, or a complement thereof, wherein said nucleotide sequence encodes a silencing element having nematocidal activity against a nematode plant pest;
(d) a nucleotide sequence that hybridizes under stringent conditions to the full length complement of the nucleotide sequence of a), wherein said stringent conditions comprise hybridization in 50% formamide, 1 M NaCl, 1% SDS at 37° C., and a wash in 0.1×SSC at 60° C. to 65° C., wherein said nucleotide sequence encodes a silencing element having nematocidal activity against a nematode plant pest; and,
(e) a nucleotide sequence encoding a polypeptide sequence comprising any one of SEQ ID NOs: 143-159, a fragment or variant thereof.

2. The nucleic acid construct of claim 1, wherein said nematode plant pest is a Heterodera nematode, a Meloidogyne nematode, and/or a Globedera nematode plant pest.

3. The nucleic acid construct of claim 2, wherein the nematode plant pest is Heterodera glycines.

4. A polypeptide encoded by a nucleic acid sequence of a nucleic acid construct, comprising a sequence selected from the group consisting of:
(a) a polypeptide sequence comprising any one of SEQ ID NOs: 143-159, a fragment or variant thereof, or a complement thereof;
(b) a polypeptide sequence comprising at least 90% sequence identity to any one of SEQ ID NOs: 143-159, a fragment or variant thereof, or a complement thereof;
(c) a polypeptide sequence of any one of SEQ ID NOs: 143-159, a fragment or variant thereof, or a complement thereof; wherein the polypeptide has nematocidal activity against a nematode plant pest; and
(d) a polypeptide encoded by a polynucleotide sequence comprising any one of SEQ ID NOs: 1-142, a fragment or variant thereof, or a complement thereof.

5. The polypeptide of claim 4, wherein the nematode plant pest is a Heterodera nematode, a Meloidogyne nematode, and/or a Globedera nematode plant pest.

6. The polypeptide of claim 4, wherein the nematode plant pest is Heterodera glycines.

7. The nucleic acid construct of claim 1, wherein the nucleotide sequence is operably linked to a heterologous promoter.

8. The nucleic acid construct of claim 1, wherein the construct is an expression cassette that expresses the nucleotide sequence as a double stranded RNA.

9. The nucleic acid construct of claim 1, wherein the construct is an expression cassette that expresses the nucleotide sequence as a hairpin RNA.

10. The nucleic acid construct of claim 9, wherein the hairpin RNA comprises, in the following order, a first segment, a second segment, and a third segment, wherein
(a) the first segment comprises at least about 19 nucleotides having at least 90% sequence complementarity to a target sequence set forth in SEQ ID NOs: 6, 7, 8, 9, 10, 11, 12, 18, 19 or 20, a fragment or variant thereof;
(b) the second segment comprises a loop of sufficient length to allow the silencing element to be transcribed as a hairpin RNA; and,
(c) the third segment comprises at least about 19 nucleotides having at least 85% complementarity to the first segment.

11. A host cell comprising a nucleic acid construct of claim 1.

12. A plant cell, comprising, at least one a heterologous nucleic acid construct, wherein the heterologous nucleic acid construct comprises
(a) a nucleotide sequence comprising any one of SEQ ID NOs: 1-142, a fragment or variant thereof, or a complement thereof, wherein the polynucleotide encodes a silencing element having nematocidal activity against a nematode plant pest;
(b) a nucleotide sequence comprising at least 90% sequence identity to any one of SEQ ID NOs: 1-142, a fragment or variant thereof, or a complement thereof, wherein the polynucleotide encodes a silencing element having nematocidal activity against a nematode plant pest;
(c) a nucleotide sequence comprising at least 19 consecutive nucleotides of any one of SEQ ID NOs: 1-142, a fragment or variant thereof, or a complement thereof, wherein the polynucleotide encodes a silencing element having nematocidal activity against a nematode plant pest; or
(d) a nucleotide sequence encoding a polypeptide sequence comprising any one of SEQ ID NOs: 143-159, a fragment or variant thereof, or a complement thereof, wherein the polynucleotide encodes a silencing element having nematocidal activity against a nematode plant pest;
wherein the silencing element, when ingested by a nematode plant pest, reduces the level of at least one target sequence in the nematode plant pest and thereby controls the nematode plant pest.

13. The plant cell of claim 12, wherein the nematode plant pest is a cyst nematode.

14. The plant cell of claim 13, wherein the nematode plant pest is H. glycines.

15. The plant cell of claim 12, wherein the silencing element comprises
(a) a polynucleotide comprising the sequence set forth in SEQ ID NOs: 1-142, a fragment or variant thereof, or a complement thereof; or
(b) a polynucleotide comprising at least 75 consecutive nucleotides of the sequence set forth in SEQ ID NOs: 1-142, a fragment or variant thereof, or a complement thereof.

16. The plant cell of claim 12, wherein the silencing element is a double stranded RNA.

17. The plant cell of claim 12, wherein the silencing element is a hairpin RNA.

18. The plant cell of claim 17, wherein the polynucleotide comprising the silencing element comprises, in the following order, a first segment, a second segment, and a third segment, wherein
(a) the first segment comprises at least about 19 nucleotides having at least 90% sequence complementarity to a target sequence set forth in SEQ ID NOs: 1-142, a fragment or variant thereof;
(b) the second segment comprises a loop of sufficient length to allow the silencing element to be transcribed as a hairpin RNA; and,
(c) the third segment comprises at least about 19 nucleotides having at least 85% complementarity to the first segment.

19. The plant cell of claim 12, wherein the at least one heterologous nucleic acid construct further comprises the silencing element operably linked to a heterologous promoter.

20. The plant cell of claim 12, wherein the plant cell is from a monocot.

21. The plant cell of claim 20, wherein said monocot is maize, barley, millet, wheat or rice.

22. The plant cell of claim 12, wherein the plant cell is from a dicot.

23. The plant cell of claim 22, wherein the plant is soybean, canola, alfalfa, sunflower, safflower, tobacco, Arabidopsis, or cotton.

24. A plant or plant part comprising a plant cell of claim 12.

25. A transgenic seed from the plant of claim 24.

26. A method for controlling a nematode plant pest, comprising, feeding to a nematode plant pest a composition comprising a silencing element, wherein said silencing element, when ingested by said nematode plant pest, reduces the level of a target nematode plant pest sequence and thereby controls the nematode plant pest, wherein said target nematode plant pest sequence comprise a nucleotide sequence comprising at least 90% sequence identity to any one of SEQ ID NOs: 1-142, a fragment or variant thereof, or a complement thereof.

27. The method of claim 26, wherein said nematode plant pest comprises a cyst nematode plant pest.

28. The method of claim 26, wherein the nematode plant pest is H. glycines.

29. The method of claim 26, wherein the silencing element comprises
(a) a fragment of at least 19 consecutive nucleotides of SEQ ID NOs: 1-142, a fragment or variant thereof, or a complement thereof; or,
(b) a nucleotide sequence comprising at least 90% sequence identity to any one of SEQ ID NOs: 1-142, a fragment or variant thereof, or a complement thereof.

30. The method of claim 29, wherein said nematode plant pest comprises a cyst nematode plant pest.

31. The method of claim 30, wherein the nematode plant pest is H. glycines.

32. The method of claim 26, wherein the composition comprises a plant or plant part having stably incorporated into its genome a polynucleotide comprising the silencing element.

33. The method of claim 32, wherein the silencing element comprises
(a) a polynucleotide comprising the sense or antisense sequence of the sequence set forth in SEQ ID NOs: 1-142, a fragment or variant thereof, or a complement thereof;
(b) a polynucleotide comprising the sense or antisense sequence of a sequence having at least 95% sequence identity to the sequence set forth in SEQ ID NOs: 1-142, a fragment or variant thereof, or a complement thereof; or
(c) a polynucleotide comprising the sense or antisense sequence of a sequence having at least 75 contiguous nucleotides of SEQ ID NOs: 1-142, a fragment or variant thereof, or a complement thereof.

34. The method of claim 29, wherein the silencing element expresses a double stranded RNA.

35. The method of claim 29, wherein said silencing element comprises a hairpin RNA.

36. The method of claim 33, wherein the silencing element comprises a double stranded RNA.

37. The method of claim 33, wherein said silencing element comprises a hairpin RNA.

38. The method of claim 35, wherein said polynucleotide comprising the silencing element comprises, in the following order, a first segment, a second segment, and a third segment, wherein
(a) the first segment comprises at least about 20 nucleotides having at least 90% sequence complementarity to the target polynucleotide;
(b) the second segment comprises a loop of sufficient length to allow the silencing element to be transcribed as a hairpin RNA; and,
(c) the third segment comprises at least about 20 nucleotides having at least 85% complementarity to the first segment.

39. The method of claim 26, wherein the silencing element is operably linked to a heterologous promoter.

40. The method of claim 38, wherein the silencing element is flanked by a first operably linked convergent promoter at one terminus of the silencing element and a second operably linked convergent promoter at the opposing terminus of the polynucleotide, wherein the first and the second convergent promoters are capable of driving expression of the silencing element.

41. The method of claim 26, wherein said plant is a monocot.

42. The method of claim 40, wherein the monocot is maize, barley, millet, wheat or rice.

43. The method of claim 26, wherein the plant is a dicot.

44. The method of claim 43, wherein the plant is soybean, canola, alfalfa, sunflower, safflower, tobacco, Arabidopsis, or cotton.

45. An isolated polynucleotide, comprising, a nucleotide sequence selected from the group consisting of:
(a) a nucleotide sequence comprising any one of SEQ ID NOs: 1-142 or 161, a fragment or variant thereof, or a complement thereof;
(b) a nucleotide sequence comprising at least 90% sequence identity to any one of SEQ ID NOs: 1-142 or 161, a fragment or variant thereof, or a complement thereof, wherein said nucleotide sequence encodes a silencing element having nematocidal activity against a nematode plant pest;
(c) a nucleotide sequence comprising at least 19 consecutive nucleotides of any one of SEQ ID NOs: 1-142 or 161, a fragment or variant thereof, or a complement thereof, wherein said nucleotide sequence encodes a silencing element having nematocidal activity against a nematode plant pest;
(d) a nucleotide sequence that hybridizes under stringent conditions to the full length complement of the nucleotide sequence of a), wherein said stringent conditions comprise hybridization in 50% formamide, 1 M NaCl, 1% SDS at 37° C., and a wash in 0.1×SSC at 60° C. to 65° C., wherein said nucleotide sequence encodes a silencing element having nematocidal activity against a nematode plant pest; and,
(e) a nucleotide sequence encoding a polypeptide sequence comprising any one of SEQ ID NOs: 143-160 or a fragment or variant thereof.

46. The polynucleotide of claim 45, wherein said nematode plant pest is a Heterodera nematode, a Meloidogyne nematode, and/or a Globedera nematode plant pest.

47. The polynucleotide of claim 46, wherein the nematode plant pest is Heterodera glycines.


SEQUENCE LISTING (159 sequences)

SEQ ID NO: 1 - DNA, 1143 bp, Artificial Sequence
  Description of artificial sequence note = synthetic construct
caaaagctcggcactccgacgtggacgagatcatacatttgcgcaaccggcttacatgcgtgacccgttgcgtgccgaccttctcgcgggctccaaactgaaggaggtgaagaagacggactacaaccagtgcaagtccatgctgctcgacctgttcgacggcacgcgcgtgattttggtgggcgaaacgcgggaccgaagcggacgcaagcggttgatctcctgctttcaactgtaccgacaaagcagagccgcggcaaatttcggcatgttcgctgtccatccctttttccaagcgtccggacttggcaagcgattgttgactgttgctgaacgctatgcccgtattgtgtggggcagtgacgagatgcatttggatgttggcgggagtttggccgaattaaagttgggcatgggacgactgcagagatactacaagcggcgcgggttcctatcaaccggcattcttcgccccttcaatggggctgtggcgcgcttcatcacggtagaccgaaacgatctgtggattgagctgatggtcaaggacatacgtggagcattggatgacatcggcggagatccagagaaacggatgaaaagagtgaacagtcgggggagattggccagagaagcagacaaagacgacggcggcagagatccacaaaaaaggatggagagagtgcgaagctttgggagattaaccatagaagcagacagggacgacatcggcagagacgcgcaaaaaaggatggagagagtgcgcagtttagggagattggcaagagaagcagacaaatcggatgagagtaaaggcaaagatggggaggaaaagaaaaagacaacacaggcagagggggaagagagtaaaggcaaagatggagaggaaaagaaaaagacaacacaggcagagggggaagagagaattaagcctttggctgattgaagaagcattcaaacagttgtgtctcctcgaaaaatacagactctgaagcttcaatacagtaaatacagtatgcttgtcccggaataatttaatgaatgtcatcgttttttttattaaaaatttttcaaatcgttgccagttggcgtttcgtcgtagttatactgtagaaagattggcaaaaataaatgtttctggcttaa

SEQ ID NO: 2 - DNA, 1188 bp, Artificial Sequence
  Description of artificial sequence note = synthetic construct
catccattgatttagcccctattattggatttatcccgcttttccttctttcgctcctccccttctgaactcttatttatacctctttttgcccccatataattattctgccaattttccattggcatggctctctctgcccttctcctcctccttcctctgcttctcaatgtgcaaaatatcccagatgagtccgttcaatcggatgtgaaagccgttgattcggccatttcgtcgctggaacaatggaaggacccgcgcaattcgttggcatcactcgactcacagctgacagagccccaacgagcactggccaaaatgttttgggaattggagaccatcgaaaaggaaaagccgaaggcaccgccacaattcgacttgggacttttcttggaagctttggaagcgatggtcgaaatgaacgaagaagcaaaggaagtgaagctgagaaaggacaaactgaccgaatgggcaggcggagagaaagcaaacgaagggaaagaagggaagacgaaggaggaggagacagtgccggaagtgagagttaatgagaatgtaaaggtggaagtgacgaacggcgccggaggggacggaaagatggaagtcaagcgaggaaaggacgagaacggaaacgagcaggtggtggtcacctttgtgaagagggacggaacggagggaaagacggaggaggaacagaagaaagaggagaaggacaacctacggaagggacgggaggaggtcaagatggagcaggacaacgtagaaggggcaccgaaaacggactcggccaacagtgccaagtcacccattccaatgcccaccattttgtcctccccggccgcaccggcagaggaggaggaaaaggcgaacgatgcgttcacagaagcaaatgtgaggaaaaaggtgaaaaaggacgaagaaatgttcataattatgactgatgacaacggaaggacgggaaatgcgaatgaaagacaaatggaatttgtcagaatgccaaaaaaagttgggagagacttcggcagcgaattgttcggtttgccacaaccttcgaacggcggacaaagcccaatggaaatgtttttcaatttgtttggacgaaaaaaaagggaaacggtgcaggaaggaagaaagaaacggagcatcgaaaatttagccaatttggggaagccgggctcagagtttgtg

SEQ ID NO: 3 - DNA, 387 bp, Artificial Sequence
  Description of artificial sequence note = synthetic construct
acgcgggaaagggaaaaatgccgctaagaaagacaaaacaaagaacaaaaaggcaccagcagcagccaagccaaaagctgagcctgttgagactgaagagccatccagtgctcaagttgtagctgaacaggacggaagcgatgagtcagctaacaaccaagaaatggatgccggcgaagagattgcagaggaggagcagactgatttggcacaggatgaacagcttgaagacgatgccacggacggtgaagaaggaaatggtatggctgaggaagaacagccggagatcaactaataaactatttttagaaaaatatttaggaaaataattttctatgggtgaaatgtagctgtagttttccactgatgtgtaaatgtatattttac

SEQ ID NO: 4 - DNA, 695 bp, Artificial Sequence
  Description of artificial sequence note = synthetic construct
aattccatcaaatctgccaaagatccttcaaaaatgtcttctccttcttcgtccgtctctctactcgccatcgtcacaattttctgtttgctgtgcaaatgttgcgtttcggcaccgcatccgtgctgtcccggcagtcaaaaagtggtttcgctgatggccaattacgttggcactttcgcccattccttttcaaaggcatcgctttgttcggatgcccaaagtgttgcgggcgcattgaaaggccaactgatcggctgctcgaagggcggcgacgcaactcttttggccgacatcgaagcatctcttgccactcattctgctgatgagtgtgcccacagcctcggcttcgtccgtgccatgttcgccattgccgcctccgcttcttcccatgccagcaacaacaacgaatggcaggcattgagtgcccagtttggtcagcaaatcagtgaaattgactcgaaatgtgccgagtttggcattggcattgccaaagtgccatatgacggccccaagggtgatcactcccaacgaaatgtgcatggcacggacagtgtaattgccatgcctggattggccggctcacacaaacaatgaatagaatcaatgggtcactgaatggaacgaaatgattgtggagttcgtttttgatattgtccttcttttagttgatgaatagtaaaaataaatttaag

SEQ ID NO: 5 - DNA, 669 bp, Artificial Sequence
  Description of artificial sequence note = synthetic construct
gacatcattaatatattttattcattattaaataaaaaatctttttgccatgttttctctgatgctctccatcttcccaattgtctttttggtctgttgcaaggcaatgccaaatttcccgtgctgcccgggaagtcagcaagtggttgctgtgatgtccaattacattggcactttcactagtgaggacaaatctacagtatgctcaaccgcaaaaaatactgtggaaggaataaaaagtgaactttcatctcgcgtgggatgcccaagcggaggagaagcacaaattgtgaacgaaatcgaccgacagctgactaacattgcgaaaatggaaatcaattatgaggacgagtgcccgtacaatttgggctttgcccgtgccatgttcgacttggccgctgctgctggccatgcgggcaacgacacagaatggcaaaacatgaaaagcaaatttgtacaggaaagccaagcaatcaaagcaattggccaagaaatgaacattgaagttacggatgtgcacattggacacccaagcaaagggatttccgcgcaccaaaatgtgccaagtccaagccatgtgattgccaaccctggccaacacagttcggttggccatggaaaggaagacacaccgttgtcatcggatttcgatttttgagggcatagaaa

SEQ ID NO: 6 - DNA, 1167 bp, Artificial Sequence
  Description of artificial sequence note = synthetic construct
tatatatttattaattctctttaaatctttaaaatgaaaataatttctattctcatcaattttattctggctatctatgaagcaaaaggtggaggaattgtttctttactatcaagaagacaagcaccaaagcgtcatttagctagttcactgcgtcaacaacgcaccgaggacaatcacatttcaattaatggacaaaattacgcggttgacggacctaatgttaatgttggtgttgaagggcatgatttgagtgtgaatgggagagtttatcaaaacagggccacagagcagtatctggaaattatacaagacaaaaacataagaaatgtaattgtcagtgtgccattatcgttattttctcgcgaaaacataatcgatgggcaaataaacgctaaatgcaatggaaatttatacatcgatcaatcgtcagatggatgttctcgcataatatgcgtcgacgataaaaagaatggcgttgaaaataactttggacagacacgtgatattttcctgaccggtgatgtcaatatttttgagtctgcaaatggaattatctacaactctatgatgggaggaactttacatatccataattcgtcacttgagtgtgctaacattgaatgtgatgcatctttaaatgtaactcactcaccaatagaacgtaatgcgcaaatgaaatgtggtgggagtttaagtattgatgagtcaccaatgggaaatattcggcttaactgtgatggatctttgcggatcgaaaaatcgaaaatggaaagcagtcagattgatgttggtggaagcattgggattgttgagtcaccaatgggaagtattgggattgactgtggtggatctttacggatcgaaaagtcgaaaatggaaattggcaacctagactgtggaggaagtttaaccattgtagaatcgacagcgcaaagtctaaagttaaactgtggaggaagtttaaatatgaaggagtcgccaatgaaaaatgttggcattaattgtgatggaagtgcaaccattaagaagtcgaaaatggaaagtggtcgcattaattgtggtggcaatttttctattgatagttcgccaacgggaagtgttcgaattgattacggtggaagaagaattaatttatgaggtcaaacgaatgatcttgttcggaac

SEQ ID NO: 7 - DNA, 687 bp, Artificial Sequence
  Description of artificial sequence note = synthetic construct
ttttacaaaaaaaagaatattttttaataaaaaccattaagcaactaacataatggccattcttctgaagtttgttctgttcatctcaataatggcaattttctgcgattgtatggaccccggcaaaaatgggaaaaacgaaaaaaaagacgttgtaaaacaaaaagtggacgaaacgaaagttgagcgcgccagtgaaatgaacaaaggcaaaagcatcgttatggctgactccaaaaaggaaggcacaacgacagtgaaaattccgcaccgttatggagcagtgtcggggatgagtggccaaaatgccagtccagaagcctctcaaattggcagtccaaaaaacagtccaaagggcactcaaattggcagtccaagatccattagcagtcctaaatcaacacaaattggaagcccaaaaggcattcaaattggcagtccacgaaaagaaaagaccaaattatcttcagctgttggctcttctgatttcaatgttatcgacgaatcaaaagaagcgaaaaaaaccaagccaattcaaaccgagtccgtccagaagccaaaataaacgcgaacagcagcgactcaatgttactattggagaagcgggaagagttcaatcatcaaaaagagtgtcgagcaaagacacctttagtccgtcaaggaaaaaaaaaaaaaaaaaaaaaaaaa

SEQ ID NO: 8 - DNA, 764 bp, Artificial Sequence
  Description of artificial sequence note = synthetic construct
acgcgggggacagattgctgactatgcaggaaattcatctgagagtgccgaaggacgtttactccgaataccacaaattggtgaaggaccccgaagacagcaaaaaaaataatcccatccaaaacgattccgggaagagttgtcgaaatccaacggaccagcgacaatttgtacagagcgttggcttatgcactgacgggcaccgaaatgcttcacaaggcgactcggatggttgtgctcgaatactttgagagtttcttcggacaatgggacaaaaagcaggcgcagccgtggatggacgaatacgaagtgcgaagtgtgcgcagacaggcggagaaaataaaggcgggcaaagccgggggcacggtcgagctgatagcggcggcgaaaaagttcaacatgaacgtgctggtctacaagacggacaaggacatgtggctgtgcatgtcgccaaagacggcgcacaaatgggacttggacaagaactgccaaagcaaggatgcgatgaccattgcgttggaattgtacgacaacgaaaattacgacgtgattatggacgtgcaacaaaagaagtgaacggagaggcggacggacggtcaactcaaaaagaagaatgaaatgagaaaatgagtgaagattttgttcgtagtgattaggggcttaatgatcgtcggatgatacaaatcactttataagcaaatgtaaagtaatcatcgaaaatcattcggcagccgtattcccaccaaataaatgagcattcgctg

SEQ ID NO: 9 - DNA, 1941 bp, Artificial Sequence
  Description of artificial sequence note = synthetic construct
caagtttgagttcgttccttttccatgcgtttttcttcattttcctccccttttctccccctctttttcctttctttgccaattgcgtttgttttgtccggccgaactttgccgttcaccggttcgcaattggccaatgaagtggccagggcattttttaattccgtcaacacttgggacatgtcaattttcggagccgggactaagcagggcgaggaccgttacaagatcagcttggacggcctggacagaatgaagaacagattcagagtgccgttgccggcggggcaggggttggaaaagctgctcagatcgtacagagtggagcctctcagagaggattaccttggggtgaacaaagccagagaaagagtgttggcaccgagtaaactgatggaactgatggaaaagctgggcaatgtgctggttacggacccaaaaatgcgccaaaagatcgacaaatacgacaaaaaaagagcggatgaggcggcgcgaagggcggcgatgatgccaccaaggcaagacccacaagcgattgcaaaacgcaggacgtggccgaaggaggacggattggcattagaaaggggccatttgcctcaaggcaacaaccagagtccgacgcgactccagtcgacgcccaggatttggattcaagaagatgaccggtggcgccaaccgatgactttctcccgaaaagacgtgcgggaaagaagttggctcgagtcggacaccgactcggacttggacagcccaacttcggtgttgcgctcgcggcgaaggagtcgagtgaacattttggacgacgaccaaccgacaagaagaacggcctggggaaggtcgccgacgccatcgccaaatggacgtgctgttgtacaacgaacaacgaccacaacgacgacgacaactgaggaggaggaaggggggcgaagaacggtcagatttggcgaagtggtggtcgttgagccggaagagagaacagtgaacagacggacggaagtacggacacaacagcgggagaccgaagtggagaggacgtcggaatataccctaattctgcgaattgatttcatcgatgcctccgtttttttggacaaatcgttggcttactttggaagtctgaacactgccaggaaagacgaaaggagtgtgcagcgattgtgctacgtactgaaggcatttgacccgaggcacgaaagactgaattcggtgctcgccactccgtcggtggccaatgctttcgtcgaatacaaaaaggcactgaacgacgtgggactgaactcacagcccgaactgcgacttgttgaaaaaagcaacgcctgtgccttcgacttggctttgatttacgaattggcccaattcaccaaagatttgctgttgaagcttaaggccgagcgaatggtggcggcggaggagttggaggacgtcaaagaagaagtgatcggacgactgctgaagcttttgcccaaagttttggaaggactgaaggcaaagcctgccgaactatcgacggaagtcgaccgacgcattcaggcacttgacgtagtggaagagcaactgaatgtggtcaaaagagctcgagcgaccgacgaaatggtgacgggggcaatggccaaagtgatggcacagctgagaaatgcgtcacgaggaatgggaacaatggacatgagcacactgagttctcttcaatcgaattgggacaatctgatgagaaaggacacccattggcaaattcggaaggcaattaacagcctggggggatgcccgaaagacccgcagggcaacacgctaatgaagcaatgcatggaggaagcgatcaccaaagtggaccgatacattgacgacgtgaacgactggttcaaatcccagcgaccaatcgacatggacgactggaagtggctggctgctgagattcaaatgataattcgttggaagagcccttga

SEQ ID NO: 10 - DNA, 697 bp, Artificial Sequence
  Description of artificial sequence note = synthetic construct
caaaggaatcaacaccagacatggccattctgctgaagtgtgtgctgctcctctcaatcatggcgattttctgcgactgtatggaccccggcaaaaaaggaaagagcaaagatccgatcccaatcccgaaacaggaaggctcagatccgatcccaatcccgaaacaggaaggctcagatccgatcccaatcccgaaacaggaaggaaagccgagcagcagtgcagcgaatagcccgacagtaacaaaaggcactccgaaacgtggcgaacttgatacccccgaattttacaaaacgagcccaaagaacaaaattaatagcccgagaaagcccaacaacggctctccgagaaaggataaaaaagctctacaaaaggaacgtcaagaagaaagaaagcaaaaagaaagagaaagagaaaaccgtttcctgcgaacgaaatcaacagcaggtaatacgactgacgcgactgacgtggaaaccgaaagcgaagtgattccgacatttgttgccgaactcgaagattctacggtggaatatccaacagacattgaatgatcatgttgcaacaaaaactgaccttggacggaaatgatcagcagaaagcactgcaagaatgaggaaaaaagaggcacggaaagaatgatttgtgatagattctttcttctgtgcattttttctgttgcgtaaatgttgagagc

SEQ ID NO: 11 - DNA, 483 bp, Artificial Sequence
  Description of artificial sequence note = synthetic construct
gattcaactttaatttgactgtgcttccgaattgtcaaaatcattaataatttatcgcgcaaataatggccaacaaatttttaattgctgcttttattttgacaattgccatttttgtcaatgggcaaagtgaggcgccgaacaattcgtcggaaatggcatcggaggagagcaattcggaagagtcgagcagtgaggagcagcagttcaacccattcaaatttcggccattttttggtccctcgtcgtccaacagttcggcaccgccgccctttgcctttttgcccttttttggacgaatgccgtcgctatttaaccgcccctccaacaagagcgtcgtctgacaattgatcactttttgagtgatttgtgggcgtcgagcagtgtgaaatgaaaccgatgatgagcaaatgaattacattccatttatcgttcatttttgacttttaaaagaaagaatacttgcataaatttattcaggcg

SEQ ID NO: 12 - DNA, 667 bp, Artificial Sequence
  Description of artificial sequence note = synthetic construct
gactcccaaataaaataaaattaattaaaataaatacaataatccacataaaataaaacaatgaacaaatttgtgggcatatttgtcgctgttttgctccaatttgtttcgccattttcggcattttcccgcgtgccaacgacgaccaccgaacgaccgataatttatgacccaaaagaaatggtggaaatccaagtgaatttggtgaacaacaccaacaacaactgcacaaatgatgttcttcgaaaataccgtgtggagatcactaattatgtgttctttttggtgtgcgatttgaaaattcgagtccaattgccggaaggggcaactttggagaatgtcgtcaacctgaaaccgttcaatggcaccaccgatcaattcatttttcccgattccttgcgctacctttacgtttccaaaacgctcgaagccgaactgagcgtcaaaggcggcgagggggaaccgaaaatcactgttttggatgcaaaggccgctttttcgccgaagaaatgccgaatttcgaaattttaatggcaatttaaagaaaggacgaaaatgaaaggagaaataggatagaaaacgtaataatttctaaagggatttgtatcaataaatatggaataaatgttgatgaaccagaaaaaaaaaaaaaaaaaaaaaaaaaaaa

SEQ ID NO: 13 - DNA, 611 bp, Artificial Sequence
  Description of artificial sequence note = synthetic construct
aagggaggcgaccgtgctgaaacacgtgggtaaccagaccaacgcggccggcatcgacgcggaatttgctgtgaacttcctcctggcacagatggaggccaacaaaatgattcagcgaggatatatcgaccggtggaattcggatcactctttcgagtcaaaatatgtgccggattttgagaaagaaattcaacctaaattttcttacgcaacgaatgcattgattttggcactgattccattggtcgatgcgggccaccaaatgcacaacgaccaaaactgtgttgagcatgtggaagacgtgttggaatcgatggagcatttgcgagccagcgaattggagccgaacggaaaggaagccatggaaaaagcggtcaaagcaatttgtgaaaaaatatcgacacatgagggacaaagcaacgcagaagatcaatcaaaatcgaaaaaacggaaacattctgacaatcacaaaatggaagagggaaagcatggggaagaaaaagaaattcgacccacaaaaagaacacggaaagcgaacacagatgaaagcaaaacaccagcagcaggggaaaataggagaaatcatcgcagagaaaactatgtggatagtg

SEQ ID NO: 14 - DNA, 1022 bp, Artificial Sequence
  Description of artificial sequence note = synthetic construct
aatgggtactgtcatatgtgtcggacaaaggctcataccctgtacttggcaaggacgcggagggaagggaacgaatgaatgctctgattgttggacattttgatggccatacgtttgagaagttgtttgaacagcaaatggactttgttggcggctcatttgcttatcagggcttccatgaccaacagtcgggcagatcatttaccatcggatggatctgcgacattggctggatcggcgacaacactggtgacgcgaactttgatggccgaggtggcgtcacgtcgatgactttgcctaaggaatttgttctgaaggacgaccatttgattgtcagaccgttgcccgagttggcccaactccgtcagagcaaacaaccgcaccaaataagaaagggtgaaaaatacagtttggaaaaagggcatgccgaacttttgttccaattcaaatggtccaataatgatgatggttcagcagaggagaaattcgtgttggacttgacccgaacacggttaaaagatggcaaattggagttcacaattgacagcaaaggcattgagctgaagaggacttgggtaaaacccaacaaacgtctggtggtgtacaatgttaagccgggtcaaatccatgtgttcatcgacttggacactgtggaatattttgcggataatggccgatggtcgggcgccgttcgggtgccaaatgcaagccaagaaaatcgaatcggaacagttgaactgaaaagtactccgctggtgcttgagcagtccagcttatggtatctgaaatacggatcacacaaatccgcgcggcttcaaccaaacggcattccatttgcaatgaacgctggaacgtcgtcattcaaacaggatgaagcctaaagaagacataaattgtgcctcataatctttgattatccaagatagaaattgatagattaatgggagatcagtagtacttttaattggatatatattaatttcctcacaatttaatggctttgtaaaatttgattgttccaa

SEQ ID NO: 15 - DNA, 876 bp, Artificial Sequence
  Description of artificial sequence note = synthetic construct
gattatccagagatgaataataattttttattgttgctcatcacctttacattcatagttggtgcacgtgctttttggatccaattgccaggcaccttttggggatatggtgatgcacgccagcaacagcaccgggggtggcttaatggatggcacagttggcacaaccaaaaacataatggtgccaataccggtggttattggcccatttatggccacgggcatggacattttggtaatggaaatgcattgccagcagatgatagatcttccaacgaagaagacgacaacgaaacatcggaggaacagcagctaacaacagatgatccgccagagaatgcttcatctgacataatggagccgaatgatgggattactgatcagccaactgatcaagatgggagtgatacagaagcaaccgattcgacgacagttggatcggatccaggaccaaatgacaatgatcagaatgccactgggccaactgatgaagatgaaacaggaacggaagcaaccgattcgacgacaacaacaactgaatcaaatgcaataggtgaagaaggtactgatcaggatgctacaaactcatctgatcagggagaaagtgatgcagaagcagaagcaaccgattcgacaacaaatggatcggatctggaaccaaatgatcaggatgaaaatggtgcggatgctgattcgacgacaacaaacggaatttgatcaaaatttactgaaaccaaactggcaatgatcagttgaattttttgatttgcagcgtggttgatcaattaatgacgaatgtcaatatcattttgatattgcaattaaaaccgatgtagttcatttgcgatacaattttttttcatgtgtacaacgaaa

SEQ ID NO: 16 - DNA, 684 bp, Artificial Sequence
  Description of artificial sequence note = synthetic construct
ggagaaaaagcaaaatgtgttcgacgatttcattgcggctgccgagtatctcatcaacaaacagtacaccaacagctcgaagctggctattttcggcgcctccaacgggggtttgttgaccgccgtctgcagtcagcagcgacctgatctcttcggagctgtgatcacccaacttggattgttggatatgctgcgcttcaacaaattaggcattggctcagattgggtgtcggagtacggcgacccggacaatgccacagacttttcgtacatttacaagtattcgccgcttcagcagctcagcgtcactcccgggaagcagtggccggcgactcttttgctctcggctgaccatgacgatcttgttgatgtgtctcacacactcaaatatacggcacaactgtatcatttgttgcgcaccaatgctgagagttggcagcgcaaccccgtggtggcaaagattttggtggaccaagggcacgcgttcaccggcacaccgaccgagaaaaaaatcaaagagaaggttgacatttacactttcatcgcgcgagcgcttgggctgaaatggaccgaatgattaagaacaaatccatctgtgtgatcactgatcagatcattatcgatgcaatatttttaggattttcttttcataaatttgcattccataaaattttgggacaactg

SEQ ID NO: 17 - DNA, 778 bp, Artificial Sequence
  Description of artificial sequence note = synthetic construct
cggggggaacaccggctgtacntgcatatgtttatgatcgaaaaggaacacattatgaaaagaaaatacgcgttgacgattgggacaatcattacattgtggatttggccactaatgatgtacaagatgtgttaaaacaaaatttggacttggaatttctaaagctaagagacagtgttgccagtggagaaacgaaagaattgacattctatggccgagtttggcccgaaggcaagtacaaacttttttgggacgtaaaaggctttgaaatggatgaagcgcaaagattgatcaaatcggaattaaatgtgccacacgattgcttcaccgatgagaatggaaaattcaaattggaatatgaaattgagaataagagcagagaagtggcacgatggcgtctcccgcctgtgcatttgtacatttttggggcaagcgtttggacaaaagaatatgtgcatgtgacagattggcatcatgtgcatatctttgatttgaaaaatgggaaaaaacatgcacttccggcggataaagtcgctgaaaaattatacgaattaagtaaaagggaccaaatgaatgaacgaacaaagttggcagaaacaaatgaaaaaaacgaaaatgagatcacgttcacgcgttcgttttgcccattcagacagtgactattagaaatttcgatgtcaccgaagtttttcgctggatgtgttggaacgggaattggagaatggctgatgaatttttggaatttataatcataaacaatttgttagattagagttca

SEQ ID NO: 18 - DNA, 630 bp, Artificial Sequence
  Description of artificial sequence note = synthetic construct
atggcccctctcttccatcgcttctcatctctctttgtctttctgatgccgttcctttccgttgtgcttctcccgtcaactgtttgtaccggctctgacagtgccgccgcgccgttcgaccgaaagaattatccgaaaatcgatttgcgactgttcgagtggcccattgcttcacattcgggctcgtccgctgaggtctcttttatcgccgtcgactgctacacccaattggaccgttctttcatctcgaccgatgccgtgctccgtctcaacaattcgttagcacttcggcaccgcgcctgtctcttgcgcattccgacggggacgcggctgacagtgaccgaaatgcaaacgaccaacagaaaggtaaataagacaaaaccaaaacttcggcccatggcacgtgccgtgccaacaggcgtatgtgctgttcaactcgcgcgggcgcaaaatggaatgggtcgaatttcgtctggacgacgaaacggaggcggacaaagagatggcgagcgcggacgaatgtttggcggacgaagaggaggacgaagaggaagaggagaagggataccgcaaaaagcgagctcattgagccgctgggcagcagactcttttggctttgatgagcattga

SEQ ID NO: 19 - DNA, 685 bp, Artificial Sequence
  Description of artificial sequence note = synthetic construct
caaaaggaaagaggaaggtgaggaagaagatgaagaagaggagggggaagaagaagagggagaggggcaacggaaacgccaaagaggcaacgaaagcacattggaacttctgcgctgtcaggacaaaaacggcaattttctgcccattgcgacagtttgccagaacagagagacgaaagatttctgcgagagagtgttcccctcgcgcgacacaaattcgcacgggcggccgcgcaattgcgacttgcccgggttgaaggaagcggtttacgggtgtgcacatcactgcaaagtgtgctgcgagttgaaggagcacggctgtggcgacgattcgggttatcagatcaactgtgctgcgcaaagacatttatgtaaaaatatgacggcaatgatgtctacgacttgtgcgtccacgtgcggtctgtgcgcgacgggcgcgtgcgcggacactcaggacggatgcatcggactaaggcacatgtgcgaccagaaggagttcgaggaggacatgcaaaagtgcgcacgcacttgcaaattctgcacaccaaaatgtgctgatctgaccaacgattgtcagatcgccgatgaaagttcgtgcgaaccgccaccgcccgatcacttggaagtgaatccctattacgaggaaatggccaaagtgtgccgcaaacggtgccatttatgtgac

SEQ ID NO: 20 - DNA, 1087 bp, Artificial Sequence
  Description of artificial sequence note = synthetic construct
ttgctccgccggccgccgccgcttcatttcttcggccattagtgaaaatcatcagcaaccgcttggctcatttgtgacatcatttggaaggcggcggagtgtgcgcgtggccaagaaaatgaataaacgaattttgaggaaaatttgggtttgtaacgatgtttggctgcacattttgccctttttggaccatgcacaactcggtctcaaaatggcattgctttcgccccgtttcaatgcgttggtggacaaacatttcgacagcaaaagcgaattgacaatttggagacgtttcaaaattcacaacaaggacaatggaacaacaccaaaactttctgtgcgtatggaaaacaaaagttttgtggattttccgctgccggagcgtccgttgcccagcaaaatccgatttgaataccttcagattgattacatcgaccacagtgtcgtcgcatttctccgttccaataagcaagcttttgaccgaggcaccaactttgatttgtcaataatacattccatcgacgaaactgctaaacacaagcagatttgggatgttatggctcaacaaatttggcccatttttgcgccaaacattcgccatttggaattttccaaaatcgaatatcgggacaatttgcttcgcctcatttcatcaacaattcagtccaatcccaatctgagttcaatttatgccggtggtcagttctccgacatgtttgctgatgatggtgggacagatggaaaaattggcaaagcgttgtccaaatggttgcacattccgtccaccgatggtcgccctaaacgattgacatgcggaatgagttgttatagcaaaggaccaccaccaaacttcgaatggatcaacaaattgaaaaaggcatttctccgtgccacctcttctgccaattacattattacaattcaacttcgcgcattggcaccaattgtgccgtttgaagtggtgaatgaaagaacccaagaaaagctggcagtgaaaaaagaacgcgaatttggctgtgtgaatgattgggtgttgaagcgaagcccaattggggagacggatcagcataaagatgaggaagattta

SEQ ID NO: 21 - DNA, 378 bp, Artificial Sequence
  Description of artificial sequence note = synthetic construct
ggtcaccaaaaggcgctgtctcagcaaaagaaccagcaaaaacaacagcagcagaagaagggtcagggcaacgatcagagagcggctgccgccaaagcactgacattcaaatgctccgtttgcatgtcattgatgcccgacccgaagacgtacaagcagcactttgagtcaaaacatnncaagaacgaactaccgcctgaattggtcggtgttgaggcatgacaattgtggaattttgtggactacgatgttttgggggaccattggaaatcatcgaatgtatttgtttggcgtacggattgttttcattgcattttctttattttttcaaacaattttattttctggtgatggtgtatttttgaatttccaaaagtt

SEQ ID NO: 22 - DNA, 483 bp, Artificial Sequence
  Description of artificial sequence note = synthetic construct
ttcatgaaagatggacgaaacatggaaaaaatgctaaaatattgtgttcaagtttcgaaaaattataaacattacatttttgagaataaaagcgaaaaacagattaattcacgaaaaacaaaaatatttcttgaaaattttgggtttcaaggagtttttttcgattttcttttggaaataatgccagaaaatggatgaaaatggaattgttttcaaaattcattattcaaatttggcattcgccttctctgtccgtcatacagttgtagcatccgtccggacaattctttgcgtatttcttcaagtcattctgtcaactattgaactgaataagaaaatcagtttttcaaattacgtgaaatttattttcataaaaacataagctcttaaaaaaacaacaatgttgtttttgagatttattgagttgaagagttgcgatcccaaaatttaaaatcctcatttgagcactaaaaatatttcttt

SEQ ID NO: 23 - DNA, 383 bp, Artificial Sequence
  Description of artificial sequence note = synthetic construct
gggaagggacgaaatggacaagggaagggacgagtcgcagcagagggaattggcggaggaggcgaaggccgacaaacgacggaagagtttttccatcgcccgtccgagtcggcacgacgactgcacttggtttggccattccatcgccgcttcacttcgtcaaatgcccattcatacaaaggaattggccaaaactcgcattcaacaggtcatttatgagtgcacttcgccaatcatccaaaatgacaaagacaaagaagaagcacaacgaaatgggaccattaaatgtgatgggacggacaatggcaaagggcgcacttcgatcatttaaaacggaatgccattctttcgcttctcatattggcagagattatttttgttat

SEQ ID NO: 24 - DNA, 1013 bp, Artificial Sequence
  Description of artificial sequence note = synthetic construct
gaagcagtgcaacaataatgctaacaacggaagcaacggctccaccattgcaaacagcaacgtcttttgcgatgatgacgacgacgacaatggtgcagctgctgatgatcatcgacaaggacaaagccaagtggagctgccaccgaaatggaaatgggcaccaccagaagaggaggcggaggaggagggaaagcagcatgaccaaggaggaggagggcaagaagcggcagcacatcgatgtcaagcggggcccggtggaaaagaagggcagacgcggtgcggttccgtgccggagtgtcggaaaggatggcaccaccaaagggtcgaaatatttcataccgaaggatgtttggcgtgactatttgggcactgaatgggtggacatggacagcctcgaattggaggaggtggacgagccgcaatatgagccgatgatgccactcaacccggacaagtcgggcgaggtcgactgttgggtcaaggaactgcaggacgttgagggcaacgggctcgccaggggatgggaggtggagagtgtcattggggtcagtgcaaaggctgcggatgggacgcgtcagtgttttgtcaaatttgtcggcttcaaattgccacagcaaattccgctggctgttgtccaggaaatggcacccgaggccttcatccagtggtgcacttggcanaacgacatggacaatttggacaaatgtggcgcctattgggaggaacagttgcggcagccgccctcctggatgtgccgacggtcgttggacgcctttgctgcatggaaggcgtccaagttgaagcagtgcaacaataatgctaacaacggaagcaacggctccaccattgcaaacagcaacgtcttttgcgatgatgacgacgacgacaatggtgcagctgctgatgatcatcgacaaggacaaagccaagtggagctgccaccgaaatggaaatgggcaccaccagaagaggaggcggaggaggaggaagagcaggaagatgacattgaggag

SEQ ID NO: 25 - DNA, 344 bp, Artificial Sequence
  Description of artificial sequence note = synthetic construct
tcgcagcatggagcgcagtctgtcccttgcgctgcccatccacaaagtcgtcggtttgggcgcccgactgttcggttttgctcccgacacattaacaggggtcgaacttcgacgagcggaccccgcgtatccgtccgaattgctttgtcgcaccagggacaatttgttgcgacaattcgacatcgacgacggggacgtactcgcctttgtttagtggttcattacgagtgacagttctcggcaaaaaacaatcccaaaatgtgattcactttaaaattgttttctcatcccttttgtttctttccgatcccttcattttttaaatggataaaatattttaaatg

SEQ ID NO: 26 - DNA, 596 bp, Artificial Sequence
  Description of artificial sequence note = synthetic construct
cggatgaaaaaagcggaagaacgattgaagggacgaaaaatggaggaggagaacgacagagaacagcggggaagggccaaaagactggtggaaaagttggccaaactgctgacagcgggggatttgccctttctgaccgccagcagaaagacaatgcccaaagccaaaaagcagaacaacacgaagaagttgcagctgcatcaacagcagcagcagcggtcacgcaattcgtcccagtcgaatctcttcgaaccgatgccgacaattagggaggagacggacaccgaactaatgggggaggacgcgcagaacggagaagagacggtgcagccacggaaaaacgacacggaaacgtggggagaatggaggacggagggagatgccaaaaagtgccacggtgacaaatattgcacaaaggcacagcaatttggcaccacccagccaatgctgcagacagccacctgattattgttgttttgtcgaagcaaatgcccaatacaattcttaattgcttcaattagtaaatactcggcgattttctttcatatcatttcaaatatttattctatttttactgtaaatacaaatgaaattgt

SEQ ID NO: 27 - DNA, 579 bp, Artificial Sequence
  Description of artificial sequence note = synthetic construct
gagccgactttttgtcaccaacaaaacaactcgataattcaattggattcgaggaagaagcatttggtggctcaaagaaccacgacacaattggcattgttttggtcgattctgagggaaatgttgcggccggcacttcttccaatggcgcaaagaacaaaatagcgggtcgtgtgggggacgcgcccattgttggtgccggggcttttgtggacaacgaagtcggcggagcagtggccacgggggacggcgatgtgatgatgcgatttgtgccaagttttttggcagttgaacaaatgcgttatggaaagtcaccttcgcaggcaacgcgcgaagccattgaaagaattaaacgaaattacccaaattttatgggggcggtggtggcggctaacgtcggaggcaaattcggagcggcatgctcaggaataaaaggaggctttgggtattcggtggtcaattcaaaccatgaaaaagtgtgggtggagagagtgaattgcgaatgaaaagaaattaatcgttttgttaggctctcaactaatttattttcgtttttatttaaaaagagaaatacctgcg

SEQ ID NO: 28 - DNA, 598 bp, Artificial Sequence
  Description of artificial sequence note = synthetic construct
gatggcccttcggatgacttgcattttccgttccattttgcaatttgttttcaaaacgacaaatgccccggggggcaatgcgtcgccgctttgtcctcagatgggtgccttagcggagcaatgcatccgcgccatcggccacttcgccgttggggacattcaaaatcagcttttctgtgtgttcggatggcgtcgttcccttctctcaatgctttgcacgtctctcccgctgaacttcgtccactccgaaccccaaaagcactttctcctccccactctgatcgccgtgctgcgcaattcgccgatcggagtgaaccaaatccgaacggagttttgtcttcaatatttggtcggatatttgaaggcagcaatttaggcgaaaacctccgaaagaaagtccgattcttccaaattcgactttctttctcttcttgagccatccgtcggaaaatggaattcagcaaaggaatttttcgaatccatcagcaaaacaaatgattttttgctttgaaatgtgttacccttttttactaaaaaaaattgctcaaaaaataattgtataattactatgttaaaataatttcaataaaaatatagc

SEQ ID NO: 29 - DNA, 309 bp, Artificial Sequence
  Description of artificial sequence note = synthetic construct
taagcagtggtatcaacgcagagtacgcggggagcgtctacatgggagcctcgcccgcgtacgagccaccagcgcaggagaagtccccggatcagagcgcctacatgtgagaagatgcaacaacgaccggcggatggatggacgaaacctgaagagcgagcgacctgtcagaagatgcaaagataaagaagatgtctcataatcgtgatctgtatttattgatgtattgtacatttgtatgcatatatcatttgctgtgtattatcactttatttccatctgtgttccgaaataaattgaattgatggc

SEQ ID NO: 30 - DNA, 674 bp, Artificial Sequence
  Description of artificial sequence note = synthetic construct
cgagccgcccgtccgttgcatccgtcccactcgcttgacgcgtcgttcgactccgatggactcgcgcttagacagccaattgagtggaggcctcaaacactcgccaattgaccaccgatacaggtccgttaagaattacgaccttgccactgcactaaaagagcgacacaatcggagtggtggcattggcattgaacatcgctattacgccgaccattcgtccgacttcctcgcgcattcgtcgtcgctcagtcttcgttttctgctgaatggcctcgcacgcagtttcactggatgtctggccgaccctgacgaggaaatgaacacgcagcagggggaaagtgacgcctcccaggaaaatactggtgagaaaaaagctggtgcggacttcaaaacctcggcggaatttctgaccgatgcttcggaaaaccgtcgcagaaatgaaatggtcgtggagtctgttctggagaacgatgccgtacagaaactgaatgccaattcgtccattgagaaagtgccgttaccgatgccgattttcgacgacgccgccactgccttttaccacgcgtagagtgacactgaccatgccattgacacttttcaattgaccataattactaactgaacctttcatgtgccctctgaaattagtgaattataaagtaaaatatttc

SEQ ID NO: 31 - DNA, 323 bp, Artificial Sequence
  Description of artificial sequence note = synthetic construct
caaagcgcatgaaactcgaggaagagccgcagcaaacgagccgaactctgcgggggatgggccatggactcagtaacaaatgtttgcgatttggatgtttggatgtaaagctctcttaaataattttcattcgcatttgtatgtgtgcttcggtggctcagtcggtagagcgtcagtctcataatctgaaggtcgagagttcgaccctctcccggagcaaaattttttgattatattttttatgctgttatatttcgaatttttttctaagtacactaattgcgctgatttgatcattgtaaacgaataaatgattcctggct

SEQ ID NO: 32 - DNA, 1355 bp, Artificial Sequence
  Description of artificial sequence note = synthetic construct
ttttgaggaggcgctttctctcacccattccctcgttttggtgcacatttcgcctgagatgtggacggtttttgaccatatttacaaggcttttctggaggaaggcacttcatttttctcagattgtgcaccagtgctccacgcttttttgaccaatgacactgacaattttctgtctgtttttgaccgagtgcaacattttctggcgatgtgtgaaaaaacattgaacgatgagggtgaggacggctgtgatgagagcacaaaggcacatgcggcaaaaatgctggaggtttttgtgctccaatgtcaaggacgtgcaagtcatttcatcccggacatattgcgtttggttttcaatcaattgcagaaagagtcggccgatttaaaattgggccaactgaagccacaactattaattattttgatcgctgctttgtattccgattttcaattatgctccaatttgtttggtcagctgcaattcaaaacggagattggcactttcgaatggcttattcatgagctctattcaaatcggaaggactttgagggtgtgcacgaccgcaaaatgctcatttggttgctctgtcgcattttggctgatggaaatttgcccgctttgttcattaatcagcctgaaaagtttatggagtggcttctgactctttttgaggaactccaacggtgcatcaaagaaatagccgaacggagggaggacgactcggactcggaggacgaggagtccagcgaggaggacgacgatcggatgaacggagagttgaaagactcagacgacgatgtggacgaagagaactcgcaatatctgatggcattggagcacgaacgaaatgagcgaaaagaacggaggacgcgaaggaagtcgagcaccaacaaaagcatggacgatcagacagagggagcaccgggcgacatcctttcccttgcatcggaaaccaccgactcggaagagcaccaccattttgaggaagagactgaccttgaggcattttctacaccattggacgaccaaggcgacaataagccatgtctgaatgtgtttgttttgttcaaacacacattggaagaaatgaatacccgcaattcgcctcttttggtcagcatttctgatcagcaacgaattggcgaggcacgagttgcaaagcttaaccatttgatggaaatttgcacgagagaggaaaatttggagaggtcaaagcgcttggcgcaggccggcggctattcttttgacgccaatgcgccggtgccgacaacattcagcttcagctgatcgaagagagagaaagaattctaatccattcattcgtttgtctttgatcactttgggtgtaaaataa

SEQ ID NO: 33 - DNA, 570 bp, Artificial Sequence
  Description of artificial sequence note = synthetic construct
gggacggagtctccctctgtctcccggtctggagtgcagtggtgtgatctcagctcactgcaacctctgcctcccgggttcaagctatgctccagcctcagcctccagagtagctgggattacagtgtgcgccactgcgtttggctaatttttgtatttttagtagagacagggtttcaccatattggccaggctgatctcaaactcctgacctcaggtgatccgcccatcttggcctcccaaagtgctgggattacaggcatgagccagtgcaccgggcctttccaaacaaatttttaaaaatcttttgtaccttatgtttttttcaacttcataaaagttttaaatttatagaaaaattgtggaaatagtagagctcccatattctccatgtccagtttcccctattaacatattagtatggtacatttgttataattaacaagccaatattgatatattaggtttctttagtttttgcctaatgtcctttttctgatctaggatcccatccaggatacctcattacatttagttgttatgtctccttaagctcatcttgattatgac

SEQ ID NO: 34 - DNA, 680 bp, Artificial Sequence
  Description of artificial sequence note = synthetic construct
caagacgaaaaggaccaacaagtgccataaatgaatgtaattcaaagtaaaactgtaattaagaagaaatcccaatggaaaccgctggagataggaagtaagtctgagagattaaggaacaaagtccggaactttcagtccaaaacgagattttttgttcagcaacgaaaattccggacccagaagaattgcctttcggaattgtacagactgcattccgagcttcagatcggatgcgcacgacctcagaagattcggccgagtcttttgacgcctatggaccggagtgggaagggaagggacggggaattgggaacagaagggaattcagttcgccaaatatgaccagttcggggagacgaatgagcatcacagaacgcttatttggacgtccagtgccccaagaacgaagaaactcattgggagaggaacaaatggggcaggaaaagccgaaaagcatcgcggagaacaaagacttcaaagaattaatgaagcgtcagcgaaaaattttgggcgatgatgagtggcaataaagaaaggcaaaagaaaagaagtcattagaggaaaacaaagtcggaatggatcaaagggtagaaaagggaatgacaatttatttatttgtttattttatttaacacttcttctgatttttcaataatgaaataaagacaaacccactt

SEQ ID NO: 35 - DNA, 773 bp, Artificial Sequence
  Description of artificial sequence note = synthetic construct
aggagcgcgtggaagtttggtttaaaaaccgacgcgccaaacaaagaaaaaaaaacgcgggagattcaaaacaaccagcaacagctcaacaaaagtcatagcatgtgctctccgaggccgtcatctgatggaactccaaaaaatggacattctgaagaggaagacgaatcaggggatgattcgttggacacatcgccaatgttgaatgtgccaacaaagcgattcaaggtgtcagcagagtgccgtgagcagccaatcgagcatgacaaaatgccacacttaaaacaactacaacaacagcagcagcagcagcagcagcagaaacatgttcccgttgcacatccccaaaaaattgtgccgatgccaccgcatcctcaacaaatgtccacaatgacaccgcagcaataccaccagcaacaacaacagtttttttgattttgccaaatgtttcggcacctttggcacggcgcctggcctagccgtcacaaccgacccaatgttaatgcatcagcagcatttggcacttgcgcattcgttgggtgtggccgctgccgcgggtggtgccggcggcgctttgatgcagcaaattccggcggcaataatggcggaacagctgctagcgttccatcatccatgatcagccgacaataaaaattccattgaaaaatgggtcaaaaatcggctggccctgctggtgggcacttgtgagcttgtgaccgatctcgaattgatttttataattgtttttggtatatctgtttcgggtgtccaat

SEQ ID NO: 36 - DNA, 250 bp, Artificial Sequence
  Description of artificial sequence note = synthetic construct
ggcgaaggcgaaaattgggaggaagcgaagaaagcgaatggacgggagcatttggacggtggaatcagtgctggacagagaagaagaatgagcggagggatggcagacaatgggaggaaatgatggaaatcaaacacggcacaaccattcgaaaacccaaataagaaagggccttttgccattgtccgccgtttcccaattattcccaaatgcttttccccctctccctccattgcttaaacctctctct

SEQ ID NO: 37 - DNA, 474 bp, Artificial Sequence
  Description of artificial sequence note = synthetic construct
cctattgattaattaacagtacttgcattaagaacaaatcattaagaagatagaagctgagtaaaatgagaaatattcatgacaaggagataaattggtaaaatgagaaatgatcgatcagtgaccagtgaaacacacccgacaataattctaaatattagaatgggtgggtattattcattcattcccaaggaatgcttgaaaacatttctaatcctttaagttgtcgggtttcttgttcattcccgtcaataatttcgcaatttgccaatatcccacagttcgaccgtttccgccgaattcccttctgtattccagccgagcgaaaagtccgaattttccacggtgttgtttcaaataggacttcaattcatctgtcggaatcaattttttgatgaacgggatatggttgttcttcggcttgaaacggcacgtccaaatttcattgtgcacttctgaatccgggtcgtagtc

SEQ ID NO: 38 - DNA, 1022 bp, Artificial Sequence
  Description of artificial sequence note = synthetic construct
ttttgaagtcagatccggatcggaccatgttggagaagaaggcagtcccggatcagttgatcatcattttgaagaagtcaatgccgaatcggatgaacgtgaagaaaaagccagtgcccgatcagttgatcatcattttgaagtcagatccggatcggaccatgttggagaagaagacagtcccggatcagttgatcatcattttgaagaagtcaaatccggatcggatgaacgtgaagaaaaagccagtgcccgatcagttgatcatcattttgaagtcagatccggatcggaccatgttggagaagaagccagtcccggatcagttgatcatcattttgaagaagtcaatgccgaatcggatgaacgtgaagaaaaagccagtgcccgatcagttgatcatcattttgaagtcagatccggatcggaccatgttggagaagaagacagtcccggatcaattgatcatcattttgaagaagtcaaatccggatcggaccatgttggagaagaaggcagtcccggatcaattgatcatcattttgaagaagtcaatgccgaatcggatgaacgtgaagaaaaagccagtgcccgatcagttgatcattttgaagaagtcaatgccggatcggatgaacgtggagaagaaatcggcgccggatctgttgatcatctttttggaacggctttntcagttgatcatcagcactttgaagatcccgattccggatcacaaaaacttgaccaatcttgggaacacaaatcatttgaagaggacaacgatgaagagcctaaaaaattgacaattccggatgaatatgaccagagcgattttttaatagaaaacaaaagtgttggagaacaagaaaaggaaattattcgagaagaaatcggatttaatggccaagcagagaacggcgaaaagccatcatttgaggaggaaaagtgtcccccggagggatgccgactttaccgagatgatttggtagagagcgaagaggttttgagaaatgagcatgatt

SEQ ID NO: 39 - DNA, 637 bp, Artificial Sequence
  Description of artificial sequence note = synthetic construct
agcgaagaaacaatgccaattcactactcattgtgaacccattggaacaacagcagaaacgaattgacaacgaggacgaaatgggagggcagaacgaaagagtggcgagggtccgaggggcaaagggaaaacagacgacggaggagtggaggaaagtgccaattgctgtgccacagcaaaggtttggcaacgcttccacaacttcgagccaaatgaggttggacactttgcaaggcgagcagagtcccaccaacagttactcgctcgacatcggttcgattgaacatttacggacagaattggattcggcccactccaaccttttccaattacacgaacgttttgaaaatctgttggagatgtatggcggttgcctggaaaccatcgaggaagtgaagtacgacaacgaggatttgcggaagctgtgcaaggagcaggctctcaaattggccgagtttcaatccgttggtcccccgtcctaacgaagaaaatcgccaaaaagagaggaagcgaatgatgacagaagaaggaacgtattgtgtgaagacacaaaaaaacatgcaatatttatttcaaagcatttatattggttgtgatatttttggaactcataattctaaaatacagcagaaaatgg

SEQ ID NO: 40 - DNA, 405 bp, Artificial Sequence
  Description of artificial sequence note = synthetic construct
tgcagaggccccgcgtgactatcggcgtcgacggctccgtgttccgcttccatccaaccttcaaattcaacctcgaccagaagatcaaggcgctgttggccgtcaaatgcgaattcttcatggtgctcagcgaggacggaagtggacgaggcgcagcagtcgcagcaacagtcgcattgcggatgaatcgccttgtgggagcgtgaacagcctgtgacgatgccgtccgatgtcagatgtgtgaatcttaggccccataatgtcatatgtattgtaatgttaggcattttgtcccatgtctgtctgtatataaggttgaattcctaagcacaatgatgttccattattcacaatttgtatcaattgttcatttgtattgtaggtgtgataaatgagaaaacattt

SEQ ID NO: 41 - DNA, 499 bp, Artificial Sequence
  Description of artificial sequence note = synthetic construct
aggctcgttgggacttgcctgagggtgaggagttgctgataattgacaagtcgaatgttggcggtggtgccgtggccacgtccccaaatgccgaattgatgggcatagagcgccaagtgcgccgggcggagttcaaacggcacgtttcggcacttgtggccaaatgcatcgacccttaccgaaggcgcttcttccacgccaacggggaatacgccaactttttgcgaaagataacgcacaaagtgttggacaatcagccaaagtcgggcaatgtcgagctgctgttcaacgagcaggtgcagaagaacacgcaaagactggtcgacgaatacatccgacacttcaaaaaccgcgaatcgcatcagttgctgcagcaccggacagattctcagggactttccccaaaatgatcattctttcaatattccatttaaactgagtgctgattttatcaaattaaataatacattttctgtattgcgtataaaatcgcgttaac

SEQ ID NO: 42 - DNA, 572 bp, Artificial Sequence
  Description of artificial sequence note = synthetic construct
gcgtccgtcgactgatccgctggcggtgctcgacggtggcggactgttgccattgggcggagtgtcggaggaggacggctcacacaaaggcaccggaattgcgatgatgggcgaacttttttgcggtcttttgggaggcgcaagttttggcaaaaacgtgcgatcgtggcgagaagtgcaaaaggcagccaacctgggccaatgcttcgtggccattgaccccgaatgctttgctccaacatttgtggacaatttgcagttgttcctggaccaaacgcgtgggcttaagccgcgcgacccctccaaatcggtgttagtgcccggtgaccccgaaagaatgaacagcgaacggagcgcaaaggctggcggagttatttactcagaaggacaaattcgggatttggagaaattggcaaaaaggcaaaacgttggcatgttcccttacaaggcaaatttgtagcagaacaaaaaaactgttttctttttgttccaaagcgatgactttcaattgaattgcattcatttccattattgaacaattaattcgttcccatttgctgctgctgataaag

SEQ ID NO: 43 - DNA, 380 bp, Artificial Sequence
  Description of artificial sequence note = synthetic construct
gggactgctgcaaacgttcaagctgccgacaggcgccccatttgtccgatctgcttgaagaagatgaccggagtccgtcaggtgcgcaccatctgtgaccacgtgttccactacgtctgcttccaccgttggctcaaatatcgcctgttttgtcccgtctgtgagcgcaactttcgcacggaattgtatcatgctggaaacgccgtggttgagggagcgtacgccgacggacacgttgtgctccgaactgatggtgaacagagcaacagtggctaattgatcattgatcggcactcacctccaattgtgatcggacaaaaatgatattaattgtatatgtacatatatatcaacactcggacaataaagtataatgtgcg

SEQ ID NO: 44 - DNA, 387 bp, Artificial Sequence
  Description of artificial sequence note = synthetic construct
taagcagtggtatcaacgcagagtgatttcttttttaactttaaaatttttttatttcccgacaaaaaacttcaaataaaatggttttatttgaaattctaaaatatttgaatgtatttggctggtcctttttcatttacaccttaaaccccgcctcattcgttgagttgcttcgatgaatcctaacgaaaatctcaatgaatttaatggattttattatgaacttatcaaacaagttttgagcaatgtcaaagatgcatttatggacgatggcgcggacagcgaggcgctgagtcagcttaaattgaggtgggagcataaactcaaaagttctgaaatgattggacgtcagcgtattattacatacaaaaaactccaaacgaaagg

SEQ ID NO: 45 - DNA, 822 bp, Artificial Sequence
  Description of artificial sequence note = synthetic construct
aacggagggcagacgcagagacgaaactgcagcagctgacagtccttgcacaacaatggcaaatcgaagctgatcgatacaagggatgggctttgcaatggcagtcctaccaaatatcgcagttgcctaacccaactgatacggtaatccaacaattggagcagcaaaaaacagagcttgaactacaaatccaatatggatggcaggcctttgaagcgcaaagtgctcaattaggcgaattagtacgaatttcggaagcaaatgcgaacaaactgaaccaggtggagcgtgaattgtccgaagttagcagtgagcgagaaactttgcggcagcaattagagagccagcaaaatgtcccgcagggatcagccgttgccacacacagcgaggagttgacactgctgaagcgtgaacacgaggacttgttgctactgttggcagagcaggacaggaaaatacatgactaccgtcggcggttggcctcccatggagaagcattaagtgacgcggacgaagagccatgaacctgcccagaagaagaagaacacgagctcttcccagtcttaatgaggcctacaaaatttgatgctgacaaagaaattctttggttccttttcctgttgtgaatcttgattcgttttttttcttttaaatcgactaacaaaaagctggactgtttacaatttattgtttccccttgttgcgaattgccttgagtttggttgtgttattacggtttcaactgaataagagacaactttgtataggcgaatcatgtctgtgattgtttatttaattttgataaagcaaatatgtgcaaaa

SEQ ID NO: 46 - DNA, 1097 bp, Artificial Sequence
  Description of artificial sequence note = synthetic construct
gcgaaattgtggaggaaggggcggaggatgccaatgagtgcaacggtaaaatcgcaaaacgccgagtggatgaagagcacgatgacgaagaaattgatggcgggagtgacgaacaggaagaggatgaaatggaagacaatgttgatgaaaaggaggagggagaagagagcggttacgaggaagacattgaggacccaaaagtggtgcagcaaaaacgcgggaaattgccaaagtcagccgtggacgacaaattcttcaatttggctgaaatgaatgcatttttggactccgaagacaaaaaagaggaggataaaatgcgacggcgaagtgtcagaaatttgggacaaatcgaaaacgctgaagagttggagcaactaatcagtgcacatgagcagtcaaacttgcagccgcgtgagtgggcgctgtcaggtgaagcaaaggcggaagaacggccgaaggacgcgctgttggagcagtatgtggacgcggactaccgaatggccgcaccaccgacaattgacgcagaaaagatggcacagcttgagggaatcatcaccaaacggatcaaagacgggttgtttgacgacgtcgttcgcaaagtgcgcgtcaacgaatcgcttcagcccgcagcgccctatcgaaacgctactgntaatggcacaacggagcaaaaagtgcgcaagtcattggcggaggtgtacggcgacaaattatctgatgggctaaacgacgaacacgaacttggaggagaggggaaaaaggaagaggaacagtccaaattggacccggcggttgaagagatcaaaagcgacttggacactctttttctgaagttggacgcgctcagtcattttcaatttcgaccccagccaatccaagaggaagtgaaaattgtcaataacatgccgagcttgcacttggaggaagttggaccccaagcagcggttggaccagaggtgaatttgttggcaccggaggaagtgaagcgacgcgtgaaaagtgcgccgaaagggacagacgaacgaacggagacggaccgaaagcggcagagacggcagaagaagaagaagcaacgcattttggcctccatcggcgca

SEQ ID NO: 47 - DNA, 271 bp, Artificial Sequence
  Description of artificial sequence note = synthetic construct
gactgaagagaaaaaagaggaaaagaaagaggaggggaagactgaaaacaaaaaagaggagggaaaggaagagaaaaaagaggaaaagaaagaagagggaaagcaggaagagaaaaaagaggagggaaaggaagagaaaaaagaggaaaagaaagaagagggaaagactgaagagaaaaaagaggagggaaaggaagagaaaaaggaggaaaagaaagaagagggaaagactgaagagaaaaaagaggaaaagaaagaggaggggaagact

SEQ ID NO: 48 - DNA, 430 bp, Artificial Sequence
  Description of artificial sequence note = synthetic construct
aataatgtttgtccatgttgaagcaataaaattctcatgaaatttgttgtgtctaaacgtcatctccatcatcgatgtcctccgcctcctcaatgtcatcttcctgctcttcctcctccgcctcctcctcctcctcttctggtggtgcccatttccatttcggtggcagctccacttggctttgtccttgtcgatgatcatcagcagctgcaccattgtcgtcgtcgtcatcatcgcaaaagacgttgctgtttgcaatggtggagccgttacttccgttgttagcattattgttgcactgcttcaacttggacgccttccatgcagcaaaggcgtccaacgaccgacggcacatccaggagggcggctgccgcaactgttcctcccaataggcgccacatttgtccaaattgtccatgccgttctgcca

SEQ ID NO: 49 - DNA, 358 bp, Artificial Sequence
  Description of artificial sequence note = synthetic construct
aattgtttgttgcttgtgtgtacgtttggtgatggacaaaaataataagacaaaatgtgttgtgtgccgtcatcaccatcatttgtaaacaccgccacccaaattgttcgttttgtccgccgagaggaccagtccgccattgaagtcgaacgccggaatttgaacgttgaacacaaaagcgccagtgttcgggtcctgtttgcacgcgtcgtacaccgcctgcatgtccgccgcggtggtaaagtcaataaagccgaacgcagtgcgatactcacgacgcaaagaattcggtttgatgcccacaaactccacaccgtcggtaatggcttgaatttcgcgcatcagctcctgctggccc

SEQ ID NO: 50 - DNA, 658 bp, Artificial Sequence
  Description of artificial sequence note = synthetic construct
atgttcctgacgatcctaagtcttcacctactgacccactttcaccatacgacccgtcccatccgcgtcctacacaaccggactatcccgacatgcgtgattatgatccgcgttcatttaaacctcctgagccggacgatgacccgcttcgactgcatccgatactgcccgctgcgccgtatgcaccaccggcacggcctcgaccttcacagcctaatgcgcccactcgaccgccacctacttaccccgacattggcagaccgtattttgatcctcttccgcaccagccgacgaacccgtacagcgactggccctatggacctgcagcaccgactggatccggcggatatatgggcggttatgatggtggagtgtatgaacctcgtcctgaccagcctgggaactcgaattatgaccatatggaagaggaggatcggacgacggcaaccacggacacgatggtccaggatcttctggtggcggtggcggcggattttttggtccttatctttaagaagttcggatgtaagtctatttgcttgttgatatgcaattgtttccattgtataatatgtaatgtggttaacgggtattcattcatttaacacatacattggcatatgtcaaccatactatttgtttcaataaaatatatcac

SEQ ID NO: 51 - DNA, 808 bp, Artificial Sequence
  Description of artificial sequence note = synthetic construct
ggtgactgattattttgatcagttgtctgatcggcttccgactgattctgatcactacttttctctgcgtcttcctcttccgctgccgtttgaccatttttatgctcgttttgcccatcttcttccgccttctgctgctgttgttcttgttcttcattctgatcactatgctcttcctgtgctgtttgactctgatcagtttgttgtccttggttttcttccttgtccttttctaatggttcttgttcttcattctgatcactatgctcttcctgtgctgttggactctgatcagtttgttgtccttgattttcttccttatcattttgactctcctcctcctctccatgttgtccttgttgatcagcatcaggtgatgtttctgttggtcgttcttccgccggttgatcaccagacttttcttcttccgctgttggctcttcatttggcttttcctccttcttctcctccccctcttcctgctgctggtgttgatcacggtctcttggtgcttgatcaggctctttccgcgtgttctgatcatcctcctccatcccatgctcttgatcatgctcttcctcatgcgtcttctggtcatcttcctccgtctgatgctcttcatttccttctgccgtttcgccattctgttgctgatcatcattgtcgtccgtgttatgctgatccggattttgttcttcgccgggtggattttgctctgctgatttggctgcttctcctcccccattgagttgcatcatcttctcctccttattcctttcctgctgcaggtgttgatcacggtctcttg

SEQ ID NO: 52 - DNA, 788 bp, Artificial Sequence
  Description of artificial sequence note = synthetic construct
acagaggcaacgaaaagcaacaagaagaggaggaggagcaggaagaagcgagacaattacagcaaatgatggctcttctgtagtcctaaaggtcaaatatttaaatttaattaaaaaagatatggcactctctctattccttctgttggtcggaacaatcattgctaattgcaatggtgacccaaagatgaaatctgttgaagagaaaagtgtgccgcctgccgccttttggccttacattttgcatccaaaaacacctcggcataaatcagaagagagggatgattactacgatgccgtacgagcagaagaggaggaggcggagaaggcaacattgacaagcagtacagcagcaaacagaggcaacgaaaagcaacaagaagaggaggaggagcaggaagaagcgagacaattacagcaaatgatggcacttctgttggccaacattgacccggtgccaatggttaccgccaacagcgaaaagccaaaaacgatagcacaaacgatggcaccgacaaaggcagcaaccgcgttgacaatgtctaaagtggacggggaaacgtacgacgaacgtacagaagcgggcaaagacgacgaagagacagacgatgatgatgacgaagagcatgaaacccgcaaaatggttgacacggaattgaagaagcacaaattggttgtgctgccgaacggatcggactctgacgatgtccgagaagcggatgcagaggcagacggagtcgaacaaatgccttcaaaagggacggtggacggacaaacgcactttttgg

SEQ ID NO: 53 - DNA, 573 bp, Artificial Sequence
  Description of artificial sequence note = synthetic construct
acgaaacaccgcnggcggcatgcggcacgatcgcgatgtgtccgcgtcgtttgatgatgacgcgaaatacttgtacatcttggacaccgaaggaatggacccaaaaacaatttntgaacagaccatcaaagcgctgcatgccaatgtgatgtcgggggagaaggaatcgatgccgggggaatacagagtggacgaagtgactgtgggcggacagaaggtggaagcaactgctgcggaagtgcctgagaaaatgacgcaatttgtggaatggctcaatgccgaagacgcccaaacaaatgacgttgccactttcgccgcaactgctcactataaaatgaggattctggccctgcaccatacgataatggtcggggaaaaggaccgtgctgccgcagcaggcgtttatcgaatgacggatgtgtttgttggtgaagacccgattggcgtgccagtatgggaaatcccgggcgccatgacggaattttgtcagtggctgaaggaggaagaggaaaagctgcatgaaggagaaggagaactggcgagatttgctgctatggctcatctccgtctgaa

SEQ ID NO: 54 - DNA, 566 bp, Artificial Sequence
  Description of artificial sequence note = synthetic construct
ggagataaaacagcttatctcatcggtgttcacaatttgtcaatttgcatttggcctttaattcacacgtgtctctccctagatggagcgtgggcccaatcgattgtttgacccggttctgcgacacaacccaatggcttattggaccccacgtcgtgttcgagctctcgaatatgtcatgcgagcgtacacacgtccgcgttatcggaccgtggcaacccagaccgagcctatgaacgtctggccaatcttctcgacaacctctccgcgatatatccgtcctcctccacaataagccaattacaccgcccgtttcccatgacacttcatcgtcccgagtacacaacccaactgtgcataattggttcagtctattctcaattcccctttcccgtgaccatactcaacatcaagtcataagtcttgttatcttgtagtccatcatcaccctatactcaactctataaaccaactgatgcattcgacaaagaaaccaatagtcaaacgttagtagaacatcagtcacaaaattatgagacccgctaatgtttatgcgtcatcatctc

SEQ ID NO: 55 - DNA, 549 bp, Artificial Sequence
  Description of artificial sequence note = synthetic construct
gagagataaaagaggagagagaaatagatatacccaaaagaaaatccaaatctctaatcagttggtcaaagtgtttccattttccgatatggtcgctgtgacgctcggcacatttttacaaggcagcattggcactgcggtggtcattgagcttaaggacgaaactgcgctcgaagggtcagtggacagtgttgacccgaagtcgctgaacacgcagctgagcaacgttgtgttgtacagacgacggcagaaagggctaaaacccgcgcatttgcccagttttttttgtaagggcaaacacattcgcttcgtgcattttgagaattacgcttgtgcgctgcatttgttgaaaaagtcgttgcgcaaattgtaaaagccatgcccaaaagaagcaacaacaaacacccgtaaagctcatctccgtgtcttctgtctaattggaaatattccatagcttttgatttttctaatttattgtctttgtgcctgagttatcatttaatcatttctttatcaaatttctctacaattcaaagacaaaatttccat

SEQ ID NO: 56 - DNA, 509 bp, Artificial Sequence
  Description of artificial sequence note = synthetic construct
acaacaacaacagcagcaatcgcatgaccaaggagcaggaggagggcacaagaagatgaagctggacggcggtgatgaccacgagggattgccgtcttcggcaacgacgacgatggctgaacaacaaagacagcagcaacaacagcaagaacagtcgcatttgatggacgaagaaatgatggtgatggacgagcatagccttggcggcgtggatgctcatggcgatgtggaggcggaagaagtgttgcaccatccggacgtgccgaacccgccgatgacgccgcctgtgccggaacgaatgtcgccctcggacagctatgggctgaagtttgacagcgatgtgcaggacattgttggtggtgacgatgatgacggagtggaggaggtggaggacggtgctgacgaagtgttgtacgctcatgaagaagttgaaggaggcgaggaggagggcgtggatgaatatgatgaagatgaagaggaggaagaagttgaggatgaagcgggtgaag

SEQ ID NO: 57 - DNA, 366 bp, Artificial Sequence
  Description of artificial sequence note = synthetic construct
acggactcgagattcgtgtgctaagtctgcagaaaacagttggacattcctctactgatccattgtcacaacaaccggggccgagcgttggatttggcgggaatcttccgtttggaatgccggccgcgaaccctaatttggccaccgcgttttcgatgtatgggtcgaaggcaactacgatgcaggggacacaggcggacccgggggttccgactgagtcccaacaggaaattcttgaccgcttgactaaaatggggctttgaaatataaagcgattgttatattttctcctttccctgttctcgcctgttgaccccatgcttcgtccagtctcgacaccaatagcgagtcatcctcgctcttaag

SEQ ID NO: 58 - DNA, 1004 bp, Artificial Sequence
  Description of artificial sequence note = synthetic construct
aagtgagaataaataaataaatatttcgcaaattcggacccatcacttttattttgttcggatccaattgtgaaggtgttctccaatctgataacggtcctcctcaacaattgccctttccacccattcggaagtgacgaaagtgccgagccaccccttctgttcttcattccacacgaacagtgcctcggcatggtcggggtccattgaactgatcacaacatgggtaactgatccgtccagcacttcgctgattttgccgtttcgtgcctcgattttgtcgttcaattggacgacgttctgttgcttctccaccgccttcaccgacccatggacaaagaacacaaagcccgagaaaaggttttccggttcgactggtgcagaatcgaagtcgtggatcatctcctcaatttggtcacacatccgattctcctcgtccaacgctctgtttgcaacttcaattgctccagtctcttcttcttcgctgccactcctctctctcttccgtctctctttcgctcccctctttctcttccgtgtctctttccctctcctcctctcccgcttccctctcgccttcttccgtctctcgctcttctcctctgtccattcttccctcgccctcgtcgtccgattcttcctcatcattttgttgatccgttgttggaagagaaaacaaatcgaacggtgcgcgtgatgaaatatgtaccatgtccgaccgttcccatggtattagccttccatgttctttgcatttgcgcagccaattgccatgtacgatgtggtaattgtccgcttttattgccgccacacagctcaccggtctgttgtccattgccacgaggaaatcggcagttttaccaggatttgaaattggcgtggcacccaacgaaatgacaattttctgcaaatcttgcgctgtcacaccgggaccaccgttcaaaacgcacactttgcgtcccctcaacgcatcactgagtgtcccctccaaagtgccattgtcg

SEQ ID NO: 59 - DNA, 751 bp, Artificial Sequence
  Description of artificial sequence note = synthetic construct
tgactgtcacttttcggctgtccctcgcctctcctccggccgttgtccctccccccgccgtctctccccctctgttcgtcattccgtccattctccacgtcttcgtcccggctccctccgtttgctgctccattcctttttcttccttttccaaagtgccatgtttcttgtcgccctcttccaactccttccgcaacattcgtcagcctctgtcgaccatttcgagcagtatatgcccaccaaatgtgaagcatgtcaactgtttgctcgggagttggaaagcaatgcccgccgattgtcttcaaaaatgccccgagatgaagcagaagcttggcttgtcgacgaattggaacaactttgccctcggatgctcgactatcgcttacacaaagaccgcaagggattggcacgttttgcgaaggagcgaaccggcacggcaaatgccattaaacggctgaaggaacgcggagtgcaggtaaaactggatgttgacgatgcgctgctcgaccgtccgtccgtcgagtcggccaaactgaaggagcactgtgagtggatggtcgaagagttcgagcaggacattgaccgatggttcatcaacctcagacataggaaaactttagaagaattcctttgttcggggcgactcgccgacgaatttgacggaacangcgcagaaagcgatagacgagaagaattgaaataagactatttccctcaacatttttataatttattttttgtaatttcgcgc

SEQ ID NO: 60 - DNA, 475 bp, Artificial Sequence
  Description of artificial sequence note = synthetic construct
gaaaacgaacaatgctatggaggcatcacacctccaattttcgcgtggtctagtccatcacccatctttgtcagactttctagcagctattctggatgatgttgacaagcaggtggacatcgcaagatctgcccgagtgttcccgcacaaacgccgcatcaaatacattttgaaggagcaattgatcggagatgcattggacgaggcggagtacaacaccgacgaagacgtcatgaacatcctttcactgctgagtctgcagatgcaaggatatgtgggtggcctgcgtgcacgaggggctcaacacgaacatgaggaccgtgaatgattgatcgctttataccattggcaaaaacccttgtcttattccgcacaagtgattggatttttaaacctcaatttccgtgattttcaacattttcatttgattcgaactattatttttgatgtttattataataaattttcgatttcc

SEQ ID NO: 61 - DNA, 747 bp, Artificial Sequence
  Description of artificial sequence note = synthetic construct
attgcactaatttttgctaagctcacgccactctccgctcctccagcagtcgttccctcgacgcggagctgttcatcatcaaacacttgctgatactgcgcgaacaaatcagtcccttccgacagcacaacaaacagcagaatcgatcagtctctacagcgccattctcaagacaatcgtcgctttatgatgtgcaaattaacccgcagtacgactactccttggacctgagcaagtacacccagtcgatgtttcagctgctgaacgccgagaacagagctcgttggttcgagttcagctccaacaatgcgtttctctccctcctccttttgtcgcccgtccacgtcagcgaactccaaacggactcacgacggatcatcgaagcacacctgagacgatggtgccatagcatgatcggacacgtctccgcaattctgttgggaccgttggccaaatttcagtcgaacattgagcaattgcaggcggagcaagaacagcgggcccaggggcagaaaagtccgttggatgtgaccaccagcgaacgcttcagccccaaggcattgcacgaatgttgcgcggacgcattcaaacggctgaaacagcactggccagaagttcgcgctgccttcaccctttacattggagtccgcgaaactgaggaaatccttctccagccaatacgaaaggcggtggccaacgcattcggcgcattgaatgcatttgctgaacgacattatgacacagagc

SEQ ID NO: 62 - DNA, 425 bp, Artificial Sequence
  Description of artificial sequence note = synthetic construct
gttcgccccgacggactacggccgactgatcgaatgcacgacgccattcagtgcccaaggggacaaccaactgagtttggcgatcggggagagagtgttgctggtgaagagcggaacgaggggatgggtgttgggacggagcacggacggagtgagaagtggttggttcccggcgaagttcgtgaagttggtctgacgaagagcggactgtgaagcatctgacctttcccaatacattcgaattgtttttcccattccattggtattttcttcacacaatggcaaatgttgtgcttttggcacactaattaacgttttccccgaagcaggtgatccccgcaagaacattcagttcccttcccttctctccccctccttaattattaatgtctttgcttatgccattaataaaaaagtccttccgt

SEQ ID NO: 63 - DNA, 525 bp, Artificial Sequence
  Description of artificial sequence note = synthetic construct
ggaggaggtggcactgtcccaggagatgccatctgcacggacaaaggcaccggatgtgagccaggcttttgtagcagcacagactttgctcgggcgcactgtgccggcacatgcaaacacgttttgcaagagtgcagtcatttggcttcggtgcccgacccagccaaatcatgcaccgaaacggccgagaactgtggcactataccggacatttgcaccgatgacactttggccgtttgtggttgtgctcacacgtgcaatcgttgccatcaccaggcttcatatatggcacaaggaaggtgcaagaatgtgcagtaatgggatcaattagcacacagattacagtaatgatgtaaaagcattcgactctaacgttccctatcgtatatttctaccgtacatacaacaaaaagcgcttttgtagttttatggcatacagtaacccattatgctattcatgctttgattcatttaaactttgaactattttcgataaacaatttaaaccatataaattaattatgt

SEQ ID NO: 64 - DNA, 404 bp, Artificial Sequence
  Description of artificial sequence note = synthetic construct
ataccgcgaggaggctcggatttacactcaattggaattggacaaacttcgccagcacattcagtctcgacaagtgtgtgacacgctgagactcatttatcaacttcacactccgaatagaacatcaagctttatcgcgggtaatgctgaacatatttcgccggaggaaagaaggagtcactgcgaattgttcggcttttccgaagcccaacgaaacggcgacgacgaaacggacgaaataacggatgaatacataaacgaatacgaaaatgatgaatacataacggatgacgacgaatgacggagaagggacacttaacacacttttgttatccgattaatataatatttatgttttttcactttacaacaaaagttgccattaattccaaaataaacacttc

SEQ ID NO: 65 - DNA, 407 bp, Artificial Sequence
  Description of artificial sequence note = synthetic construct
gaatgcgacatgttggagctgtacacaaaggcccaagcgcatcaggcgaaccaaggtcctttgtccaaaatccccaacatggagccttcgcgggtccgcgcatcgttcattcgctttgagaagttcctcgactgccccgagagttacaactgtcctcagatgataaaaatcacggctgcaagaatccgcgagtccgtccaaagacgcacgtttgaacacatcgtcggcgcttatcgcactatttgggagaaggtgacgacgccagagaatgagtaccaacaaatggagcagatgagaagcgttgaagaggtggaaaagacgctcttgaagaagtgatttttaatatgaacactcccgtttaactgtgatgtttttaaatggtcgctataataaattatttctccgcc

SEQ ID NO: 66 - DNA, 1123 bp, Artificial Sequence
  Description of artificial sequence note = synthetic construct
acaatgaccaaaacgaaggtcagggagacggagcaattgctgncggaggtgttaacctcgacgatattgacgtggatttaattgacggagaaattgattaccaagccacttgggggcataacccttttgagcatggaggcggtaatttgttgcagaacctgcaagagcaaaacattgacgagcaagaggaggagaaagatccgtgttgtcccggcagtcaaaaaatggtttcgctgatggccaattacgttgacactttcgctcattccttttccaagtcatcgctttttgatcgaatgtttccccaatctctttctctctccgtcctttggcttttggcactgtccaatttcgctaccgcttcgggtgccgttcaacactacgatggtttcaaattgcttcgtgtcatcccacaaacattggaacagctcgccgcccttcgcaacttcagcgaatatgtcggccttcagcccaattcgggtgccgaagtttggaactttcgcccattcgttggccaaccgtccgaattttttgccgcgcctgacaatgccaaaagagtcaccgatttcatcaaattcgactccatcggcaaaacctccgagggccgtgaaattcccttcctgacgctcggctacccctcgaaaacctccaaaaagcccgctctgttcctcgatgctggcatccacgcccgcgaatggattgcgcccgcgattgcccttcactttatcaacgcgctgatcaatgagcccaaattccattctctgctctccgacatcgatgtgcacgtccttccgtcgcttaacccggacggatacactcacagtgcgaattcacagacccaagccggcgttaacaaatgcccgtgcagtttcgtcaatttgctggtcgaccgttccgtcaatttcgacgccgacgcgatgcagctcaaatacgcgctcatttgtcgcttggttgaggccgcgccgtccgcgctgaacgacggacagatggaaatgctgcgcgactattgcgcgaaagggcctttttggggggcgccggtggtggaagtcgcaaaggaagaggcggcataaatgaaaaggaacgaatggatggacaaagaggcggagatgttcacaaataaaat

SEQ ID NO: 67 - DNA, 1042 bp, Artificial Sequence
  Description of artificial sequence note = synthetic construct
ttggacaatggcagtcgcataccattgcccgacgttaaaccgggatatatccgcgcgctgatcccagacgaagcgccaaaaacagccgaagaatgggaaaggattttcgcggacattgaaccgattgtgttgcgagggaacacccattggcatcatcccaatttcttcgcttattactcaaccgcgtgcagttacgccgccattattggcgacattctaagcggcggaatctcatcgcttggctttacctggaattcgagccctgcaattacagaattggagcagaaaatgttggattggctggccaaggcaatcggattgcccaaggccttttggaattcggaccctgggcccggcatcggaatgatccaatgtaccgcaagcgacgcaactttagtcgctttgctcaacgccagggcccgagccgtggagaaaatgaaacgcaatggcagcggcacattgttggcatcgatgggtgccaacagcagtgttttgatcccgaatttgctgagagatccgatcgcaaaggcaatgaatcgattgaatggaatgagcgagacgcttcggaacagaataaaaacgaatggaaatgtattgacacgaatgtttggagttgaaatgaaaggggaagaaagttacgcggcaacaaacggacaactgacaaccttcgaggctcacgacccgaagtatttcagccgattggtcgcttactgttccgatcagtcccattcatccgttgacaaaggaataatgttaagcggcgtcaaaatgcgaaaattgccaacaaaccgagaaaagggcggaaatttcgtgctgagcgcagaagtgttggaggcggcgataaaagaggacaaagccagcggactgacccctttcgttttggtggtcagcgtcggcacgacaaacacttgcgcggtggaatcgtgccgcgagttggggccaatttgcaacagagagggcatttggctgcacgtcgacgccgcttatgcaggcagttttttgatttgcgatgaattccgccatttgtcggacggtgttgaat

SEQ ID NO: 68 - DNA, 305 bp, Artificial Sequence
  Description of artificial sequence note = synthetic construct
tgcagaggcaggggagcagaaagaaggaggagaaaagaaaggagaagaaaagcccaagggaaagaaggagaagcgcgctgcagagaaagaggaaaagaagacggaaaacaaagaagcagagaaaaaagagaatgaggagcaaaagcctgctggcaagaaggagaagcgcgccgcagagaaggaggaaaagaagtcagaaagcaaggaagcagagaaaaaggagaatgaggagcaaaagcctgctggtaagaaggagaagcgcgccgcagagaaagaggaaaagaagtcagaaagcaatgaagcag

SEQ ID NO: 69 - DNA, 782 bp, Artificial Sequence
  Description of artificial sequence note = synthetic construct
ccgcccatcattccatcatcttgtggggaagaaggcaacagtggtaagtgttccggaatgtcgcgcatcggactgccactgccacctccggcggtgcagcaataatggtgatgatgatggtggtgatgattatttgttgtggtggaagagtgatgtggtgatgatgaggatgaacaacaacggtacggtgaggagggggcgtacggtagcggagcagtaccagcagcagtaccaccaccaccgccactgtgctgtggcgagttgccgttgcttcggccagaaccactggccaagtttaacgccaaacttctgcttgatctgttcaacctcagatggctcttctgtagtcctaaagccgccaataaatttgacgcgcttatcgctcttcaaaattttggactgggcaaaatcgatttcgggcagcgaattgatgaagaaaaagtggctcttggcgaacagtgcgtcccaacccgggaaattcaaatggaatttttcctccgccattttttgctttttggcactttttacccgtcggcattccttccgactgtttcattttgctcaaatggcccatcaaatgcagctgaaacggcgacatcggctggctttgggtggcgattgtgaccgcaatttgcagaaagtggaaaagtgccaacgaatactccgtcaaacagaatgcctctgccattcccacgtcaaacttttcggcacgtaatccgtccatcagttcaaagtcctcggcaatttctganttgattcaaaaattgcattaaacaattttc

SEQ ID NO: 70 - DNA, 345 bp, Artificial Sequence
  Description of artificial sequence note = synthetic construct
gggggggtacgaaccccgcgtacctcccccagggaccatccctgggtgaaagtgcatccaaagatttttttgtgccaacccattcagatgttcctttctgtccgagccggacatgcccaccaggagtttttctttgtcgtgtgatcatcaccctacggcttcgtccgcaaaaccaatgagattagcgcatgagaaagaacaatttgcttcctctgcgaattcgttgtgtgcttccccattgccaaagcaatcggccgattcggccagtgcctttttgcggaaaccaaaacaattggcggattcgcagccgaatcagacacatgcccgaaacacagttgggatccc

SEQ ID NO: 71 - DNA, 762 bp, Artificial Sequence
  Description of artificial sequence note = synthetic construct
aggaaatcggccgaagacgatgacgacgatcttccagaggaaaatgctgtcaatttggtcgttttagatgaagtgactgcggcagctggaggaaaagcattctgcaaaggagttttggcagggcacagtcccacttcaacttcaatggaccaccctttgcgaaagcgacacgcgactttcgagagggattcgctgaaaatggaggtgaaaagtcgcgaaagcgggccggccaacgcggaggaaaagggcaaaaatgaatttgaggaggctgagggaaagttggaggacgacggagggaggggcggagagataaacggaagcgacactttggctgacaaaaaagatcgatcgcagaaccgcgagcaatgtcaaaagtcaattgtgaagtcaatgagcgatttgtttggaaatcttcaaaaattggaaactgttgcctttccgattgacaattacacggatgggcgcagtgacgggaattttttagaggatatgacgcaacgcgtaaatgaacttaaactagaggaaggacaagcaacggttgggcatggaagaggcgaatgggcaaagcaattggtggaggagaggaagacaaaagcggaacaaatgcaacaacggaatgagtacggaaacagcgaaggtagtgggctcaattgcacatcggcgaatgccatgcgaattcccttgtcatcggttttcgagggtatttcaacggaaggtcaaaaaattgacaacgaagaaaaggaacgaagaaatgaagaggaa

SEQ ID NO: 72 - DNA, 891 bp, Artificial Sequence
  Description of artificial sequence note = synthetic construct
atttccaagcaaatcaactccaaattctgaacactcgcggcgaattaacgcacgccgtcccctttgaaaaggccaagcaaatttcggctattgtttacggcactcaattcgtcgccattggcaattcccacggtgtcatttcgttgctcacttcgcccgccctccaatccctttacagcatcgaagcccattcgatgaaagtgcgctgcttaacttttctcactgaccattgcaaattgctgagcggttccgacgacaaaaccatcaaactctttgcgttgggcgaaacgcgtgcacagcttttgcgcattttctgtggccacaaaggcattgtcacggggttggccgtctgcgaagcatccgaaagcgaacggtttgcgagttgcgggacggacaattgcgcaattgtatgggacacggagagcggagagcaaagacatgtgttttccgaatgcacgggaatggccaacgacgtgccccgttgtgtcgcatttactcccaacggtcgatttttggttgccggttccgaggaggcgagcattttggcctttcgcgtcccgcaacccaaaaattatgtggaacaattgccattgtggacagaggaacaacagagggaatcggttggagaggcaaacggcgaaggaatggccgacgaatgggcggaagagagaatgtcgccatttgccgaattcgactcgccgcatgcaaattcgaagcaaaaccgacaaaaaagggagaatgggcgccacttcttccggtggagagacgccgaatgatgcggcggaatttggggatgacaatggaggagacgacaatcggcaaacggaagaggcggcggcgatggacgtggaagagatggaaagacgcgagttggaaatgcaattgggcatc

SEQ ID NO: 73 - DNA, 442 bp, Artificial Sequence
  Description of artificial sequence note = synthetic construct
taagcagtggtatcaacgcagagtacgcggggcggcgggtgacgacgtggtgatggtgacggccgttgagggggaggacgcaaacggagagaaagtggttgttgaaaagttggagacgcgggaggaaatgacggggagcagtgacagtcagccgaagctgacggtggagatgcgcaaggaaagcactgacaacgaatcgctcacggccgcctgcacatccgctgttgcaatgatgctgaacatcaaggaaaaccatccttcgatgtcgactgtgacgccgggcgctaccatcagtccggtgatcggtggctttggtcggcgtcgtaaataatttgttggtgtcgtcgacagaaaatcgggcgtaatctttgatcatcaattgttgattatctttattcaataaatacctatatttaatgcccaaaagagagataaaagccat

SEQ ID NO: 74 - DNA, 84 bp, Artificial Sequence
  Description of artificial sequence note = synthetic construct
tttatttggccttttgattcttttttattgtggatgatcgaatgttgaacgcttttgctgaccatttgtttgaaactagttcct

SEQ ID NO: 75 - DNA, 911 bp, Artificial Sequence
  Description of artificial sequence note = synthetic construct
aacgaatagatattattgtcctgtgtcactgtgcggaaaccgttttccgaaacggttcgaatgaaattccattaggaaaaaagcggagagacaatgggatcgaggggactgagcgaaattctgatgctgatcgacgattatgccgaaacacttccattgcacgtcgaaccttacaactataaaaaggcagaactggcgcaaaaacgtccgatttcggctttgtgcacgccgctggtcggctccattcctctcccggacacggaggaagttccaattggcagtttagtggcggtgtggaagaaggaggaggaccagcgggaatcgaaatggattttggccgaagtcattgaccaaagcgcgggagtgcgcggacgaggccgttacacactgttggaccatgtcgcggaatacgaatattaccgcaactatttcatactcagtcggacgcctcccgtcacgccaaatgtcaaatattcgctagtgcgccaaaagctcccctttctgctgaagaaagttccccgccaagacattatcccattgccccgttttcgtgccgatcctcggcacaatgccagcgcattatttggtcccggttcactcgtgatggcacgcttcccaaaaacgtcggtgttctatcgcgcttgtgtgatcgcgccacctgagcgtttacgtgacgggtattgtgttacatttgacatgaaatctgaattcaattgtcaagggaatggaagtaaaaacgaaactgtgcaaagttacgtcattccccagctgtatgttgttcagaatccgccggggaagcgccactcgcgaatgccgcacgagaggcaaactgatgaggaataaggcttttgtgttgtgttttcttaatggttacactgtgctttccggatcaccaattttgtacttcctga

SEQ ID NO: 76 - DNA, 363 bp, Artificial Sequence
  Description of artificial sequence note = synthetic construct
gagggaaaggaaggcgttgtcaatttggtcatttccttttcgcctgtcccacagcaacaccaacaacaaacggaacaggtgcccgcgcctccgcagcaaagcgacggacaacaaacggcggctgccgcgcagactcaagttgctttctcggagaaggatttggacgaaatgcaggaaatgtttccgaccattgaccgagaagtgatccgatcagttttggaggccaatcgaggggccaaagactcgacggtgaacgcactgatcgaaatggccaattgaatggacagaaaaagagacgaacggaggaaggggggggggacttgtgagaaattgaattgtgattggaccaatgctttttaaaag

SEQ ID NO: 77 - DNA, 645 bp, Artificial Sequence
  Description of artificial sequence note = synthetic construct
acaaggaacgaccattgaggagctgttcggcgatggcatttattgggcgggctgtgccatcgttcgtctgctgggccaacatcggcgctttgaagtgctcgacttctcctaccatttgttgcgcgtgaatcgggcggttgggtcggcgcctaatcagcagcagcagcaacaacacggcacaacggcagggacaaaggaagggggaagcggcaacaaagcgcagcaacagcagcagcaacaacagcgaaatattgtgcggctcatcgaccgaatccgtcgagttcaggcgcaacacaaccaggtgttcgccctgctcggcaatttctgcgttcacttggaggaacaggagcagaaaattcggcattttgcaccgcccgtctatcagccgctgcaaaatccgtacgcaaatggccacgaaggcattgcgttgtgacaaatgggcggcgctgtgaatgaacacggcaaaaaagaagcgacagcaataaaataataaataataatgcacatacgtaaacataattaattacacactgcctaattaattactaattaattaacattattcccgctttaactgttcactttttaatttattattttgtaattatttttaacacatgaaaattaaatgccatacaaaaacct

SEQ ID NO: 78 - DNA, 637 bp, Artificial Sequence
  Description of artificial sequence note = synthetic construct
taagcagtggtatcaacgcagagtacgggggacaaccccgtcagcaacagaaccgcatgccgggcatgggcggaggtcatcagcagcaaggaatgcgctaccagggacagccgaaaggaatgcagcaacaacaccaccagcaggctcagcaaccacaaattgcctattcgtcgtatccgcagcagcagagccgtggcatggcaccgcaaatgggtggcggaggcggtggaggagtcaaagctggccatgcgatcactgcaacacaccaggaaccgttgaacacacagatacttaccgaggctgacatgaccggacaaaagcaaatgcttggtgagcgtctgtacgcgatggttgcgcgttgcttccgggacggtgatgtcgagaaagttggcaagatcacgggaatgcttctcgagatggagaatgccgagattttgctgttgcttggagacgaggaaatgttgcgtttgcgcgtggacgaagcagcaacggtgctttaccaggctacggggcagaaggaagcgcaataggatgaatgaaggaaagaatggatgaataaattgtgagttaaaaaaagaaaattcataaaaatcgatatgctatttggtttctttgtctgaagtaaatgtttttctgt

SEQ ID NO: 79 - DNA, 367 bp, Artificial Sequence
  Description of artificial sequence note = synthetic construct
ctggcacaagtagaagaaaagggttttattcttgtccgttgtccaattgcgcttgctgaggaaaaatgtgggaatgagatggctgaatcgcttaatggacaaaagaacaaaaaaattggggttgctgtcagcaaggacaaagtcatcattggttattacgaccctaattccactatggtgattcaccagttggagcacgagatgcagtgtttgaagcacgaggtgcagaagtgttatattggttgatgcgtttatgcgatctaaatgttattctctgcaatttacgtgcaattgtatttgatttttgcataagaacattattggtttctcccaaattttaaaagtactttgtcactagtaaatcgag

SEQ ID NO: 80 - DNA, 387 bp, Artificial Sequence
  Description of artificial sequence note = synthetic construct
cgaagaaagccaaaaaggccgtcccgaagaagtcgccggctgcgaagaaggcgaagcccaccgctgctgcaaaacccgaagttcctctgcccgtttcgccggcagtgacaaagtctaagaccgcgaaggcactgaagaaagacgtcccgaaaaagtctaagatggccaagagatctcctaagatcgctaagaagtcgaagactccgaaaaaggcgacggggggtgcgaaaacttcgcggaaggtcaagaaggtggtggcgtcaaaatctgccaagaaggatgtcggcgttgatggtgcttcgtgaatatttcttttcctcccttccatcgatctccaaaatgtaaaattcgtttatgtatctctcaattaccttgcattttccactc

SEQ ID NO: 81 - DNA, 721 bp, Artificial Sequence
  Description of artificial sequence note = synthetic construct
ggaacagtgcagcgtcgatcaggcaattgtcgagtttcagcgatgccgtaacgacaacaaattgacacgtttgctcaaccagctcaaagggatgatggaatgccaaatccgtgctgtgcaacaagcagaggaatcaatgcgagtgaccaacagcaaattggtggacgaaatcaacgagttggagttcagcaaagagcagcttctgtcaaagcacaagcttgaactggaacgggccaagcgcaagtttggggacaatcagcgttcattggtcgacgcgaagcacagtttggatgtgatcaggcagaaacaccaggccaccgtcgacgaactgggcaaaattgacctgagattgggcaaactccatgacgaattggccgagaagcacaaaaattatttggactacaaaaatcgtttggacgcacaatacaacgaattgttgacggcggtgctggagaaagtcacgaaactttgcgaccactttcagcagatcgaggaccagaagaaacgcttcgcagaattggccaatgaaatgctcacgaagaacaaggaggatttgaagcaattggagacgaagaaaaaggccgaaacggaagattaattggccgtcatggctggaattatataatgtctgactttattacctattttgtatcgtgattgtagaaccatatttatgtgtcctgactttttttttgctgtgtataaaaatgaagcatccaaa

SEQ ID NO: 82 - DNA, 519 bp, Artificial Sequence
  Description of artificial sequence note = synthetic construct
gaccaaaaaaaaaaaaattttgtttatcgatggcagaaattttcttaccatttttttagcgaaaaatgaagtccggtggtgccattaggcaaagccaggaaattcttcaaaatttatggatttgtatatatttttaccaaaaattatgttttttacaaaaactatcacccaataataggcaaaaattttattctgactttttgaggtattctcaatccatcggagccaatttctgtgagtgctcagccaaaaccaacgaaggggtcagcgaactgttctcgaagttggcaattgaaatgttaaataaatcttccgaagagacggaagacaccgatggaatcggcacgactcctttccaacggcattacgggtcgaggcggagccttagaattgcggacgaagatgaaacacatgcggaacggaaacgacgcggaaaatgttgccgatgatgcataaaaggaaataagatagacgaacattccaactaattgtattatacttatggacaaagttctataa

SEQ ID NO: 83 - DNA, 335 bp, Artificial Sequence
  Description of artificial sequence note = synthetic construct
ccgtacgtgacatcaccgaccttatggtcaaacagctcgagtccaaggaccgtcaaatcgtcgacaaagactttgaactggcacaaaaagatgtgctgctggaagaaaaagaccggctgttacgcgagaaggacgagatgatcgcacgtcttcagggttatatcaacggacttggtgttccattgcccgcgccggcagaacagcagcagcagggcggcggcggccaatgagagagagaggcctctgacagagtccgactgaccggaagaaaaaaattcgcggacttttctttgatgtggaatgtttttgttttggttttttgatgctttgcgcct

SEQ ID NO: 84 - DNA, 433 bp, Artificial Sequence
  Description of artificial sequence note = synthetic construct
taagcagtggtatcaacgcagagtacgcgggccgcgggtggacacgctgaaaaagacggccaaaccattattggacatagtagggctcaaatcggtcgcactgtccgtacacaacagagaaattcccttccgacccgtgccgataggggcggaagcatttcgggaaatattcggcgacgcggggggagccatggcggagagaaatgaatcgaacgaaactgaggaggaggaggaattgatggagttgggggaagaagagcaaatcatctgctgacgaatggaggaacaacgaaaaatgattgggccaaaacaaatggcagagggaacgatttgggccgaaagtgaccacgagtcgaggctcctcacctttttattttccaaccggctgtttgtactgttttgaaccattccgtcgataaatttctctgtgtac

SEQ ID NO: 85 - DNA, 669 bp, Artificial Sequence
  Description of artificial sequence note = synthetic construct
taagcagtggtatcaacgcagagtacgcgggggagccaatcggacgacgacgaggaggacggccgattggcacgacgacaaaagaccgaaccgcaattcgtcatttcgcttggattttctccgcctcatcagcacaattccgaaagtgtcatcaatttttgtgagagtgacgaaagcggcgacgatgacaaatttggacaaccgcagcagaggatcgtttgcattttgccctctccgtctccaacagcgacaattgtcgtcggaattccctctgctgatgagagtcagccaggcgacgaggaggaggacaaccgcagcaaactgagaactcctcctccgactgatcattacgaatttgaagagcaatcagtgaaagttcaaataacagtacctgacgatggggaggaggacgaaaatgaattggacgaggaagagagagggaggaggcagcggaaggggagtgccgaaccgacagacggagcggaagaagctcccgccgcaagggaaaatgttaaaggtgacaaaacgccaacagaggaacaaattgaagatgatgatgatgacttgctgatcgaactggttgatgaatgagtcctcaattcattttgtccaaattacattttgcatattaattgtacccttttaattacgaaatatttgttgaaacgt

SEQ ID NO: 86 - DNA, 545 bp, Artificial Sequence
  Description of artificial sequence note = synthetic construct
catggacgggaacagcacaatacacaggacagacattcgagcatgttacaacgtctctacacttatttctacacgcccatctttcctgtgtcgcgggatgagcagcaaaggaatcagttggacttgccttcttgggtaaattcgccgcgaatgggaggagaagccgaggcgaatgggccaagcgtacaaagtgccagcaatgccgcgcaactccgacagtcttcttcgtctcatgtgggcaccttcgcccaccaaccagggccatcatcaaatgcacaagcgacttcttcctcaatggtcactttggaggacgactccgacgacgatgaggcgggggacgcggtggaatttttgcaagagataaaacgatccaaaagcttgcacaatctcgaggaggaagagtcggaaggcgacgaagaaatggacgtagaaaatggtcatgggaatgacagtgatgacgaagatgatgataataatgacgaaattgtcgacacggtctctgctagggatgtatgaatttaatgcaaataaatcgcttgagattg

SEQ ID NO: 87 - DNA, 733 bp, Artificial Sequence
  Description of artificial sequence note = synthetic construct
aagtggacattgtgtcaaaaacggagtttggcacaatgccaaagggtgccagtgaagaaccatcctctggttcaacatcagaagccgaccaggaaatgacaattacgccaggggaaaacacgaaatgggcattgtgccaaaaagggaattggacacaatgccaaaagatgccagtgaagaagcttcatcgggctcaacatcagaagccgattccattctcgaaactgagtttgatttcgttttgcccgctgaagagcatccgatgcaagaagagattgtaaatgctgaccatcatcatttggacaatgaaaccaaaaacgacggcattttgtcaaaagagaagtgggaaatggacaaaatgccaaaaaaaagagattggcattgtgccaaaaaaggaaattattttgattgttgtgccgccaaaggaccaacccgtgaatggagaaagagatgcagcagcaatgactttctcctcctccatttcaacatcgagtgccgattcagtcgaaaacgacgacgaatcagaagccgatttctgtgatgaatttgattttaaaaaaagtgccaaaagaagctgaagaagatggggaaaaagccgatcagaaagcattagcactaaatgatggccaagaaaaaaaagaagccgagaaaaatgagggaaaaagcgcgacggattttgattggatcaaagaaattgtggaagcaaccctaaacgaagaggaaagcaaaaaaa

SEQ ID NO: 88 - DNA, 666 bp, Artificial Sequence
  Description of artificial sequence note = synthetic construct
catgatgcacaagaaggacgatttcatctattttgaggacgatcagcttcaactgctcggcatgccttaccagggcgaaaatgtgttcatgttcgtgatgctgcccaaggaacgcttcgggttggccaaactgttggccgaattggacggcaaaaagttgctggaactgaccaaaaagcggggaaaacgcgaagtgcaggtggtgttgcccaagttcaagttggaatccacgcaccaattgaacaaaccgttggccaacatgggcatggccaccgctttctccgacagtgccaattttgagggcattgccaatgggccgttgaaaatcagcgaagtggtgcagaaggcgttcattgaggttaacgagcagggcactgaggccgcggctgccacaattgtccatgttatggcacttagcttaatgatagagccaccgcctccccaatttgtggccgaccgcccattcgtcgcttttctcgtcaatcacagccaaactgtgcttttcaactccattttctttggctgaacgaagagaacaaaaagagctttttgttggatctgtcctccaattttcgaaaacttcgttgctattttatttttggatataattttccttattttgtaatgtattaattggcttttccataataaattggctttgtaaaa

SEQ ID NO: 89 - DNA, 734 bp, Artificial Sequence
  Description of artificial sequence note = synthetic construct
tggcgcacaacgcgtgaacatttctgcgctggaccttttgagttgcgagccgcacagtttcggatgccgtggcgggtgggaggacaaagcgttcgaacattacgtgaagcggggcctttgcacgggctccgacttcggggccaaccgcggctgcaagccgtacccattcgcaccggtgccgcatccgagcaacgtgccattgcacaaaacgccaaaatgtacacaccgttgcccaaatggcgagtacaattcgacctatgccaaggacaaattctacggccaaaacatgggagtgcttgacgacggcaatgtcgaggcaatccaagcggaaataatgcgggcgggccccgtcaccgccgccttccgcgtctacgaggactttggccactacgcaagtggcgtttatcagcacgtggcgggcaaatatcgcgggggccacgcggtgcgagtcatcggctggggatacgacacggacagcaaattgccatattggctggtggccaattcgtggaataccgcgtggggcgatggcggcttcttcaaaattcggatgggctccgacgagtgcggcttcgaaacttcgggcatttgctttgcggacccggaccaatccaactgaaatcctcctccaaataaacgctttaattatggaggaaattaagattaattgttataatttaatggatggaataaataatccacataattagttaaagtcaaataaaaagagacga

SEQ ID NO: 90 - DNA, 717 bp, Artificial Sequence
  Description of artificial sequence note = synthetic construct
agaattttttggacaaaaaagtgttgaaaatgttgaaggactttgagactgaacagcatgccaaaaaggaaaagcgtaaagaaatcagccgtaaaagcattgaatatactcgaagcaaacaggaagaagacgatcaggaggatgcaacagacgaaaagaacgaaagttggtttggcattaagccggatgaaggcacgctcgaacagaggcgactttttgtacctgaccgacgtctctccaagacagaaaagttattgaccgaaattggttcatcaccaggagtgcgaatattgcgaagacatttgaaagaacattctgttgaacgtttaatttcacctaaaattgaggaatcgtctccgcaaaaatgggaaattcataaaccaagaaagagaagacgtaacgatgagatttttacatctgagtcgtcagcacaagaagtcttcgaaacaaatgaaaagggtgcagttgctatcccggaaaagcccaaaggaatatcccaaaaaatgccaaatgacaagaagcggattaaagaagaagtgggaactcatgagatgaaaatggtatttcacaaaaggccaaggcttggcgaggaggaaactcacaattccgaattgggcatcagtaaaagtctgaagccgaagaaattagccaaactcggtcaaaaattgaagttaaaagccgcttcaaaaagagaagacattattcacatgaagaaga

SEQ ID NO: 91 - DNA, 417 bp, Artificial Sequence
  Description of artificial sequence note = synthetic construct
agcgtcacctccaccatcgtccaatccaacgtcacttcccccacgccgtccagcacggcccaacaggaccttcacgcactcgccctccatggcactttgccctccccaacggcactcccaccgacgaacggaggcacgagcagtacaaaagcagaagaaaggcggcaacagcagacgaactccccgtgggtgttggtcgcgcccactccgctccacccagccaattctctgctgttcactacgagtgccaatgctatccatcacaaaattggcaaatgaatgtggcaaacgatgggcgaaccaacggccaataacgtcaaaggaaataaggatttttggaagatctttagctggctattaaactcagcaaaaatccgtacttttagtttttaaaggggctcagtatgggataaatct

SEQ ID NO: 92 - DNA, 715 bp, Artificial Sequence
  Description of artificial sequence note = synthetic construct
atgaccgtcgttctttgccaacctatttaagttctcctccggcgcgttcttcggcctttttcccctccggtagtgttcatatgcatttcagcgcgggaaaggcggaagatgcgcgattgctttacacaattggccgtcccgaggctgaatggagttttggcgcgggcgaatcgattcagattagtgacagcgaaacgaaaaaacgcgaggcattccccgcgacaatgggcagcacaataatagcaaaatcgccgcaagaaacaacgacgacgaaacagccgcgaggaatgggacaaatggaacacacagcccggaggggagggagagcacaaaaacagacaacgcagcagcagcccacgatgagaagggggagaggagaaagtgagcagaggggacccccctcgtcttcgatgtttggcgggccgtcttcgccacaatcgcagcctttcggcgcttcttctcctcaatttcggcctggtcccatcctcagccaccatcagacacgttcctatgaaagcaatgaagaatcgaaaatgtcggaagagaaggctggaagaagagcaaatgaacagatttatagaggaagagcagataaggaaagaagaggaggaatataaaatcagaatgtgggaagagaaaaggaaggtggaagatgagaggcggagacggatagagaaagagaagcaagaagaagaagggagaaggatggaagact

SEQ ID NO: 93 - DNA, 1048 bp, Artificial Sequence
  Description of artificial sequence note = synthetic construct
aagtacggaaacatgggctgcaacggaggcatcatggacaacgccttccaatacattaaggacaacaaaggcatcgacaaagagacggcctacccctacaaggccaagaccggcaaaaagtgtttgttcaagcacaacgaggcgtacaaagagggcaaagtgtccttccgagtgggagagactcatattgccgacctgcccttttccgaataccaaaagctgaacggattccgtcgtttgatgggcgacagtttgcgccgcaatgcatccacttttctggcgccaatgaatgtgggcgatttgccggaatcggtggactggcgggacaaaggatgggtgaccgaagtgaaaaaccagggaatgtgcggctcgtgctgggcattcagtgccaccggcgcattggagggacaacacgtgcgcgacaagggacatcttgtttcactgtcggaacaaaatctgatcgactgctcgaagaagtacggaaacatgggctgcaacggaggcatcatggacaacgccttccaatacattaaggacaacaaaggcatcgacaaagagacggcctacccctacaaggccaagaccggcaaaaagtgtttgttcaagcgcaacgacgtgggggcaaccgactcgggttataacgacatagccgaaggggacgaggaggacctgaagatggctgttgcaacgcaagggcccgtctcagttgccattgatgctggtcaccgttcctttcaattgtacaccaacggcgtttactttgagaaggaatgcgacccggaaaatttggaccatggtgtgctcgtaaaatttggaccatggtgtgctcgtggtgggctacggcaccgacccaacccaaggcgactattggattgtgaagaacagctggggcacccgctggggcgagcagggatacattcgcatggcacgcaatcgcaacaacaattgcggcatcgcttcccacgcctctttcccattggtctgatcggagtgaatttgttgcccttgcgctgattcagagacatttcatttgattaatcgtgcaaaa

SEQ ID NO: 94 - DNA, 509 bp, Artificial Sequence
  Description of artificial sequence note = synthetic construct
gatcattgtttttttccactataaagtataaattttaattattctaaagtgatcactgatcagttcattttctgcctattagaggccatatccgtcccatatttcctccaattgttgcatcatttggtagtatttttcgctatcttttatccttattgtatttctggtgggatattggatatattccgaaaagtccgtccagccaatggcaccttccaaaagcatcctgatgaatacgatcagtaccggcgcctcgcttccgaaagcgatgaatgccctgtcgatgatgccgtaaaaagggatgtttcggcgtctccttttccccttttgccagtcccaatacattctgagcagttcgaacttctcgtgcagttcgttgtacttctccaactcgacgcctttaaatgcggtgtctttgaatgttttgtcctccaatcgttcaaacagttcgaattccttctcaattttctcttgcattcccgcgtactctgcgttgataccactgctta

SEQ ID NO: 95 - DNA, 466 bp, Artificial Sequence
  Description of artificial sequence note = synthetic construct
aacacaccaaaaaaaagaaaaggaagtcgctgctttcttcggccggtcgtattcaaatcggtcttaaatacgacggggaccgtttcaaactgatcgtttcagtgattgcggcaaaagatttgagtccaatagaaaaagagggccacgcggacccatacgtaacacttcggctgatgccttccgcaaatggccacccggccacgacaaaagtgcaaaagggcagaaagcacacggaaatggtgcccaattcgttggacccgcaatttaaccaaaacttcgagtttgacattcactgctccgacttccccaatttcaagctgcacttggctgtgaaagacggcataaattacggtcttttgcacagtacgcccactttcggtgttgctgaagtgccgttgcataacttcgacccgttgaaacccatcgtcagccaatggttggatttgtcgtcgcctcagagatgaac

SEQ ID NO: 96 - DNA, 562 bp, Artificial Sequence
  Description of artificial sequence note = synthetic construct
atcaaatcggtgaggggaaggggtgcaaagtgatcagacgatcgacgacactccaaaagcgggacagcaaaacgcatgagaacggatttgcacgggtggcgccacaatttgtgttccctttgctccatttctacaatttcgatagtttttgcgatgtcgcgcagtccaacgtctttttggggaaattggcgctgacccttggcgagttggttggatgtgcggcccattcgccctccattctctccattttgagctcctccttcgcttggctgtccgtcctccgtcgtttcggtgcccttcgccaccctttcgtccgccactgttccatctgcgcttatttggccatttgtgatacattttggccatcgtcggaagtgctgcgcgaacatttcactgacgaactaagagattgcagagaatggttgggagagatggcgacggaatttgtacttggcgaaatggacgaaacaattcgacaatccatcggaattttgtccgttaaaattgacaacattctcagactgatttaacttttgtttaatccaattaataaatggaaatc

SEQ ID NO: 97 - DNA, 162 bp, Artificial Sequence
  Description of artificial sequence note = synthetic construct
tcaacgcagagtgcgcggggagcccggacaacccggagagaagggagagcacggacactgcgaccattgcccaccgccacggactgcgcctggctattgagcattgggaagccaattttggagatgacaattgggaagaggagaagaaaagtgggagaaaaa

SEQ ID NO: 98 - DNA, 913 bp, Artificial Sequence
  Description of artificial sequence note = synthetic construct
gaagtgattggcaagctgacacaacaattccagcagttggacagtgccgaggagcgccgggcagtgcgggacaaatttgtttctcgttttgcaaaggagagcaggcaatgtttggcggcgcaaagcgggcacaaagagacggcggcgccggcactgaaacgatcgaaccgccgcgtggagacgatcaaaccggcaaacaaacggcttggtgactgcccgaacattttatggggagagccgatcccagacgatgtcataactgcttttttggctgactgcaatcaaaggcgcaacgaggaatcttgggttccgggcctggaggagcgcctgccaaacaactccctcatttggttgagtaaacgggggcagtggctcgacgcgccgatggtcgattattatctcgacctgatctgcaagcactcttcgacgaagcgcgccgtccacattccggtcgtggacattttgtgttttcggcaaaagagggccgtaaaaacaccatggtattgggacttgagcaatgttgagctcatcttcgccccgggccaccacggcaaccattggattatggttgtgtgtgacatggcgaatcgaacattgacgcttttcgactcattgagcaacaacgacggcggcggcacaaccgaaaatcgtgcgttcgcagaggacgtaatgtgcattttgcgcacaatttcgctcaagcaacgaacccaaattgtacgcgaacagtggagggtgatcctggatcggaaggcgcccagacaggccaactcgaccgactgtgccgtatttgctctcctctacgcgcaatactcactgacgggcgcaagaatggactttgggcagcagcacattcgtgagatgaggcgacaaatgtgtatgaatgtgatctcttcaattgttgttagtgatcagt

SEQ ID NO: 99 - DNA, 1026 bp, Artificial Sequence
  Description of artificial sequence note = synthetic construct
tttcgccgccgcgcgttctttcgccctctccgccgcccctaaaaaagtcgaagaaagagaagcgaaagaaggagaagcggcagcgtcgcagcaagcacatgtcgccttcgttggccgtccaccgccagccccactcgggctcggacatggaaatgggcggggaggaggaggacggactgtccaacggcggagggcgcaaaatgctcaaaatggagtcatcgtcgcccgtcaacaacagcaagatgattttcattggtccggtcaagcccattgtccaacagtcgccgactgcaccacaaaagctgcccaaggtggagctgcgcaaccagtccatcatgttggacctttccgatccaaagactgtttcgccaccgaaattcaaaccgattggcgaactgtcggactatcagcagcaaatcaacggatcaaaaggcatgcaagaactttccaacattcgattggccactcctccgccacaaccgccgccggtgcttcgcaatgttccgttgcctcccgtgacgtcgaataacaacgaaatgatggtggaccgcaattacattgcaaagggagcctcgccgccgaagttcaaaccgattggcgaactgtcggattaccagcaacaaatcaacggatcacaagaagtttcaaacattcgattcgccactcctccgccacaaccgccgccggtgcttcgcaatgttccgttgcctcccgtgacgtcnaataacaacgaaatgatggtggaccgcacttgtcgaaacaacacagcaaagccgtccgcttcggattttgggctcgctgcttcatcatcatcgacatcgacaggagaggaggatgatgcgcaactgttcggacggcgagtggtcaaatttctgcgcagtttaaacaacggacgccgtaggcgccgcgcatgcattggcattgagcaagtgatgatcgaatttgaaacggaggaggaagaggagcaaaagcgatnatcacctaattatttgttttgatcggaatcacctctgctggatatttttg

SEQ ID NO: 100 - DNA, 611 bp, Artificial Sequence
  Description of artificial sequence note = synthetic construct
agaagattcagctaagctcagtgaaatcagcgacaacatcgagcaggaggattacgcgggacgtccaaaaggcaaaggactttcgggatggatccaaatcatcaaaccactggtacagggccgagtcatgttgacattgacggttgtcaatttcattttgttcattttgacgctcattctgctcatatacctcctcgttttcgcggcgctcatcagcacgtcgagtgagaagagaaaggagcttcagggcaagatcaatacggtggattactgttcggtgagttggcaccctctgtcggcatgttctgcaaagtgtaaaggagtcggcgatcgggtggaagactatcctacgcgctcttcttacatcgaccatgtgactggccaatgtcctgaatatttcaacaaagcgcctgaagaccttgagcagattaagtacagtgttccgtgcaatgtttgggcgtgtgaatgaacgacgaatgcttatattatacaatatttatcttaatgtttgtgttttctgtgttgctgtctaaatatctgtgttcgatatttacattgatagtaaatgttctgttgttaataaattctatatttgataaaacatattatcc

SEQ ID NO: 101 - DNA, 848 bp, Artificial Sequence
  Description of artificial sequence note = synthetic construct
gagaagcagcaggaagaaaaggaaacacaagtggaggagaagaaaatgcagttggatgaggaggaaaagcagcgggaagaggaagagaagaagcaggaagagaaggaaacgcaacagaaagaggaagagaagaagcagaacgaggaagagaaaatgcatgaagagaaggaggttgaagaaataattatgttggacagcaacgacgatgaagccatggaaaaggatggggaggagaaggaaaagcaattggaagataaggaaaagccggaggaagcgcaaggggagcaggaggaagagaaggagaagcagttgggagagaaggaaatgcaaattgatgaattaattgtgttagacagcgatgatgaagagaaggaaaagcaaggggaagagaaggaggagacgcaaaggaaaaagttggaagagaaggaaaagcaattggaagagagggagatgccggaggaagagagcgcaatgcaagggaaggtgaaggaaaaacaagcggaagagaaggagacacaaggggaaaaggagaagcagcaggaagaaaaggaaacgcaagtggaggagaagaaaatgcagttggatgaggaggaaaagcagcaggaagagaaggaaaagaagcagacagagaaggatgttgaagaaataattatgttggacagcaacgatgatgaagaaatagaaaaggaaggggaagacaaggaaaggcaagaggaagagaaggagacgcaaggagaggagaaggaaaagcgagaggaagagaaggagaagcaatcggaagaggaggaaaaaacggagaaaatggaaaagcagcaggaagagaaggaaatgcaagttgaagaagtaat

SEQ ID NO: 102 - DNA, 253 bp, Artificial Sequence
  Description of artificial sequence note = synthetic construct
aagggaccgactgttgctgatcaggaagaagagcaacagcaacggctaactgatcagccgaggagttctgatggcggacacggtgctatttgccgacatgacagccaaacaatggcaatgcgtgcgacagatttgcgggtcgaatgtgcgaatacccgtggtcgaagccgtgaagcgaaggacggacaaaagtggcaaaagacgggggaaacagcgggggcagagaagaagaagcacagcaaaacgaatggcg

SEQ ID NO: 103 - DNA, 367 bp, Artificial Sequence
  Description of artificial sequence note = synthetic construct
taagcagtggtatcaacgcagagtccgtgaaaccattgaaaaacaaaagaaaattaccagtgaaattgaccgattttacaaagaggtcgaagaattggaggttcagcgagaagatgaacacgaggaattgaatgaacagtcggcactccgttcgggcatcgaaatgattgacgaacaaatcgaacggtggaaaatggtcaacgaactcaagaagaaaaaggaaaacattgtcgagtcggtggcaaccaaatttgagcaaaagccgaactttgaccctgtggaaatgtctgatgacgatgattccgatattgttgattttgataggataagttggagaacaaaagctttttaaaggataaatttttct

SEQ ID NO: 104 - DNA, 373 bp, Artificial Sequence
  Description of artificial sequence note = synthetic construct
gcagggcacgtccactgagaaacggatggaaagggacgcagaagcaatgcgcctaaaacagcagaaagctgcagcgaaaaaagccgaagaggaaaaggcaaatgcacaagcgccgaaggtggtgaaagtggaccctttgaagggactgtgacaagcgaaaagaaccactcggctatgggatgcacaaactgacgcttttccttgctttatttagtcaatttttcgaattcttttcagcaaaaaccataattaacaaaacttctgcccataacaaaagcatcgcatttaagctatgtagttgaacgccttcatattctttcaaatgcttcatgtttttatatgtgtgttacgcttcaataaaggctatccgttt

SEQ ID NO: 105 - DNA, 503 bp, Artificial Sequence
  Description of artificial sequence note = synthetic construct
ctgtttgtatttcttccttctccatttgctgtctttctccttgcagttgttctccttgctgttcgttttgctctttacgttgttcaacatgcccttcttcttcttgctgtacgttttgttcttcttggcgatccccttgttgttctacttcttgttcggtttcttcttgccaatcccttcttggatttcatctacttggtgttcaacttgtctcggttgttcttgctgctgcccttcttgctgttctaattgttggtgctcatctgcttgttgttcaacttgacgatgtccgtcttgctcttcttgttgttgcgcaattggctttgctcttctgagaattgaaattttaaatgtctgtgtgcgacaaattggacaattattgtgttgtttaacccatttatcgatacagtcggtgtgaaatttgtgctgacatggcggaatcggccgaactttctgatccttttcaaagggattcaaacagattgcacattcttcttcgccgt

SEQ ID NO: 106 - DNA, 580 bp, Artificial Sequence
  Description of artificial sequence note = synthetic construct
gctgtccaaatggttgcacacgccgagcaaagatggtcaacagcagaagcgattgttttgcgttggttttgacgaagaaggaaacttcaactgggtcaacagtttcaaggagacatttctgcgtgccaccacttctgtcagttacaaaattgaatttaaagcgcgggcaacatcaattgagccttttgaatcggtgaatgaacgaaccaaagaaaagctgacactggacaaaatgccaagttgtgatatttactggctgttgaagcgatgcccaattagtgagaaggcgacggcgttcccatgggacgacgacgaaaattgggatgtcacattgaacagtgtccaatttgatttgcggggtggcaaaagttcgactggccattgcagccaccagcggacgaaaaaagaagaagcaggtcaaagcatcgaaacgtcgtatgactgtgcggaagcgaattaattaattaaatttgagaaatgctcgacgatctcaacagttggaatttgaatttatgctctgtcatttttttctaagaaatgctttgttgattctttttgttcgaatatatgtttatttatg

SEQ ID NO: 107 - DNA, 311 bp, Artificial Sequence
  Description of artificial sequence note = synthetic construct
agtttgaagtagctttatcatttaaatttaaagcacaatgcatttcgttaaagtttgctcatcttttctcaatcagagaagtcaggtcatttcttctcactctcctctctttctctccccttctctcatctctcttcttatcctctcctctcacttcctcactctcctctctttctctcatcttctctcactctcctctccccttctctcatctctcttcttatcctctcctccacccatttttctcattctttcacttcctcactctcctctcttatcttctctcatctctcttcttatcctctcctcca

SEQ ID NO: 108 - DNA, 799 bp, Artificial Sequence
  Description of artificial sequence note = synthetic construct
gtgtggacgcctaacgtcggatgcacgctcttaaaggaggtgatccagccgcaccttccgatacggctaccttgttacgacttcaccccagtcatgaaccctaccgtggtaatcgccctccttgcggttaggctaactacttctggtaaagcccactcccatggtgtgacgggcggtgtgtacaagacccgggaacgtattcaccgcggcatgctgatccgcgattactagcgattccagcttcacgtagtcgagttgcagactacgatccggactacgatgcattttctgggattagctccacctcgcggcttggcaaccctctgtatgcaccattgtatgacgtgtgaagccctacccataagggccatgaggacttgacgtcatccccaccttcccccggtttgtcaccggcagtctctctagagtgccctttcgtagcaactagagacaagggttgcgctcgttgcgggacttaacccaacatctcacgacacgagctgacgacagccatgcagcacctgtgtccactttctctttcgagcacctaatgcatctctgcttcgttagtggcatgtcaagggtaggtaaggtttttcgcgttgcatcgaattaatccacatcatccaccgcttgtgcgggtccccgccaattcctttgagttttaatcttgcgaccgtactccccaggcggtcaacttcacgcgttagctacgttactaaggaaatgaatccccaacaactagttgacatcgtttagggcgtggactaccagggtatctaatcctgtttgctcccca

SEQ ID NO: 109 - DNA, 991 bp, Artificial Sequence
  Description of artificial sequence note = synthetic construct
gccgtaacgggcaaatgccaattcaaaaatgagaccgtgggcggcactgtcgttagcttcaaagacttgaagaaaggcgacgaagagcagctgaagattgccgtcgccacaattgggcccatttccgttgcgctcgatgccagcaatttgtccttccaattttacaaagccggcgtttattacgagcggtggtgcagcaaccgataacggcacaacatggcaactctggcgggaaacagcagtactttgccggaaaagttggactggcgcgagaaaggggcggtgaccgaggtcaaagatcagggggactgcggctcgtgttgggcattcagtgccaccggtgccattgagggagcattggcacagaaaaaagcgtcgaaaattatttcattgtccgaacaaaacctggtcgactgttcgtccaagtacggtaacgagggctgtgacggtggactgatggacagcgcatttgaatatgtgcgagacaacaacgggttggacacggaggagtcgtacccgtacgaggccgtaacgggcaaatgccaattcaaaaatgagaccgtgggcggcactgtcgttagcttcaaagacttgaagaaaggcgacgaagagcagctgaagattgccgtcgccacaattgggcccatttccgttgcgctcgatgccagcaatttgtccttccaattttacaaaaccggcgtttattacgagcggtggtgcagcaaccgatacttggaccacggcgttctcctcgtcggctacggtaccgacgaaacgcacggtgactattggctggtgaagaacagttggggcccgcattggggagagaacggttacattcgaattgcgcgcaacaaacaaaaccattgtggcattgcgacgatggcatcgtaccccgtggtctgagaaagcgtgggaatgaatgggacgagaagggatcagaagaagaagcaggcagaccaaatagaagcaattcacaatcattatcatt

SEQ ID NO: 110 - DNA, 793 bp, Artificial Sequence
  Description of artificial sequence note = synthetic construct
gcgaatttttgtacaacagcagcagcagcaacagatggtgcctacattgccaccccaaagtgcgcatgacccgtccctgcacccgccccctcttccgcacccgcacctttacatcggatcgcaacggtttaccgctgcgataatggccgaaatggaagcgcaaccgaacgtttccccgaagcagaaatatcgggacttgaagaagaagttcaaataccttgtttatgagaatgaatattaccaagaagagctaaggaacctgcagcggaaattgcttaaactgtcgcgtgacaaaaacttcctcctcgaccgtcttggccaatatgaacagctcagcgagtccagcgacgattcggacgcgtcgacgaaaacactcgaagaacgcggagtcacaaaacagaaaaggaaaccaaagccttccaacaaccgaaaaagggcagccccaaatccgagcggagggcccacaggacaaccgaagcgaatcggcaacaaaacgacgccagcaaaatgcaaagtttctggagacgcattcaaagaaatgatgcaaatgcatcagccaattcattcgcaagtgaaggaggaaatggaccaattcggaagtgagcccccggcaaaacgccgtgccgacgattcgttggcatcgccaccgacgacgacgacccaaaggcaaagcgatggtcacganggttcgctggaaagtggggacaaaacgaacgaagttgcgaattgttcgtcggtgatcagtgcgatttctgtggaatgatttgaattttggcacttccattttaaagtt

SEQ ID NO: 111 - DNA, 259 bp, Artificial Sequence
  Description of artificial sequence note = synthetic construct
aaaatttcgaattttttttttcgctaattgtcaacaacaaacaggtggcaaagtgtcgtcgtccaatttccatgaaattgtaataaaggggaacaaaacaaaaagaaaaaaaatgaaattggtaaagttgatgatcattggtgtttggttggtattatttgttcaattttcggcgcgctgaattttcgattcacttcttcgcagcagccttcttcttcccacccgtggccttcttcttcggcgatttcttggcagcctt

SEQ ID NO: 112 - DNA, 272 bp, Artificial Sequence
  Description of artificial sequence note = synthetic construct
ttcccaacaataaatttgtttgatcgtttttcccagtgatcaagtgatccatcgatgtttcacagtaaaaatgagccaagccgcgcattcattcggtccaactccctcaccaattcctcaattgtaccgcgcaacggtgcacgcgtggaacacgcggtgcaccgtccgttgtgcaacatttgggtcagcaccagtcggcactgggcattcacccgtctctgttcctgcatttcccgtcgttcccacgtactctgcgttgataccactgctta

SEQ ID NO: 113 - DNA, 550 bp, Artificial Sequence
  Description of artificial sequence note = synthetic construct
tgaacgacgtgctgttgaccaactccaacgccacctcctcctccacggccgccaccgtacggttcaacaaacagcgcgaggcgctggcactggacggatgccatgccaaactgttgtacgacgcgttgtgccaactgttacggagtgacctgaaccggcacttaaccaccaacgaggtggtgcgcgaactgttcgacttgggccccgtgctgaatgaggaggaacaggcacaaaagatgtccaaggcacagaagttggagcggcgaacccaattgggtgagcagcaaaagcagcggaacatcagccgatgcaagggccgcaacaagaaaatgggtggcaaacacgactttgaggacgactgactgatcaatctgatcggaccggaccatttgattgattgatcacttttactgatcctatacaaaaattatatattattttcacccaatttttcccgccttaattttgggcactttccccccatcacatattaactactattatctgtctcttctctgttctgtgctttctctgtagtaaataggtattg

SEQ ID NO: 114 - DNA, 500 bp, Artificial Sequence
  Description of artificial sequence note = synthetic construct
aaggctaatggggcgccgagtgacccggtgcagccgaggaaggcggacaagttcagaaaggaggtgttagtgccaaagaaatgcatcgtgcacgtaatcggaaatggtggtgagaacatccgccatttgcaggagaaattcggggtcaaaatgcactttttgggcaacaattatttggagtacccaaacggacgcactttggccataattggggacacggaggagaaggtggaaagtgcgcgggaccacgtggacagggaattcattttgaagagatgggaggcatggaacacgggccaacacgacaatgttgaggaagaagcgacgacctacgaagaaatttaccaattgtcaccgaaattcgcactgcgcgaggacctcgaattggtgatgaagcaaattaaggaccaatccggcatcgtctcctattgctaccgccaattcagcacgggccatcgtccgattattctcagagggactgaacaggcagtggcggaagc

SEQ ID NO: 115 - DNA, 302 bp, Artificial Sequence
  Description of artificial sequence note = synthetic construct
ggaagagagcggaaatgccgaaatggtggacatttttcgtgcaaccgcgacggacattgggcggcacgcggcggagggcaccgatggacagcaaaacgatcaacagcagatgtgacagcacagagagagtgaatcaatggccaaaagcggcggatggatttcttcggaagacattaattgatcactaattgtattgtatttgattatgctcatcattcccatttgatccgatttgtctgtaatatgttccaaatatctctgattgtacagtgagtcggtgtataaatgtcggatgaatttgg

SEQ ID NO: 116 - DNA, 415 bp, Artificial Sequence
  Description of artificial sequence note = synthetic construct
cggcatatcctcaacatgcctccttgcatctctacctaattgggcaatgcgcttttcatgagcctgcaaccacatcctctgtcaatagctgttgccggacaaggctacaatcatgactgtgcctgcgattacaggcgcctgtatcaatactcgactgaagctttggtcgtgtgccctttttctacttcaatcgtgctctggcgtcttttgattcactttctctgttgcatcactatgcaaaactgtctactgctatagtagataccacccggtgatggccatcgaaataatcctcttcaatccggtagataagaaaggctaaccactatcttagcggccangagcaatctatcgccagatcccgcaacccattcatagaaaccgctctgacatatgcattaacatatatccacgc

SEQ ID NO: 117 - DNA, 583 bp, Artificial Sequence
  Description of artificial sequence note = synthetic construct
cggtttaatttttatctatgcaaatattatgaattaaatcgcatctttgctcttttattctccgtaaattgtcatttttccatttttttcggccattaattttcgaattcgacttcgctgtagacaaattgttataatgattgaagtcaccgtaacggtggctccgaaacttacagcgacaacggcgagcatttcgaagctgccgatgcgttgagagtccaagtgggcattgagcggactgtactcggcccgtttgatggacaacacgcgaacaaaagggtctgttgtccctcctcttccgtgctccatccttgccgtgctttctccgtttccccgactttcgcccatttgcggcccttccgcgtcttcaccttcccttggctttggcacaaccgtcaaatccgcgctcaaatccgtctccattcggcccatcactgctccaaatgcgtcgtcttcttcgatttgcgcgtcccttccggcgcgacttcgatttgtctcagaaagcacttcgaagagcagcaactgatcggcgattcgctcgtcactcgcccccccgcgtactctgcgttgataccactgctta

SEQ ID NO: 118 - DNA, 908 bp, Artificial Sequence
  Description of artificial sequence note = synthetic construct
ggacttcgaatttccgttcgccttgggtccgtccgctgtggacaaagacatttccaatgtgttggcacctccgcccattttcaccgcctcaattagttacgatgggatgagcgactcgtggggagggcgcagttattcagacgaaggcacaacaaactccacttcgtacactgagccctccgcggatgaggtggaagttggcttcacgttggtccaacagtgtgcgatgcgcgggtcggacgaggagttcaccagcagcagcagcacttcgtccggttcctacacttcgggcacttatacttcatcctcgtcaatcgacgaggacgaagaagaggaggaggaagatgaggaggtggaggaggaagaagtctcaggcgatgaacacggcagcagaccctcttctcgcgcagtttcgccctctcatcgtagtcggtccgtttcatcgacttcttcgtccggagaaagtgccgaaagtgtgtgtagcgaagaagagcagcagaagccggcggaagagacggagctgaaagccatcgtggaggatgaggaaaagcccgttgcgacggaagagacttctcccattgcaaagaaaagtccttctccaatgtttgtccaaatggcggaagagtcagaacaaatgttggaaggacacgcgacggtcgaagaattggacggagaagagatgaacatggaagaaatggaacagatcgaagaggagcaaagcattgatgggagcaaagagatttgccgagaggagacttatgttcgggtgcaagaactcagggatggccgaacggaagaagcgacacgtccgctgacaggacagagcaaaccgcgcacagattttgctaagaaagcggtggtacaaccgatgcgccaagagaagctaagcgtgacagaacagaagaccc

SEQ ID NO: 119 - DNA, 850 bp, Artificial Sequence
  Description of artificial sequence note = synthetic construct
aatgccgccggcattttgtctttgctctcctccgcttccttctcttctccttcatccatctgctgctgtttttggttttcctcggaaaatttgaacagattgccactgtcctccgatgtcgccaaatttcgtccgtcttcctcctgttcttcctccttcaactgtcgatggtcgtcaacggattttgccagtgccggtgcgtcgatgccggcattgtccgttgccgatggcaatttctgtttgtccgatttttcgtcgccattcttttgtcgcccattttccttttctttgacgtcttcggacgcaaccaaagcggcattgtccagcagatcagtgtccattggctgctgcttctggcccatctcttccagtttttccgattcttgtgccaatgaaagttcttgttccaatgtgaactgttcattttttgctccgattggtgccaattcgtccagcgccttttcattttgttcttcttcttcttccggtgatgaaatggtcctggtggaagtcgtgaacgaaaagtggccaattgccgaatgagaacgttgctaatgaatggcgctggtgacatgctgaaccaattctaggtcgctttccaaacggcgaatttcagccgagttttgcgcatcattctcggctttctgtcgtcggactttgtcgagatcatctttcagttccttgttgtttttatttgctttctcaagagctgcttccactttgccaagtttgtcagcgtagtcacacttttgttggcattcctcggcgactgcctgtctgagcgctgccttcgcgacttcgtctttgtgtgccttctgttgcaattcttccaattttttgctctgctcct

SEQ ID NO: 120 - DNA, 599 bp, Artificial Sequence
  Description of artificial sequence note = synthetic construct
gggaccggaatatcgtagcaaagtgtttgctatgatcccacagctgaaatacttggacggatttgacataaacgatgtcgaggcagaaatttcggatgaggaagaggaggagggagctgaggacgcgctcgaagatgaagacgactcagaggaagaggaggagggagtggacacggacgacgaggcggcgcttgcctatttgaactcatcgaaagctctcaatgatgaggacgaatcagaggactatgtggaacaacggaagaaaccaaatgacactgtgaaagaggcaacgaacggggaacagaaagccaatgccaacaaaaattctggcgataacagaaagcgtaaactcagcgacaatggcgaggcggccgatggtgagccgggaacgaagcaggcgcagtgagcgggggaaaagaaagtgctacggattggtcttgcgctatcattttgttgtggccgttgtgaggctgcattttattcgaattgtttttgttttggagcactttcttccccaccgtaatttatttgtctcttctctgaaccgtcgtccaaccgattatgttctgaattgtcagatgaataataaaatgtttccg

SEQ ID NO: 121 - DNA, 554 bp, Artificial Sequence
  Description of artificial sequence note = synthetic construct
gcattggccaaatggctgttcactccacttcaaaacaatgtgccaaagatgctcaactgctcgttgaatacggatgatggaattttgtcgtcgaatattgaaccgttcaaagcggcttttgcctcggcttcttcccccgtcaatttcatcatttacatttcgtttgcgtcgtcttttgctgcttccgttgtgccatttgatctgaccaacgaaatgactcgggaacaattggcattgaaaaggactaacaataaccgccgttttctgttggtccgttgtccaattgcgcgagacgaaagtaaatggacaaaatgggaaaaggaagcgattgcctggcgaatttatgatcaatggaacaaaattgagattcaaatttatgatgagggcgaaatcggagatgggcttctcgacgcaacttccggcccaagtgatcagcagaagtgaatgaattgttgggaagtgatcgatcaatttgaaattgcgaagttggagaatgtgaatttgatgtttgtaaaatggacggattatatatgtaaataaattgttttaaatgg

SEQ ID NO: 122 - DNA, 494 bp, Artificial Sequence
  Description of artificial sequence note = synthetic construct
gcgctttcgaaattgaagttcagcttcggtgagatgtcgactggttccatcagttcccatgggacaaattcgtttcccattttgatttgctgattttgagtgtcgcactcattttgttgtttggagaatgaagtgccagacaaatcattggcctcctgtttcaatggtaaatccgcggcctttttttgtcgcatttggatcgtccgcagttgagttgattgtttggttgagccgcgacgtggacgcatctctcttttttgccgaatcagcaacaggacgtgaagagagattcgcattggcaaagagctcaacacgatcattcgctgtatttggagagtccgcgaaggccttgtcacggtattcagcaatcttggccattttgttcgcatctttggccaattcgggtgaaatgaaacagcccaaggccttttccccaatgcatttcataatggcacccaacgcacccgcgtactctgcgttgataccactgctta

SEQ ID NO: 123 - DNA, 440 bp, Artificial Sequence
  Description of artificial sequence note = synthetic construct
ggacagcaatagcgaggagcgcactctgttcaactacgagttgtctgtcatgttgaacagtgcgcacaagttggagctggaggcgctctgtgccatttcggccaattatttgagcaccgtttatctggacaacaaattaatgccgctgaatgtcgccgtcgcttacccacacaactgtcaattcaacaacaacggtgatcaacaacagcaacagaaccatcaaaaagacttctcagaggacagcgattggagtgataataacggtgatcaacaacagcaacagaaccatcaaaaagacttctcagaagacagcgattggagtgataataacgacgacgaagacgatgattttggaagtgattggtcgtgattgtctattttcttttattattcctgtgattttttaattggtaaatttatataaattatgctttctttac

SEQ ID NO: 124 - DNA, 501 bp, Artificial Sequence
  Description of artificial sequence note = synthetic construct
gccgacggaaggagtcacagggacggaagaagagggcaaaaacggaagggaagaggagcaactggcgagagcaacggaataggaagagagcgcgataatggctgagagtatggaagaaatggacagatcgacagatgttgaccgagaaattgacaagggcgagtttcgccaagcgcaggtcaattaacctcatattaaaagtgtgtggaaagccaatcatttgacgagtcggggtatgccaagttggaacatttgacgctggataataacctgaagaaagaggtggtcaacatggcgagacgcttgcagaaggctcgcatttccttgaattctttgccggacactgaggccattgcgccggtaattgcgaaaattgatgaaacgttcggccagctgatcgcactttccaaggagtccagcgaattttccgctcgaaacatcaaattaccggagtacagcagggcggacgtggaagcactgctcggcaaaatgggacctgag

SEQ ID NO: 125 - DNA, 651 bp, Artificial Sequence
  Description of artificial sequence note = synthetic construct
caagcaatcccttatttgttaccaaaaccatggcttgtaaacaaaatacatatcattgagcattcattcgggtttttgtgacatcaaagaaatgaaacaataaataggtgacacaatttcaacataatgataaggcaatgggtcaccaaaaaggcgaaagtcgtggaacaaaaatcggtgacgaactgccaaaagacatcaaaaaatcagcgcacccaaacggacactgcgttcattgaggccacagacgagcaaaaaaatttgtcatcagcgaatcctgggcattgagtgtgacgccatttgaaaaacggaagcgcaggaaaaattggcaagcgatgaacacggggagaaaagtcaatggaacggatcggaccaagtcaaagtggacgagcaaacgcaacaaaacgccaaacgaagcggcggcggacaaacggtggcagtcgaaacgctcgcggcaattgaccaggcggcactgcacgtcaccgtcatccatcactgacgacccctcgttgctggtcagttccaccggtgcagtttcgccaccactgtccgccctttcttccccgtacattccatcaatcgttggcgcctgtcttctttcgccaccatgcgctgtcaaaattttcgagattcgatcctccgcattctgaa

SEQ ID NO: 126 - DNA, 528 bp, Artificial Sequence
  Description of artificial sequence note = synthetic construct
acattggcaacgcatttatacgcagaattttgacagtgccgaagtgaaatccgacttgttgtcgtttatccaccaattcgttgtcacattgtccgtggacccatcggccgaccaaaattctgcgtctgaccaattggcaataatcgcaagtgcaaatgaattgcttgacataatagagcaaatgttggaggaggaggaatctgaacaatgcctacagaaaggagctattctgtgtactgaattgtccaaatgcattccttcgacggacgaaggtcaaagagtgcgtggagaacagcgcaaaatggcatttcaatgcagacaagaagaattggcacggcaaaaacagtggcgacggaaggaacaacagagggaggatttgagtgcaatggtggaaacacttataccaattgtgaataacagctgcacagtgagcatttcaagcggagaagaggcaatgagcaaagattgctattaatatcgttttgcaataaataattttatgtttgattcaataaaaggttcacataa

SEQ ID NO: 127 - DNA, 386 bp, Artificial Sequence
  Description of artificial sequence note = synthetic construct
ggggctggcatttgccaaagtgtctgcgaaagttcacggccaacatttgaatgaatgaaaagccattgcatgccaaccgaaaatgccatggcatctataccaactgctgtccctacgaatttcatttattttaataattttagtaccaattccaaacccccataaaaaacaggtcttaaaaaggagcgagaaacaacaaaaacccttttcataattgtaataaaaaagagagcattttgtgccatttttgttactacactcatcacactgatcacttaattgggtgcggatttatatttangaaaaataatttttaaaaataattaaaatggttgaaaatttgccggaaatgccttagaattaatgccattatcatcataaaatcc

SEQ ID NO: 128 - DNA, 614 bp, Artificial Sequence
  Description of artificial sequence note = synthetic construct
ggagggacaattctttggagagtcaaaatggcacgaaacggcagagaaagtgagggaacagatcagccaggcatgtgaagaaggcgaagagagtgccgccgttgaaggcggagaaagtgaggggacgacgaagaagacaagcgaagacattcagtcggaagtcacaaattacatggaaactgcccgtctcgagttgggccaaccgtccaccagcggcaactgcgtcgacccgtcctctccaccactcgtcacacattttaactcaatggccgaactgctcttttggaggcggattaatgccgaacgcttcccacgccttgtgcagctcgcccgtcagttctgcgcagttccaatggccaacagcagtgaccagaggaaggcactaagcaacgacaatgcggaggaagagaaagtgacgcggagatacgcagccgaacagatggcagaagatggcgacatggcacaattggagagcggacggacagaacagttacagctgctcacacagctgatgaccgtgagaatggcactcagagagggggcgacagaaacgaacgaaaatggcaaaaaacgaaacgaatgcatggcaccaaatgaagaaattacaactatgg

SEQ ID NO: 129 - DNA, 514 bp, Artificial Sequence
  Description of artificial sequence note = synthetic construct
cggtgcttatttgtggggacacgcattctgacttaccgatggtggaatttggcaagtcgaaaaactcgacgggtgtgatggcgctgtttgtcacttgcgacaaagggttgcaggaaagcgtgagggaaattgtggaggacagtgaccgctgctgtttcgtttcgactcccgacgttattcatgctgcgatgatgactgttcttttgaaggcgaagaaaatggcagaagttgacagcgaattgggagggaaaaattgaaggagacgaagagaatccgatggaatggaagtgaagggaatctgtgtggtctcataaagtcgtagaaatccgaattgacttctaaacataatgctatatttttgtttgtttaattggtttaagattcttcgttcgtttgttcttttcttttgacgattggttgttatgacatttctgttcgggaacaatttcatgattacggtttaatcgagttattcttgtcttttcatgtttttttctaattgaaattaaaaagt

SEQ ID NO: 130 - DNA, 489 bp, Artificial Sequence
  Description of artificial sequence note = synthetic construct
aagcaacgcgtcgacgacaagtggcggaagctgtgccggtctttacaacggtgtacgaaagtcggcagcggctgttagttcggacgaggcggggaactgtgtgtcggaggacgatgacgtcgacagccatgaagatgagaacgaatgtgttgtaaatggcagcgacgacggaggcaggacgaataatcacgtcgaagatggtcagcagcagcaacaacatgaggaagatgacggagaggcgccgcagaggcaaattcaatcgaagggtccgtcatccgattacctgcccttctcttccgttcttcatcactgacaggaccgttcagcggtgtgcagcacctgtctataagaaaaacaacagcaaaacatagcatcatcaacgctccaatgtcgaagagtctcaccattttcaattgttttgttttcctttgttgtgcccttttgtgctccactttaaatttaaattattatataaatttttgttttacg

SEQ ID NO: 131 - DNA, 578 bp, Artificial Sequence
  Description of artificial sequence note = synthetic construct
gtccgcattttctcttttagcaaacgtccaaattcgtttagcatttttttatggaaactctgctggacaaaaccattttttcggtcatcgacccgcccaatttgcgcattaatttgccgcgatggtttacgttcccctcgcccatgcaaacctttttcttcattcttctcacttatttcctcgtctccggtggcattgtttacgatgtgatcaacgaacctccgtccatcggttccacggtggatgaacgcggaaacagtcggccagtggccataatgccctaccgcgtcaatggccaatacattatggagggcctcgtcgcttcgttgatgttttgtctcggaggccttggcattatcattttggacaagtgcacccatccgttgactgccaaaaacaaccgaatgatgcttttcggactcggcttctccttactgtgcatcggcttcttcaccacgcgaatgtttatgaagatgaaattgcccgattaccttcagtcctaatttgaaatctgtaataaaaactgttaaatttgatttttgtaattttttatttaatataataaattgttcattttt

SEQ ID NO: 132 - DNA, 362 bp, Artificial Sequence
  Description of artificial sequence note = synthetic construct
gaaattgtaagaggaacagaaagtggaatataaagtggaagagaaagtggaagaggaacagaaagaggaagaggagcagaaagtggaagagaaagtggaagagaaagtggaagagaaagtggaagaggaacagaaagaggaacagaaagtggaagaggaacagaaagtggaagaggaacagaaagcggaagagaaagtggaagataaagtggaagaggaacagaaagtggaagataaagtggaagaggaacagaaagtggaagataaagtggaagaggaacagaaagtggaagagaaagtggaagaggaacagaaagaggaacagaaagtggaagagaaattgtaagaggaacagaaagtgg

SEQ ID NO: 133 - DNA, 850 bp, Artificial Sequence
  Description of artificial sequence note = synthetic construct
tacgcggggcacaccaaaagtttttggacgagaaaaaaatttgccctccgactgacgagcaaatgttgcattcggattttcaaaaaacaatgaacaaaattgtgcccgaaatttccggcactctcccgcaaaagcctgtcgaaactagctttcgcgacactgacattagaagctttttgcagagtgtaaagccaaataaaaacgcaaaacgagaaaagaccccggaaaaagggactttttccatttcgaagtcggaaccgcagacgccggtcaaaacaaatttgggtaacgtaaaggcggagccacaaactccaaagacaccgatggaacaacgggtgatgccaaaaaaaggaaatccgaaataaaaatgacccgttcagttgagggccaaatttgccgcgaaagccgcctttcaaaatgtcgatgaagatggaattcgaaagcgacgggcgatgctttaccgattgaaggaagacattttggaagtgattagagcgcatttgaatttgaacaaagcttcaacggttgcgcttggtaatcgcgaattgttgcagaaattgggacgtgaagtcaatcctggaatccttttggaacatttgcaacttctttgtgaaattgtgcctaaaaatgtttgtcgaattgagtcaactaattcttcggctgaccaacatcacttcaaactccacagcgacgtgggccctgattggctacaaaaagtgctaaacccgataaaggaagaaattgacagtttggatctaaaattgggcccgccaacaattccgaaatcgccttccagtcttttctgaatatcgatacccaaaaattaacattttgaattttgtttgatttt

SEQ ID NO: 134 - DNA, 761 bp, Artificial Sequence
  Description of artificial sequence note = synthetic construct
gacggggttcggaggcacggcacactttgggtgttgacgtgagttggctgaagcgtttggtgaccagcaaacatgagcaaaaagagcaacaacaacaacaaatgggcataaatgaagtcaatgggagcggatgtggagaagttctgttgaatgggcagccccaagcgaattgcaacgggaagtgtccaaaggggtggccatcggcaaatggtggtcttatgaaaaatggcgataactacagcatgaatttctcattacgaaagttgcgattgtttggacgacctcagcaacagcctattgcatcggttgacaatgagttgcaacatcaaagacatacacatgaaaaggaggagacagatcaagaacagttggacgaccaaatcactgcttgtacggaccagcagaattgacaacatttggcatcaaatggcgccgtgaaaacagcaacacatccaccatctgtgccgcggatgggattggggcaactgataacgatggaagacgaacgccaccagcaggactattgtgaaagcgaaatgatgacacaacaaatggcagcgaatggcgaagacgaaccgttgcgggaaagagaggaaaatggagggagagaaaatccatttgacaagaaataatgccgaacattctctgtattagtcaaacccacaatactttcatttaaactttaaatcacctctctgataatctcaaccatttcatctttcaacaaaaagttttgtataaagtataatagcgtgtggata

SEQ ID NO: 135 - DNA, 526 bp, Artificial Sequence
  Description of artificial sequence note = synthetic construct
tggaggaggaagagcaacaaaaagaagtggaggaaagggagcgaaaacaaccgccgacggagggacgacaacggcggagcacactttcccatcgaattttcaccctttgcgattcggaagcaacgctaatatgtgctaaacaagcgcaaaatgagaagcaaatcccggaaaaaaagaaaagtgggccaaaaaggaacgttttcatcgactctgaacaattcaattcaatttttgtcttctgaactgcgaagccaaaaccgttcaatcgtcggaagtgaacaaagaatgcaaattgtggcatatttgggcagcgatcacatctacgacccgtcggaggagtatttgctgtggaatttgcgagtggcgaacggtcgccgattggtcaccgattgggccatggacagaacacgcggcgtacaatctgccaaaacagggaaagtgttcagggcacggctgacagtcaaagagccgacactttcggacgaaattggggagaaaggacagcaaaaaggggccgaagaagcgg

SEQ ID NO: 136 - DNA, 187 bp, Artificial Sequence
  Description of artificial sequence note = synthetic construct
gatggcaccgttttccgagtcagtagtgacagaagagatcgttgaggtggattgatgctgttaagttttacggatgaatatgaccctatgtgttactctatttccctcatcaattcatttgatgtatctgtaaagtattttgtagtccgatacacgttcttttaaattaaataaacaaaatgtcagc

SEQ ID NO: 137 - DNA, 726 bp, Artificial Sequence
  Description of artificial sequence note = synthetic construct
caggatcataaaatgattatacacgcggcttatggaagcagtaaagacatatctgtttatagtgctttggatttaaaacccaaacagatttttattataggcaaagtcggtcgtaaacatcacagtatggccactgtgttggccgatggttatgctgcacatttgtctgctctacagtgtcatggaggatctagaccagctcaggggaatgcccgaatacttttgacctcgcgtggaagatttggacacaatgcttctatgaggcgtagaaggtatgtattttatgtaattcattatcaataatacattcatggatgatttaagataagtatttttcttttctttttgaaaacaaagttttgaattagtcaagaaattagaaatgtggtatttatgggaaaaaccatatatagactataataatgcatttcagtattaatattcatcaaatatattgttaacaacttgaattatacaagttaaatcaagtgttaaatcaaaatatttcttagggcgttcaaaagataggttcattttttttccttttttcaagaataaaccaatttaatctgagtaaaaaaattaataattaaaggcttctcttaaaattatcgttacttaaacttgtcttaatcaggtgtccagagaagagatctggcgatacacatggttccaataattgatccatacttgaccccgcgtactctgcgttgataccactgctta

SEQ ID NO: 138 - DNA, 398 bp, Artificial Sequence
  Description of artificial sequence note = synthetic construct
gcggtgggaccagcgctggagccggcggaggagtgatgacgggtggtcaggacgcagcgctcgttgcagtgagcgcccaggacagattggcaatcacccggatcgcttcaatgggatttccagaagcgttggtggttgaagcttatttcgcctgcgacaaaaacgaggatttggctgtcaattacatcttggcgaggatggacgagtctcagaatggacgtgcgggtgccgggcagcagggcggacgataagaagtgcaacagagatgccgcagtgatcgcaaattcctcatgtcgtttccctaaattatgatcattgtttgcccctaaagtgcatgttctgttctcgccctttggctatttgttgtgtttgattatgaccatattaaattgtttatg

SEQ ID NO: 139 - DNA, 982 bp, Artificial Sequence
  Description of artificial sequence note = synthetic construct
taattagggggtgacaaattatcaaaataataattaaacaaaaaacccaaaaacggaggtctaaacaaatttagaaggagcccgtgtgcgatgcgcacgaccaaatccgcccatgtcatcattgtcggcatcaccaccatccggcaccacttcatcttctggcacagcagcgcctttttcttccaccagacggcccattcgttggtcccccctcatcggctgattgtccctcgtcatcggctgattgtccctcgtcatcggctggttgtccctcgtcatcggctggttatgagtggcatttcgtcctccgccaaagccccgctgctgtccaatccgtcggcctctgtcccctctctgtccctccgcctcttctccgttcgggcgactgtcttggccataacgcatataatttcccctggacgacttcggtccttgttctcgctgctctctgtacatgtcgtgctggttccaaccgcctttttcttccatcagacggcccattcgttggtcccccctcatcggctgattgtccctcgtcatcggctggttatgagtggcatttcgtcctccgccaaagccccgctgctgtccaatccgtcggcctctgtcccctctctgtccctccgcctcttctccgttcgggcgactgtcttggccataacgcatataatttcccctggacgacttcngtccttgttctcgctgctctctgtacatgtcgtgctggttccaaccgcctttttcttccaccagacggcccattcgttggtcccccctcatcggctgattgtccctcgtcatcggctgattgtccctcgtcatcggctggttatgagtgacatttcgtcctccgccaaagccccgctgctgtccaatccgtcggcctctgtcccctttctgtccctccgcctcttctccgttcgggggactgtcttggccataacgcatataatttcccctggacgacttcggtccttgttct

SEQ ID NO: 140 - DNA, 514 bp, Artificial Sequence
  Description of artificial sequence note = synthetic construct
agaccactgtcacttctctgctcaacaacaaccaaaatgacatctcaattctgaagagcttgcaattagaacaagaggcgaatgccggattactggtccaaaaagttgacggacttctggctggaaatgcagcggatataactgccatggttttgtcgaatggcttcgaagcgaagactcatcaaaatttattgaaacaacttcgtgacgcaactgactctgccaatgatgaggctgatcgtttggaaaacgaatacttcgcattacaggaacatatttctgcaatgaagcagcgtctgatggaaaagaaacgtcgtcagctcgagcagaaacaaaagatggaggaggaagagcgaaagatgagggaggaggaagagcggaagaagtgggaggaggaagagcggaagaagagggaggaggaagagcggaagaagtgggaggaggaagagcgtaaaaagagtgaggaggaagagcggaagaagtgggaggaggaagagcggaagaagtgggaaag

SEQ ID NO: 141 - DNA, 393 bp, Artificial Sequence
  Description of artificial sequence note = synthetic construct
tggcgacgccgctcacaataagcgaagtttgtgcgtgtacattctccggttgaacaacgcactgacaaaccaccgggtcagatgggaacagttcgatgttgaggaggaagcgccggacgacaaattgctttattccttcgtggcctgctgcggcgaaattgttttatttggcggaatgcaaagcgatgggagcggaatggaaagtctgaatgttggactggaaaggcgcgcaatgagctccgacacttacattttgcgaccgcgttacaacgaaatgttctgctgatcgggatgattttgaaaaagggaattagatactacctgtagtaactaaatggaataaaactttcgtctttctaataattgtaatttttgataaattctttttattac

SEQ ID NO: 142 - DNA, 1055 bp, Artificial Sequence
  Description of artificial sequence note = synthetic construct
cacaacggctggcaatcatccacagcagcaaatgctaggctgcgccggacagccacaggacccgaaggcgcgcaagttgatccaacaacagttggtgctgctgctgcacgcacacaagtgtcagcagatcgagcggtctgaaccgctacaaaaccgtgcgccctgcacattgccctactgctcggtgatgaagggcgttttggaccatatggtcgactgttcggccggccggcagtgtcagtacgcgcactgcgcctcctcccggcaaatcattgcgcattggaagaactgtaacaaggacgactgtccggtgtgcaacgttcacatcaacgagacaatggtggtcgacccgcgacaagctggcattatgctgagtgctgtcggttttccctctgtaactttggctcaaggcgcgattggccaacagcagcagtcgatgaacaatgcaaacagtggaggaccaccgcaaatgcgcgggggtggcataacgcagcaacaacaaacggctggcaatcatccacagcaaatgctctgcgcgggcagcggcggtggacagccgcaggaaacggtgaagcgcaagctgatccagcaacagttggtgctgctgctgcacgcacacaagtgtcagcagatcgaacggtctgaactgcgacaaaaccgtgcgccctgcacattgccctactgctcggtgatgaaggccgttttggagcatatggtcggctgttcggccgggcggcagtgtcagtacgcgcactgcgcctcctcccgggaaatcattgcgcattggaaggactgtcacaaggacgactgtccggtgtgcaacatggtcaaacggtacaccaacggaacagcggctgaccggcgacaagctgacattatgctgggtgctatcggttttccctctgtgactttgcctcaagacgcggttgggcaacagcaaccctcaagttcggcaagtgtttgtagtggaccgttctctgtcggaagcactcctattttattgaagaatttacatttatagaatttcacttttgtatttggagaaagtgatcggc

SEQ ID NO: 143 - protein, 313 aa, Artificial Sequence
  Description of artificial sequence note = synthetic construct
KSSALRRGRDHTFAQPAYMRDPLRADLLAGSKLKEVKKTDYNQCKSMLLDLFDGTRVILVGETRDRSGRKRLISCFQLYRQSRAAAYFGMFAVHPFFQASGLGKRLLTVAERYARIVWGSDEMHLDVGGSLAELKLGMGRLQRYYKRRGFLSTGILRPFNGAVARFITVDRNDLWIELMVKDIRGALDDIGGDPEKRMKRVNSRGRLAREADKDDGGRDPQKRMERVRSFGRLTIEADRDDIGRDAQKRMERVRSLGRLAREADKSDESKGKDGEEKKKTTQAEGEESKGKDGEEKKKTTQAEGEERIKPLAD

SEQ ID NO: 144 - protein, 292 aa, Artificial Sequence
  Description of artificial sequence note = synthetic construct
WVLSYVSDKGSYPVLGKDAEGRERMNALIVGHFDGHTFEKLFEQQMDFVGGSFAYQGFHDQQSGRSFTIGWICDIGWIGDNTGDANFDGRGGVTSMTLPKEFVLKDDHLIVRPLPELAQLRQSKQPHQIRKGEKYSLEKGHAELLFQFKWSNNDDGSAEEKFVLDLTRTRLKDGKLEFTIDSKGIELKRTWVKPNKRLVVYNVKPGQIHVFIDLDTVEYFADNGRWSGAVRVPNASQENRIGTVELKSTPLVLEQSSLWYLKYGSHKSARLQPNGIPFAMNAGTSSFKQDEA

SEQ ID NO: 145 - protein, 234 aa, Artificial Sequence
  Description of artificial sequence note = synthetic construct
MNNNFLLLLITFTFIVGARAFWIQLPGTFWGYGDARQQQHRGWLNGWHSWHNQKHNGANTGGYWPIYGHGHGHFGNGNALPADDRSSNEEDDNETSEEQQLTTDDPPENASSDIMEPNDGITDQPTDQDGSDTEATDSTTVGSDPGPNDNDQNATGPTDEDETGTEATDSTTTTTESNAIGEEGTDQDATNSSDQGESDAEAEATDSTTNGSDLEPNDQDENGADADSTTTNGI

SEQ ID NO: 146 - protein, 191 aa, Artificial Sequence
  Description of artificial sequence note = synthetic construct
EKKQNVFDDFIAAAEYLINKQYTNSSKLAIFGASNGGLLTAVCSQQRPDLFGAVITQLGLLDMLRFNKLGIGSDWVSEYGDPDNATDFSYIYKYSPLQQLSVTPGKQWPATLLLSADHDDLVDVSHTLKYTAQLYHLLRTNAESWQRNPVVAKILVDQGHAFTGTPTEKKIKEKVDIYTFIARALGLKWTE

SEQ ID NO: 147 - protein, 218 aa, Artificial Sequence
  Description of artificial sequence note = synthetic construct
GGTPAVXAYVYDRKGTHYEKKIRVDDWDNHYIVDLATNDVQDVLKQNLDLEFLKLRDSVASGETKELTFYGRVWPEGKYKLFWDVKGFEMDEAQRLIKSELNVPHDCFTDENGKFKLEYEIENKSREVARWRLPPVHLYIFGASVWTKEYVHVTDWHHVHIFDLKNGKKHALPADKVAEKLYELSKRDQMNERTKLAETNEKNENEITFTRSFCPFRQ

SEQ ID NO: 148 - protein, 426 aa, Artificial Sequence
  Description of artificial sequence note = synthetic construct
MALSALLLLLPLLLNVQNIPDESVQSDVKAVDSAISSLEQWKDPRNSLASLDSQLTEPQRALAKMFWELETIEKEKPKAPPQFDLGLFLEALEAMVEMNEEAKEVKLRKDKLTEWAGGEKANEGKEGKTKEEETVPEVRVNENVKVEVTNGAGGDGKMEVKRGKDENGNEQVVVTFVKRDGTEGKTEEEQKKEEKDNLRKGREEVKMEQDNVEGAPKTDSANSAKSPIPMPTILSSPAAPAEEEEKANDAFTEANVRKKVKKDEEMFIIMTDDNGRTGNANERQMEFVRMPKKVGRDFGSELFGLPQPSNGGQSPMEMFFNLFGRKKRETVQEGRKKRSIENLANLGKPGSEFVTKMAEQAKNDDKQDEKAEIKQYLEKGVATAEGNKKAEKLAYVWYSELLYWTNKWIEVDTPAEPQKFSTFLRH

SEQ ID NO: 149 - protein, 97 aa, Artificial Sequence
  Description of artificial sequence note = synthetic construct
RGKGKNAAKKDKTKNKKAPAAAKPKAEPVETEEPSSAQVVAEQDGSDESANNQEMDAGEEIAEEEQTDLAQDEQLEDDATDGEEGNGMAEEEQPEIN

SEQ ID NO: 150 - protein, 187 aa, Artificial Sequence
  Description of artificial sequence note = synthetic construct
MSSPSSSVSLLAIVTIFCLLCKCCVSAPHPCCPGSQKVVSLMANYVGTFAHSFSKASLCSDAQSVAGALKGQLIGCSKGGDATLLADIEASLATHSADECAHSLGFVRAMFAIAASASSHASNNNEWQALSAQFGQQISEIDSKCAEFGIGIAKVPYDGPKGDHSQRNVHGTDSVIAMPGLAGSHKQ

SEQ ID NO: 151 - protein, 202 aa, Artificial Sequence
  Description of artificial sequence note = synthetic construct
MFSLMLSIFPIVFLVCCKAMPNFPCCPGSQQVVAVMSNYIGTFTSEDKSTVCSTAKNTVEGIKSELSSRVGCPSGGEAQIVNEIDRQLTNIAKMEINYEDECPYNLGFARAMFDLAAAAGHAGNDTEWQNMKSKFVQESQAIKAIGQEMNIEVTDVHIGHPSKGISAHQNVPSPSHVIANPGQHSSVGHGKEDTPLSSDFDF

SEQ ID NO: 152 - protein, 368 aa, Artificial Sequence
  Description of artificial sequence note = synthetic construct
MKIISILINFILAIYEAKGGGIVSLLSRRQAPKRHLASSLRQQRTEDNHISINGQNYAVDGPNVNVGVEGHDLSVNGRVYQNRATEQYLEIIQDKNIRNVIVSVPLSLFSRENIIDGQINAKCNGNLYIDQSSDGCSRIICVDDKKNGVENNFGQTRDIFLTGDVNIFESANGIIYNSMMGGTLHIHNSSLECANIECDASLNVTHSPIERNAQMKCGGSLSIDESPMGNIRLNCDGSLRIEKSKMESSQIDVGGSIGIVESPMGSIGIDCGGSLRIEKSKMEIGNLDCGGSLTIVESTAQSLKLNCGGSLNMKESPMKNVGINCDGSATIKKSKMESGRINCGGNFSIDSSPTGSVRIDYGGRRINL

SEQ ID NO: 153 - protein, 92 aa, Artificial Sequence
  Description of artificial sequence note = synthetic construct
MANKFLIAAFILTIAIFVNGQSEAPNNSSEMASEESNSEESSSEEQQFNPFKFRPFFGPSSSNSSAPPPFAFLPFFGRMPSLFNRPSNKSVV

SEQ ID NO: 154 - protein, 638 aa, Artificial Sequence
  Description of artificial sequence note = synthetic construct
MRFSSFSSPFLPLFFLSLPIAFVLSGRTLPFTGSQLANEVARAFFNSVNTWDMSIFGAGTKQGEDRYKISLDGLDRMKNRFRVPLPAGQGLEKLLRSYRVEPLREDYLGVNKARERVLAPSKLMELMEKLGNVLVTDPKMRQKIDKYDKKRADEAARRAAMMPPRQDPQAIAKRRTWPKEDGLALERGHLPQGNNQSPTRLQSTPRIWIQEDDRWRQPMTFSRKDVRERSWLESDTDSDLDSPTSVLRSRRRSRVNILDDDQPTRRTAWGRSPTPSPNGRAVVQRTTTTTTTTTEEEEGGRRTVRFGEVVVVEPEERTVNRRTEVRTQQRETEVERTSEYTLILRIDFIDASVFLDKSLAYFGSLNTARKDERSVQRLCYVLKAFDPRHERLNSVLATPSVANAFVEYKKALNDVGLNSQPELRLVEKSNACAFDLALIYELAQFTKDLLLKLKAERMVAAEELEDVKEEVIGRLLKLLPKVLEGLKAKPAELSTEVDRRIQALDVVEEQLNVVKRARATDEMVTGAMAKVMAQLRNASRGMGTMDMSTLSSLQSNWDNLMRKDTHWQIRKAINSLGGCPKDPQGNTLMKQCMEEAITKVDRYIDDVNDWFKSQRPIDMDDWKWLAAEIQMIIRWKSP

SEQ ID NO: 155 - protein, 177 aa, Artificial Sequence
  Description of artificial sequence note = synthetic construct
MAILLKCVLLLSIMAIFCDCMDPGKKGKSKDPIPIPKQEGSDPIPIPKQEGSDPIPIPKQEGKPSSSAANSPTVTKGTPKRGELDTPEFYKTSPKNKINSPRKPNNGSPRKDKKALQKERQEERKQKERERENRFLRTKSTAGNTTDATDVETESEVIPTFVAELEDSTVEYPTDIE

SEQ ID NO: 156 - protein, 209 aa, Artificial Sequence
  Description of artificial sequence note = synthetic construct
MAPLFHRFSSLFVFLMPFLSVVLLPSTVCTGSDSAAAPFDRKNYPKIDLRLFEWPIASHSGSSAEVSFIAVDCYTQLDRSFISTDAVLRLNNSLALRHRACLLRIPTGTRLTVTEMQTTNRKVNKTKPKLRPMARAVPTGVCAVQLARAQNGMGRISSGRRNGGGQRDGERGRMFGGRRGGRRGRGEGIPQKASSLSRWAADSFGFDEH

SEQ ID NO: 157 - protein, 170 aa, Artificial Sequence
  Description of artificial sequence note = synthetic construct
MAILLKFVLFISIMAIFCDCMDPGKNGKNEKKDVVKQKVDETKVERASEMNKGKSIVMADSKKEGTTTVKIPHRYGAVSGMSGQNASPEASQIGSPKNSPKGTQIGSPRSISSPKSTQIGSPKGIQIGSPRKEKTKLSSAVGSSDFNVIDESKEAKKTKPIQTESVQKPK

SEQ ID NO: 158 - protein, 203 aa, Artificial Sequence
  Description of artificial sequence note = synthetic construct
REATVLKHVGNQTNAAGIDAEFAVNFLLAQMEANKMIQRGYIDRWNSDHSFESKYVPDFEKEIQPKFSYATNALILALIPLVDAGHQMHNDQNCVEHVEDVLESMEHLRASELEPNGKEAMEKAVKAICEKISTHEGQSNAEDQSKSKKRKHSDNHKMEEGKHGEEKEIRPTKRTRKANTDESKTPAAGENRRNHRRENYVDS

SEQ ID NO: 159 - protein, 156 aa, Artificial Sequence
  Description of artificial sequence note = synthetic construct
MNKFVGIFVAVLLQFVSPFSAFSRVPTTTTERPIIYDPKEMVEIQVNLVNNTNNNCTNDVLRKYRVEITNYVFFLVCDLKIRVQLPEGATLENVVNLKPFNGTTDQFIFPDSLRYLYVSKTLEAELSVKGGEGEPKITVLDAKAAFSPKKCRISKF